(19) United States
(12) Patent Application Publication
(10) Pub. No.: US 2007/0185152 A1
(43) Pub. Date: Aug. 9, 2007

(54) Inhibitors of akt activity

(75) Inventors: Dennis S. Yamashita, Collegeville, PA (US); Hong Lin, Collegeville, PA (US); Wenyong Wang, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Smithkline Beecham Corporation

(21) Appl. No.: 10/591,270

(22) PCT Filed: Mar. 2, 2005
(86) PCT No.: PCT/US05/06711

§ 371 Date: Aug. 31, 2006

Related U.S. Application Data
(60) Provisional application No. 60/549,384, filed on Mar. 2, 2004.
(60) Provisional application No. 60/561,973, filed on Apr. 14, 2004.
(60) Provisional application No. 60/589,772, filed on Jul. 21, 2004.
(60) Provisional application No. 60/605,585, filed on Aug. 30, 2004.
(60) Provisional application No. 60/628,473, filed on Nov. 16, 2004.

(51) Int. Cl. C07D 471/02; A61K 31/4745; A61K 31/4439; A61K 31/444; A61K 31/4709; C07D 401/02; C07D 409/02

(52) U.S. Cl. 514/300; 514/307; 514/314; 514/332; 514/338; 514/342; 514/337; 546/113; 546/148; 546/176; 546/256; 546/275.7; 546/278.1; 546/280.4; 546/281.4

(57) ABSTRACT

Invented are novel pyridine compounds, the use of such compounds as inhibitors of PKB/AKT kinase activity and in the treatment of cancer and arthritis.

### FIELD OF THE INVENTION

This invention relates to novel pyridine compounds, the use of such compounds as inhibitors of protein kinase B (hereinafter PKB/Akt, PKB or Akt) activity and in the treatment of cancer and arthritis.

### BACKGROUND OF THE INVENTION

The present invention relates to pyridine containing compounds that are inhibitors of the activity of one or more of the isoforms of the serine/threonine kinase, Akt (also known as protein kinase B). The present invention also relates to pharmaceutical compositions comprising such compounds and methods of using the instant compounds in the treatment of cancer and arthritis (Liu et al. Current Opin. Pharmacology 3:317-22 (2003)).

Apoptosis (programmed cell death) plays essential roles in embryonic development and pathogenesis of various diseases, such as degenerative neuronal diseases, cardiovascular diseases and cancer. Recent work has led to the identification of various pro- and anti-apoptotic gene products that are involved in the regulation or execution of programmed cell death. Expression of anti-apoptotic genes, such as Bcl2 or Bcl-x_{L}, inhibits apoptotic cell death induced by various stimuli. On the other hand, expression of pro-apoptotic genes, such as Bax or Bad, leads to programmed cell death (Adams et al. Science, 281:1322-1326 (1998)). The execution of programmed cell death is mediated by caspase-1 related proteinases, including caspase-3, caspase-7, caspase-8 and caspase-9 etc (Thornberry et al. Science, 281:1312-1316 (1998)).

The phosphatidylinositol 3′-OH kinase (Pl3K)/Akt/PKB pathway appears important for regulating cell survival/cell death (Kulik et al. Mol. Cell. Biol. 17:1595-1606 (1997); Franke et al, Cell, 88:435-437 (1997); Kauffmann-Zeh et al. Nature 385:544-548 (1997) Hemmings Science, 275:628-630 (1997); Dudek et al., Science, 275:661-665 (1997)). Survival factors, such as platelet derived growth factor (PDGF), nerve growth factor (NGF) and insulin-like growth factor-1 (IGF-1), promote cell survival under various conditions by inducing the activity of Pl3K (Kulik et al. 1997, Hemmings 1997). Activated Pl3K leads to the production of phosphatidylinositol (3,4,5)-triphosphate (Ptdlns (3,4,5)-P3), which in turn binds to, and promotes the activation of, the serine/threonine kinase Akt, which contains a pleckstrin homology (PH)-domain (Franke et al Cell, 81:727-736 (1995); Hemmings Science, 277:534 (1997); Downward, Curr. Opin. Cell Biol. 10:262-267 (1998), Alessi et al., EMBO J. 15: 6541-6551 (1996)). Specific inhibitors of Pl3K or dominant negative Akt/PKB mutants abolish survival-promoting activities of these growth factors or cytokines. It has been previously disclosed that inhibitors of Pl3K (LY294002 or wortmannin) blocked the activation of Akt/PKB by upstream kinases. In addition, introduction of constitutively active Pl3K or Akt/PKB mutants promotes cell survival under conditions in which cells normally undergo apoptotic cell death (Kulik et al. 1997, Dudek et al. 1997).

Analysis of Akt levels in human tumors showed that Akt2 is overexpressed in a significant number of ovarian (J. Q. Cheung et al. Proc. Natl. Acad. Sci. U.S.A. 89:9267-9271(1992)) and pancreatic cancers (J. Q. Cheung et al. Proc. Natl. Acad. Sci. U.S.A. 93:3636-3641 (1996)). Similarly, Akt3 was found to be overexpressed in breast and prostate cancer cell lines (Nakatani et al. J. Biol. Chem. 274:21528-21532 (1999). It was demonstrated that AKT2 was over-expressed in 12% of ovarian carcinomas and that amplification of AKT was especially frequent in 50% of undifferentiated tumors, suggestion that AKT may also be associated with tumor aggressiveness (Bellacosa, et al., Int. J. Cancer, 64, pp. 280-285, 1995). Increased Akt1 kinase activity has been reported in breast, ovarian and prostate cancers (Sun et al. Am. J. Pathol. 159: 431-7 (2001)).

The tumor suppressor PTEN, a protein and lipid phosphatase that specifically removes the 3′ phosphate of Ptdlns(3,4,5)-P3, is a negative regulator of the Pl3K/Akt pathway (Li et al. Science 275:1943-1947 (1997), Stambolic et al. Cell 95:29-39 (1998), Sun et al. Proc. Natl. Acad. Sci. U.S.A. 96:6199-6204 (1999)). Germline mutations of PTEN are responsible for human cancer syndromes such as Cowden disease (Liaw et al. Nature Genetics 16:64-67 (1997)). PTEN is deleted in a large percentage of human tumors and tumor cell lines without functional PTEN show elevated levels of activated Akt (Li et al. supra, Guldberg et al. Cancer Research 57:3660-3663 (1997), Risinger et al. Cancer Research 57:4736-4738 (1997)).

These observations demonstrate that the Pl3K/Akt pathway plays important roles for regulating cell survival or apoptosis in tumorigenesis.

Three members of the Akt/PKB subfamily of second-messenger regulated serine/threonine protein kinases have been identified and termed Akt1/PKBα, Akt2/PKBβ, and Akt3/PKBγ respectively. The isoforms are homologous, particularly in regions encoding the catalytic domains. Akt/PKBs are activated by phosphorylation events occurring in response to Pl3K signaling. Pl3K phosphorylates membrane inositol phospholipids, generating the second messengers phosphatidyl-inositol 3,4,5-trisphosphate and phosphatidylinositol 3,4-bisphosphate, which have been shown to bind to the PH domain of Akt/PKB. The current model of Akt/PKB activation proposes recruitment of the enzyme to the membrane by 3′-phosphorylated phosphoinositides, where phosphorylation of the regulatory sites of Akt/PKB by the upstream kinases occurs (B. A. Hemmings, Science 275:628-630 (1997); B. A. Hemmings, Science 276:534 (1997); J. Downward, Science 279:673-674 (1998)).

Phosphorylation of Akt1/PKBα occurs on two regulatory sites, Thr³⁰⁸ in the catalytic domain activation loop and on Ser⁴⁷³ near the carboxy terminus (D. R. Alessi et al. EMBO J. 15:6541-6551 (1996) and R. Meier et al. J. Biol. Chem. 272:30491-30497 (1997)). Equivalent regulatory phosphorylation sites occur in Akt2/PKBβ and Akt3/PKBγ. The upstream kinase, which phosphorylates Akt/PKB at the activation loop site has been cloned and termed 3′-phosphoinositide dependent protein kinase 1 (PDK1). PDK1 phosphorylates not only Akt/PKB, but also p70 ribosomal S6 kinase, p90RSK, serum and glucocorticoid-regulated kinase (SGK), and protein kinase C. The upstream kinase phosphorylating the regulatory site of Akt/PKB near the carboxy terminus has not been identified yet, but recent reports imply a role for the integrin-linked kinase (ILK-1), a serine/threonine protein kinase, or autophosphorylation.

Inhibition of Akt activation and activity can be achieved by inhibiting Pl3K with inhibitors such as LY294002 and wortmannin. However, Pl3K inhibition has the potential to indiscriminately affect not just all three Akt isozymes but also other PH domain-containing signaling molecules that are dependent on Pdtlns(3,4,5)-P3, such as the Tec family of tyrosine kinases. Furthermore, it has been disclosed that Akt can be activated by growth signals that are independent of Pl3K.

Alternatively, Akt activity can be inhibited by blocking the activity of the upstream kinase PDK1. The compound UCN-01 is a reported inhibitor of PDK1. Biochem. J. 375(2):255 (2003). Again, inhibition of PDK1 would result in inhibition of multiple protein kinases whose activities depend on PDK1, such as atypical PKC isoforms, SGK, and S6 kinases (Williams et al. Curr. Biol. 10:439-448 (2000).

Small molecule inhibitors of AKT are useful in the treatment of tumors, especially those with activated AKT (e.g. PTEN null tumors and tumors with ras mutations). PTEN is a critical negative regulator of AKT and its function is lost in many cancers, including breast and prostate carcinomas, glioblastomas, and several cancer syndromes including Bannayan-Zonana syndrome (Maehama, T. et al. Annual Review of Biochemistry, 70: 247 (2001)), Cowden disease (Parsons, R.; Simpson, L. Methods in Molecular Biology (Totowa, N.J., United States), 222 (Tumor Suppressor Genes, Volume 1): 147 (2003)), and Lhermitte-Duclos disease (Backman, S. et al. Current Opinion in Neurobiology, 12(5): 516 (2002)). AKT3 is up-regulated in estrogen receptor-deficient breast cancers and androgen-independent prostate cancer cell lines and AKT2 is over-expressed in pancreatic and ovarian carcinomas. Akt1 is amplified in gastric cancers (Staal, Proc. Natl. Acad. Sci. USA 84: 5034-7 (1987) and upregulated in breast cancers (Stal et al. Breast Cancer Res. 5: R37-R44 (2003)). Therefore a small molecule AKT inhibitor is expected to be useful for the treatment of these types of cancer as well as other types of cancer. AKT inhibitors are also useful in combination with further chemotherapeutic agents.

It is an object of the instant invention to provide novel compounds that are inhibitors of Akt/PKB.

It is also an object of the present invention to provide pharmaceutical compositions that comprise a pharmaceutical carrier and compounds useful in the methods of the invention.

It is also an object of the present invention to provide a method for treating cancer that comprises administering such inhibitors of Akt/PKB activity.

It is also an object of the present invention to provide a method for treating arthritis that comprises administering such inhibitors of Akt/PKB activity.

### SUMMARY OF THE INVENTION

This invention relates to compounds of Formula (I): 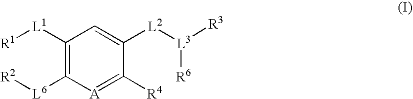
wherein:

A is selected from: nitrogen, —C-halogen and —CH;

L¹ is selected from the group consisting of a bond, —O—, —N(R⁵)—, —S—, —S(O)—, —S(O₂)—, alkyl, and —N(R⁵)C(O)—;

L² is selected from the group consisting of a bond, —O—, heterocycle, —N(R⁵)—, —N(R⁵)C(O)—, —S—, —S(O)—, —S(O₂)—, and —C(O)N(R⁵)—;

L³ is alkyl, wherein the alkyl is optionally substituted with one or two substituents independently selected from the group consisting of amino, methylamino, dimethylamino, oxo, and hydroxy;

L⁶ is selected from the group consisting of a bond, —O—, —N(R⁵)—, —S—, —S(O)—, —S(O₂)—, alkyl, and —N(R⁵)C(O)—;

R¹ is selected from the group consisting of aryl, substituted aryl, cycloalkyl, substituted cycloalkyl, heterocycle and substituted heterocycle;

R² is selected from alkyl, substituted alkyl, cycloalkyl, substituted cycloalkyl, heterocycle, substituted heterocycle, and a cyclic or polycyclic aromatic ring containing from 3 to 16 carbon atoms and optionally containing one or more heteroatoms, provided that.when the number of carbon atoms is 3 the aromatic ring contains at least two heteroatoms and when the number of carbon atoms is 4 the aromatic ring contains at least one heteroatom, and optionally substituted with one or more substituents selected from the group consisting of: alkyl, substituted alkyl, trifluoroalkoxy, C₁-C₁₂aryl, aryloxy, —O(CH₂)_{q}R³¹, —NHC(O)—NHR⁴¹, —C(O)R⁴³, substituted cycloalkyl, substituted C₁-C₁₂aryl, heterocycle, substituted heterocycle, oxo, hydroxy, alkoxy, cycloalkyl, acyloxy, amino, N-acylamino, nitro, cyano, halogen, —C(O)OR⁷, —C(O)NR⁸R⁹, —S(O)₂NR⁸R⁹, and —S(O)ₙR⁷, where n is 0-2, q is 1-6,
R⁷ is hydrogen, alkyl, cycloalkyl, C₁-C₁₂aryl, substituted alkyl, substituted cycloalkyl and substituted C₁-C₁₂aryl,
R³¹ is C₁-C₁₂aryl, cycloalkyl and heterocycle, each of which is optionally substituted with from 1 to 4 substituents selected from: halogen, alkyl, hydroxyalkyl, alkoxy, acyloxy, amino, methylamino, dimethylamino, N-acylamino, hydroxy, nitro, tetrazole, cyano, oxo and trifluoromethyl, R⁴¹ is selected from hydrogen, C₁-C₁₂aryl, cycloalkyl and heterocycle, wherein C₁-C₁₂aryl, cycloalkyl and heterocycle are optionally substituted with from 1 to 4 substituents selected from: halogen, alkyl, hydroxyalkyl, alkoxy, amino, methylamino, dimethylamino, hydroxy, nitro, tetrazole, cyano, oxo and trifluoromethyl,
R⁴³ is selected from C₁-C₁₂aryl, cycloalkyl and heterocycle, each of which is optionally substituted with from 1 to 4 substituents selected from: halogen, hydroxyalkyl, alkoxy, amino, methylamino, dimethylamino, hydroxy, nitro, tetrazole, cyano, oxo and trifluoromethyl, and
R⁸ and R⁹ are independently hydrogen, cycloalkyl, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, —C(O)OR¹⁰, —S(O)ₙR¹⁰, —C(O)NR¹⁰R¹¹, —S(O)₂NR¹⁰R¹¹, nitro, cyano, cycloalkyl, substituted cycloalkyl, halogen, aryl, and substituted aryl,
or R⁸ and R⁹ taken together with the nitrogen to which they are attached represent a 5 to 6 member saturated ring containing up to one other heteroatom selected from oxygen and nitrogen, where the ring is optionally subtituted with one or more substituents selected from amino, methylamino and dimethylamino,
   where R¹⁰ and R¹¹ are independently hydrogen, alkyl, cycloalkyl, C₁-C₁₂aryl, substituted alkyl, substituted cycloalkyl and substituted C₁-C₁₂aryl, and n is 0-2,

and when L⁶ is a bond, R² can additionally be halogen;

R³ and R⁶ are independently selected from the group consisting of hydrogen, amino, methylamino, dimethylamino, aryl, substituted aryl, heterocycle, substituted heterocycle, cycloalkyl, substituted cycloalkyl, —S—C₁-C₁₂aryl, —O—C₁-C₁₂aryl, —OalkylC₁-C₁₂aryl, aryloxy, substituted aryloxy and arylalkoxy; and

R⁴ is selected from the group consisting of hydrogen and halogen;

where R⁵ is selected from the group consisting of hydrogen, —S(O)₂CH₃, —S(O)₂H and alkyl;

provided that when,

R¹ is azaindazole, substituted azaindazole, 1H-thienopyrazole, substituted 1H-thienopyrazole, benzamide, substituted benzamide, phenylethanone, substituted phenylethanone, thiophene, substituted thiophene, furan or substituted furan,

R² may additionally be hydrogen;

and/or pharmaceutically acceptable salts, hydrates, solvates and pro-drugs thereof;

further provided that when

R¹ is isoquinoline,

R² is not furyl or alkyl.

This invention relates to a method of treating cancer, which comprises administering to a subject in need thereof an effective amount of an Akt/PKB inhibiting compound of Formula (I).

This invention relates to a method of treating arthritis, which comprises administering to a subject in need thereof an effective amount of an Akt/PKB inhibiting compound of Formula (I).

The present invention also relates to the discovery that the compounds of Formula (I) are active as inhibitors of Akt/PKB.

In a further aspect of the invention there is provided novel processes and novel intermediates useful in preparing the presently invented Akt/PKB inhibiting compounds.

Included in the present invention are pharmaceutical compositions that comprise a pharmaceutical carrier and compounds useful in the methods of the invention.

Also included in the present invention are methods of co-administering the presently invented Akt/PKB inhibiting compounds with further active ingredients.

### DETAILED DESCRIPTION OF THE INVENTION

This invention relates to compounds of Formula (I) as described above.

The presently invented compounds of Formula (I) inhibit Akt/PKB activity. In particular, the compounds disclosed herein inhibit each of the three Akt/PKB isoforms.

Included among the presently invented compounds of Formula (I) are those having Formula (I):

wherein

A is selected from: nitrogen, —C-halogen and —CH;

L¹ is selected from the group consisting of a bond, —O—, —N(R⁵)—, —S—, —S(O)—, —S(O₂)—, alkyl, and —N(R⁵)C(O)—;

L² is selected from the group consisting of a bond, —O—, heterocycle, —N(R⁵)—, —N(R⁵)C(O)—, —S—, —S(O)—, —S(O₂)—, and —C(O)N(R⁵)—;

L³ is alkyl, wherein the alkyl is optionally substituted with one or two substituents independently selected from the group consisting of amino, methylamino, dimethylamino, oxo, and hydroxy;

L⁶ is a bond;

R¹ is selected from the group consisting of C₁-C₁₂aryl and substituted C₁-C₁₂aryl;

R² is selected from alkyl, substituted alkyl, halogen, cycloalkyl, substituted cycloalkyl, heterocycle, substituted heterocycle, and C₁-C₁₂aryl optionally substituted with one or more substituents selected from the group consisting of: alkyl, substituted alkyl, trifluoroalkoxy, C₁-C₁₂aryl, aryloxy, —O(CH₂)_{q}R³¹, —NHC(O)—NHR⁴¹, —C(O)R⁴³, hydroxy, alkoxy, cycloalkyl, N-acylamino, nitro and halogen,
where q is 1-6,
R³¹ is C₁-C₁₂aryl, cycloalkyl and heterocycle, each of which is optionally substituted with from 1 to 4 substituents selected from: halogen, alkyl, hydroxyalkyl, alkoxy, acyloxy, amino, methylamino, dimethylamino, N-acylamino, hydroxy, nitro, tetrazole, cyano, oxo and trifluoromethyl,
R⁴¹ is selected from hydrogen, C₁-C₁₂aryl, cycloalkyl and heterocycle, wherein C₁-C₁₂aryl, cycloalkyl and heterocycle are optionally substituted with from 1 to 4 substituents selected from: halogen, alkyl, hydroxyalkyl, alkoxy, amino, methylamino, dimethylamino, hydroxy, nitro, tetrazole, cyano, oxo and trifluoromethyl,
R⁴³ is selected from C₁-C₁₂aryl, cycloalkyl and heterocycle, each of which is optionally substituted with from 1 to 4 substituents selected from: halogen, hydroxyalkyl, alkoxy, amino, methylamino, dimethylamino, hydroxyl, nitro, tetrazole, cyano, oxo and trifluoromethyl,

R³ and R⁶ are independently selected from the group consisting of hydrogen, amino, methylamino, dimethylamino, aryl, substituted aryl, heterocycle, substituted heterocycle, cycloalkyl, substituted cycloalkyl, —S—C₁-C₁₂aryl, aryloxy and arylalkoxy; and

R⁴ is selected from the group consisting of hydrogen and halogen;

where R⁵ is selected from the group consisting of hydrogen, —S(O)₂CH₃, —S(O)₂H and alkyl;

provided that when,

R¹ is azaindazole, substituted azaindazole, 1H-thienopyrazole, substituted 1H-thienopyrazole, benzamide, substituted benzamide, phenylethanone, substituted phenylethanone, thiophene, substituted thiophene, furan or substituted furan,

R² may additionally be hydrogen;

and/or pharmaceutically acceptable salts, hydrates, solvates and pro-drugs thereof;

further provided that when

R¹ is isoquinoline,

R² is not furyl or alkyl.

Included among the presently invented compounds of Formula (I) are those having Formula (II): 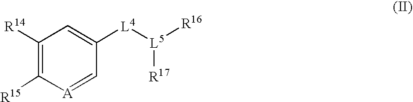
wherein:

A is selected from nitrogen, —CF and —CH;

L⁴ is selected from the group consisting of a bond, heterocycle, —O—, and —NH—;

L⁵ is alkyl, wherein the alkyl is optionally substituted with one or two substituents independently selected from the group consisting of amino, oxo, and hydroxy;

R¹⁴ is selected from the group consisting of C₁-C₁₂aryl, and substituted C₁-C₁₂aryl;

R¹⁵ is selected from alkyl, substituted alkyl, halogen, cycloalkyl, substituted cycloalkyl, heterocycle, substituted heterocycle, C₁-C₁₂aryl and C₁-C₁₂aryl optionally substituted with one or more substituents selected from the group consisting of: alkyl, substituted alkyl, trifluoroalkoxy, aryloxy, —O(CH₂)_{q}R³¹, —NHC(O)—NHR⁴¹, —C(O)R⁴³, hydroxy, alkoxy, acyloxy, amino, cycloalkyl, N-acylamino, nitro, cyano and halogen,
where q is 1-6,
R³¹ is C₁-C₁₂aryl optionally substituted with from 1 to 4 substituents selected from: halogen, alkyl, hydroxyalkyl, alkoxy, and hydroxy,
R⁴¹ is selected from hydrogen and C₁-C₁₂aryl optionally substituted with from 1 to 4 substituents selected from: halogen, alkyl, hydroxyalkyl, alkoxy, and hydroxy,
R⁴³ is C₁-C₁₂aryl substituted with from 1 to 4 substituents selected from: halogen, hydroxyalkyl, alkoxy, and hydroxy, and

R¹⁶ and R¹⁷ are independently selected from the group consisting of hydrogen, C₁-C₁₂aryl, substituted C₁-C₁₂aryl, heterocycle, cycloalkyl, —S—C₁-C₁₂aryl, and C₁-C₁₂arylalkoxy;

provided that when,

R¹⁴ is azaindazole, substituted azaindazole, 1H-thienopyrazole, substituted 1H-thienopyrazole, benzamide, substituted benzamide, phenylethanone, substituted phenylethanone, 2-pyridinecarboxamide, substituted 2-pyridinecarboxamide, (methylsulfonyl)benzene, substituted (methylsulfonyl)benzene, thiophene, substituted thiophene, furan or substituted furan,

R¹⁵ may additionally be hydrogen;

and/or pharmaceutically acceptable salts, hydrates, solvates and pro-drugs thereof;

further provided that when

R¹⁴ is isoquinoline,

R¹⁵ is not furyl or alkyl.

Included among the presently invented compounds of Formula (II) are those in which:

A is selected from nitrogen, —CF and —CH;

L⁴ is selected from the group consisting of a bond, —O—, heterocycle, and —NH—;

L⁵ is alkyl, wherein the alkyl is substituted with one or two substituents independently selected from the group consisting of amino, oxo, and hydroxy;

R¹⁴ is selected from the group consisting of C₁-C₁₂aryl, and substituted C₁-C₁₂aryl;

R¹⁵ is selected from alkyl, substituted alkyl, cycloalkyl, substituted cycloalkyl, heterocycle, substituted heterocycle, C₁-C₁₂aryl and C₁-C₁₂aryl substituted with one or more substituents selected from the group consisting of: alkyl, substituted alkyl, aryloxy, hydroxy, alkoxy, acyloxy, amino, N-acylamino, nitro, cyano and halogen; and

R¹⁶ and R¹⁷ are independently selected from the group consisting of hydrogen, C₁-C₁₂aryl and substituted C₁-C₁₂aryl;

provided that when,

R¹⁴ is azaindazole, substituted azaindazole, 1H-thienopyrazole, substituted 1H-thienopyrazole, benzamide, substituted benzamide, phenylethanone, substituted phenylethanone, 2-pyridinecarboxamide, substituted 2-pyridinecarboxamide, (methylsulfonyl)benzene, substituted (methylsulfonyl)benzene, thiophene, substituted thiophene, furan or substituted furan,

R¹⁵ may additionally be hydrogen;

and/or pharmaceutically acceptable salts, hydrates, solvates and pro-drugs thereof;

further provided that when

R¹⁴ is isoquinoline,

R¹⁵ is not furyl or alkyl.

Included among the presently invented compounds of Formula (II) are those in which:

A is selected from nitrogen, —CF and —CH;

L⁴ is selected from the group consisting of a bond, heterocycle, —O—, and —NH—;

L⁵ is alkyl, wherein the alkyl is optionally substituted with one or two substituents independently selected from the group consisting of amino, oxo, and hydroxy;

R¹⁴ is selected from the group consisting of C₁-C₁₂aryl, and substituted C₁-C₁₂aryl;

R¹⁵ is selected from alkyl, substituted alkyl, halogen, cycloalkyl, and C₁-C₁₂aryl optionally substituted with one or more substituents selected from the group consisting of: alkyl, substituted alkyl, trifluoroalkoxy, C₁-C₁₂aryloxy, —O(CH₂)_{q}R³¹, —NHC(O)—NHR⁴¹, —C(O)R⁴³, hydroxy, alkoxy, cycloalkyl, N-acylamino, nitro and halogen,
where q is 1-6,
R³¹ is C₁-C₁₂aryl optionally substituted with from 1 to 4 substituents selected from: halogen, alkyl, hydroxyalkyl, alkoxy, and hydroxy,
R⁴¹ is selected from hydrogen and C₁-C₁₂aryl optionally substituted with from 1 to 4 substituents selected from: halogen, alkyl, hydroxyalkyl, alkoxy, and hydroxy,
R⁴³ is C₁-C₁₂aryl substituted with from 1 to 4 substituents selected from: halogen, hydroxyalkyl, alkoxy, and hydroxy, and

R¹⁶ and R¹⁷ are independently selected from the group consisting of hydrogen, C₁-C₁₂aryl, substituted C₁-C₁₂aryl, heterocycle, cycloalkyl, —S—C₁-C₁₂aryl, and C₁-C₁₂arylalkoxy;

provided that when,

R¹⁴ is 7-azaindazole, 4-azaindazole, 1H-thieno[3,2-c]pyrazole, benzamide, 1-phenylethanone, 2-furancarboxamide, 1-(2-furanyl)ethanone, 2-thienylcarboxamide, 1-(2-thienyl)ethanone, substituted 7-azaindazole, substituted 4-azaindazole, substituted 1H-thieno[3,2-c]pyrazole, substituted benzamide, substituted 1-phenylethanone, substituted 2-furancarboxamide, substituted 1-(2-furanyl)ethanone, substituted 2-thienylcarboxamide or substituted 1-(2-thienyl)ethanone, 2-pyridinecarboxamide, substituted 2-pyridinecarboxamide, (methylsulfonyl)benzene, substituted (methylsulfonyl)benzene,

R¹⁵ may additionally be hydrogen;

and/or pharmaceutically acceptable salts, hydrates, solvates and pro-drugs thereof;

further provided that when

R¹⁴ is isoquinoline,

R¹⁵ is not furyl or alkyl.

Included among the presently invented compounds of Formula (II) are those in which:

A is selected from nitrogen, —CF and —CH;

L⁴ is selected from the group consisting of a bond, —O—, and —NH—;

L⁵ is alkyl, wherein the alkyl is substituted with one or two substituents independently selected from the group consisting of amino, oxo, and hydroxy;
R¹⁴ is selected from phenyl, pyridine, indazole, 7-azaindole, quinoline, isoquinoline, substituted phenyl, substituted pyridine, substituted indazole, substituted 7-azaindole, substituted quinoline and substituted isoquinoline;
R¹⁵ is selected from cycloalkyl, substituted cycloalkyl, phenyl, pyridine, thiophene, furan, pyrrole, indazole, quinoline, isoquinoline, 7-azaindole, substituted phenyl, substituted pyridine, substituted thiophene, substituted furan, substituted indazole, substituted quinoline, substituted 7-azaindole and substituted isoquinoline; and
R¹⁶ and R¹⁷ are independently selected from the group consisting of hydrogen, indole, substituted indole, azaindole, substituted azaindole, naphthalene, substituted naphthalene, benzofuran, substituted benzofuran, phenyl, pyridine, thiophene, furan, pyrrole, substituted phenyl, substituted pyridine, substituted thiophene, substituted furan, and substituted pyrrole;
   provided that when,

R¹⁴ is 7-azaindazole, 4-azaindazole, 1H-thieno[3,2-c]pyrazole, benzamide, 1-phenylethanone, 2-furancarboxamide, 1-(2-furanyl)ethanone, 2-thienylcarboxamide, 1-(2-thienyl)ethanone, substituted 7-azaindazole, substituted 4-azaindazole, substituted 1H-thieno[3,2-c]pyrazole, substituted benzamide, substituted 1-phenylethanone, substituted 2-furancarboxamide, substituted 1-(2-furanyl)ethanone, substituted 2-thienylcarboxamide or substituted 1-(2-thienyl)ethanone, 2-pyridinecarboxamide, substituted 2-pyridinecarboxamide, (methylsulfonyl)benzene, substituted (methylsulfonyl)benzene,

R¹⁵ may additionally be hydrogen;

and/or pharmaceutically acceptable salts, hydrates, solvates and pro-drugs thereof;

further provided that when

R¹⁴ is isoquinoline,

R¹⁵ is not furyl or alkyl.

Included among the presently invented compounds of Formula (II) are those having Formula (II):

wherein

A is selected from nitrogen, —CF and —CH;

L⁴ is selected from the group consisting of a bond, —O—, and —NH—;

L⁵ is alkyl, wherein the alkyl is substituted with one or two substituents independently selected from the group consisting of amino, oxo, and hydroxy;

R¹⁴ is selected from the group consisting of C₁-C₁₂aryl, and substituted C₁-C₁₂aryl;

R¹⁵ is selected from cycloalkyl and substituted cycloalkyl; and

R¹⁶ and R¹⁷ are independently selected from the group consisting of hydrogen, C₁-C₁₂aryl and substituted C₁-C₁₂aryl;

provided that when,

R¹⁴ is 7-azaindazole, 4-azaindazole, 1H-thieno[3,2-c]pyrazole, benzamide, 1-phenylethanone, 2-furancarboxamide, 1-(2-furanyl)ethanone, 2-thienylcarboxamide, 1-(2-thienyl)ethanone, substituted 7-azaindazole, substituted 4-azaindazole, substituted 1H-thieno[3,2-c]pyrazole, substituted benzamide, substituted 1-phenylethanone, 2-pyridinecarboxamide, substituted 2-pyridinecarboxamide, (methylsulfonyl)benzene, substituted (methylsulfonyl)benzene, substituted 2-furancarboxamide, substituted 1-(2-furanyl)ethanone, substituted 2-thienylcarboxamide or substituted 1-(2-thienyl)ethanone,

R¹⁵ may additionally be hydrogen;

and/or pharmaceutically acceptable salts, hydrates, solvates and pro-drugs thereof;

further provided that when

R¹⁴ is isoquinoline,

R¹⁵ is not furyl or alkyl.

Included among the compounds useful in the present invention are:
(S)-1-Benzyl-2-[5-(3-methyl-1H-indazol-5-yl)-6-phenyl-pyridin-3-yloxy]-ethylamine;
(S)-1-Benzyl-2-[6-furan-2-yl-5-(3-methyl-1H-indazol-5-yl)-pyridin-3-yloxy]-ethylamine;
(S)-1-Benzyl-2-[5,6-bis-(3-methyl-1H-indazol-5-yl)-pyridin-3-yloxy]-ethylamine;
(S)-1-Benzyl-2-[6-thiophen-2yl-5-(3-methyl-1H-indazol-5-yl)-pyridin-3-yloxy]-ethylamine;
(S)-1-Benzyl-2-[6-(4-chlorophenyl)-5-(3-methyl-1H-indazol-5-yl)-pyridin-3-yloxy]-ethylamine;
(S)-1-Benzyl-2-[6-(3-chlorophenyl)-5-(3-methyl-1H-indazol-5-yl)-pyridin-3-yloxy]-ethylamine;
(S)-1-Benzyl-2-[6-benzyl-5-(3-methyl-1H-indazol-5-yl)-pyridin-3-yloxy]-ethylamine;
(S)-1-Benzyl-2-[6-cyclopent-1-enyl-5-(3-methyl-1H-indazol-5-yl)-pyridin-3-yloxy]-ethylamine;
(S)-1-Benzyl-2-[6-cyclopentyl-5-(3-methyl-1H-indazol-5-yl)-pyridin-3-yloxy]-ethylamine;
(S)-1-Benzyl-2-[6-cyclohex-1-enyl-5-(3-methyl-1H-indazol-5-yl)-pyridin-3-yloxy]-ethylamine;
(S)-1-Benzyl-2-[6-cyclohexyl-5-(3-methyl-1H-indazol-5-yl)-pyridin-3-yloxy]-ethylamine;
3-Methyl-5-[2-phenyl-5-(piperidin-4-ylmethoxy)-pyridin-3-yl]-1H-indazole;
3-[5-(3-Methyl-1H-indazol-5-yl)-6-phenyl-pyridin-3-yloxy]-propylamine;
(S)-1-Benzyl-2-[5-(3-methyl-1H-indazol-5-yl)-6-(5-methyl-thiophen-2-yl)-pyridin-3-yloxy]-ethylamine;
(S)-1-Benzyl-2-[5-(3-methyl-1H-indazol-5-yl)-6-(5-methyl-furan-2-yl)-pyridin-3-yloxy]-ethylamine;
3-Methyl-5-[2-phenyl-5-(4-pyridin-3-yl-methyl-piperazin-1-yl)-pyridin-3-yl]-1H-indazole;
3-Methyl-5-[2-phenyl-5-(4-pyridin-4-ylmethyl-piperazin-1-yl)-pyridin-3-yl]-1H-indazole;
[(1S)-2-{[6-(3-furanyl)-5-(3-methyl-1H-indazol-5-yl)-3-pyridinyl]oxy}-1-(phenylmethyl)ethyl]amine;
[(1S)-2-{[5-(3-methyl-1H-indazol-5-yl)-6-(5-chloro-2-thienyl)-3-pyridinyl]oxy}-1-(phenylmethyl)ethyl]amine;
[(1S)-2-{[6-(3-aminophenyl)-5-(3-methyl-1H-indazol-5-yl)-3-pyridinyl]oxy}-1-(phenylmethyl)ethyl]amine;
(S)-1-Benzyl-2-[5-(1H-indazol-5-yl)-6-phenyl-pyridin-3-yloxy]-ethylamine;
(S)-1-Benzyl-2-{6-[3-(3-fluoro-benzyloxy)phenyl]-5-(3-methyl-1H-indazol-5-yl)-pyridin-3-yloxy}-ethylamine;
(S)-1-Benzyl-2-[5-(3-phenyl-1H-indazol-5-yl)-6-phenyl-pyridin-3-yloxy]-ethylamine;
[(1S)-2-{[5-(3-methyl-1H-indazol-5-yl)-6-(1H-pyrrol-2-yl)-3-pyridinyl]oxy}-1-(phenylmethyl)ethyl]amine;
N-{3-[5-{[(2S)-2-amino-3-phenylpropyl]oxy}-3-(3-methyl-1H-indazol-5-yl)-2-pyridinyl]phenyl}benzamide;
N-{3-[5-{[(2S)-2-amino-3-phenylpropyl]oxy}-3-(3-methyl-1H-indazol-5-yl)-2-pyridinyl]phenyl}-2,6-difluorobenzamide;
N-{3-[5-{[(2S)-2-amino-3-phenylpropyl]oxy}-3-(3-methyl-1H-indazol-5-yl)-2-pyridinyl]phenyl}cyclohexanecarboxamide;
[(1S)-2-({5-[3-(2-furanyl)-1H-indazol-5-yl]-6-phenyl-3-pyridinyl}oxy)-1-(phenylmethyl)ethyl]amine;
{(1S)-2-phenyl-1-[({6-phenyl-5-[3-(2-thienyl)-1H-indazol-5-yl]-3-pyridinyl}oxy)methyl]ethyl}amine;
[(1S)-2-({5-[3-(3-furanyl)-1H-indazol-5-yl]-6-phenyl-3-pyridinyl}oxy)-1-(phenylmethyl)ethyl]amine;
[(1S)-2-({5-[3-(3-thienyl)-1H-indazol-5-yl]-6-phenyl-3-pyridinyl}oxy)-1-(phenylmethyl)ethyl]amine;
3-[5-{[(2S)-2-amino-3-phenylpropyl]oxy}-3-(3-methyl-1H-indazol-5-yl)-2-pyridinyl]phenol;
[(1S)-2-{[5-(2,3-dimethyl-2H-indazol-5-yl)-6-phenyl-3-pyridinyl]oxy}-1-(phenylmethyl)ethyl]amine;
[(1S)-2-{[5-(3-cyclopropyl-1H-indazol-5-yl)-6-(3-furanyl)-3-pyridinyl]oxy}-1-(phenylmethyl)ethyl]amine;
[(1S)-2-{[5-(3-methyl-1H-indazol-5-yl)-6-(1-methyl-1H-pyrazol-4-yl)-3-pyridinyl]oxy}-1-(phenylmethyl)ethyl]amine;
[(1S)-2-{[6-{1-[(3-fluorophenyl)methyl]-1H-pyrazol-4-yl}-5-(3-methyl-1H-indazol-5-yl)-3-pyridinyl]oxy}-1-(phenylmethyl)ethyl]amine;
((1S)-2-phenyl-1-{[(6-phenyl-5-{3-[5-(1-piperazinylmethyl)-2-furanyl]-1H-indazol-5-yl}-3-pyridinyl)oxy]methyl}ethyl)amine;
[(1S)-2-({6-(3-furanyl)-5-[3-(2-furanyl)-1H-indazol-5-yl]-3-pyridinyl}oxy)-1-(phenylmethyl)ethyl]amine;
[(1S)-2-({5-(3-methyl-1H-indazol-5-yl)-6-[3-(phenyloxy)phenyl]-3-pyridinyl}oxy)-1-(phenylmethyl)ethyl]amine;
3-[({5-[5-(5-{[(2S)-2-amino-3-phenylpropyl]oxy}-2-phenyl-3-pyridinyl)-1H-indazol-3-yl]-2-furanyl}methyl)amino]propanenitrile;
[(1S)-2-({6-(2-furanyl)-5-[3-(2-furanyl)-1H-indazol-5-yl]-3-pyridinyl}oxy)-1-(phenylmethyl)ethyl]amine;
{5-[5-{[(2S)-2-amino-3-phenylpropyl]oxy}-3-(3-methyl-1H-indazol-5-yl)-2-pyridinyl]-2-thienyl}methanol;
{(1S)-2-phenyl-1-[({6-phenyl-5-[3-(phenylmethyl)-1H-indazol-5-yl]-3-pyridinyl}oxy)methyl]ethyl}amine;
[(1S)-2-{[5-(3-methyl-1H-indazol-5-yl)-6-(1-methyl-1H-pyrrol-2-yl)-3-pyridinyl]oxy}-1-(phenylmethyl)ethyl]amine;
5-(5-{[(2S)-2-amino-3-phenylpropyl]oxy}-2-phenyl-3-pyridinyl)-1H-indazol-3-amine;
[(1S)-2-({5-[3-(1-methylethenyl)-1H-indazol-5-yl]-6-phenyl-3-pyridinyl}oxy)-1-(phenylmethyl)ethyl]amine;
[(1S)-2-{[5-(3-methyl-1H-indazol-5-yl)-6-(1H-pyrazol-4-yl)-3-pyridinyl]oxy)-1-(phenylmethyl)ethyl]amine;
(2S)-N,N-dimethyl-1-{[5-(3-methyl-1H-indazol-5-yl)-6-phenyl-3-pyridinyl]oxy}-3-phenyl-2-propanamine;
[(1S)-2-{[3-(3-methyl-1H-indazol-5-yl)-2,4′-bipyridin-5-yl]oxy}-1-(phenylmethyl)ethyl]amine;
[(1S)-2-{[3-(3-methyl-1H-indazol-5-yl)-2,3′-bipyridin-5-yl]oxy}-1-(phenylmethyl)ethyl]amine;
[(1S)-2-{[5-(3-iodo-1H-indazol-5-yl)-6-phenyl-3-pyridinyl]oxy}-1-(phenylmethyl)ethyl]amine;
[(1S)-2-[(5-(3-methyl-1H-indazol-5-yl)-6-{3-[(trifluoromethyl)oxy]phenyl}-3-pyridinyl)oxy]-1-(phenylmethyl)ethyl]amine;
[(1S)-2-{[6-(3,5-dimethyl-4-isoxazolyl)-5-(3-methyl-1H-indazol-5-yl)-3-pyridinyl]oxy}-1-(phenylmethyl)ethyl]amine;
4-[5-{[(2S)-2-amino-3-phenylpropyl]oxy}-3-(3-methyl-1H-indazol-5-yl)-2-pyridinyl]phenol;
2-[5-{[(2S)-2-amino-3-phenylpropyl]oxy}-3-(3-methyl-1H-indazol-5-yl)-2-pyridinyl]phenol;
[(1S)-2-{[6-[3-(ethyloxy)phenyl]-5-(3-methyl-1H-indazol-5-yl)-3-pyridinyl]oxy}-1-(phenylmethyl)ethyl]amine;
[(1S)-2-({5-(3-methyl-1H-indazol-5-yl)-6-[3-(methyloxy)phenyl]-3-pyridinyl}oxy)-1-(phenylmethyl)ethyl]amine;
{3-[5-{[(2S)-2-amino-3-phenylpropyl]oxy}-3-(3-methy-1H-indazol-5-yl)-2-pyridinyl]phenyl}(phenyl)methanone;
[(1S)-2-{[6-{3-[(1-methylethyl)oxy]phenyl}-5-(3-methyl-1H-indazol-5-yl)-3-pyridinyl]oxy}-1-(phenylmethyl)ethyl]amine;
[(1S)-2-{[5-[3-(2-furanyl)-1H-indazol-5-yl]-6-(1H-pyrrol-2-yl)-3-pyridinyl]oxy}-1-(phenylmethyl)ethyl]amine;
[(1S)-2-{[6-(2-{[(3-fluorophenyl)methyl]oxy}phenyl)-5-(3-methyl-1H-indazol-5-yl)-3-pyridinyl]oxy}-1-(phenylmethyl)ethyl]amine;
[(1S)-2-{[6-(4-{[(3-fluorophenyl)methyl]oxy}phenyl)-5-(3-methyl-1H-indazol-5-yl)-3-pyridinyl]oxy}-1-(phenylmethyl)ethyl]amine;
[(1S)-2-({5-[3-(5-chloro-2-thienyl)-1H-indazol-5-yl]-6-phenyl-3-pyridinyl}oxy)-1-(phenylmethyl)ethyl]amine;
[(1S)-2-({5-[3-(4-methyl-2-thienyl)-1H-indazol-5-yl]-6-phenyl-3-pyridinyl}oxy)-1-(phenylmethyl)ethyl]amine;
[(1S)-2-({5-[3-(5-methyl-2-furanyl)-1H-indazol-5-yl]-6-phenyl-3-pyridinyl}oxy)-1-(phenylmethyl)ethyl]amine;
[(1S)-2-({5-[3-(5-methyl-2-thienyl)-1H-indazol-5-yl]-6-phenyl-3-pyridinyl}oxy)-1-(phenylmethyl)ethyl]amine;
[(1S)-2-{[6-ethenyl-5-(3-methyl-1H-indazol-5-yl)-3-pyridinyl]oxy}-1-(phenylmethyl)ethyl]amine;
{(1S)-2-phenyl-1-[({6-phenyl-5-[3-(1H-pyrrol-2-yl)-1H-indazol-5-yl]-3-pyridinyl}oxy)methyl]ethyl}amine;
[(1S)-2-(1H-indol-3-yl)-1-({[5-(3-methyl-1H-indazol-5-yl)-6-phenyl-3-pyridinyl]oxy}methyl)ethyl]amine;
5-(3-methyl-1H-indazol-5-yl)-6-phenyl-N-(3-phenylpropyl)-3-pyridinamine;
5-(3-methyl-1H-indazol-5-yl)-6-phenyl-N-(3-phenylbutyl)-3-pyridinamine;
[(2S)-2-amino-3-phenylpropyl][5-(3-methyl-1H-indazol-5-yl)-6-phenyl-3-pyridinyl]amine;
[(2S)-2-amino-3-phenylpropyl][6-(3-furanyl)-5-(3-methyl-1H-indazol-5-yl)-3-pyridinyl]amine;
((1S)-2-{[6-(3-furanyl)-5-(3-methyl-1H-indazol-5-yl)-3-pyridinyl]oxy}-1-{[(phenylmethyl)oxy]methyl}ethyl)amine;
N-[(2S)-2-amino-3-phenylpropyl]-N-[5-(3-methyl-1H-indazol-5-yl)-6-phenyl-3-pyridinyl]methanesulfonamide;
5-(3-methyl-1H-indazol-5-yl)-N-[2-methyl-2-(phenylthio)propyl]-6-phenyl-3-pyridinamine;
[(1S)-2-{[6-(3-furanyl)-5-(3-methyl-1H-indazol-5-yl)-3-pyridinyl]oxy}-1-(1H-indol-3-ylmethyl)ethyl]amine;
((1S)-2-{[5-(3-methyl-1H-indazol-5-yl)-6-phenyl-3-pyridinyl]oxy}-1-{[(phenylmethyl)oxy]methyl}ethyl)amine;
(2S)-2-amino-3-{[5-(3-methyl-1H-indazol-5-yl)-6-phenyl-3-pyridinyl]oxy}-1-propanol;
5-(3-methyl-1H-indazol-5-yl)-6-phenyl-N-[(2S)-2-pyrrolidinylmethyl]-3-pyridinamine;
((2S)-2-amino-3-{4-[(phenylmethyl)oxy]phenyl}propyl)[5-(3-methyl-1H-indazol-5-yl)-6-phenyl-3-pyridinyl]amine;
[(2S)-2-amino-3-phenylpropyl][5-(1H-indazol-5-yl)-6-phenyl-3-pyridinyl]amine;
[(2S)-2-amino-3-phenylpropyl][6-(3-furanyl)-5-(1H-indazol-5-yl)-3-pyridinyl]amine;
[(2S)-2-amino-3-phenylpropyl][5-(1H-indazol-5-yl)-6-(3-thienyl)-3-pyridinyl]amine;
2-[5-{[(2S)-2-amino-3-phenylpropyl]amino}-3-(1H-indazol-5-yl)-2-pyridinyl]phenol;
2-[5-{[(2S)-2-amino-3-phenylpropyl]amino}-3-(3-methyl-1H-indazol-5-yl)-2-pyridinyl]phenol;
[(2S)-2-amino-3-phenylpropyl][5-(3-methyl-1H-indazol-5-yl)-6-(1H-pyrrol-2-yl)-3-pyridinyl]amine;
[(2S)-2-amino-3-phenylpropyl][5-(3-methyl-1H-indazol-5-yl)-6-(5-methyl-2-thienyl)-3-pyridinyl]amine;
[(2R)-2-amino-3-phenylpropyl][5-(1H-indazol-5-yl)-6-(3-thienyl)-3-pyridinyl]amine;
2-[5-{[(2S)-2-amino-3-(1H-indol-3-yl)propyl]oxy}-3-(3-methyl-1H-indazol-5-yl)-2-pyridinyl]phenol;
[(1S)-2-(1H-indol-3-yl)-1-({[5-(3-methyl-1H-indazol-5-yl)-6-(1H-pyrrol-2-yl)-3-pyridinyl]oxy}methyl)ethyl]amine;
[(1S)-2-(1H-indol-3-yl)-1-({[5-(3-methyl-1H-indazol-5-yl)-6-(5-methyl-2-thienyl)-3-pyridinyl]oxy}methyl)ethyl]amine;
[(1S)-2-{[6-ethyl-5-(3-methyl-1H-indazol-5-yl)-3-pyridinyl]oxy}-1-(phenylmethyl)ethyl]amine;
[(1S)-2-{[6-(3-furanyl)-5-(1H-indazol-5-yl)-3-pyridinyl]oxy}-1-(phenylmethyl)ethyl]amine;
[(1S)-2-{[5-(3-ethenyl-1H-indazol-5-yl)-6-(3-furanyl)-3-pyridinyl]oxy}-1-(phenylmethyl)ethyl]amine;
[(1S)-2-{[5-(3-ethyl-1H-indazol-5-yl)-6-(3-furanyl)-3-pyridinyl]oxy}-1-(phenylmethyl)ethyl]amine;
[(1S)-2-({6-(3-furanyl)-5-[3-(3-pyridinyl)-1H-indazol-5-yl]-3-pyridinyl}oxy)-1-(phenylmethyl)ethyl]amine;
[(1S)-2-{[6-methyl-5-(3-methyl-1H-indazol-5-yl)-3-pyridinyl]oxy}-1-(phenylmethyl)ethyl]amine;
[(1S)-2-({5-(3-methyl-1H-indazol-5-yl)-6-[2-(methyloxy)phenyl]-3-pyridinyl}oxy)-1-(phenylmethyl)ethyl]amine;
[(1S)-2-{[6-[2-(ethyloxy)phenyl]-5-(3-methyl-1H-indazol-5-yl)-3-pyridinyl]oxy}-1-(phenylmethyl)ethyl]amine;
[(1S)-2-{[6-[5-chloro-2-(methyloxy)phenyl]-5-(3-methyl-1H-indazol-5-yl)-3-pyridinyl]oxy}-1-(phenylmethyl)ethyl]amine;
[(1S)-2-{[6-[5-fluoro-2-(propyloxy)phenyl]-5-(3-methyl-1H-indazol-5-yl)-3-pyridinyl]oxy}-1-(phenylmethyl)ethyl]amine;
[(1S)-2-({5-[3-(1-methylethyl)-1H-indazol-5-yl]-6-phenyl-3-pyridinyl}oxy)-1-(phenylmethyl)ethyl]amine;
[(1S)-2-{[5-(6-fluoro-3-methyl-1H-indazol-5-yl)-6-(3-furanyl)-3-pyridinyl]oxy}-1-(phenylmethyl)ethyl]amine;
N-[6-(3-furanyl)-5-(3-methyl-1H-indazol-5-yl)-3-pyridinyl]-L-phenylalaninamide;
N-[6-(2-hydroxyphenyl)-5-(3-methyl-1H-indazol-5-yl)-3-pyridinyl]-L-phenylalaninamide;
2-[5-{[(2S)-2-amino-3-(1-benzothien-3-yl)propyl]oxy}-3-(1H-indazol-5-yl)-2-pyridinyl]phenol;
[(1S)-2-(1-benzothien-3-yl)-1-({[6-(2-furanyl)-5-(1H-indazol-5-yl)-3-pyridinyl]oxy}methyl)ethyl]amine;
[(1S)-2-{[5-(3-methyl-1H-indazol-5-yl)-6-phenyl-3-pyridinyl]oxy}-1-(2-naphthalenylmethyl)ethyl]amine;
N-[5-(3-methyl-1H-indazol-5-yl)-6-(1H-pyrrol-2-yl)-3-pyridinyl]-L-phenylalaninamide;
[(2S)-2-amino-3-(1H-indol-3-yl)propyl][6-(3-furanyl)-5-(3-methyl-1H-indazol-5-yl)-3-pyridinyl]amine;
(2S)-1-{[6-(3-furanyl)-5-(3-methyl-1H-indazol-5-yl)-3-pyridinyl]oxy}-3-phenyl-2-propanol;
1-{3-[5-{[(2S)-2-amino-3-(1H-indol-3-yl)propyl]oxy}-2-(3-furanyl)-3-pyridinyl]phenyl}ethanone;
[(1S)-2-{[6-cyclopentyl-5-(3-methyl-1H-indazol-5-yl)-3-pyridinyl]oxy}-1-(1H-indol-3-ylmethyl)ethyl]amine;
[(1S)-2-(1-benzothien-3-yl)-1-({[5-(1H-indazol-5-yl)-6-phenyl-3-pyridinyl]oxy}methyl)ethyl]amine;
[(1S)-2-(1-benzothien-3-yl)-1-({[6-(3-furanyl)-5-(1H-indazol-5-yl)-3-pyridinyl]oxy}methyl)ethyl]amine;
[(1S)-2-(1-benzothien-3-yl)-1-({[5-(1H-indazol-5-yl)-6-(3-thienyl)-3-pyridinyl]oxy}methyl)ethyl]amine;
[(1S)-2-(1-benzothien-3-yl)-1-({[5-(1H-indazol-5-yl)-6-(1H-pyrrol-2-yl)-3-pyridinyl]oxy}methyl)ethyl]amine;
[(1S)-2-{[5-(1H-indazol-5-yl)-6-phenyl-3-pyridinyl]oxy}-1-(1H-pyrazol-1-ylmethyl)ethyl]amine;
[(1S)-2-(1-benzothien-3-yl)-1-({[5-(1H-indazol-5-yl)-6-(5-methyl-2-thienyl)-3-pyridinyl]oxy}methyl)ethyl]amine;
[(1S)-2-{[6-(3-furanyl)-5-(3-methyl-1H-thieno[3,2-c]pyrazol-5-yl)-3-pyridinyl]oxy}-1-(1H-indol-3-ylmethyl)ethyl]amine;
5-[5-{[(2S)-2-amino-3-phenylpropyl]oxy}-2-(3-furanyl)-3-pyridinyl]-N-4-pyridinyl-1H-indazol-3-amine;
N-{5-[5-{[(2S)-2-amino-3-phenylpropyl]oxy}-2-(3-furanyl)-3-pyridinyl]-1H-indazol-3-yl}benzamide;
(1E)-1-{3-[5-{[(2S)-2-amino-3-(1H-indol-3-yl)propyl]oxy}-2-(3-furanyl)-3-pyridinyl]phenyl}ethanone oxime;
[(1S)-2-{[6-(3-furanyl)-5-(3-methyl-1H-indazol-5-yl)-3-pyridinyl]oxy}-1-(phenylmethyl)propyl]amine;
(2S)-N-methyl-1-{[5-(3-methyl-1H-indazol-5-yl)-6-phenyl-3-pyridinyl]oxy}-3-phenyl-2-propanamine;
[(1S)-2-{[6-[5-fluoro-2-(methyloxy)phenyl]-5-(3-methyl-1H-indazol-5-yl)-3-pyridinyl]oxy}-1-(phenylmethyl)ethyl]amine;
[(1S)-2-{[6-[3,5-difluoro-2-(methyloxy)phenyl]-5-(3-methyl-1H-indazol-5-yl)-3-pyridinyl]oxy}-1-(phenylmethyl)ethyl]amine;
[(1S)-2-({6-(3-furanyl)-5-[3-(4-pyridinyl)-1H-indazol-5-yl]-3-pyridinyl}oxy)-1-(phenylmethyl)ethyl]amine;
2-[5-{[(2S)-2-amino-3-phenylpropyl]oxy}-3-(3-methyl-1H-indazol-5-yl)-2-pyridinyl]-4-fluorophenol;
2-[5-{[(2S)-2-amino-3-phenylpropyl]oxy}-3-(3-methyl-1H-indazol-5-yl)-2-pyridinyl]-4,6-difluorophenol;
2-[5-{[(2S)-2-amino-3-(1H-indol-3-yl)propyl]oxy}-3-(6-fluoro-3-methyl-1H-indazol-5-yl)-2-pyridinyl]phenol;
2-[5-{[(2S)-2-amino-3-(1H-indol-3-yl)propyl]oxy}-3-(3-ethyl-1H-indazol-5-yl)-2-pyridinyl]phenol;
[(1S)-2-{[5-(3-ethyl-1H-indazol-5-yl)-6-(3-furanyl)-3-pyridinyl]oxy}-1-(1H-indol-3-ylmethyl)ethyl]amine;
[(1S)-2-{[5-(3-ethyl-1H-indazol-5-yl)-6-(2-furanyl)-3-pyridinyl]oxy}-1-(1H-indol-3-ylmethyl)ethyl]amine;
[(1S)-2-{[5-(3-ethyl-1H-indazol-5-yl)-6-(1H-pyrrol-2-yl)-3-pyridinyl]oxy}-1-(1H-indol-3-ylmethyl)ethyl]amine;
[(1S)-2-({6-(3-furanyl)-5-[3-(1-methyl-1H-pyrazol-4-yl)-1H-indazol-5-yl]-3-pyridinyl}oxy)-1-(phenylmethyl)ethyl]amine;
[(1S)-2-({6-(3-furanyl)-5-[3-(1H-pyrrol-2-yl)-1H-indazol-5-yl]-3-pyridinyl}oxy)-1-(phenylmethyl)ethyl]amine;
[(1S)-2-({6-(3-furanyl)-5-[3-(1H-pyrazol-4-yl)-1H-indazol-5-yl]-3-pyridinyl}oxy)-1-(phenylmethyl)ethyl]amine;
[(1S)-2-{[5-(6-fluoro-3-methyl-1H-indazol-5-yl)-6-(2-furanyl)-3-pyridinyl]oxy}-1-(1H-indol-3-ylmethyl)ethyl]amine;
[(1S)-2-{[5-(6-fluoro-3-methyl-1H-indazol-5-yl)-6-(1H-pyrrol-2-yl)-3-pyridinyl]oxy}-1-(1H-indol-3-ylmethyl)ethyl]amine;
[(1S)-2-{[5-(6-fluoro-3-methyl-1H-indazol-5-yl)-6-(3-furanyl)-3-pyridinyl]oxy}-1-(1H-indol-3-ylmethyl)ethyl]amine;
[(1S)-2-{[6-(1-benzothien-2-yl)-5-(3-methyl-1H-indazol-5-yl)-3-pyridinyl]oxy}-1-(phenylmethyl)ethyl]amine;
[(1S)-2-{[6-(1-benzofuran-2-yl)-5-(3-methyl-1H-indazol-5-yl)-3-pyridinyl]oxy}-1-(phenylmethyl)ethyl]amine;
[(1S)-2-({6-(3-furanyl)-5-[3-(methylsulfonyl)phenyl]-3-pyridinyl}oxy)-1-(1H-indol-3-ylmethyl)ethyl]amine;
5-[5-{[(2S)-2-(1-azetidinyl)-3-(1H-indol-3-yl)propyl]oxy}-2-(3-furanyl)-3-pyridinyl]-3-methyl-1H-indazole;
[(1S)-2-({6-(3-furanyl)-5-[3-(1H-pyrazol-4-yl)-1H-indazol-5-yl]-3-pyridinyl}oxy)-1-(1H-indol-3-ylmethyl)ethyl]amine;
3-[5-{[(2S)-2-amino-3-(1H-indol-3-yl)propyl]oxy}-2-(3-furyl)pyridin-3-yl]benzamide;
4-[5-{[(2S)-2-amino-3-(1H-indol-3-yl)propyl]oxy}-2-(3-furyl)pyridin-3-yl]benzamide;
5-(5-{[(2S)-3-(1H-indol-3-yl)-2-(1-piperidinyl)propyl]oxy}-2-phenyl-3-pyridinyl)-3-methyl-1H-indazole;
5-(2-(3-furanyl)-5-{[(2S)-3-(1H-indol-3-yl)-2-(4-morpholinyl)propyl]oxy}-3-pyridinyl)-3-methyl-1H-indazole;
[(1S)-2-({6-(3-furanyl)-5-[3-(1H-pyrazol-4-yl)-1H-indazol-5-yl]-3-pyridinyl}oxy)-1-(1H-indol-3-ylmethyl)ethyl]amine;
[(1S)-2-{[6-(3-furanyl)-5-(3-methyl-1H-indazol-5-yl)-3-pyridinyl]oxy}-1-(1H-indol-3-ylmethyl)ethyl]dimethylamine;
(3S)-3-({[6-(3-furanyl)-5-(3-methyl-1H-indazol-5-yl)-3-pyridinyl]oxy}methyl)-2-methyl-2,3,4,9-tetrahydro-1H-carboline;
1-{5-[5-{[(2S)-2-amino-3-(1H-indol-3-yl)propyl]oxy}-2-(3-furanyl)-3-pyridinyl]-2-thienyl}ethanone;
(2S)-1-{[6-(3-furanyl)-5-(3-methyl-1H-indazol-5-yl)-3-pyridinyl]oxy}-3-(1H-indol-3-yl)-N-methyl-2-propanamine;
5-[5-{[(2S)-2-amino-3-(1H-indol-3-yl)propyl]oxy}-2-(3-furanyl)-3-pyridinyl]-N,N-dimethyl-2-furancarboxamide;
5-[5-{[(2S)-2-amino-3-(1H-indol-3-yl)propyl]oxy}-2-(3-furanyl)-3-pyridinyl]-N-methyl-2-furancarboxamide;
5-[5-{[(2S)-2-amino-3-(1H-indol-3-yl)propyl]oxy}-2-(3-furanyl)-3-pyridinyl]-2-furancarboxamide;
[(2S)-2-amino-3-phenylpropyl][6-(3-furanyl)-5-(3-methyl-1H-indazol-5-yl)-3-pyridinyl]methylamine;
[(1S)-2-(3,4-dichlorophenyl)-1-({[5-(3-methyl-1H-indazol-5-yl)-6-phenyl-3-pyridinyl]oxy}methyl)ethyl]amine;
N-[5-(3-methyl-1H-indazol-5-yl)-6-phenyl-3-pyridinyl]-L-phenylalaninamide;
N-[5-(3-methyl-1H-indazol-5-yl)-6-phenyl-3-pyridinyl]-L-phenylalaninamide;
2-[5-{[(2S)-2-amino-3-(1H-indol-3-yl)propyl]amino}-3-(3-methyl-1H-indazol-5-yl)-2-pyridinyl]-4-fluorophenol;
((1S)-3-{[6-(3-furanyl)-5-(3-methyl-1H-indazol-5-yl)-3-pyridinyl]oxy}-1-{[4-(trifluoromethyl)phenyl]methyl}propyl)amine;
[(1S)-3-{[6-(3-furanyl)-5-(3-methyl-1H-indazol-5-yl)-3-pyridinyl]oxy}-1-(1H-indol-3-ylmethyl)propyl]amine;
{(1S)-2-{[6-(3-furanyl)-5-(3-methyl-1H-indazol-5-yl)-3-pyridinyl]oxy}-1-[(5-methyl-1H-indol-3-yl)methyl]ethyl}amine;
[(1S)-2-(1H-indol-3-yl)-1-({[5-(3-methyl-1H-indazol-5-yl)-6-(1H-pyrrol-3-yl)pyridin-3-yl]oxy}methyl)ethyl]amine;
[(1S)-2-{[6-(3-furanyl)-5-(3-methyl-1H-pyrazolo[4,3-b]pyridin-5-yl)-3-pyridinyl]oxy}-1-(1H-indol-3-ylmethyl)ethyl]amine;
[(1S)-2-(1H-indol-3-yl)-1-({[5-(3-methyl-1H-pyrazolo[4,3-b]pyridin-5-yl)-3-pyridinyl]oxy}methyl)ethyl]amine;
5-[5-{[(2S)-2-amino-3-phenylpropyl]oxy}-2-(3-furanyl)-3-pyridinyl]-1H-indazole-3-carboxamide;
5-[5-{[(2S)-2-amino-3-phenylpropyl]oxy}-2-(3-furanyl)-3-pyridinyl]-1H-indazole-3-carbonitrile;
(2S)-1-{[6-(2-furanyl)-5-(3-methyl-1H-indazol-5-yl)-3-pyridinyl]oxy}-3-(1H-indol-3-yl)-2-propanamine;
2-[5-{[(2S)-2-amino-3-(1-benzothien-3-yl)-3-propyl]oxy}-3-(1H-indazol-5-yl)-2-pyridinyl]-4-flurophenol;
2-[5-{[(2S)-2-amino-3-(1-benzothien-3-yl)-3-propyl]oxy}-3-(1H-indazol-5-yl)-2-pyridinyl]-4,6-diflurophenol;
[(1S)-2-(1-benzothien-3-yl)-1-({[5,6-bis(3-methyl-1H-indazol-5-yl)-3-pyridinyl]oxy}methyl)ethyl]amine;
[(1S)-2-(1-benzothien-3-yl)-1-({[4-(3-furanyl)-3-(3-methyl-1H-indazol-5-yl)phenyl]oxy}methyl)ethyl]amine;
4′-{[(2S)-2-amino-3-(1-benzothien-3-yl)propyl]oxy}-3,5-difluoro-2′-(3-methyl-1H-indazol-5-yl)-2-biphenylol;
4′-{[(2S)-2-amino-3-(1-benzothien-3-yl)propyl]oxy}-5-fluoro-2′-(3-methyl-1H-indazol-5-yl)-2-biphenylol;
2-[5-{[(2S)-2-amino-3-(1H-indol-3-yl)propyl]oxy}-3-(3-methyl-1H-indazol-5-yl)-2-pyridinyl]-4,6-difluorophenol;
[(2S)-2-amino-3-(1H-indol-3-yl)propyl][5-(3-methyl-1H-indazol-5-yl)-6-(1H-pyrrol-2-yl)-3-pyridinyl]amine;
[(2S)-2-amino-3-(1H-indol-3-yl)propyl][6-[5-fluoro-2-(methyloxy)phenyl]-5-(3-methyl-1H-indazol-5-yl)-3-pyridinyl]amine;
2-[5-{[(2S)-2-amino-3-(1H-indol-3-yl)propyl]amino}-3-(3-methyl-1H-indazol-5-yl)-2-pyridinyl]phenol;
2-[5-{[(2S)-2-amino-3-(1H-indol-3-yl)propyl]amino}-3-(3-methyl-1H-indazol-5-yl)-2-pyridinyl]phenol;
[(2S)-2-amino-3-(5-fluoro-1H-indol-3-yl)propyl][6-(3-furanyl)-5-(3-methyl-1H-indazol-5-yl)-3-pyridinyl]amine;
[(2S)-2-amino-4-pentyn-1-yl][6-(3-furanyl)-5-(3-methyl-1H-indazol-5-yl)-3-pyridinyl]amine;
[(2S)-2-amino-3-(5,6,7-trifluoro-1H-indol-3-yl)propyl][6-(3-furanyl)-5-(3-methyl-1H-indazol-5-yl)-3-pyridinyl]amine;
[(2S)-2-amino-3-(5,7-difluoro-1H-indol-3-yl)propyl][6-(3-furanyl)-5-(3-methyl-1H-indazol-5-yl)-3-pyridinyl]amine;
[(1S)-2-{[6-(3-furanyl)-5-(3-methyl-1H-indazol-5-yl)-3-pyridinyl]oxy}-1-(1H-pyrrolo[2,3-b]pyridin-2-ylmethyl)ethyl]amine;
[(2R)-2-amino-3-phenylpropyl][3-fluoro-4-(3-furanyl)-5-(3-methyl-1H-indazol-5-yl)phenyl]amine;
[(2R)-2-amino-3-(1H-indol-3-yl)propyl][3-fluoro-4-(3-furanyl)-5-(3-methyl-1H-indazol-5-yl)phenyl]amine;
[(1S)-2-{[6-(3-furanyl)-5-(3-methyl-1H-pyrazolo[3,4-b]pyridin-5-yl)-3-pyridinyl]oxy}-1-(1H-indol-3-ylmethyl)ethyl]amine;
[(1S)-2-(1H-indol-3-yl)-1-({[6-(2-methyl-3-furanyl)-5-(3-methyl-1H-pyrazolo[3,4-b]pyridin-5-yl)-3-pyridinyl]oxy}methyl)ethyl]amine;
[(1S)-2-(1H-indol-3-yl)-1-({[5-(3-methyl-1H-pyrazolo[3,4-b]pyridin-5-yl)-6-phenyl-3-pyridinyl]oxy}methyl)ethyl]amine;
[(1S)-2-{[6-(3-furanyl)-5-(3-methyl-1H-pyrazolo[3,4-b]pyridin-5-yl)-3-pyridinyl]oxy}-1-(1H-indol-3-ylmethyl)ethyl]methylamine;
2-[5-{[(2S)-2-amino-3-(1H-indol-3-yl)propyl]oxy}-3-(3-methyl-1H-pyrazolo[3,4-b]pyridin-5-yl)-2-pyridinyl]phenol;
2-[5-{[(2S)-2-amino-3-(1H-indol-3-yl)propyl]oxy}-3-(3-methyl-1H-pyrazolo[3,4-b]pyridin-5-yl)-2-pyridinyl]-6-fluorophenol;
[(1S)-2-{[5-[3-(3,5-dimethyl-4-isoxazolyl)-1H-indazol-5-yl]-6-(3-furanyl)-3-pyridinyl]oxy}-1-(phenylmethyl)ethyl]amine;
[(1S)-2-({6-(3-furanyl)-5-[3-(2-pyridinyl)-1H-indazol-5-yl]-3-pyridinyl}oxy)-1-(phenylmethyl)ethyl]amine;
[(1S)-2-{[6-(2-chlorophenyl)-5-(3-methyl-1H-indazol-5-yl)-3-pyridinyl]oxy}-1-(phenylmethyl)ethyl]amine;
[(1S)-2-{[5-(3-methyl-1H-indazol-5-yl)-6-(2-methylphenyl)-3-pyridinyl]oxy}-1-(phenylmethyl)ethyl]amine;
[(1S)-2-{[6-(2-fluorophenyl)-5-(3-methyl-1H-indazol-5-yl)-3-pyridinyl]oxy}-1-(phenylmethyl)ethyl]amine;
2-[5-{[(2S)-2-amino-3-phenylpropyl]oxy}-3-(3-methyl-1H-indazol-5-yl)-2-pyridinyl]-4-chlorophenol;
[(1S)-2-{[6-(1-benzothien-3-yl)-5-(3-methyl-1H-indazol-5-yl)-3-pyridinyl]oxy}-1-(phenylmethyl)ethyl]amine;
3-[5-{((2S)-2-amino-3-phenylpropyl]oxy}-3-(3-methyl-1H-indazol-5-yl)-2-pyridinyl]benzamide;
3-[5-{[(2S)-2-amino-3-phenylpropyl]oxy}-3-(3-methyl-1H-indazol-5-yl)-2-pyridinyl]benzonitrile;
[(1S)-2-{[5-(3-methyl-1H-indazol-5-yl)-6-(3-nitrophenyl)-3-pyridinyl]oxy}-1-(phenylmethyl)ethyl]amine;
[(1S)-2-{[5-(3-methyl-1H-indazol-5-yl)-6-(4-methyl-2-thienyl)-3-pyridinyl]oxy}-1-(phenylmethyl)ethyl]amine;
N-{3-[5-{[(2S)-2-amino-3-phenylpropyl]oxy}-3-(3-methyl-1H-indazol-5-yl)-2-pyridinyl]phenyl}-N′-phenylurea;
[(1S)-2-{[5-(3-methyl-1H-indazol-5-yl)-6-(2-thienyl)-3-pyridinyl]oxy}-1-(phenylmethyl)ethyl]amine;
[(1S)-2-(1H-indol-3-yl)-1-({[6-(2-methyl-3-furanyl)-5-(3-methyl-1H-indazol-5-yl)-3-pyridinyl]oxy}methyl)ethyl]amine;
{2-[5-{[(2S)-2-amino-3-(1H-indol-3-yl)propyl]oxy}-3-(3-methyl-1H-indazol-5-yl)-2-pyridinyl]phenyl}amine;
2-[5-{[(2S)-2-amino-3-(1H-indol-3-yl)propyl]oxy}-3-(3-methyl-1H-indazol-5-yl)-2-pyridinyl]-6-fluorophenol;
2-[5-{[(2S)-2-amino-3-(1H-indol-3-yl)propyl]oxy}-3-(3-methyl-1H-indazol-5-yl)-2-pyridinyl]-4-chlorophenol;
2-[5-{[(2S)-2-amino-3-(1H-indol-3-yl)propyl]oxy}-3-(3-methyl-1H-indazol-5-yl)-2-pyridinyl]-4-fluorophenol;
[(1S)-2-{[6-[3,5-difluoro-2-(methyloxy)phenyl]-5-(3-methyl-1H-thieno[3,2-c]pyrazol-5-yl)-3-pyridinyl]oxy}-1-(1H-indol-3-ylmethyl)ethyl]amine;
2-[5-{[(2S)-2-amino-3-(1H-indol-3-yl)propyl]oxy}-3-(3-methyl-1H-thieno[3,2-c]pyrazol-5-yl)-2-pyridinyl]-4,6-difluorophenol;
2-[5-{[(2S)-2-amino-3-(1H-indol-3-yl)propyl]oxy}-3-(3-methyl-1H-thieno[3,2-c]pyrazol-5-yl)-2-pyridinyl]phenol;
2-[5-{[(2S)-2-amino-3-(1H-indol-3-yl)propyl]oxy}-3-(3-methyl-1H-thieno[3,2-c]pyrazol-5-yl)-2-pyridinyl]-4-chlorophenol;
3-(5-{[(2S)-2-amino-3-(1H-indol-3-yl)propyl]oxy}-3-pyridinyl)benzamide;
1-[3-(5-{[(2S)-2-amino-3-(1H-indol-3-yl)propyl]oxy}-3-pyridinyl)phenyl]ethanone; and
5-{[(2S)-2-amino-3-(1H-indol-3-yl)propyl]oxy}-2-(3-furanyl)-3,4′-bipyridine-2′-carboxamide
   and/or pharmaceutically acceptable salts, hydrates, solvates and pro-drugs thereof.

Compounds of Formula (I) are included in the pharmaceutical compositions of the invention and used in the methods of the invention.

By the term “aryl” as used herein, unless otherwise defined, is meant a cyclic or polycyclic aromatic ring containing from 1 to 14 carbon atoms and optionally containing from one to five heteroatoms, provided that when the number of carbon atoms is 1 the aromatic ring contains at least four heteroatoms, when the number of carbon atoms is 2 the aromatic ring contains at least three heteroatoms, when the number of carbons is 3 the aromatic ring contains at least two heteroatoms and when the number of carbon atoms is 4 the aromatic ring contains at least one heteroatom.

By the term “C₁-C₁₂aryl” as used herein, unless otherwise defined, is meant phenyl, naphthalene, 3,4-methylenedioxyphenyl, pyridine, biphenyl, indazole, quinoline, isoquinoline, azaindazole, 1H-thienopyrazole, pyrimidine, quinazoline, thiophene, furan, pyrrole, pyrazole, imidazole, benzothiophene, benzofuran, isoxazole, indole and tetrazole.

The term “substituted” as used herein, unless otherwise defined, is meant that the subject chemical moiety has one or more substituents selected from the group consisting of: —CO₂R²⁰, C₁-C₁₂aryl, C₁-C₁₂arylamino, C₁-C₁₂arylalkyl, cycloalkyl, heterocyclealkylC₁-C₁₂aryl, cyanoalkylaminoalkylC₁-C₁₂aryl, —C(O)NHS(O)₂R²⁰, —NHS(O)₂R²⁰, —NHC(O)—NHR⁴¹, hydroxyalkyl, alkoxy, —C(O)NR²¹R²², acyloxy, alkyl, R⁴², —NR²¹R²², —C(O)R⁴³, —CHO, C₁-C₁₂aryloxy, amino, methylamino, dimethylamino, N-acylamino, hydroxy, —(CH₂)_{g}C(O)OR²³, —S(O)ₙR²³, —O(CH₂)_{q}R³¹, —O(CH₂)_{y}CH(R³¹)(CH₂)_{z}(CH₃), nitro, tetrazole, cyano, oxo, halogen, trifluoromethoxy, trifluoroalkoxy and trifluoromethyl;

where

n is 0-2, g is 0-6, q is 1-6, y is 0-6, z is 0-6,
R⁴¹ is selected from hydrogen, C₁-C₁₂aryl, cycloalkyl and heterocycle, wherein C₁-C₁₂aryl, cycloalkyl and heterocycle are optionally substituted with from 1 to 4 substituents selected from: halogen, alkyl, hydroxyalkyl, alkoxy, amino, methylamino, dimethylamino, hydroxy, nitro, tetrazole, cyano, oxo and trifluoromethyl,
R⁴² is selected from C₁-C₁₂aryl, C₁-C₆alkyl, cycloalkyl and heterocycle, each of which is substituted with from 1 to 4 substituents selected from: halogen, hydroxyalkyl, alkyl, alkoxy, amino, methylamino, dimethylamino, hydroxy, nitro, tetrazole, cyano, oxo and trifluoromethyl,
R⁴³ is selected from C₁-C₆alkyl, C₁-C₁₂aryl, cycloalkyl and heterocycle, each of which is optionally substituted with from 1 to 4 substituents selected from: halogen, hydroxyalkyl, alkoxy, amino, methylamino, dimethylamino, hydroxyl, nitro, tetrazole, cyano, oxo and trifluoromethyl,
R³¹ is C₁-C₁₂aryl, cycloalkyl and heterocycle, each of which is optionally substituted with from 1 to 4 substituents selected from: halogen, alkyl, hydroxyalkyl, alkoxy, acyloxy, amino, methylamino, dimethylamino, N-acylamino, hydroxy, nitro, tetrazole, cyano, oxo and trifluoromethyl,
R²³ is hydrogen or alkyl,
R²⁰ is selected form hydrogen, C₁-C₄alkyl, aryl and trifluoromethyl, and
R²¹ and R²² are independently selected form hydrogen, C₁-C₄alkyl, aryl and trifluoromethyl.

By the term “alkoxy” as used herein is meant —Oalkyl where alkyl is as described herein including —OCH₃ and —OC(CH₃)₂CH₃.

The term “cycloalkyl” as used herein unless otherwise defined, is meant a nonaromatic, unsaturated or saturated, cyclic or polycyclic C₃-C₁₂.

Examples of cycloalkyl and substituted cycloalkyl substituents as used herein include: cyclohexyl, 4-hydroxy-cyclohexyl, 2-ethylcyclohexyl, cyclohexene, propyl 4-methoxycyclohexyl, 4-methoxycyclohexyl, 4-carboxycyclohexyl, cyclopropyl, cyclopentene and cyclopentyl.

The term “heterocycle,” as used herein, unless otherwise defined, is meant a cyclic or polycyclic, non-aromatic, three-, four-, five-, six-, or seven-membered ring containing at least one atom, selected from the group consisting of oxygen, nitrogen, and sulfur. The five-membered rings have zero or one double bond and the six- and seven-membered rings have zero, one, or two double bonds.

Examples of heterocyclic groups as used herein include: dihydroisoindolyl, dihydroisoquinolinyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl.

By the term “acyloxy” as used herein is meant —OC(O)alkyl where alkyl is as described herein. Examples of acyloxy substituents as used herein include: —OC(O)CH₃, —OC(O)CH(CH₃)₂ and —OC(O)(CH₂)₃CH₃.

By the term “N-acylamino” as used herein is meant a substituent selected from: —N(H)C(O)alkyl, —N(H)C(O)cycloalkyl and —N(H)C(O)aryl; where alkyl and cycloalkyl are as described herein and aryl is C₁-C₁₂aryl as described herein and where the alkyl, cycloalkyl, and aryl are optionally substituted with from 1 to 4 substituents selected from: halogen, hydroxyalkyl, alkoxy, amino, methylamino, dimethylamino, hydroxy, nitro, tetrazole, cyano, oxo and trifluoromethyl. Examples of N-acylamino substituents as used herein include: —N(H)C(O)CH₃, —N(H)C(O)CH(CH₃)₂ and —N(H)C(O)(CH₂)₃CH₃.

By the term “aryloxy” as used herein is meant —Oaryl where aryl is phenyl, naphthyl, 3,4-methylenedioxyphenyl, pyridyl or biphenyl, each of which is optionally substituted with one or more substituents selected from the group consisting of: alkyl, hydroxyalkyl, alkoxy, trifuloromethyl, acyloxy, amino, N-acylamino, hydroxy, —(CH₂)_{g}C(O)OR²⁵, —S(O)ₙR²⁵, nitro, cyano, halogen and protected —OH, where g is 0-6, R²⁵ is hydrogen or alkyl, and n is 0-2. Examples of aryloxy substituents as used herein include: phenoxy, 4-fluorophenyloxy and biphenyloxy.

By the term “heteroatom” as used herein is meant oxygen, nitrogen or sulfur.

By the term “halogen” as used herein is meant a substituent selected from bromide, iodide, chloride and fluoride.

By the term “alkyl” and derivatives thereof and in all carbon chains as used herein is meant a linear or branched, saturated or unsaturated hydrocarbon chain, and unless otherwise defined, the carbon chain will contain from 1 to 12 carbon atoms. Examples of alkyl and substituted alkyl substituents as used herein include: —CH₃, —CH₂—CH₃, —CH₂—CH₂—CH₃, —CH(CH₃)₂, —CH₂—CH₂—C(CH₃)₃, —CH₂—CF₃, —C≡C—C(CH₃)₃, —C≡C—CH₂—OH, cyclopropylmethyl, phenylmethyl, —CH₂#C(CH₃)₂—CH₂—NH₂, —CH₂—C(CH₃)₂—, —C≡C—C₆H₅, —C≡C—C(CH₃)₂—OH, —CH₂—CH(OH)—CH(OH)—CH(OH)—CH(OH)—CH₂—OH, piperidinylmethyl, methoxyphenylethyl, —C(CH₃)₃, —(CH₂)₃—CH₃, —CH₂—CH(CH₃)₂, —CH(CH₃)—CH₂—CH₃, —CH═CH₂, and —C≡C—CH₃.

By the term “treating” and derivatives thereof as used herein, is meant prophylatic and therapeutic therapy.

As used herein, the term “effective amount” and derivatives thereof means that amount of a drug or pharmaceutical agent that will elicit the biological or medical response of a tissue, system, animal or human that is being sought, for instance, by a researcher or clinician. Furthermore, the term “therapeutically effective amount” and derivatives thereof means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

Compounds of Formula (I) are included in the pharmaceutical compositions of the invention and used in the methods of the invention. Where a —COOH or —OH group is present, pharmaceutically acceptable esters can be employed, for example methyl, ethyl, pivaloyloxymethyl, and the like for —COOH, and acetate maleate and the like for —OH, and those esters known in the art for modifying solubility or hydrolysis characteristics, for use as sustained release or prodrug formulations.

The novel compounds of Formulas I and II are prepared as shown in Schemes 1 through 31 below, or by analogous methods, wherein the ‘L’ and ‘R’ substituents are as defined in Formulas I and II respectively and provided that the ‘L’ and ‘R’ substituents do not include any such substituents that render inoperative the processes of Schemes 1 through 31. All of the starting materials are commercially available or are readily made from commercially available starting materials by those of skill in the art.

Ethers such as 1(b) can be prepared by Mitsunobu coupling with hydroxy-pyridines such as 2-chloro-3-bromo-5-hydroxy-pyridine and alcohols such as N-Boc-(2S)-2-amino-3-phenyl-1-propanol (Scheme 1). An aryl moiety such as a 6-(3-methyl-indazole) can be selectively introduced by stoichiormetric use of the Suzuki reaction (Pd-mediated cross coupling between aryl boronic acids or aryl boronic esters and aryl halides or triflates, Chem Rev, 1995, 95(7), 2457-83) or a Stille reaction (Pd-mediated cross coupling between aryltrialkyls tannanes and aryl halides or triflates, Angewandte Chemie, International Edition 2004, 43(36), 4704-4734) to produce intermediates such as 1(d) (Scheme 1). A second aryl moiety such as a phenyl group can be introduced at the adjacent position on the pyridine by a second Suzuki or Stille reaction forming trisubstituted pyridines such as 1(e) (Scheme 1), followed by deprotection steps.

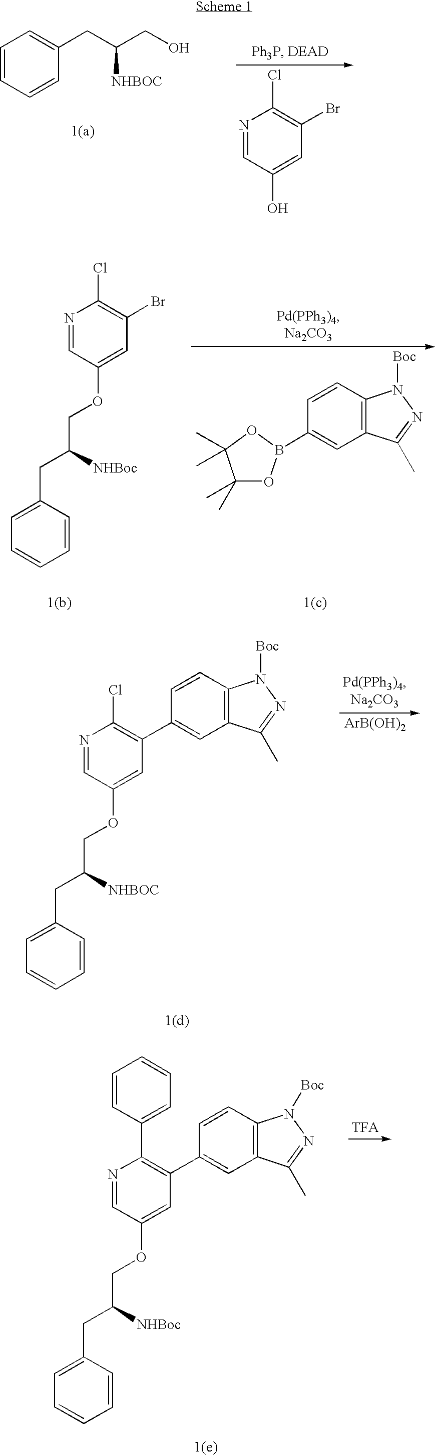

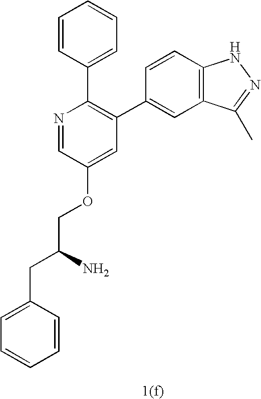

Alternatively, an alkyl or substituted alkyl group such as a benzyl moiety can be introduced by Pd-mediated coupling with an organometallic reagent such as benzyl zinc bromide (Scheme 2) to produce intermediates such as 7(a), followed by deprotection steps.

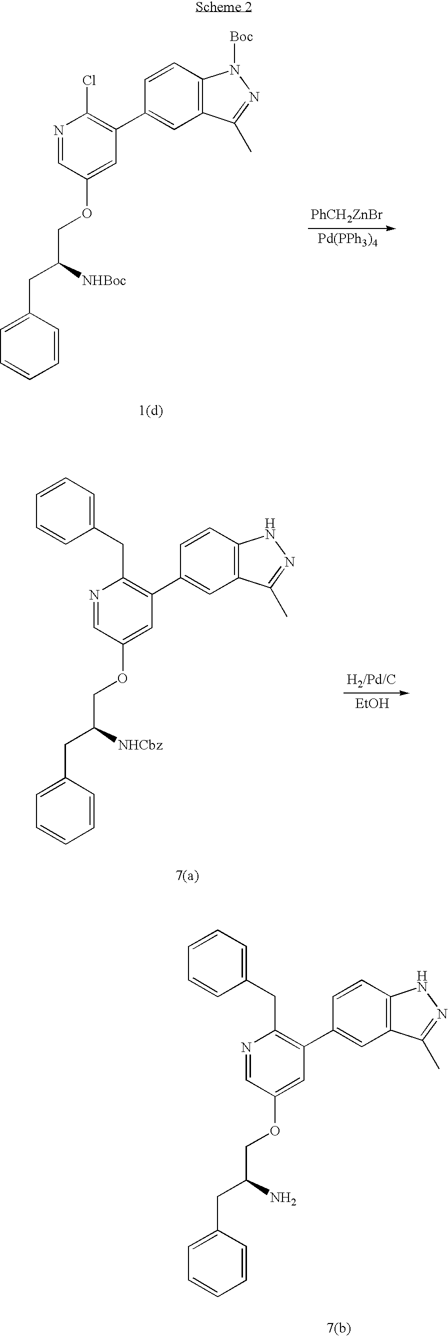

Alternatively, the Pd-mediated cross coupling steps may precede the etherification or Mitsunobu reaction steps as shown in Scheme 3, followed by deprotection steps.

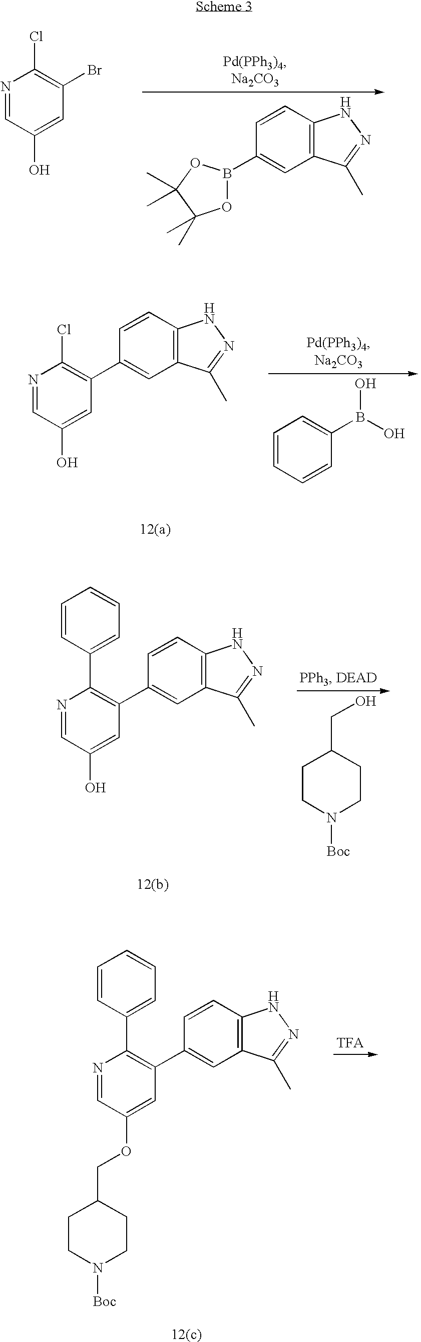

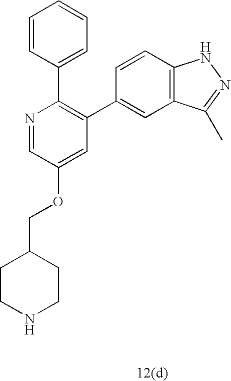

Another variant on the synthesis is to introduce alternative linker groups such as amines in place of ethers as exemplified in Scheme 4. For example, ipso-addition of an amine such as 1-(3-pyridinylmethyl)piperazine to a pyridine trifluoromethylsulfonate (triflate or TfO) intermediate such as 16(a) and elimination under microwave conditions in a solvent such as N-methyl2-pyrollidone (NMP) produces amine analogs such as 16(b).

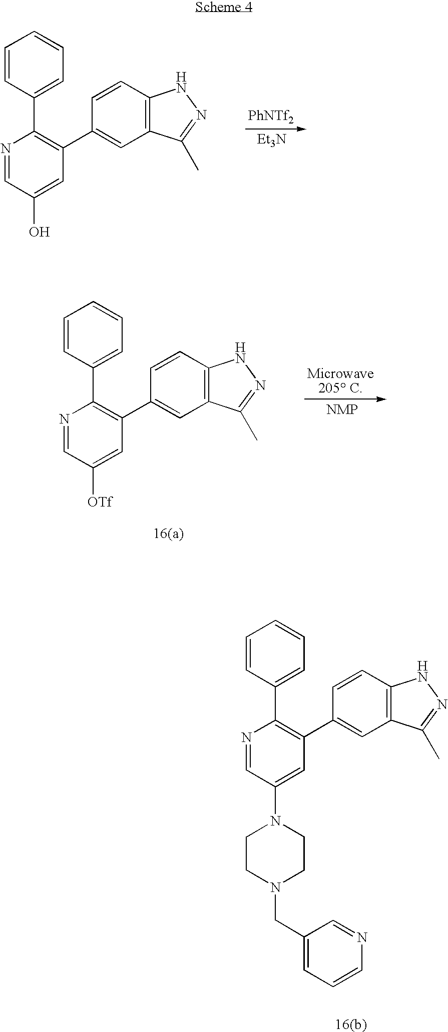

In addition, the aryl groups on the substituted pyridine may be further functionalized by further reactions such as acylation of a intermediate amines such as 25(b) to form amides such 25(c) as shown in Scheme 5, followed by deprotection steps.

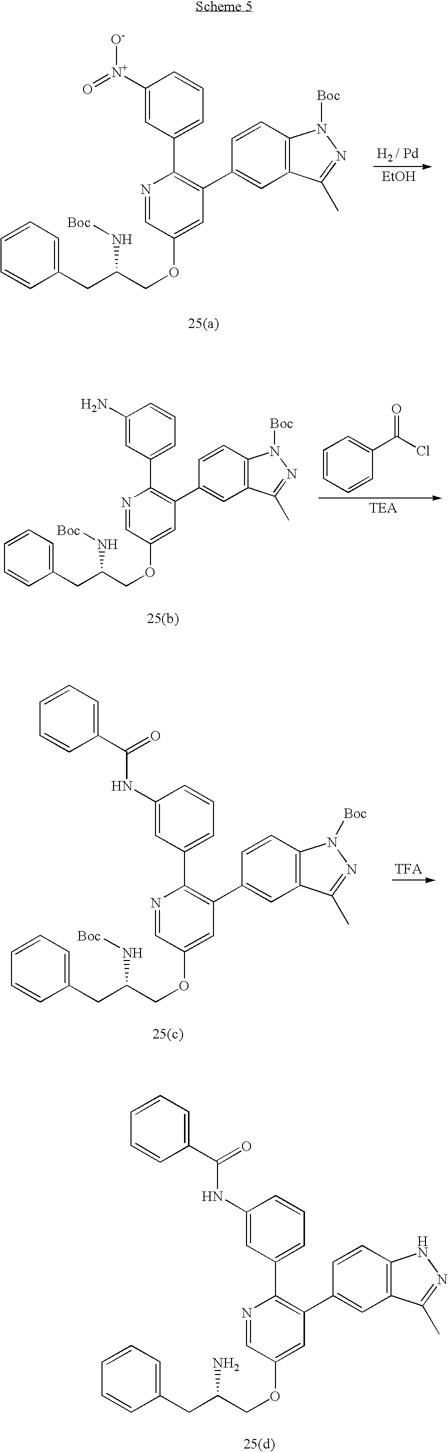

3-Substituted indazole analogs can be prepared by selective iodination of the parent indazole and Pd-mediated cross coupling steps (Scheme 6).

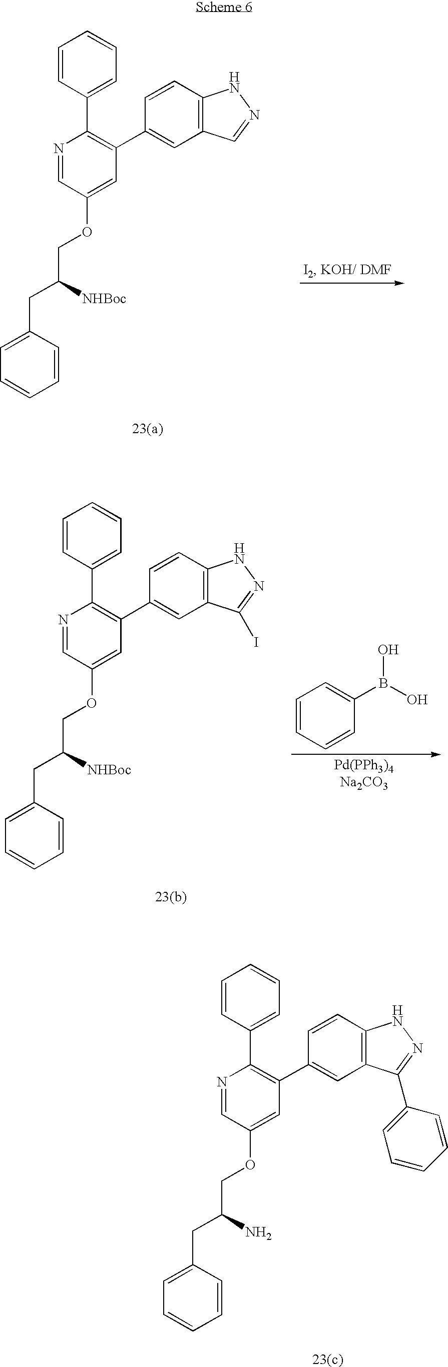

Also, N-alkylated analogs of the indazole such as 33(d) can be prepared by treating intermediate indazoles such as 16(a) with electrophilic reagents such as Meerwein's reagent followed by a Mitsunobu reaction as described above (Scheme 7), followed by deprotection steps.

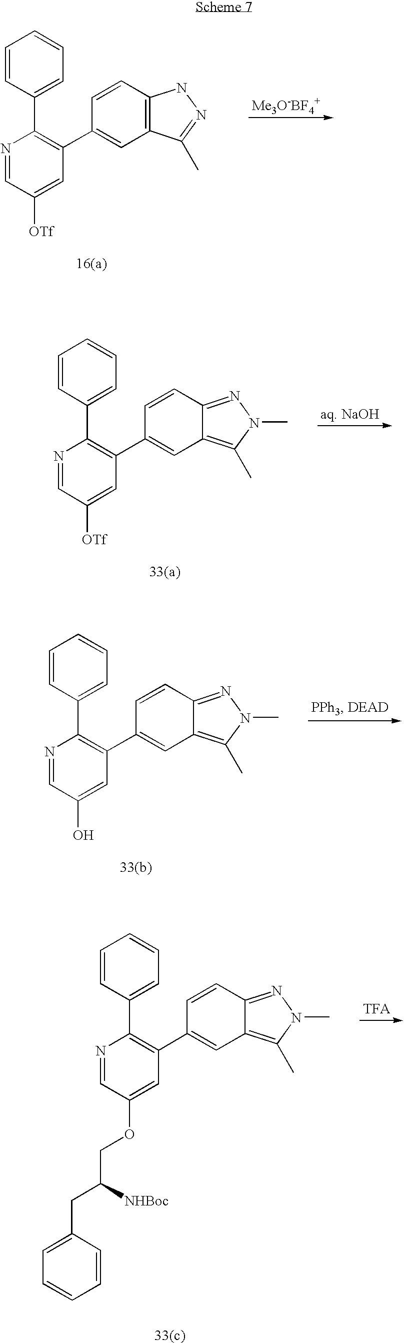

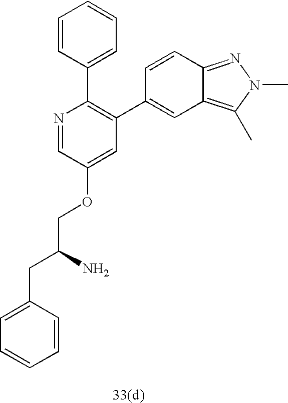

Indazoles may be further substituted by iodinating the 3-position using an iodinating reagent such as iodine and a base such as potassium hydroxide followed by a Pd-mediated cross coupling step such as Suzuki, Stille, Buchwald/Hartwig (JOC 2000, 65(4), 1158-1174), Negishi (Aus J Chem 2004, 57(1), 107), followed by deprotection steps.

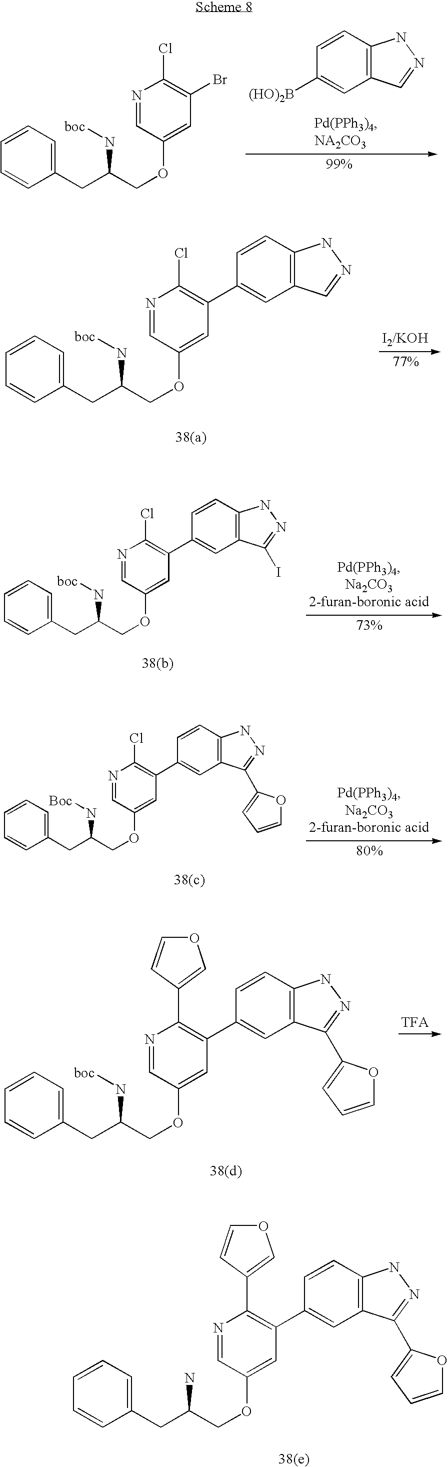

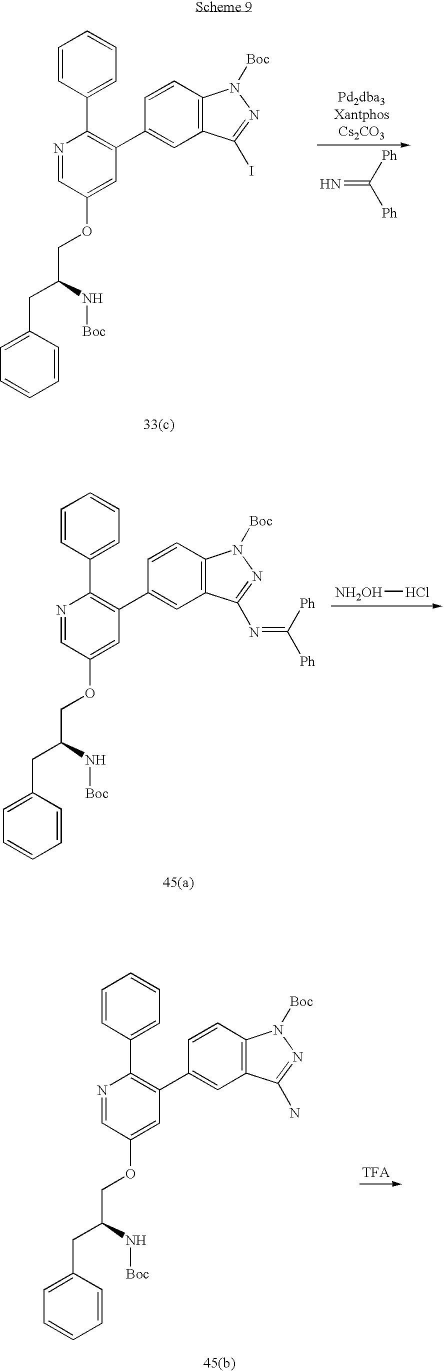

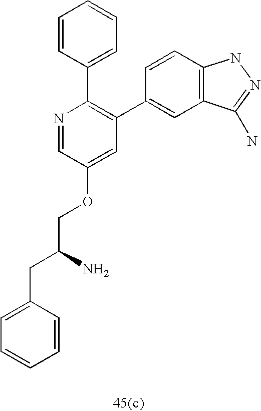

Ethers such as 69(a) can be prepared by Mitsunobu coupling with hydroxy-pyridines such as 2-chloro-3-bromo-5-hydroxy-pyridine and alcohols such as Boc-(2S)-2-amino-3-(3-indole)-1-propanol (Scheme 10). Then, using Pd-mediated cross coupling methods and deprotection steps, desired compounds such as 69(b) can be prepared.

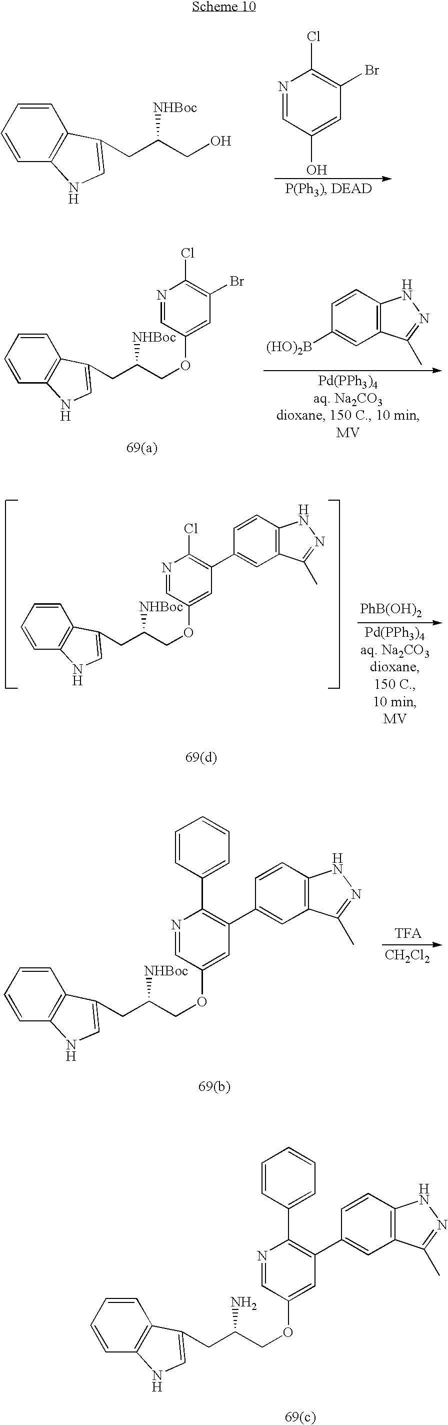

Amines such as 70(b) can be prepared by reductive amination using aldehydes such as 3-phenyl-propanal and a reducing agent such as triacetoxyborohydride (Scheme 11).

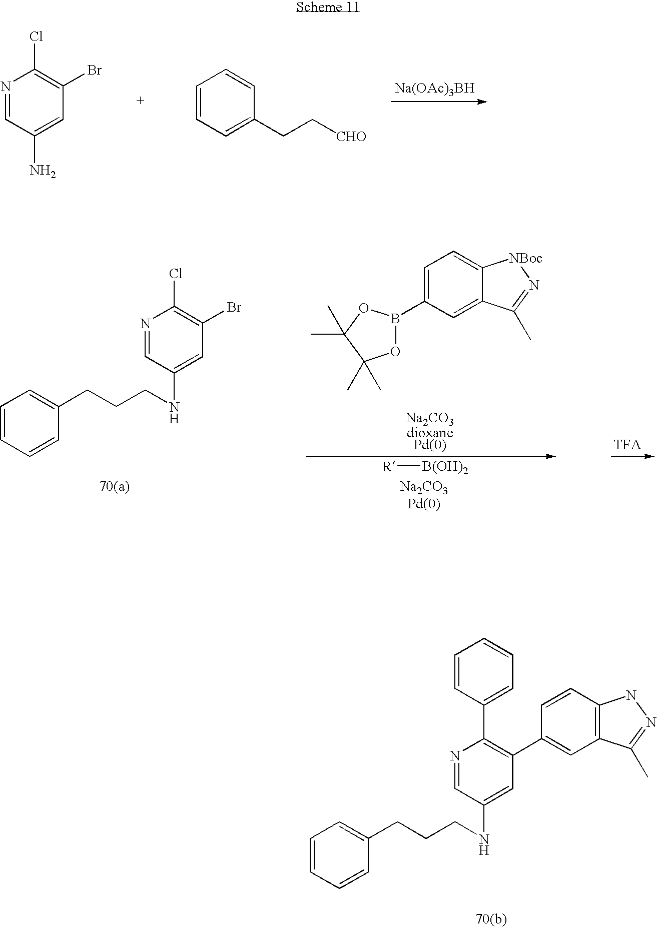

The amine may be further functionalized with sulfonylating agents such as methylsulfonyl chloride (Scheme 12), followed by Pd-mediated cross coupling and deprotection steps.

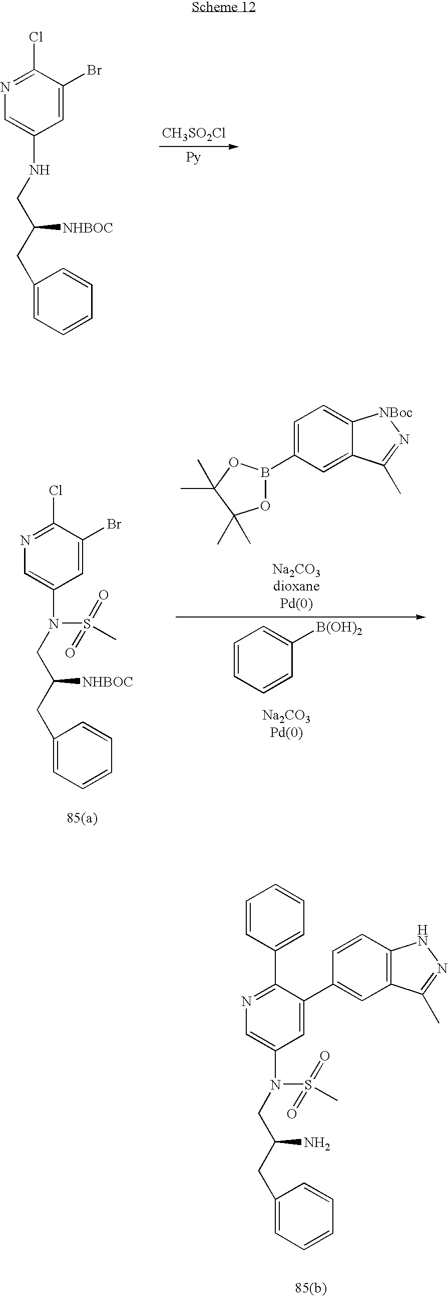

Amines such as 82(c) may also be prepared by reductive amination between amines such as 2-chloro-3-bromo-5-amino-pyridine and aldehydes such as 1,1-dimethylethyl [(1S)-1-formyl-2-(1H-indol-3-yl)ethyl]carbamate with reducing agents such as sodium triacetoxyborohydride or sodium borohydride, followed by Pd-mediated cross coupling reactions using the methods of Suzuki, Stille, Buchwald, or Negishi, and final deprotection steps such as Boc removal with trifluoroacetic acid or HCl (Scheme 13).

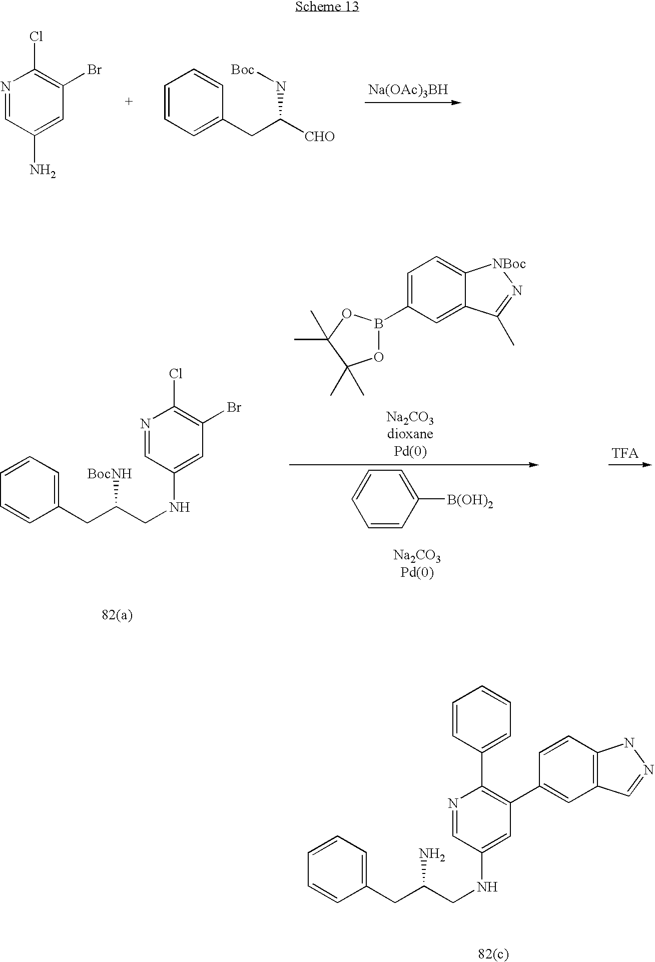

Amides such as 105(d) can be prepared by amide forming coupling reactions between carboxylic acids and amines such as 2-chloro-3-bromo-5-amino-pyridine using a coupling reagent such as EDC (1-(3-Dimethylaminopropyl)-3-ethylcarbodiimide hydrochloride)/HOAT (1-Hydroxy-7-azabenzotriazole), DCC (1,3-Dicyclohexylcarbodiimide), DIC (1,3-Diisopropylcarbodiimide), HBTU (O-Benzotriazol-1-yl-N,N,N′N′-tetramethyluronium hexafluorophosphate), HATU (O-7-Azabenzotriazol-1-yl-N,N,N′N′-tetramethyluronium hexafluorophosphate), etc. (Scheme 14).

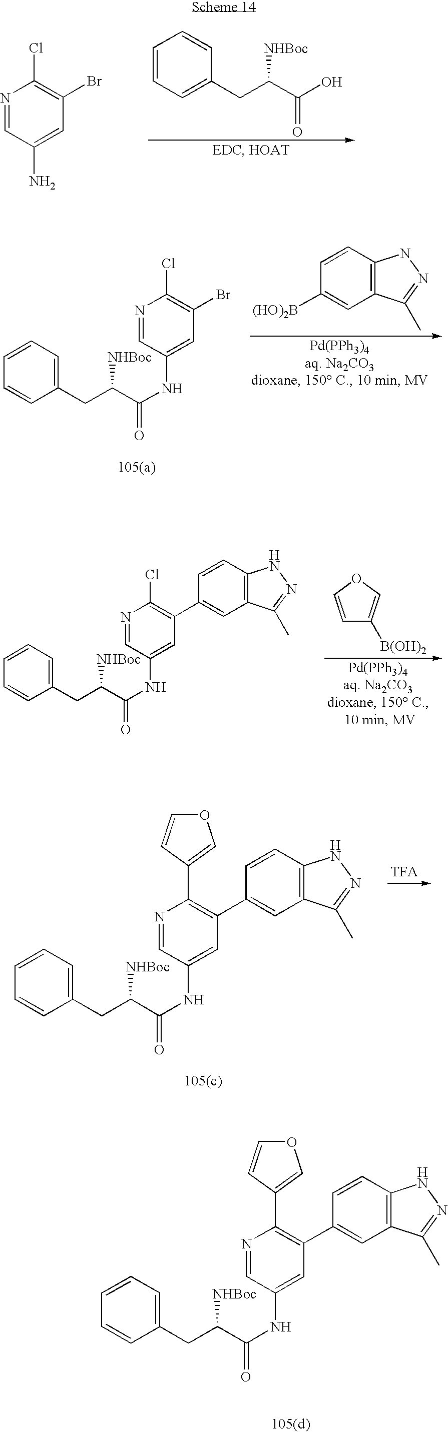

Ethers such as 107(b) can be prepared by Mitsunobu coupling with hydroxy-pyridines such as 2-chloro-3-bromo-5-hydroxy-pyridine and alcohols such as Boc-(2S)-2-amino-3-(3-thiophene)-1-propanol (Scheme 15). Then, using the methods described in Scheme 1, the desired compounds can be prepared.

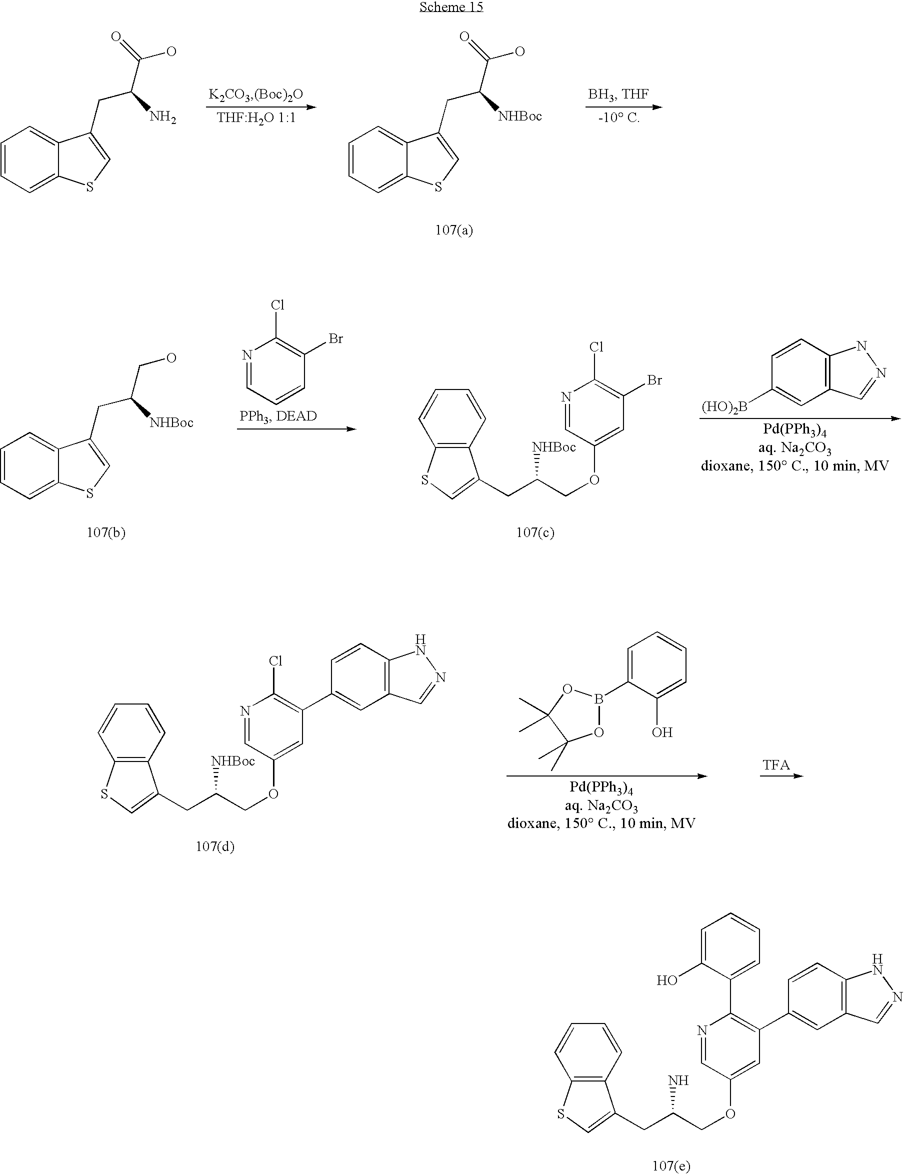

Ethers such as 109(b) can be prepared by Mitsunobu coupling with hydroxy-pyridines such as 2-chloro-3-bromo-5-hydroxy-pyridine and alcohols such as Boc-(2S)-2-amino-3-(t-butyl-dimethylsilyloxy)-1-propanol (Scheme 16). Then, using the Pd-mediated cross coupling reactions, the pyridine can be substituted. Deprotection of the silyl ether protecting group with a fluoride such as tetrabutylammonium fluoride and Mitsunobu cyclization reaction forms the intermediate Boc-aziridine 109(f). The aziridine then reacts with Grignard reagents such as 2-naphthyl magnesium bromide to form the 3-aryl substituted-2-Boc-amino-propyl ethers, which are then deprotected to provide desired compounds such as as 109(g).

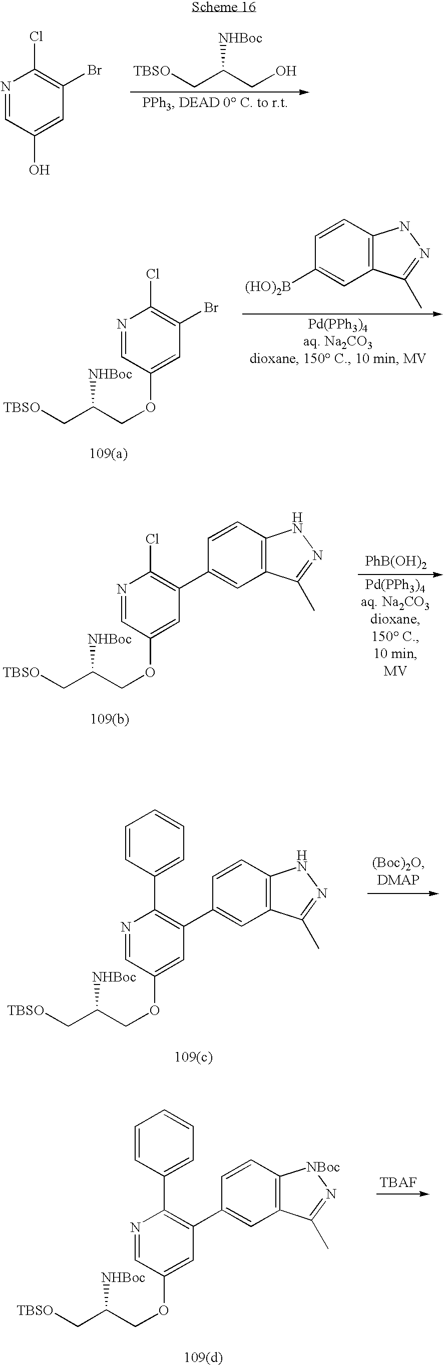

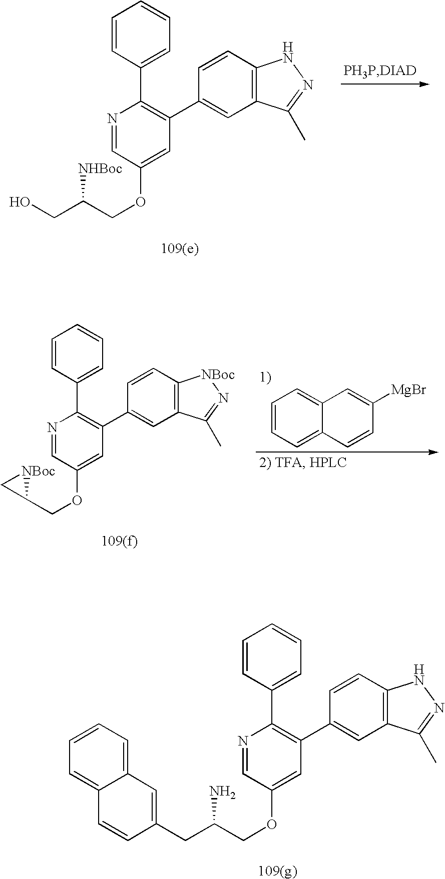

Amines such as 111(b) can be prepared by reductive amination using aldehydes such as Boc-(2S)-2-amino-3-(3-indole)-1-propanal and a reducing agent such as triacetoxyborohydride (Scheme 17). Then, Pd-mediated cross coupling reactions and standard deprotection steps provide the desired compounds such as 111(d).

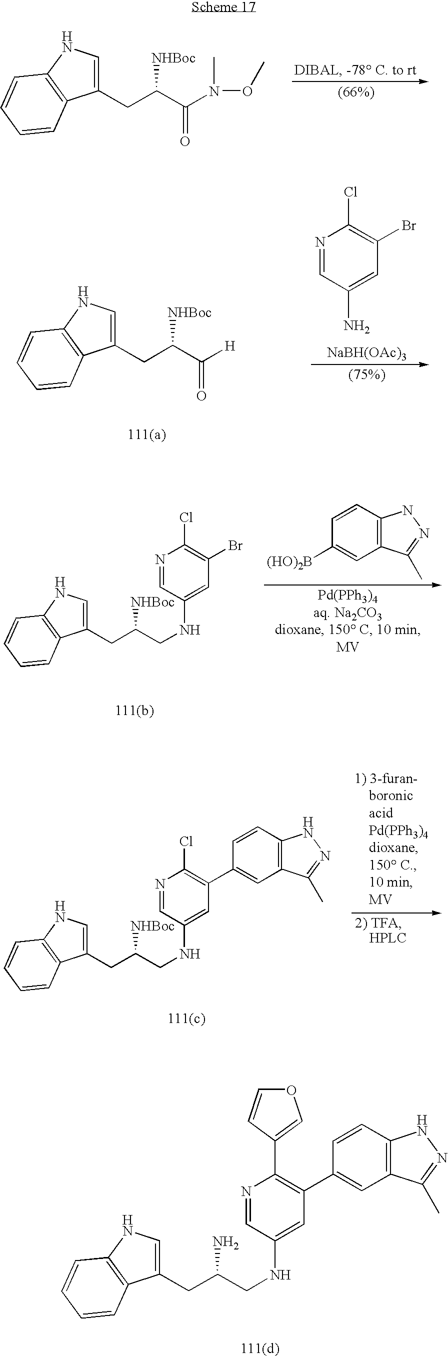

Ethers such as 112(a) can also be prepared by alkylation with (2S)-2-oxiranylmethyl 2-nitrobenzenesulfonate (Scheme 18). The epoxide can then be opened by Grignard reagents such as phenyl magnesium chloride to provide alcohol intermediates such as 112(b). Pd-mediated cross-coupling reactions and deprotection steps provide the desired compounds such as 112(c).

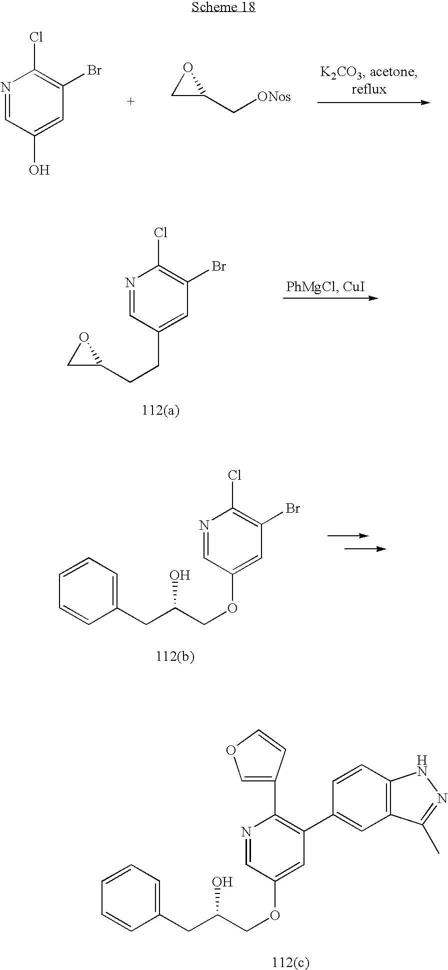

1H-thieno[3,2-c]pyrazole intermediates 121(c) and (d) can be prepared by cyclization of Boc-protected hydrazone 121(b) (Scheme 19). Stannylation and Pd-mediated cross coupling to halogenated pyridine intermediate 69(a), followed by a second Pd-mediated cross coupling step and deprotection steps provide the desired compounds such as 121(i).

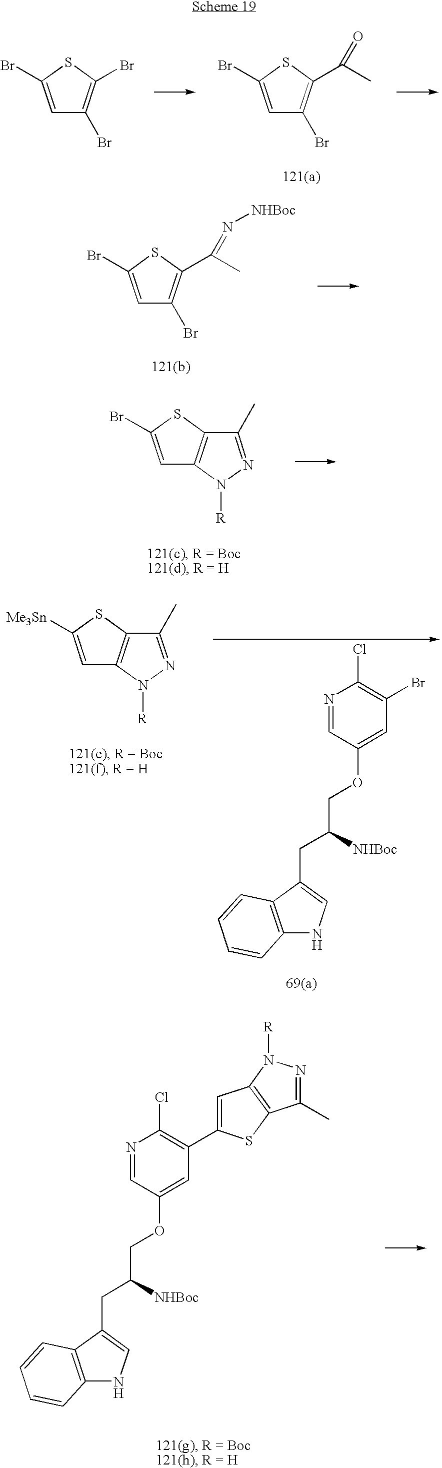

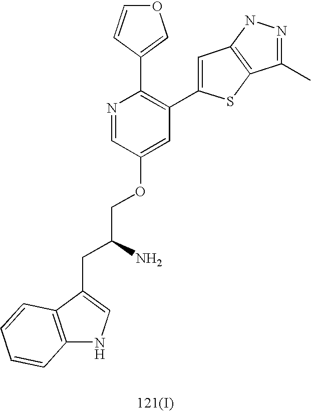

Palladium-mediated Buchwald/Hartwig reactions can be used to functional the 3-position of indazoles such as 122(b) to introduce substituted amines such as 4-amino-pyridine (Schemes 20) or amides such as benzamide (Scheme 21, JOC, 2004, 69(17), 5578-5587). Following deprotection steps, desired compounds such as 122(d) or 123(b) can be prepared.

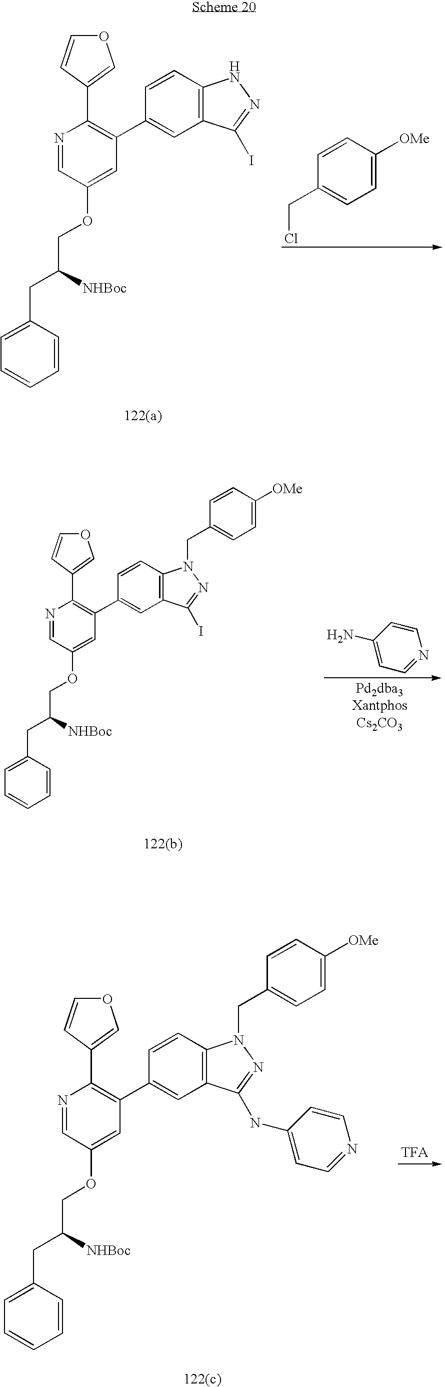

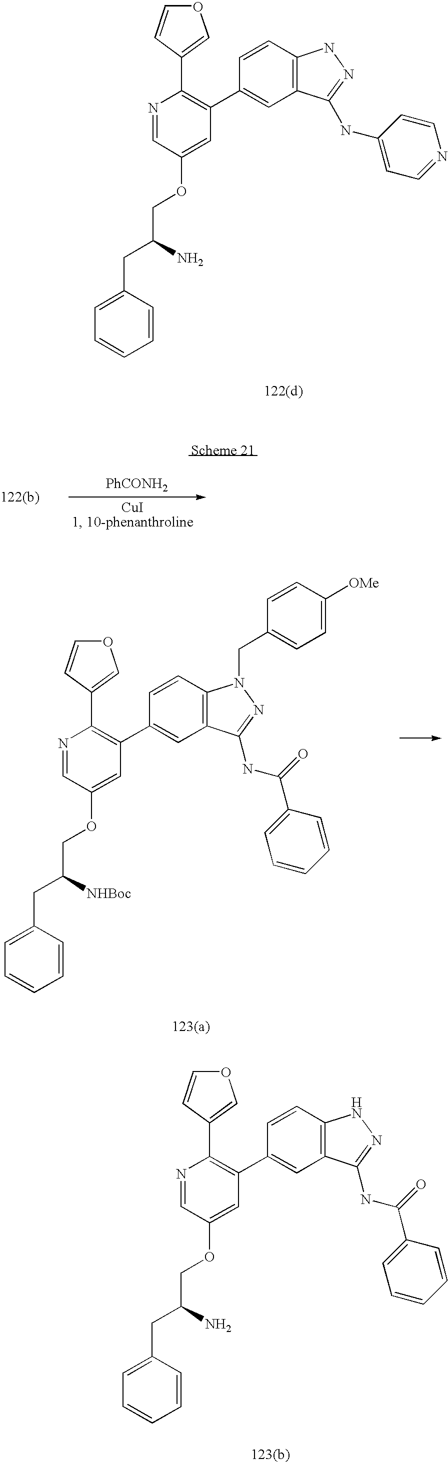

3-Ethyl-indazole intermediate 133(d) can be prepared by addition of ethyl magnesium bromide to 5-bromo-2-fluoro-benzaldehyde to form alcohol intermediate 133(a), followed by oxidation with an oxidant such as Dess-Martin periodinane to produce ketone 133(b), hydrazone formation, and cyclization (Scheme 22).

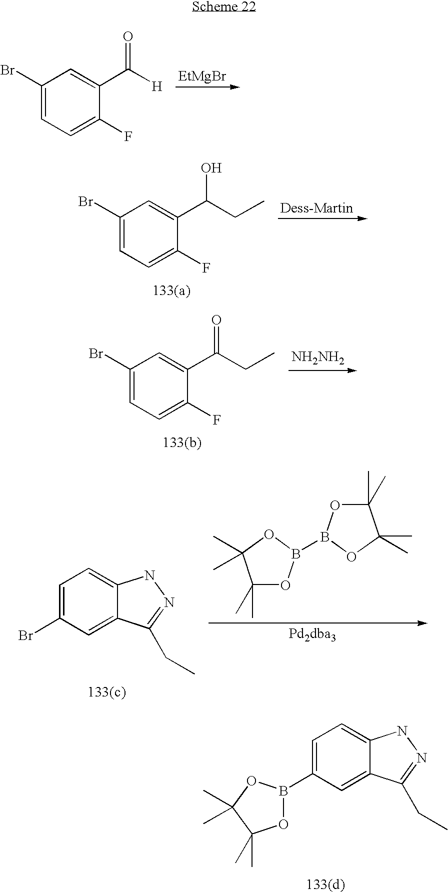

Methylation of the nitrogen can be conducted by alkylation of nosyl-protected amine 156(a) using methyl iodide and base (Scheme 23). Pd-mediated cross-coupling reactions followed by deprotection of the nosyl group with a mercaptan such as phenyl mercaptan provides the desired compounds such as 156(d).

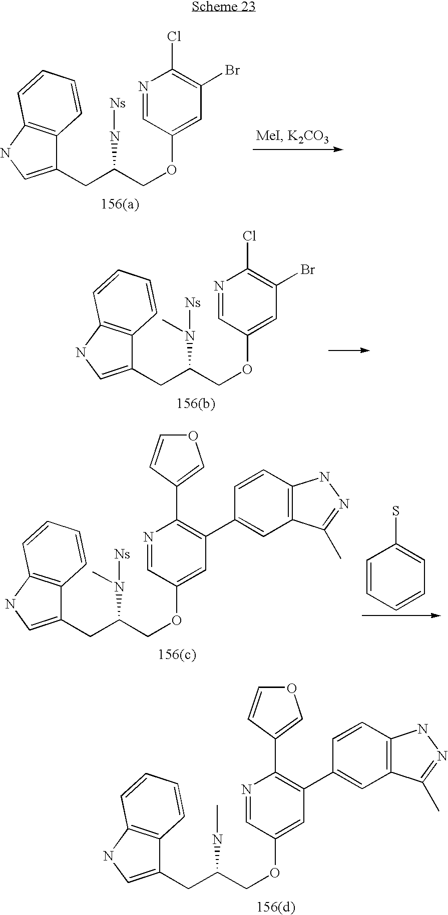

Ethers such as 165(b) can be prepared by Mitsunobu coupling with hydroxy-pyridines such as 2-chloro-3-bromo-5-hydroxy-pyridine and alcohols such as Boc-(2S)-3-amino-4-(4-trifluoromethylphenyl)-1-butanol (Scheme 24). Then, using Pd-mediated cross coupling methods and deprotection steps, desired compounds such as 165(d) can be prepared.

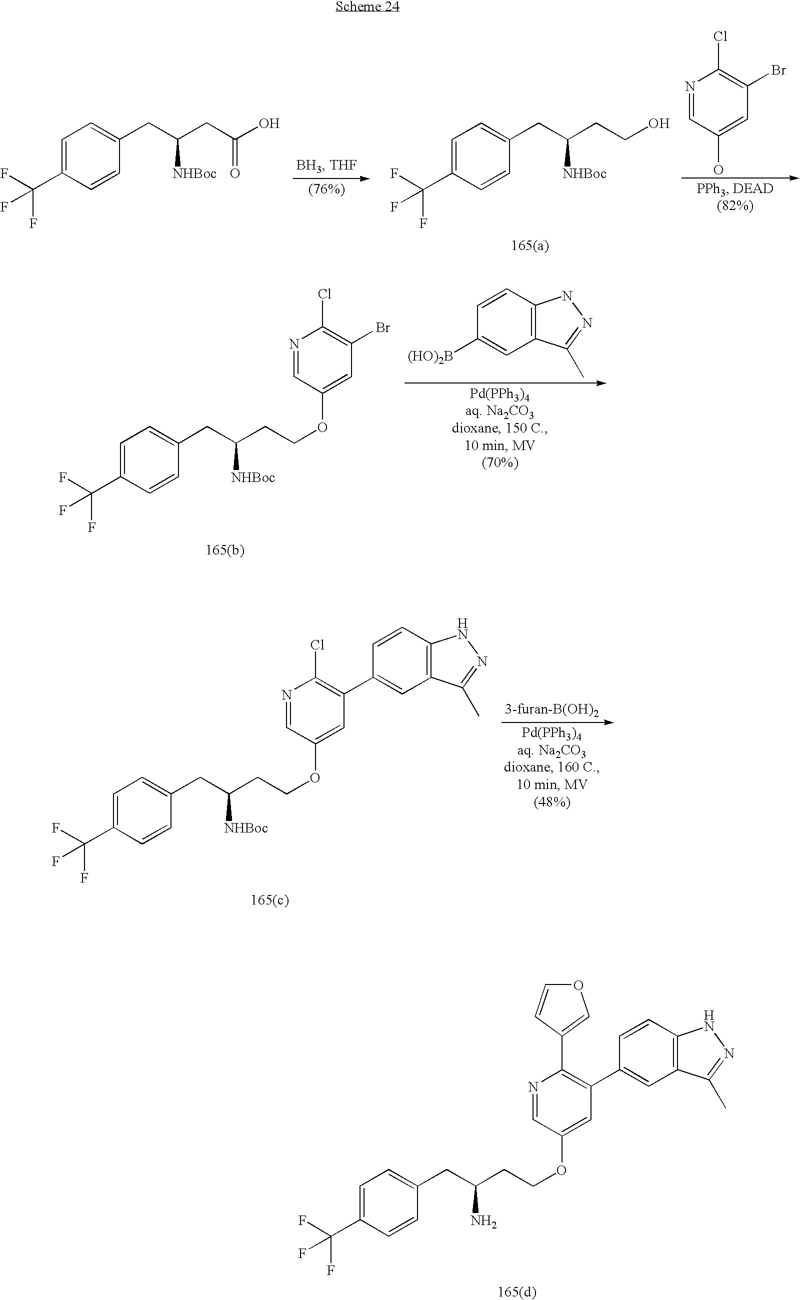

Ether intermediate 167(b) can be prepared by Mitsunobu coupling with hydroxy-pyridines such as 2-chloro-3-bromo-5-hydroxy-pyridine and alcohols such as Boc-(2S)-2-amino-pent-4-yn-1-ol (Scheme 25 and Scheme 26). Silylation of the alkyne followed by a Pd-mediated cross coupling reaction provides intermediate 167(d), which is then subjected to the indole formation reaction of R. Larock (JOC 1998, 63(22), 7652-7662), followed by a second Pd-mediated cross coupling reaction, and deprotection steps to provide desired compounds such as 167(e).

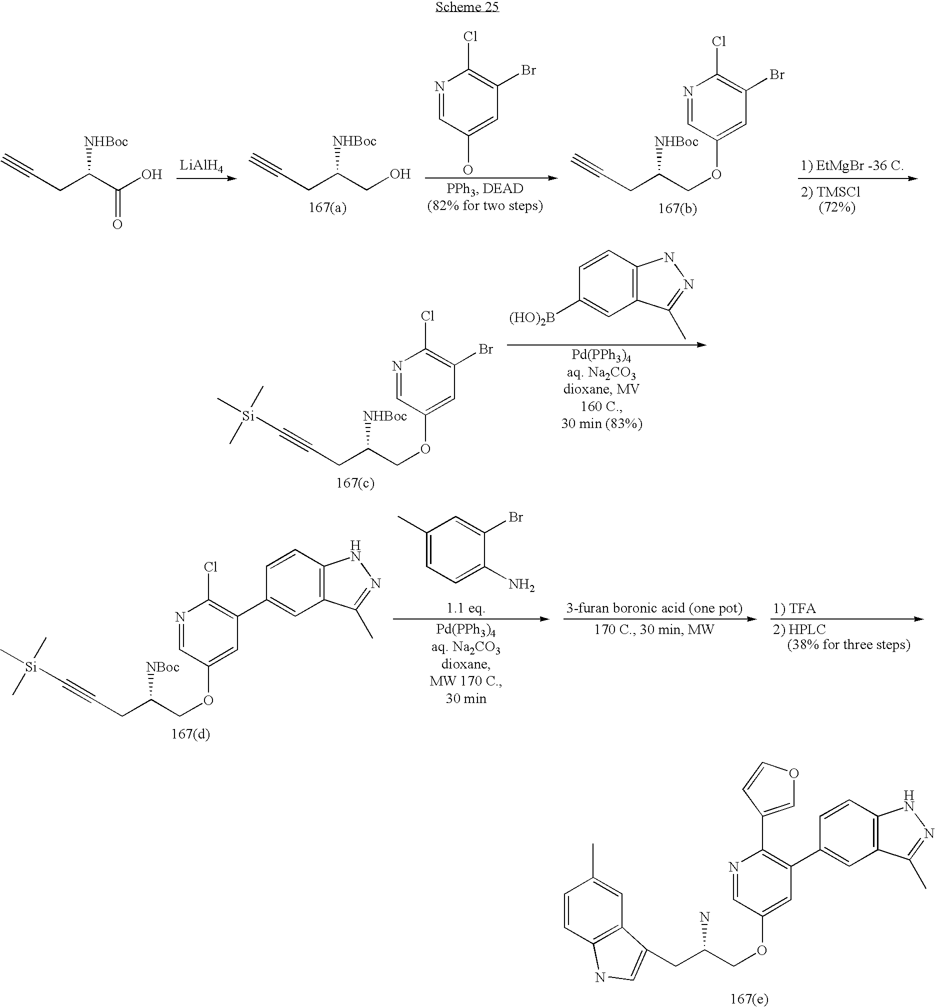

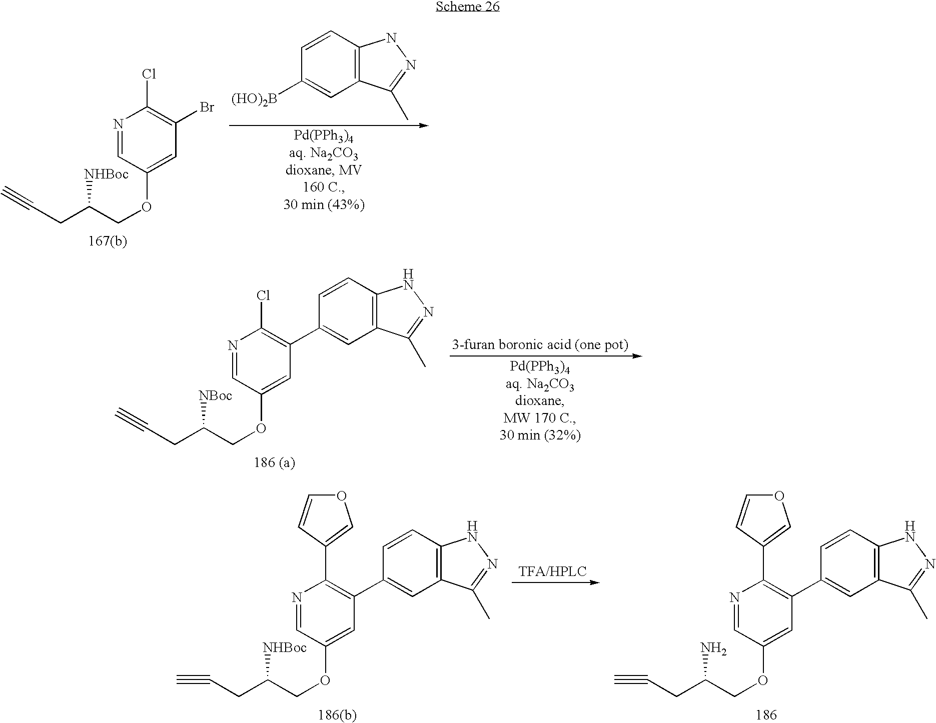

The 4-aza-indazole intermediate 169(b) is prepared by cyclization of hydrazone generated from 1-(3-fluoro-2-pyridinyl)ethanone (Scheme 27). N-oxidation of the pyridine followed by treatment with phosphorus oxychloride provides chloro-4-aza-indazole intermediate 169(e).

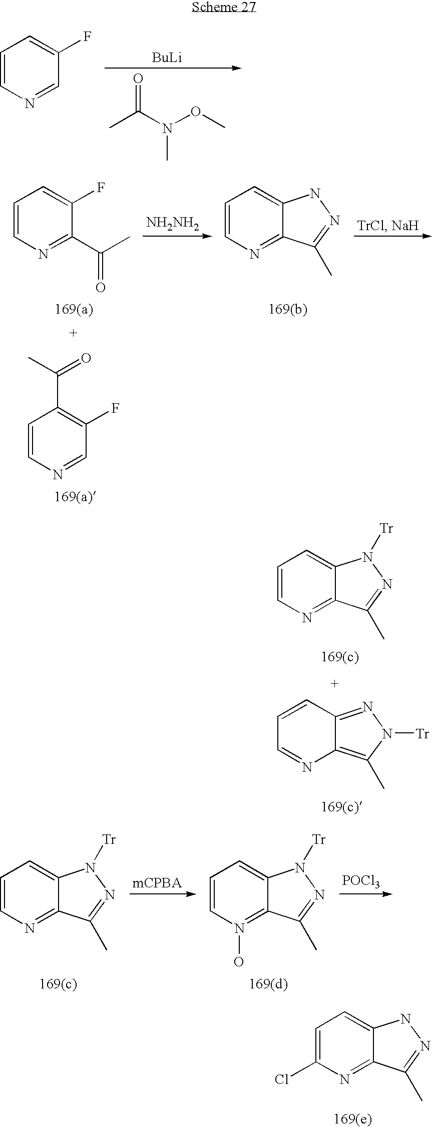

Halogenated pyridine intermediate 70(a) is selectively borylated and coupled to 169(e) to produce the 3-substituted pyridine intermediate 169(f) (Scheme 28). A second Pd-mediated cross coupling reaction, and deprotection step provide desired compounds such as 170.

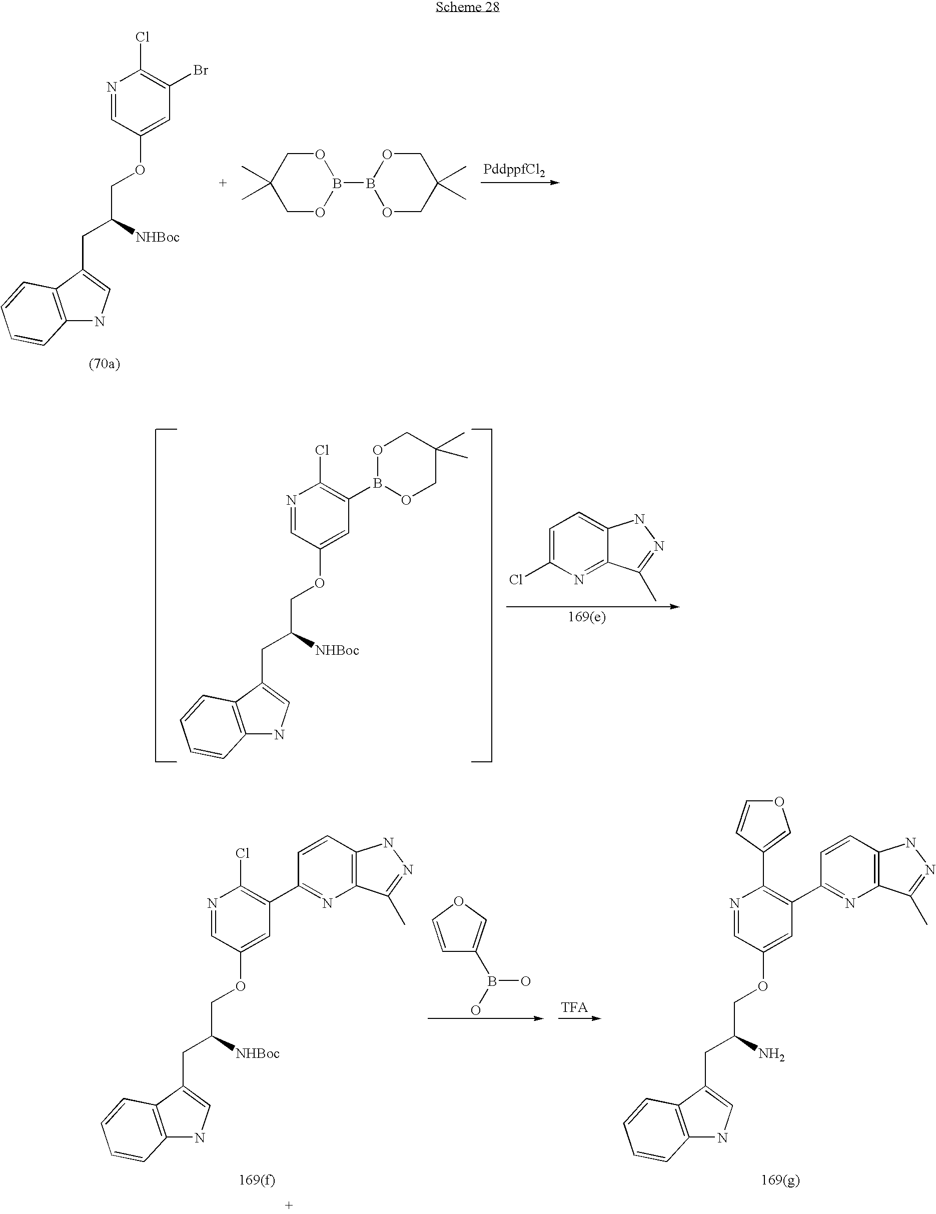

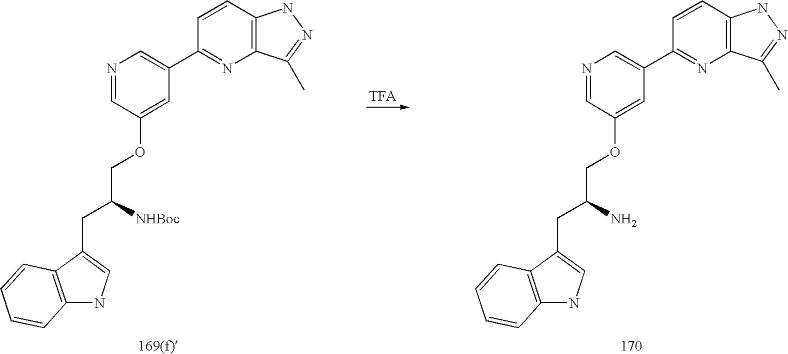

Zinc cyanide addition to 3-iodo-indazole intermediate 122(b), followed by treatment with trifluoroacetic acid provides 3-nitrile 171(b) and 3-amide 172 (Scheme 29).

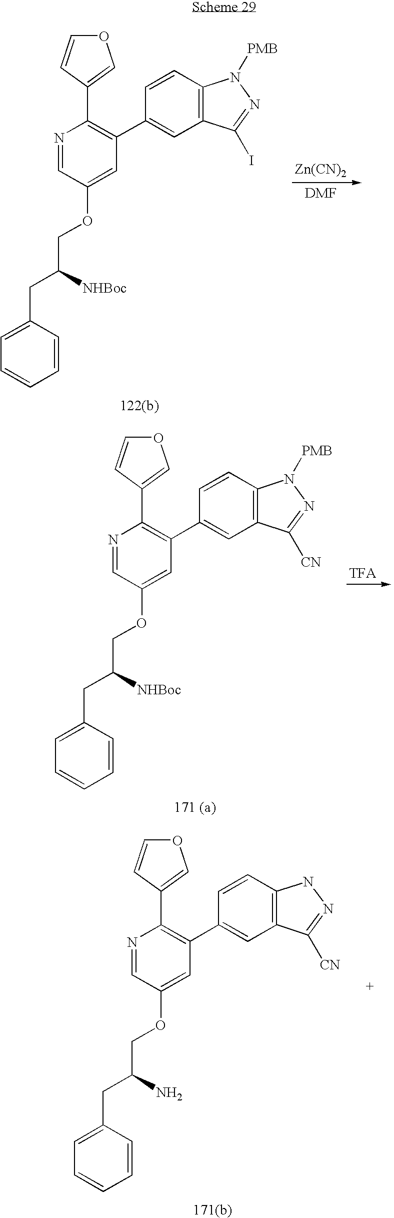

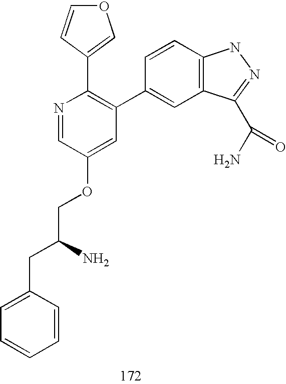

Nitro phenol intermediate 190(a) can be prepared by selective bromination of 2-fluoro-4-nitro-phenol. Protection of the phenol as a benzyl ether followed by Pd-mediated cross coupling reaction provides intermediate 190(c). The benzyl group is removed under the Suzuki reaction conditions. Triflate formation with N-phenyltriflimide followed by a second Pd-mediated cross-coupling reaction provides aniline intermediate 190(e). Reduction of the nitro group occurs under the Suzuki reaction conditions. Reductive amination and final deprotection provides desired compounds such as 190(g).

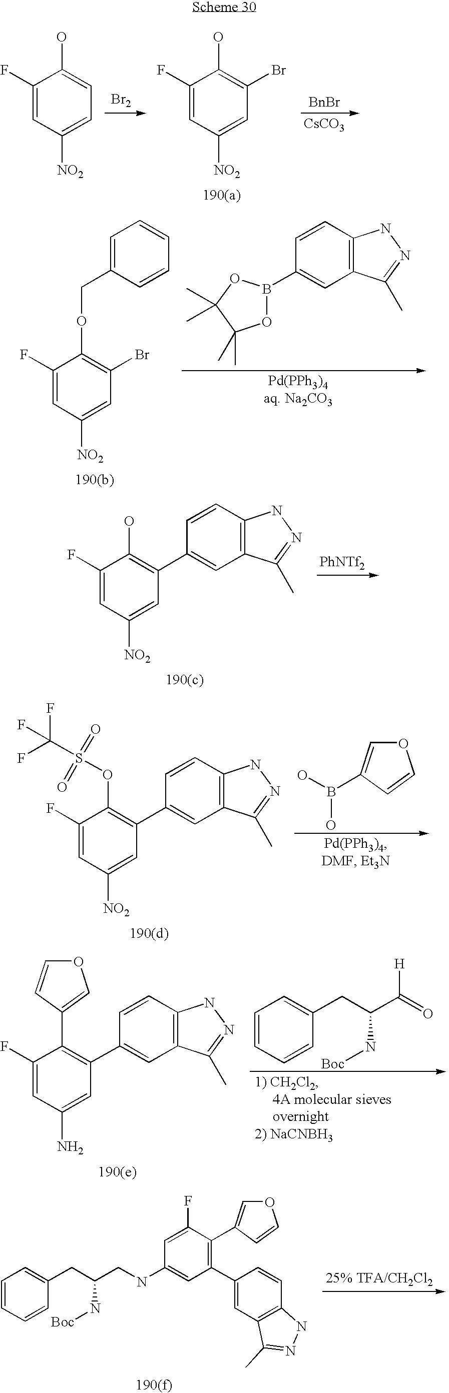

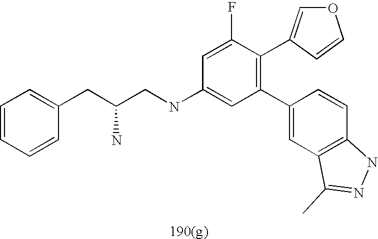

Stille coupling with [1-(ethyloxy)ethenyl](triethyl)stannane and halogenated pyridine intermediate 192(a), followed by treatment to dilute acid, then hydrazine provides 7-aza-indazole intermediate 192(b). Deprotection of the phenol, triflate formation, and boronic acid formation, followed by Pd-mediated cross coupling reactions to the halogenated pyridine intermediate 70(a) and deprotection steps provide desired compounds such as 192(g).

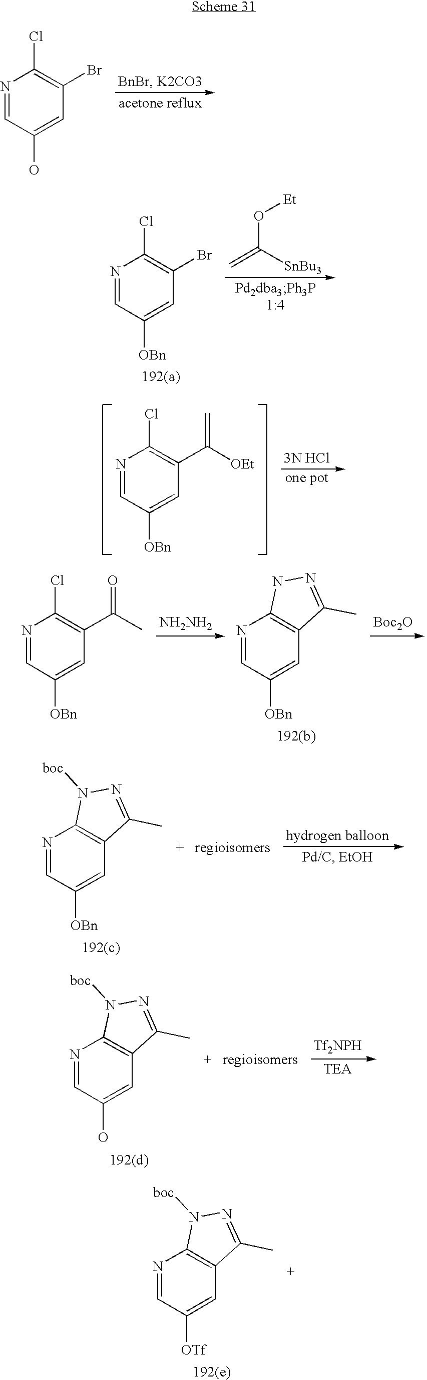

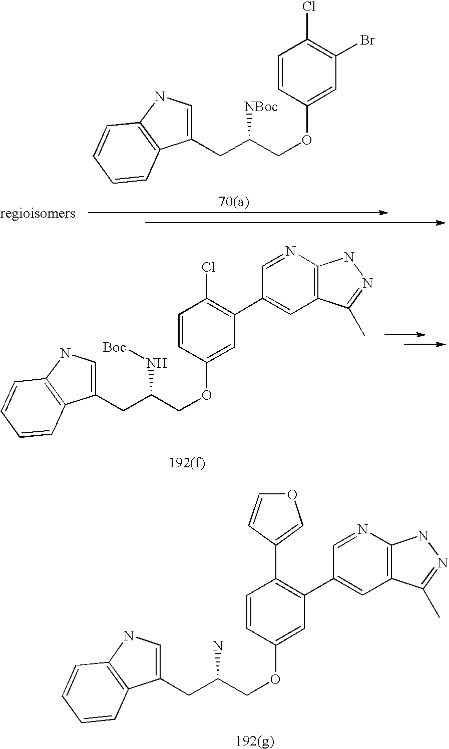

By the term “co-administering” and derivatives thereof as used herein is meant either simultaneous administration or any manner of separate sequential administration of an AKT inhibiting compound, as described herein, and a further active ingredient or ingredients, known to be useful in the treatment of cancer, including chemotherapy and radiation treatment, or to be useful in the treatment of arthritis. The term further active ingredient or ingredients, as used herein, includes any compound or therapeutic agent known to or that demonstrates advantageous properties when administered to a patient in need of treatment for cancer or arthritis. Preferably, if the administration is not simultaneous, the compounds are administered in a close time proximity to each other. Furthermore, it does not matter if the compounds are administered in the same dosage form, e.g. one compound may be administered topically and another compound may be administered orally.

Typically, any anti-neoplastic agent that has activity versus a susceptible tumor being treated may be co-administered in the treatment of cancer in the present invention. Examples of such agents can be found in Cancer Principles and Practice f Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors.

Examples of a further active ingredient or ingredients for use in combination with the presently invented AKT inhibiting compounds are chemotherapeutic agents.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)-N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani et al. J. Am. Chem, Soc., 93:2325. 1971), who characterized its structure by chemical and X-ray crystallographic methods. One mechanism for its activity relates to paclitaxel's capacity to bind tubulin, thereby inhibiting cancer cell growth. Schiff et al., Proc. Natl, Acad, Sci. USA, 77:1561-1565 (1980); Schiff et al., Nature, 277:665-667 (1979); Kumar, J. Biol, Chem, 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986”, Attaur-Rahman, P. W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. Intem, Med., 111:273, 1989) and for the treatment of breast cancer (Holmes et al., J. Nat. Cancer Inst., 83:1797, 1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p.16-23, 1995).

Docetaxel, (2R,3S)-N-carboxy-3-phenylisoserine,N-tert-butyl ester, 13-ester with 5β-20-epoxy-1,2β,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine [R—(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine [1,1-cyclobutane-dicarboxylate(2-)—O,O′], is commercially available as PARAPLATIN® as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias. Alopecia, nausea, vomiting and leukopenia are the most common dose limiting side effects of cyclophosphamide.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also know as Actinomycin D, is commercially available in injectable form as COSMEGEN®. Dactinomycin is indicated for the treatment of Wilm's tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma. Myelosuppression is the most common dose limiting side effect of daunorubicin.

Doxorubicin, (8S, 10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid turnors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers. Myelosuppression is the most common side effect of etoposide. The incidence of leucopenia tends to be more severe than thrombocytopenia.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leucopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mecaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine). Cytarabine induces leucopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leucopenia, thrombocytopenia, and anemia, is the most common dose limiting side effect of thioguanine administration. However, gastrointestinal side effects occur and can be dose limiting. Other purine analogs include pentostatin, erythrohydroxynonyladenine, fludarabine phosphate, and cladribine.

Gemcitabine, 2′-deoxy-2′,2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer. Myelosuppression, including leucopenia, thrombocytopenia, and anemia, is the most common dose limiting side effect of gemcitabine administration.

Methotrexate, N-[4[[(2,4-diamino-6-pteridinyl) methyl]methylamino] benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder. Myelosuppression (leucopenia, thrombocytopenia, and anemia) and mucositis are expected side effect of methotrexate administration.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR®.

Irinotecan is a derivative of camptothecin which binds, along with its active metabolite SN-38, to the topoisomerase I-DNA complex. It is believed that cytotoxicity occurs as a result of irreparable double strand breaks caused by interaction of the topoisomerase I:DNA:irintecan or SN-38 ternary complex with replication enzymes. Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I-DNA complex and prevents religation of singles strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

Also of interest, is the camptothecin derivative of formula A following, currently under development, including the racemic mixture (R,S) form as well as the R and S enantiomers: 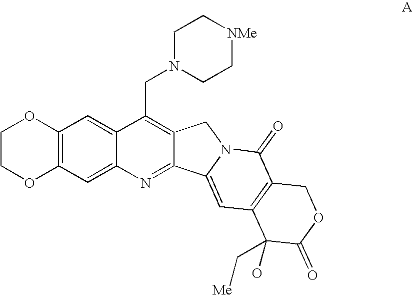
known by the chemical name “7-(4-methylpiperazino-methylene)-10,11-ethylenedioxy-20(R,S)-camptothecin (racemic mixture) or “7-(4-methylpiperazino-methylene)-10,11-ethylenedioxy-20(R)-camptothecin (R enantiomer) or “7-(4-methylpiperazino-methylene)-10,11-ethylenedioxy-20(S)-camptothecin (S enantiomer). Such compounds as well as related compounds are described, including methods of making, in U.S. Pat. Nos. 6,063,923; 5,342,947; 5,559,235; 5,491,237 and pending U.S. patent application Ser. No. 08/977,217 filed Nov. 24, 1997.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

Signal transduction pathway inhibitors are those inhibitors, which block or inhibit a chemical process which evokes an intracellular change. As used herein this change is cell proliferation or differentiation. Signal tranduction inhibitors useful in the present invention include inhibitors of receptor tyrosine kinases, non-receptor tyrosine kinases, SH2/SH3domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, vascular endothelial growth factor receptor (VEGFr), tyrosine kinase with immunoglobulin-like and epidermal growth factor homology domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine ki nase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth factor receptors as targets”, New Molecular Targets for Cancer Chemotherapy, ed. Workman, Paul and Kerr, David, CRC press 1994, London.

Tyrosine kinases, which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S. and Corey, S. J., (1999) Journal of Hematotherapy and Stem Cell Research 8 (5): 465-80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, P13-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, akt kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60.1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; U.S. Pat. No. 6,268,391; and Martinez-lacaci, L., et al, Int. J. Cancer (2000), 88(1), 44-52.

Inhibitors of Phosphotidyl inositol-3 Kinase family members including blockers of PI3-kinase, ATM, DNA-PK, and Ku are also useful in the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Also useful in the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras , thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9 (2) 99-102; and BioChim. Biophys. Acta, (19899) 1423(3):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al, Monoclonal Antibody Therapy for Solid Tumors, Cancer Treat. Rev., (2000), 26(4), 269-286); Herceptin® erbB2 antibody (see Tyrosine Kinase Signalling in Breast cancer:erbB Family Receptor Tyrosine Kniases, Breast cancer Res., 2000, 2(3), 176-183); and 2CB VEGFR2 specific antibody (see Brekken, R. A. et al, Selective Inhibition of VEGFR2 Activity by a monoclonal Anti-VEGF antibody blocks tumor growth in mice, Cancer Res. (2000) 60, 5117-5124).

Non-receptor kinase angiogenesis inhibitors may also find use in the present invention. Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Thus, the combination of an erbB2/EGFR inhibitor with an inhibitor of angiogenesis makes sense. Accordingly, non-receptor tyrosine kinase inhibitors may be used in combination with the EGFR/erbB2 inhibitors of the present invention. For example, anti-VEGF antibodies, which do not recognize VEGFR (the receptor tyrosine kinase), but bind to the ligand; small molecule inhibitors of integrin (alphaᵥ beta₃) that will inhibit angiogenesis; endostatin and angiostatin (non-RTK) may also prove useful in combination with the disclosed erb family inhibitors. (See Bruns C J et al (2000), Cancer Res., 60: 2926-2935; Schreiber A B, Winkler M E, and Derynck R. (1986), Science, 232: 1250-1253; Yen L et al. (2000), Oncogene 19: 3460-3469).

Agents used in immunotherapeutic regimens may also be useful in combination with the compounds of formula (I). There are a number of immunologic strategies to generate an immune response against erbB2 or EGFR. These strategies are generally in the realm of tumor vaccinations. The efficacy of immunologic approaches may be greatly enhanced through combined inhibition of erbB2/EGFR signaling pathways using a small molecule inhibitor. Discussion of the immunologic/tumor vaccine approach against erbB2/EGFR are found in Reilly R T et al. (2000), Cancer Res. 60: 3569-3576; and Chen Y, Hu D, Eling D J, Robbins J, and Kipps T J. (1998), Cancer Res. 58: 1965-1971.

Agents used in proapoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family (i.e., mcl-1). Therefore, strategies designed to downregulate the expression of bcl-2 in tumors have demonstrated clinical benefit and are now in Phase II/III trials, namely Genta's G3139 bcl-2 antisense oligonucleotide. Such proapoptotic strategies using the antisense oligonucleotide strategy for bcl-2 are discussed in Water J S et al. (2000), J. Clin. Oncol. 18: 1812-1823; and Kitada S et al. (1994), Antisense Res. Dev. 4: 71-79.

Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania et al, Exp. Opin. Ther. Patents (2000) 10(2):215-230.

In one embodiment, the cancer treatment method of the claimed invention includes the co-administration a compound of formula I and/or a pharmaceutically acceptable salt, hydrate, solvate or pro-drug thereof and at least one anti-neoplastic agent, such as one selected from the group consisting of anti-microtubule agents, platinum coordination complexes, alkylating agents, antibiotic agents, topoisomerase 11 inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, and cell cycle signaling inhibitors.

Because the pharmaceutically active compounds of the present invention are active as AKT inhibitors they exhibit therapeutic utility in treating cancer and arthritis.

Suitably, the present invention relates to a method for treating or lessening the severity of a cancer.

Suitably, the present invention relates to a method for treating or lessening the severity of a cancer selected from brain (gliomas), glioblastomas, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

Suitably, the present invention relates to a method for treating or lessening the severity of a cancer selected from ovarian, pancreatic and prostate.

Isolation and Purification of His-tagged AKT1 (aa 136-480)

Insect cells expressing His-tagged AKT1 (aa 136-480) were lysed in 25 mM HEPES, 100 mM NaCl, 20 mM imidazole; pH 7.5 using a polytron (5 mLs lysis buffer/g cells). Cell debris was removed by centrifuging at 28,000×g for 30 minutes. The supernatant was filtered through a 4.5-micron filter then loaded onto a nickel-chelating column pre-equilibrated with lysis buffer. The column was washed with 5 column volumes (CV) of lysis buffer then with 5 CV of 20% buffer B, where buffer B is 25 mM HEPES, 100 mM NaCl, 300 mM imidazole; pH 7.5. His-tagged AKT1 (aa 136-480) was eluted with a 20-100% linear gradient of buffer B over 10 CV. His-tagged AKT1 (136-480) eluting fractions were pooled and diluted 3-fold with buffer C, where buffer C is 25 mM HEPES, pH 7.5. The sample was then chromatographed over a Q-Sepharose HP column pre-equilibrated with buffer C. The column was washed with 5 CV of buffer C then step eluted with 5 CV 10% D, 5 CV 20% D, 5 CV 30% D, 5 CV 50% D and 5 CV of 100% D; where buffer D is 25 mM HEPES, 1000 mM NaCl; pH 7.5. His-tagged AKT1 (aa 136-480) containing fractions were pooled and concentrated in a 10-kDa molecular weight cutoff concentrator. His-tagged AKT1 (aa 136-480) was chromatographed over a Superdex 75 gel filtration column pre-equilibrated with 25 mM HEPES, 200 mM NaCl, 1 mM DTT; pH 7.5. His-tagged AKT1 (aa 136-480) fractions were examined using SDS-PAGE and mass spec. The protein was pooled, concentrated and frozen at −80° C.

His-tagged AKT2 (aa 138-481) and His-tagged AKT3 (aa 135-479) were isolated and purified in a similar fashion.

AKT Enzyme Assay

Compounds of the present invention are tested for AKT 1, 2, and 3 protein serine kinase inhibitory activity in substrate phosphorylation assays. This assay examines the ability of small molecule organic compounds to inhibit the serine phosphorylation of a peptide substrate. The substrate phosphorylation assays use the catalytic domains of AKT 1, 2, or 3. AKT 1, 2 and 3 are also commercially available from Upstate USA, Inc. The method measures the ability of the isolated enzyme to catalyze the transfer of the gamma-phosphate from ATP onto the serine residue of a biotinylated synthetic peptide SEQ. ID NO: 1 (Biotin-ahx-ARKRERAYSFGHHA-amide). Substrate phosphorylation is detected by the following procedure:

Assays are performed in 384well U-bottom white plates. 10 nM activated AKT enzyme is incubated for 40 minutes at room temperature in an assay volume of 20 ul containing 50 mM MOPS, pH 7.5, 20 mM MgCl₂, 4 uM ATP, 8 uM peptide, 0.04 uCi [g-³³P] ATP/well, 1 mM CHAPS, 2 mM DTT, and 1 ul of test compound in 100% DMSO. The reaction is stopped by the addition of 50 ul SPA bead mix (Dulbecco's PBS without Mg²⁺ and Ca²⁺, 0.1% Triton X-100, 5 mM EDTA, 50 uM ATP, 2.5 mg/ml Streptavidin-coated SPA beads.) The plate is sealed, the beads are allowed to settle overnight, and then the plate is counted in a Packard Topcount Microplate Scintillation Counter (Packard Instrument Co., Meriden, Conn.).

The data for dose responses are plotted as % Control calculated with the data reduction formula 100*(U1-C2)/(C1-C2) versus concentration of compound where U is the unknown value, C1 is the average control value obtained for DMSO, and C2 is the average control value obtained for 0.1 M EDTA. Data are fitted to the curve described by: y=((Vmax*x)/(K+x)) where Vmax is the upper asymptote and K is the IC50.

The compound of Example 1, (S)-1-Benzyl-2-[5-(3-methyl-1H-indazol-5-yl)-6-phenyl-pyridin-3-yloxy]-ethylamine, was tested in the above AKT1 kinase inhibition assay and had an IC50=182 nM.

The pharmaceutically active compounds within the scope of this inventi on are useful as AKT inhibitors in mammals, particularly humans, in need thereof.

The present invention therefore provides a method of treating cancer, arthritis and other conditions requiring AKT inhibition, which comprises administering an effective compound of Formula (I) or a pharmaceutically acceptable salt, hydrate, solvate or pro-drug thereof. The compounds of Formula (I) also provide for a method of treating the above indicated disease states because of their demonstrated ability to act as Akt inhibitors. The drug may be administered to a patient in need thereof by any conventional route of administration, including, but not limited to, intravenous, intramuscular, oral, subcutaneous, intradermal, and parenteral.

The pharmaceutically active compounds of the present invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid;. Liquid carriers include syrup, peanut oil, olive oil, saline, and water. Similarly, the carrier or diluent may include any prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will be in the form of a syrup, elixir, emulsion, soft gelatin capsule, sterile injectable liquid such as an ampoule, or an aqueous or nonaqueous liquid suspension.

The pharmaceutical preparations are made following conventional techniques of a pharmaceutical chemist involving mixing, granulating, and compressing, when necessary, for tablet forms, or mixing, filling and dissolving the ingredients, as appropriate, to give the desired oral or parenteral products.

Doses of the presently invented pharmaceutically active compounds in a pharmaceutical dosage unit as described above will be an efficacious, nontoxic quantity preferably selected from the range of 0.001-100 mg/kg of active compound, preferably 0.001-50 mg/kg. When treating a human patient in need of an Akt inhibitor, the selected dose is administered preferably from 1-6 times daily, orally or parenterally. Preferred forms of parenteral administration include topically, rectally, transdermally, by injection and continuously by infusion. Oral dosage units for human administration preferably contain from 0.05 to 3500 mg of active compound. Oral administration, which uses lower dosages is preferred. Parenteral administration, at high dosages, however, also can be used when safe and convenient for the patient.

Optimal dosages to be administered may be readily determined by those skilled in the art, and will vary with the particular Akt inhibitor in use, the strength of the preparation, the mode of administration, and the advancement of the disease condition. Additional factors depending on the particular patient being treated will result in a need to adjust dosages, including patient age, weight, diet, and time of administration.

The method of this invention of inducing Akt inhibitory activity in mammals, including humans, comprises administering to a subject in need of such activity an effective Akt inhibiting amount of a pharmaceutically active compound of the present invention.

The invention also provides for the use of a compound of Formula (I) in the manufacture of a medicament for use as an Akt inhibitor.

The invention also provides for the use of a compound of Formula (I) in the manufacture of a medicament for use in therapy.

The invention also provides for the use of a compound of Formula (I) in the manufacture of a medicament for use in treating cancer.

The invention also provides for the use of a compound of Formula (I) in the manufacture of a medicament for use in treating arthritis.

The invention also provides for a pharmaceutical composition for use as an Akt inhibitor which comprises a compound of Formula (I) and a pharmaceutically acceptable carrier.

The invention also provides for a pharmaceutical composition for use in the treatment of cancer which comprises a compound of Formula (I) and a pharmaceutically acceptable carrier.

The invention also provides for a pharmaceutical composition for use in treating arthritis which comprises a compound of Formula (I) and a pharmaceutically acceptable carrier.

No unacceptable toxicological effects are expected when compounds of the invention are administered in accordance with the present invention.

In addition, the pharmaceutically active compounds of the present invention can be co-administered with further active ingredients, such as other compounds known to treat cancer or arthritis, or compounds known to have utility when used in combination with an Akt inhibitor.

Without further elaboration, it is believed that one skilled in the art can, using the preceding description, utilize the present invention to its fullest extent. The following Examples are, therefore, to be construed as merely illustrative and not a limitation of the scope of the present invention in any way.

### EXPERIMENTAL DETAILS

The compounds of Examples 1 to 222 are readily made according to Schemes 1 to 31 or by analogous methods.

### Example 1

### Preparation of (S)-1-Benzyl-2-[5-(3-methyl-1H-indazol-5-yl)-6-phenyl-pyridin-3-yloxy]-ethylamine

a) ((S)-1-Hydroxymethyl-2-phenyl-ethyl)-carbamic acid tert-butyl ester

Saturated NaHCO₃ aqueous solution (3 mL) was added to a solution of (−)-phenylalaninol (1.007 g, 6.66 mmol) and di-t-butyl dicarbonate (2.18 g, 9.99 mmol) in CH₂Cl₂ and the resulting mixture was stirred at room temperature for 3 h. The reaction was complete indicated by TLC. The organic layer was separated and the aqueous layer was extracted with CH₂Cl₂ (2 times). The combined the organic layers were dried (Na₂SO₄), concentrated, and the residue was purified by flash column chromatography (hexane/EtOAc 3:1) to give a white solid (1.64 g , 98%).

b) 3-Bromo-2-chloro-5-((S)-2-methyl-3-phenyl-propoxy)-pyridine

DEAD (0.30 mL, 1.87 mmol) was added to a solution of 4-bromo-5-chloro-3-hydroxypyridine (243 mg, 1.17 mmol, Koch, V. Schnatterer, S. Synthesis, 1990, 499-501), compound of Example 1 (a) (440 mg, 1.80 mmol) and Ph₃P (460 mg, 1.80 mmol) in THF (10 mL) at 0° C. The resulting mixture was warmed up to room temperature and stirred for 1 h. The reaction was complete indicated by TLC. The reaction mixture was concentrated and the residue was purified by flash column chromatography (hexane/EtOAc 9:1) to give a white solid (450 mg, 87%).

c) 3-Methyl-5-(4,4,5,5-tetramethyl-[1,3,2]dioxaborolan-2-yl)-indazole-1-carboxylic acid tert-butyl ester

A mixture of N-Boc-3-methyl-5-bromoindazole (1.11 g, 3.58 mmol), bis(pinacola)diboron (1.0 g, 3.94 mmol), KOAc (527 mg, 5.37 mmol), Pd₂dba₃ (49 mg, 0.054 mmol) and PCy₃ (72 mg, 0.26 mmol) in dioxane (21.5 mL) was purged with N₂ and heated at 80° C. under N₂ for 24 h. The reaction mixture was filtered through celite, which was rinsed with EtOAc. The combined filtrates were concentrated and the residue was purified by flash column chromatography (hexane/EtOAc 9:1) to give a light yellow solid (1.046 g, 74%).

d) 5-[5-((S)-2-tert-Butoxycarbonylamino-3-phenyl-propoxy)-2-chloro-pyridin-3-yl]-3-methyl-indazole-1-carboxylic acid tert-butyl ester

A mixture of the compound of Example 1 (b) (550 mg, 1.24 mmol), compound of Example 1(c) (550 mg, 1.53 mmol), (Ph₃P)₄Pd (143 mg, 0.12 mmol), 2N Na₂CO₃ aqueous solution (0.84 mL) and 1,4-dioxane (10 mL) was degassed and heated at 100° C. under N₂ overnight. The reaction mixture was filtered through celite, which was rinsed with EtOAc. The combined filtrates were concentrated and the residue was purified by flash column chromatography (hexane/EtOAc 3:1 to 1:1) to give a light yellow solid (585 mg, 80%).

e) {(S)-1-Benzyl-2-[5-(3-methyl-1H-indazol-5-yl)-6-phenyl-pyridin-3-yloxy]-ethyl}-carbamic acid tert-butyl ester

A mixture of the compound of Example 1(d) (196 mg, 0.33 mmol), phenylboronic acid (80.6 mg, 0.66 mmol), (Ph₃P)₄Pd (19 mg, 0.016 mmol), 2N Na₂CO₃ aqueous solution (0.73 mL) and 1,4-dioxane (3 mL) was degassed and irradiated under microwave at 160° C. for 20 min. The reaction mixture was filtered through celite, which was rinsed with EtOAc. The combined the filtrates were concentrated and the residue was purified by flash column chromatography (hexane/EtOAc 3:1 to 1:1) to give a light yellow solid (101 mg, 57%).

f) (S)-1-Benzyl-2-[5-(3-methyl-1H-indazol-5-yl)-6-phenyl-pyridin-3-yloxy]-ethylamine

A solution of the compound of Example 1(e) and 0.5 mL of TFA in CH₂Cl₂ (1.5 ml) was stirred at room temperature for 30 min, diluted with toluene and concentrated. The residue was taken up into DMSO and purified on reversed phase HPLC (MeCN, H₂O, 0.1% TFA) to give a white solid (78 mg, 78%). ¹H NMR (CD₃OD, 400 MHz) δ 8.49 (d, J=2.8 Hz, 1H), 7.92 (d, J=2.8 Hz, 1H), 7.66 (d, J=0.7 Hz, 1H), 7.40-7.32 (m, 11H), 7.11 (dd, J=8.7, 1.6 Hz), 4.46 (dd, J=10.6, 3.0 Hz, 1H), 4.31 (dd, J=10.6, 5.6 Hz, 1H), 4.03-3.95 (m, 1H), 3.19 (d, J=7.4 Hz, 2H), 2.50 (s, 3H); MS (M+H): 435.2

### Example 2

### Preparation of (S)-1-Benzyl-2-[6-furan-2-yl-5-(3-methyl-1H-indazol-5-yl)-pyridin-3-yloxy]-ethylamine

Following the procedure of Example 1(a)-1(f), except substituting 2-furanboronic acid for phenylboronic acid, the title compound was prepared. ¹H NMR (CD₃OD, 400 MHz) δ 8.40 (d, J=2.8 Hz, 1H), 7.72 (dd, J=1.4, 0.9 Hz, 1H), 7.61 (d, J=2.8 Hz, 1H), 7.56-7.54 (m, 2H), 7.41-7.31 (m, 7H), 7.28 (dd, J=8.6, 1.6 Hz, 1H), 6.36 (dd, J=3.5, 1.8 Hz, 1H), 5.91 (dd, J=3.5, 0.6 Hz, 1H), 4.48 (dd, J=10.6, 3.0 Hz, 1H), 4.23 (dd, J=10.6, 5.6 Hz, 1H), 4.00-3.90 (m, 1H), 3.16 (d, J=7.6 Hz, 2H), 2.58 (s, 3H); MS (M+H): 425.2

### Example 3

### Preparation of (S)-1-Benzyl-2-[5,6-bis-(3-methyl-1H-indazol-5-yl)-pyridin-3-yloxy]-ethylamine

Following the procedure of Example 1(a)-1(f), except substituting the compound of Example 1(c) for phenylboronic acid, the title compound was prepared. ¹H NMR (CD₃OD, 400 MHz) δ 8.46 (s, 1H), 7.81-7.78 (m, 2H), 7.71 (s, 1H), 7.40-7.27 (m, 13H), 7.19 (dd, J=8.7, 1.5 Hz, 1H), 7.07 (d, J=8.6 Hz, 1H), 4.45-4.42 (m, 1H), 4.30-4.25 (m, 1H), 4.01-3.92 (m, 1H), 3.19 (d, J=6.7 Hz, 2H), 2.50 (s, 3H), 2.45 (s, 3H) MS (M+H): 489.2

### Example 4

### Preparation of (S)-1-Benzyl-2-[6-thiophen-2yl-5-(3-methyl-1H-indazol-5-yl)-pyridin-3-yloxy]-ethylamine

Following the procedure of Example 1(a)-1(f), except substituting 2-thiopheneboronic acid for phenylboronic acid, the title compound was prepared. ¹H NMR (CD₃OD, 400 MHz) δ 8.47 (d, 1H), 7.90 (s, 1H), 7.68 (d, 1H), 7.48-7.30 (m, 8H), 7.17 (d, 1H), 6.88 (dd, 1H), 4.45 (dd, 1H), 4.32 (dd, 1H), 4.00 (m, 1H), 3.19 (d, 2H), 2.54 (s, 3H). MS (M+H): 441.2

### Example 5

### Preparation of (S)-1-Benzyl-2-[6-(4-chlorophenyl)-5-(3-methyl-1H-indazol-5-yl)-pyridin-3-yloxy]-ethylamine

Following the procedure of Example 1(a)-1(f), except substituting 4-chlorophenylboronic acid for phenylboronic acid, the title compound was prepared. ¹H NMR (CD₃OD, 400 MHz) δ 8.46 (d, 1H), 7.68 (dd, 2H), 7.40-7.29 (m, 6H), 7.22 (m, 4H), 7.06 (m, 1H), 4.40 (dd, 1H), 4.25 (dd, 1H), 3.99-3.95 (m, 1H), 3.19 (d, 2H), 2.53 (s, 3H). MS (M+H): 469.2

### Example 6

### Preparation of (S)-1-Benzyl-2-[6-(3-chlorophenyl)-5-(3-methyl-1H-indazol-5-yl)-pyridin-3-yloxy]-ethylamine

Following the procedure of Example 1(a)-1(f), except substituting 3-chlorophenylboronic acid for phenylboronic acid, the title compound was prepared. ¹H NMR (CD₃OD, 400 MHz) δ 8.42 (d, 1H), 7.65 (s, 1H), 7.60 (s, 1H), 7.42-7.28 (m, 8H), 7.19 (t, 1H), 7.08 (m, 2H), 4.39 (dd, 1H), 4.26 (dd, 1H), 3.97 (m, 1H), 3.18 (d, 2H), 2.50 (s, 3H). MS (M+H): 469.2

### Example 7

### Preparation of (S)-1-Benzyl-2-[6-benzyl-5-(3-methyl-1H-indazol-5-yl)-pyridin-3-yloxy]-ethylamine

a) {(S)-1-Benzyl-2-[6-benzyl-5-(3-methyl-1H-indazol-5-yl)-pyridin-3-yloxy]-ethyl}-carbamic acid benzyl ester

A mixture of 1(d) (35 mg, 0.059 mmol), BrZnPh (0.59 mL, 0.5 M in THF), and Pd(Ph₃P)₄ (6.8 mg, 0.0059 mmol) was purged with N₂, stirred at 75° C. overnight and cooled to room temperature. Saturated NH₄Cl aqueous solution was added and the aqueous layer was extracted with EtOAc. The combined organic layers were dried (Na₂SO₄), concentrated and the residue was purified by flash column chromatography (hexane/EtOAc 1:1) to give a mixture of 7(a) and {(s)-1-Benzyl-2-[6-chloro-5-(3-methyl-1H-indazol-5-yl)-pyridin-3-yloxy]-ethyl}-carbamic acid benzyl ester (18 mg).

b) (S)-1-Benzyl-2-[6-benzyl-5-(3-methyl-1H-indazol-5-yl)-pyridin-3-yloxy]-ethylamine

A mixture of 7(a) and {(s)-1-Benzyl-2-[6-chloro-5-(3-methyl-1H-indazol-5-yl) -pyridin-3-yloxy]-ethyl}-carbamic acid benzyl ester (18 mg), 10% Pd/C (5 mg) and 0.5 mL of MeOH was stirred under a balloon pressure of H₂ overnight. The reaction mixture was filtered through celite, which was rinsed with MeOH. The combined filtrates were concentrated and the residue was purified by reversed phase HPLC (MeCN, H₂O, 0.1% TFA) to give 2.3 mg of the title compound. ¹H NMR (CD₃OD, 400 MHz) δ 8.40 (d, 1H), 7.62 (dd, 1H), 7.53 (d, 1H), 7.46 (s, 1H), 7.40-7.27 (m, 6H), 7.18 (m, 3H), 6.88 (m, 2H), 4.35 (dd, 1H), 4.20 (m, 3H), 3.82 (m, 1H), 3.13 (d, 2H), 2.49 (s, 3H), MS (M+H): 449.2

### Example 8

### Preparation of (S)-1-Benzyl-2-[6-cyclopent-1-enyl-5-(3-methyl-1H-indazol-5-yl)-pyridin-3-yloxy]-ethylamine

Following the procedure of Example 1(a)-1(f), except substituting cyclopent-1-enylboronic acid for phenylboronic acid, the title compound was prepared. ¹H NMR (CD₃OD, 400 MHz) δ 8.46 (d, 1H), 8.14 (d, 1H), 7.86 (s, 1H), 7.60 (d, 1H), 7.53-7.38 (m, 6H), 6.30 (s, 1H), 4.49 (dd, 1H), 4.34 (dd, 1H), 4.00 (m, 1H), 3.17 (d, 2H), 2.60 (s, 3H), 2.52 (m, 2H), 2.24 (m, 2H), 1.90 (m, 2H), MS (M+H): 425.4

### Example 9

### Preparation of (S)-1-Benzyl-2-[6-cyclopentyl-5-(3-methyl-1H-indazol-5-yl)-pyridin-3-yloxy]-ethylamine

To the solution of Example 8 (7.8 mg, 0.012 mol) in MeOH (0.5 ml) was added 5 mg of 10% Pd/C. The mixture was stirred under a balloon pressure of H₂ for 1 hr. The reaction mixture was filtered through celite, which was rinsed with MeOH. The combined filtrates were concentrated and the residue was purified by reversed phase HPLC (MeCN, H₂O, 0.1% TFA) to give 6 mg (77%) of the title compound. ¹H NMR (CD₃OD, 400 MHz) δ 8.46 (d, 1H), 8.05 (d, 1H), 7.80 (s, 1H), 7.65 (dd, 1H) 7.55-7.29 (m, 6H), 4.44-4.40 (dd, 1H), 4.30-4.26 (dd, 1H), 3.97 (m, 1H), 3.54-3.45 (m, 1H), 3.15 (d, 2H), 2.61 (s, 3H), 2.10-1.59 (m, 8H), MS (M+H):427.4

### Example 10

### Preparation of (S)-1-Benzyl-2-[6-cyclohex-1-enyl-5-(3-methyl-1H-indazol-5-yl) -pyridin-3-yloxy]-ethylamine

Following the procedure of Example 1(a)-1(f), except substituting cyclohex-1-enylboronic acid for phenylboronic acid, the title compound was prepared. ¹H NMR (CD₃OD, 400 MHz) δ 8.44 (d, 1H), 8.25 (d, 1H), 7.90 (s, 1H), 7.62 (d, 1H), 7.53 (d, 1H), 7.42-7.30 (m, 5H), 6.27 (t, 1H), 4.49 (m, 1H), 4.35 (m, 1H), 4.00 (m, 1H), 3.17 (d, 2H), 2.61 (s, 3H), 2.26 (m, 2H), 1.83 (m, 2H), 1.61 (m, 2H), 1.53 (m, 2H). MS (M+H): 439.2

### Example 11

### Preparation of (S)-1-Benzyl-2-[6-cyclohexyl-5-(3-methyl-1H-indazol-5-yl)-pyridin-3-yloxy]-ethylamine

Following the procedure of Example 9, except substituting Example 8 with Example 10, the title compound was prepared. ¹H NMR (CD₃OD, 400 MHz) δ 8.41 (d, 1H), 7.83 (d, 1H), 7.74 (s, 1H), 7.63 (d, 1H), 7.40-7.29 (m, 6H), 4.41 (dd, 1H), 4.24 (dd, 1H), 3.96 (m, 1H), 3.14 (d, 2H), 2.98 (m, 1H), 1.90-1.62 (m, 7H), 1.48-1.11 (m, 3H). MS (M+H): 441.2

### Example 12

### Preparation of 3-Methyl-5-[2-phenyl-5-(piperidin-4-ylmethoxy)-pyridin-3-yl]-1H-indazole

a) 6-chloro-5-(3-methyl-1H-indazol-5-yl)-3-pyridinol

A mixture of 5-bromo-6-chloro-3-pyridinol (1.40 g, 6.70 mmol), 3-methyl-5-(4,4,5,5-tetramethyl-1,3,2-dioxaborolan-2-yl)-1H-indazole (2.08 g, 8.04 mmol), (Ph₃P)₄Pd (385 mg, 0.34 mmol), 2N Na₂CO₃ aqueous solution (7.7 mL) and DME (20 mL) was degassed and heated at 80° C. under N₂ overnight. The reaction mixture was filtered through celite, which was rinsed with EtOAc. The combined filtrates were concentrated and the residue was purified by flash column chromatography (hexane/EtOAc 1:1) to give a light yellow foamy solid (1.23 g, 71%).

b) 5-(3-methyl-1H-indazol-5-yl)-6-phenyl-3-pyridinol

A mixture of compound of Example 12(a) (1.03 g, 4.75 mmol), phenylboronic acid (695 mg, 5.70 mmol), (Ph₃P)₄Pd (274 mg, 0.24 mmol), 2N Na₂CO₃ aqueous solution (8.5 mL) and 1,4-dioxane (20 mL) was degassed and heated at 100° C. overnight. The reaction mixture was filtered through celite, which was rinsed with EtOAc. The combined the filtrates were concentrated and the residue was purified by flash column chromatography (hexane/EtOAc 1:1) to give a light yellow solid (846 mg, 70%).

c) 1,1-dimethylethyl 4-({[5-(3-methyl-1H-indazol-5-yl)-6-phenyl-3-pyridinyl]oxy}methyl)-1-piperidinecarboxylate

DEAD (0.033 mL, 0.2 mmol) was added to a solution of the compound of Example 12(b) (40 mg, 0.13 mmol), 1,1-dimethylethyl 4-(hydroxymethyl)-1-piperidinecarboxylate (42.8 mg, 0.2 mmol) and Ph₃P (52 mg, 0.2 mmol) in THF (1 mL) at 0° C. The resulting mixture was warmed up to room temperature and stirred for 1 h. The reaction was complete indicated by TLC. The reaction mixture was concentrated and the residue was purified by flash column chromatography (hexane/EtOAc 1:1) to give a white solid (45 mg, 69%).

d) 3-methyl-5-{2-phenyl-5-[(4-piperidinylmethyl)oxy]-3-pyridinyl}-1H-indazole

A solution of compound of Example 12(c) and 0.5 mL of TFA in CH₂Cl₂ (1.5 ml) was stirred at room temperature for 30 min, diluted with toluene and concentrated. The residue was taken up into DMSO and purified on reversed phase HPLC (MeCN, H₂O, 0.1% TFA) to give a white solid (35 mg, 62%). ¹H NMR (CD₃OD, 400 MHz) δ 8.56 (d, 1H), 8.23 (d, 1H), 7.74 (s, 1H), 7.52-7.35 (m, 6H), 7.13 (d, 1H), 4.27 (d, 2H), 3.50 (d, 2H), 3.12 (m, 2H), 2.51 (s, 3H), 2.30 (m, 1H), 2.17 (d, 2H), 1.73 (m, 2H), MS (M+H): 399.4

### Example 13

### Preparation of 3-[5-(3-Methyl-1H-indazol-5-yl)-6-phenyl-pyridin-3-yloxy]-propylamine

Following the procedure of Example 12, except substituting (2-Hydroxy-ethyl)-carbamic acid tert-butyl ester for 1,1-dimethylethyl 4-(hydroxymethyl)-1-piperidinecarboxylate the title compound was prepared. ¹H NMR (CD₃OD, 400 MHz) δ 8.57 (d, 1H), 8.25 (d, 1H), 7.74 (s, 1H), 7.50-7.34 (m, 6H), 7.15 (d, 1H), 4.78 (t, 2H), 3.26 (t, 2H), 2.50 (s, 3H), 2.30 (m, 2H), MS (M+H): 359.2

### Example 14

### Preparation of (S)-1-Benzyl-2-[5-(3-methyl-1H-indazol-5-yl)-6-(5-methyl-thiophen-2-yl)-pyridin-3-yloxy]-ethylamine

Following the procedure of Example 1(a)-1(f), except substituting 5-methylthiophen-2-ylboronic acid for phenylboronic acid, the title compound was prepared. ¹H NMR (CD₃OD, 400 MHz) δ 8.31 (d, 1H), 7.70 (s, 1H), 7.51 (d, 1H), 7.49-7.24 (m, 7H), 6.47 (m, 1H), 6.31 (d, 1H), 4.31 (dd, 1H), 4.17 (dd, 1H), 3.95 (m, 1H), 3.15 (d, 2H), 2.57 (s, 3H), 2.39 (s, 3H). MS (M+H): 455.0

### Example 15

### Preparation of (S)-1-Benzyl-2-[5-(3-methyl-1H-indazol-5-yl)-6-(5-methyl-furan-2-yl)-pyridin-3-yloxy]-ethylamine

Following the procedure of Example 1(a)-1(f), except substituting 5-methylfuran-2-ylboronic acid for phenylboronic acid, the title compound was prepared. ¹H NMR (CD₃OD, 400 MHz) δ 8.37(s, 1H), 7.70 (m, 2H), 7.62 (m, 1H), 7.49-7.30 (m, 5H), 5.97 (m, 1H), 5.80 (s, 1H), 5.73 (s, 1H), 4.37 (dd, 1H), 4.22 (dd, 1H), 3.96 (m, 1H), 3.17 (d, 2H), 2.55 (s, 3H), 2.26 (s, 3H). MS (M+H): 439.2

### Example 16

### Preparation of 3-Methyl-5-[2-phenyl-5-(4-pyridin-3-yl-methyl-piperazin-1-yl)-pyridin-3-yl]-1H-indazole

a) Trifluoro-methanesulfonic acid 5-(3-methyl-1H-indazol-5-yl)-6-phenyl-pyridin-3-yl ester

A solution of compound 12(b) (150 mg, 0.50 mmol) and PhNTf₂ (213 mg, 1.2 eq.) in CH₂Cl₂ (5 mL) was added Et₃N (0.14 mL, 2.0 eq.). The resulting mixture was stirred at rt overnight, washed with water, brine, and dried (Na₂SO₄). Removal of the solvent followed by flash column chromatography of the residue on silica gel afforded 198 mg (92%) of the titled compound.

b) 3-Methyl-5-[2-phenyl-5-(4-pyridin-3-ylmethyl-piperazin-1-yl)-pyridin-3-yl]-1H-indazole

A solution of compound Example 16(a) (13.8 mg, 0.032 mmol) and 1-pyridin-3-ylmethyl-piperazine (14 mg, 2.5 eq.) in NMP (0.2 mL) was irradiated with microwave (personal choice synthesizer) at 205° C. for 30 min. The reaction mixture was loaded on the reversed phase HPLC column and purified (MeCN, H₂O, 0.1% TFA) to give 17.2 mg of white solid (67%). ¹H NMR (CD₃OD, 400 MHz) δ 9.04 (s, 1H), 8.90 (s, 1H), 8.58 (d, 1H), 8.46 (s, 1H), 8.26 (s, 1H), 8.00 (m, 1H), 7.77 (s, 1H), 7.50-7.34 (m, 6H), 7.15 (d, 1H), 4.59 (s, 2H), 3.88 (t, 4H), 3.51 (t, 4H), 2.51 (s, 3H). MS (M+H): 461.4

### Example 17

### Preparation of 3-Methyl-5-[2-phenyl-5-(4-pyridin-4-ylmethyl-piperazin-1-yl)-pyridin-3-yl]-1H-indazole

Following the procedure of Example 16, except substituting 1-pyridin-4-ylmethyl-piperazine for 1-pyridin-3-ylmethyl-piperazine the title compound was prepared. ¹H NMR (CD₃OD, 400 MHz) δ 8.88 (d, 2H), 8.41 (d, 1H), 8.21 (d, 1H), 8.13 (d, 2H), 7.76 (s, 1H), 7.48-7.34 (m, 6H), 7.12 (d, 1H), 4.31 (s, 2H), 3.78 (t, 4H), 3.15 (t, 4H), 2.51 (s, 3H). MS (M+H): 461.4

### Example 18

### Preparation of [(1S)-2-{[6-(3-furanyl)-5-(3-methyl-1H-indazol-5-yl)-3-pyridinyl]oxy}-1-(phenylmethyl)ethyl]amine

Following the procedure of Example 1(a)-1(f), except substituting 3-furanboronic acid for phenylboronic acid, the title compound was prepared. ¹H NMR (CD₃OD, 400 MHz) δ 8.39 (d, J=2.4 Hz, 1H), 7.72 (s, 1H), 7.57 (s, 1H), 7.53 (d, J=8.8 Hz, 1H), 7.41-7.15 (m, 8H), 6.31 (dd, J=3.5, 1.8 Hz, 1H), 4.36 (d, J =10.4, 1H), 4.22 (dd, J=10.6, 5.6 Hz, 1H), 4.00-3.94 (m, 1H), 3.16 (m, 2H), 2.57 (s, 3H); MS (M+H): 425.2.

### Example 19

### Preparation of [(1S)-2-{[5-(3-methyl-1H-indazol-5-yl)-6-(5-chloro-2-thienyl) -3-pyridinyl]oxy}-1-(phenylmethyl)ethyl]amine

Following the procedure of Example 1(a)-1(f), except substituting 5-chloro-2-thiopheneboronic acid for phenylboronic acid, the title compound was prepared. ¹H NMR (CD₃OD, 400 MHz) δ 8.33 (d, 1H), 7.16 (d, 1H), 7.49 (d, 1H), 7.41-7.28 (m, 6H), 7.26 (d, 1H), 6.92 (d, 1H), 6.46 (d, 1H), 4.32 (dd, 1H), 4.18 (dd, 1H), 3.95 (m, 1H), 3.14 (m, 2H), 2.58 (s, 3H), 2.01 (s, 3H); MS (M+H): 475.2/477.2.

### Example 20

### Preparation of [(1S)-2-{[6-(3-aminophenyl)-5-(3-methyl-1H-indazol-5-yl)-3-pyridinyl]oxy}-1-(phenylmethyl)ethyl]amine

Following the procedure of Example 1(a)-1(f), except substituting (3-aminophenyl)boronic acid for phenylboronic acid, the title compound was prepared. ¹H NMR (CD₃OD, 400 MHz) δ 8.46 (d, 1H), 7.65 (m, 2H), 7.42-7.22 (m, 10H), 7.11 (d, 1H), 4.39 (m, 1H), 4.26 (dd, 1H), 3.98 (m, 1H), 3.19 (m, 2H), 2.52 (s, 3H); MS (M+H): 450.2.

### Example 21

### Preparation of (S)-1-Benzyl-2-[5-(1H-indazol-5-yl)-6-phenyl-pyridin-3-yloxy]-ethylamine

Following the procedure of Example 1(a)-1(f), except substituting 5-(4,4,5,5-tetramethyl-[1,3,2]dioxaborolan-2-yl)-indazole-1-carboxylic acid tert-butyl ester for compound Example 1(C), the title compound was prepared. ¹H NMR (CD₃OD, 400 MHz) δ 8.53 (d, 1H), 8.06 (s, 1H), 7.98 (d, 1H), 7.75 (s, 1H), 7.46-7.30 (m, 10 H), 7.13 (d, 1H), 4.49 (dd, 1H), 4.33 (dd, 1H), 4.01 (m, 1H), 3.19 (d, 2H); MS (M+H): 421.2.

### Example 22

### Preparation of (S)-1-Benzyl-2-{6-[3-(3-fluoro-benzyloxy)phenyl]-5-(3-methyl-1H -indazol-5-yl)-pyridin-3-yloxy}-ethylamine

a) 2-[3-(3-fluoro-benzyloxy)phenyl]-4,4,5,5-tetramethyl-[1,3,2]dioxaborolane

A mixture of 3-(4,4,5,5-tetramethyl-[1,3,2]dioxaborolan-2-yl)-phenol (110 mg, 0.50 mmol), 3-fluorobenzyl bromide (0.074 mL, 1.2 eq.), Cs₂CO₃ (179 mg, 1.1 eq) and DMF (3 mL) was stirred at rt for 3 hr, and taken up into EtOAc and water.

The organic was separated, dried (Na₂SO₄) and concentrated. The residue was purified by flash column chromatography on silica gel to give 91 mg (55%) of the titled compound.

b) (S)-1-Benzyl-2-{6-[3-(3-fluoro-benzyloxy)phenyl]-5-(3-methyl-1H-indazol-5-yl)-pyridin-3-yloxy}-ethylamine

Following the procedure of Example 1(a)-1(f), except substituting compound of Example 22 (a) for phenylboronic acid, the title compound was prepared. ¹H NMR (CD₃OD, 400 MHz) δ 8.46 (s, 1H), 7.80 (s, 1H), 7.65 (s, 1H), 7.40-6.87 (m,15H), 4.85 (s, 2H), 4.45 (dd, 1H), 4.29 (dd, 1H), 3.99 (m, 1H), 3.18 (d, 2H), 2.52 (s, 3H); MS (M+H): 559.4

### Example 23

### Preparation of (S)-1-Benzyl-2-[5-(3-phenyl-1H-indazol-5-yl)-6-phenyl-pyridin-3-yloxy]-ethylamine

a) {(S)-1-Benzyl-2-[5-(1H-indazol-5-yl)-6-phenyl-pyridin-3-yloxy]-ethyl}-carbamic acid tert-butyl ester

Following the procedure of Example 1(a)-1(e), except substituting 5-(4,4,5,5-tetramethyl-[1,3,2]dioxaborolan-2-yl)-indazole-1-carboxylic acid tert-butyl ester for the compound of Example 1(c), the title compound was prepared.

b)_{(S)-1-Benzyl-2-[5-(3-iodo-1H-indazol-5-yl)-6-phenyl-pyridin-3-yloxy]-ethyl}-carbamic acid tert-butyl ester

Iodine (53 mg, 1.5 eq.) and KOH (20 mg, 2.5 eq., grounded) were added to a solution of the compound of Example 23(a) (71 mg, 0.14 mmol) in DMF (1.5 mL). The reaction mixture was stirred at rt for 30 min, and taken up into EtOAc and water. The organic layer was separated, washed with brine, dried (Na₂SO₄), and concentrated. The residue was purified by flash column chromatography on silica gel (2:1 hexane/EtOAc) to give a white solid (37 mg, 42%).

c) (S)-1-Benzyl-2-[5-(3-phenyl-1H-indazol-5-yl)-6-phenyl-pyridin-3-yloxy]-ethylamine

Following the procedure of Example 1(e), except substituting compound of Example 23(b) for compound of Example 1(d), the title compound was prepared. ¹H NMR (CD₃OD, 400 MHz) δ 8.54 (d, 1H), 8.04 (d, 1H), 7.81 (s, 1H), 7.65-7.29 (m, 17H), 4.49 (dd, 1H), 4.36-4.32 (m, 1H), 4.03-3.99 (m, 1H), 3.20 (d, 2H); MS (M+H): 497.2.

### Example 24

### Prepatation of [(1S)-2-{[5-(3-methyl-1H-indazol-5-yl)-6-(1H-pyrrol-2-yl)-3-pyridinyl]oxy}-1-(phenylmethyl)ethyl]amine

Following the procedure of Example 1(a)-1(f), except substituting (1-{[(1,1-dimethylethyl)oxy]carbonyl}-1H-pyrrol-2-yl)boronic acid for phenylboronic acid, the title compound was prepared. ¹H NMR (CD₃OD, 400 MHz) δ 8.35 (d, I H), 7.71 (s, 1H), 7.59 (d, 1H), 7.52 (d, 2H), 7.40-7.25 (m, 7H), 6.82 (d, 2H), 5.98 (m, 1H), 5.65 (m, 1H), 4.35 (dd, 1H), 4.21 (dd, 1H), 3.95 (m, 1H), 3.20 (d, 2H), 2.67 (s, 3 H); MS (M+H): 424.2.

### Example 25

### Prepatation of N-{3-[5-{[(2S)-2-amino-3-phenylpropyl]oxy}-3-(3-methyl-1H-indazol-5-yl)-2-pyridinyl]phenyl}benzamide

a) {(S)-1-Benzyl-2-[5-(3-methyl-1H-indazol-5-yl)-6-(3-nitro-phenyl)-pyridin-3-yloxy]-ethyl}-carbamic acid tert-butyl ester

Following the procedure of Example 1(a)-1(e), except substituting 3-nitrophenylboronic acid for phenylboronic acid, the title compound was prepared.

b) {(S)-1-Benzyl-2-[5-(3-methyl-1H-indazol-5-yl)-6-(3-amino-phenyl)-pyridin-3-yloxy]-ethyl}-carbamic acid tert-butyl ester

To a solution of the compound of Example 25(a) (260 mg, 0.38 mmol) in EtOH was added 10% Pd/C (26 mg) and the reaction mixture was stirred under a H₂ balloon overnight. The reaction mixture was filtered through celite, which was rinsed with EtOH. The combined filtrates were concentrated to give the titled product (240 mg, 97%).

c) N-{3-[5-{[(2S)-2-amino-3-phenylpropyl]oxy}-3-(3-methyl-1H-indazol-5-yl)-2-pyridinyl]phenyl}benzamide

A solution of the compound of Example 25(b) (90 mg, 0.14 mmol), benzoyl chloride (30 mg, 0.21 mmol) and TEA (0.04 ml, 0.28 mmol) in 3 ml CH₂Cl₂ was stirred at rt for 20 min. Solvent was removed and the residue was dissolved in EtOAc, which was washed with NaHCO₃, brine and dried. Removal of the solvent followed by flash column chromatography purification of the residue on silica gel afforded the titled compound (78 mg, 75%).

d) N-{3-[5-{[(2S)-2-amino-3-phenylpropyl]oxy}-3-(3-methyl-1H-indazol-5-yl)-2-pyridinyl]phenyl}benzamide

A solution of the compound of Example 25(c) (78 mg, 0.10 mmol) in 0.6 ml TFA and 2 ml CH₂Cl₂ was stirred at rt for 20 min, diluted with toluene, and concentrated. The residue was taken up into DMSO and purified on reversed phase HPLC (MeCN, H₂O, 0.1% TFA) to give a white solid (40 mg, 72%). ¹H NMR (CD₃OD, 400 MHz) δ 8.46 (d, 1H), 7.93 (s, 1H), 7.86 (m, 2H), 7.75 (d, 1 H), 7.67 (s, 1H), 7.62-7.45 (m, 4H), 7.40-7.30 (m, 6H), 7.22 (t, 1H), 7.16 (d, 1H), 6.98 (d, 1H), 4.45 (dd, 1H), 4.29 (dd, 1H), 4.02 (m, 1H), 3.18 (d, 2H), 2.52 (s, 3 H); MS (M+H): 554.4.

### Example 26

### Prepatation of N-{3-[5-{[(2S)-2-amino-3-phenylpropyl]oxy}-3-(3-methyl-1H-indazol-5-yl)-2-pyridinyl]phenyl}-2,6-difluorobenzamide

Following the procedure of Example 25, except substituting 2,6-difluorobenzoyl chloride for benzoyl chloride, the title compound was prepared. ¹H NMR (CD₃OD, 400 MHz) δ 8.44 (d, 1H), 7.90 (d, 1H), 7.72 (d, 2H), 7.52 (m, 2 H), 7.41-3.33 (m, 6H), 7.22 (t, 1H), 7.15-7.11 (m, 3H), 6.96 (d, 1H), 4.43 (dd, 1 H), 4.25 (dd, 1H), 3.99 (m, 1H), 3.17 (d, 2H), 2.52 (s, 3H); MS (M+H): 590.4.

### Example 27

### Prepatation of N-{3-[5-{[(2S)-2-amino-3-phenylpropyl]oxy}-3-(3-methyl-1H-indazol-5-yl)-2-pyridinyl]phenyl}cyclohexanecarboxamide

Following the procedure of Example 25, except substituting cyclohexane carbonyl chloride for benzoyl chloride, the title compound was prepared. ¹H NMR (CD₃OD, 400 MHz) δ 8.40 (d, 1H), 7.70 (s, 1H), 7.65 (s, 2H), 7.43-7.36 (m, 7H), 7.14 (t, 1H), 7.09 (d, 1H), 6.90 (d, 1H), 4.12 (d, 1H), 4.26 (d, 1H), 3.98 (m, 1H), 3.17 (d, 2H), 2.51 (s, 1H), 2.29 (m, 1H), 1.80 (m, 4H), 1.47-1.28 (m, 6H); MS (M+H):560.4.

### Example 28

### Preparation of [(1S)-2-({5-[3-(2-furanyl)-1H-indazol-5-yl]-6-phenyl-3-pyridinyl}oxy)-1-(phenylmethyl)ethyl]amine

Following the procedure of Example 23(a)-23(c), except substituting 2-furanylboronic acid for phenylboronic acid, the title compound was prepared. ¹H NMR (CD₃OD, 400 MHz) δ 8.47(d, 1H), 8.03(s, 1H), 7.80(d, 1H), 7.66(d, 1H), 7.45-7.20 (m, 11H), 7.18 (dd, 1H), 6.85 (d, 1H), 6.61 (dd, 1H), 4.43 (dd, 1H), 4.29 (dd, 1H), 3.99-3.07 (m, 1H), 3.18 (d, 2H); MS (M+H): 487.4.

### Example 29

### Preparation of {(1S)-2-phenyl-1-[({6-phenyl-5-[3-(2-thienyl)-1H-indazol-5-yl]-3-pyridiny}yloxy)methyl]ethyl}amine

Following the procedure of Example 23(a)-23(c), except substituting 2-thienylboronic acid for phenylboronic acid, the title compound was prepared. ¹H NMR (CD₃OD, 400 MHz) δ 8.45(d, 1H), 7.88(s, 1H), 7.75(d, 1H), 7.48-7.15(m, 14 H), 4.44 (dd, 1H), 4.28 (dd, 1H), 3.97-3.90 (m, 1H), 3.18 (d, 2H); MS (M+H): 503.2.

### Example 30

### Preparation of [(1S)-2-({5-[3-(3-furanyl)-1H-indazol-5-yl]-6-phenyl-3-pyridinyl}oxy)-1-(phenylmethyl)ethyl]amine

Following the procedure of Example 23(a)-23(c), except substituting 3-furanylboronic acid for phenylboronic acid, the title compound was prepared. ¹H NMR (CD₃OD, 400 MHz) δ 8.48(d, 1H), 7.93(d, 1H), 7.85(s, 1H), 7.77(d, 1H), 7.64 (s, 1H), 7.46 (d, 1H), 7.44-7.25 (m, 9H), 7.22 (dd, 1H), 6.82 (d, 1H), 4.46 (dd, 1H), 4.30 (dd, 1H), 4.28-4.25 (m, 1H), 3.19 (d, 2H); MS (M+H): 487.4.

### Example 31

### Preparation of [(1S)-2-({5-[3-(3-thienyl)-1H-indazol-5-yl]-6-phenyl-3-pyridinyl}oxy)-1-(phenylmethyl)ethyl]amine

Following the procedure of Example 23(a)-23(c), except substituting 3-thienylboronic acid for phenylboronic acid, the title compound was prepared. ¹H NMR (CD₃OD, 400 MHz) δ 8.46 (d, 1H), 7.87 (d, 1H), 7.82 (s, 1H), 7.67 (d, 1H), 7.58 (s, 1H), 7.44 (d, 1H), 7.44-7.25 (m, 10H), 7.22 (dd, 1H), 4.45 (dd, 1H), 4.31 (dd, 1H), 4.28-4.25 (m, 1H), 3.18 (d, 2H); MS (M+H): 503.2.

### Example 32

### Preparation of 3-[5-{[(2S)-2-amino-3-phenylpropyl]oxy}-3-(3-methyl-1H-indazol-5-yl)-2-pyridinyl]phenol

Following the procedure of Example 1(a)-1(f), except substituting 3-(4,4,5,5-tetramethyl-1,3,2-dioxaborolan-2-yl)phenol for phenylboronic acid, the title compound was prepared. ¹H NMR (CD₃OD, 400 MHz) δ 8.42(d, 1H), 7.81 (s, 1H), 7.68 (d, 1H), 7.42-7.33 (m, 6H), 7.14-7.11 (m, 2H), 6.78-6.72 (m, 3H), 4.44 (dd, 1H0, 4.29(dd, 1H), 3.99-3.97 (m, 1H), 3.18 (d, 2H), 2.52 (s, 3H); MS (M+H): 451.2.

### Example 33

### Preparation of [(1S)-2-{[5-(2,3-dimethyl-2H-indazol-5-yl)-6-phenyl-3-pyridinyl]oxy}-1-(phenylmethyl)ethyl]amine

a) 5-(2,3-dimethyl-2H-indazol-5-yl)-6-phenyl-3-pyridinyl trifluoroacetate

To a solution of the compound of Example 16(a) (33 mg, 0.076 mmol) in EtOAc was added Me₃OBF₄ (17 mg, 0.115 mmol) and stirred for 3 h at rt. The reaction was completed indicated by LC/MS. Aqueous NaHCO₃ was added. Organic layer was separated and concentrated, and the residue was purified by flash column chromatography (hexane/EtOAc 2:1) to give a white foaming solid (14.7 mg, 43%).

b) 5-(2,3-dimethyl-2H-indazol-5-yl)-6-phenyl-3-pyridinol

To a solution of the compound of the Example 33(a) (14.7 mg, 0.033 mmol) in 0.5 ml MeOH was added 2N NaOH 0.1 mL. The resulting mixture was stirred at rt for 30 min and concentrated. The residue was dissolved in 1 mL of water and neutralized with HOAc. The resulting mixture was extracted by CH₂Cl₂ (5 mL×3). The organic layers were combined and concentrated, and the residue was purified by flash column chromatography (Hexane/EtOAc 1:1) to give a white solid (10 mg).

c) 1,1-dimethylethyl [(1S)-2-{[5-(2,3-dimethyl-2H-indazol-5-yl)-6-phenyl-3-pyridinyl]oxy}-1-(phenylmethyl)ethyl]carbamate

DEAD (10.4 uL, 0.066 mmol) was added to a solution of the compound of Example 33(b) (10.8 mg, 0.033 mmol), compound of Example 1(a) (12.4 mg, 0.049 mmol) and Ph₃P (13.0 mg, 0.049 mmol) in THF (2 mL) at rt. The resulting mixture was stirred at rt overnight. Excess of DEAD and Ph3P were added. The reaction mixture was concentrated and the residue was purified by flash column chromatography (CH₂Cl₂/EtOAc 1:1) to give a white solid (100 mg, coeluted with Ph₃P═O).

d) [(1S)-2-{[5-(2,3-dimethyl-2H-indazol-5-yl)-6-phenyl-3-pyridinyl]oxy}-1-(phenylmethyl)ethyl]amine

A solution of the compound of Example 33(c) and 0.2 mL of TFA in CH₂Cl₂ (0.8 ml) was stirred at room temperature for 20 min, diluted with toluene and concentrated. The residue was taken up into DMSO and purified on reversed phase HPLC (MeCN, H₂O, 0.1% TFA) to give a white solid (4.5 mg, 20% over 3 steps). ¹H NMR (CD₃OD, 400 MHz) δ 8.52 (d, 1H), 7.99 (d, 1H), 7.66 (s, 1H), 7.42-7.31 (m, 1H), 7.01 (d, 1H), 4.48 (dd, 1H), 4.33 (dd, 1H), 4.12 (s, 3H), 4.02-3.99 (m, 1H), 3.19 (d, 2H), 2.62 (s, 3H); MS (M+H): 449.2

### Example 34

### Preparation of [(1S)-2-{[5-(3-cyclopropyl-1H-indazol-5-yl)-6-(3-furanyl)-3-pyridinyl]oxy}-1-(phenylmethyl)ethyl]amine

a) 1,1-dimethylethyl 5-[5-{[(2S)-2-amino-3-phenylpropyl]oxy}-2-(3-furanyl)-3-pyridinyl]-3-iodo-1H-indazole-1-carboxylate

A solution of 122(a) (271.5 mg, 0.427 mmol), Boc2O (112 mg, 1.2 eq.), Et3N (0.12 mL, 2.0 eq.) and DMAP (10 mg, 20 mol %) in CH2Cl2 (4 mL) was stirred at room temperature for 2 h, concentrated and the residue was purified by flash column chromatography (hexane/EtOAc 3:1) to give a white solid (312 mg, 99%).

b) [(1S)-2-{[5-(3-cyclopropyl-1H-indazol-5-yl)-6-(3-furanyl)-3-pyridinyl]oxy}-1-(phenylmethyl)ethyl]amine

Cyclopropylmagnesium bromide (0.6 mL, 0.5 M in THF) was added dropwise to a solution of ZnCl2 (0.6 mL, 0.5 M in THF) at 0° C. and the resulting mixture was stirred at 0° C. for 30 min. To this reaction mixture was added 34 (a) (74 mg, 0.1 mmol). The resulting solution was heated at 50° C. overnight, cooled down to room temperature, and taken up into EtOAc, which washed with NH4Cl saturated aqueous solution, water, brine, and dried (Na2SO4). The solvent was removed and the residue was treated with TFA following the procedure described in Example 1(f) to give a off-white solid (5.9 mg). ¹H NMR (400 MHz, MeOD) δ ppm 8.43 (d, J=2.8 Hz, 1H), 7.74-7.82 (m, 2H), 7.50 (d, J=8.6 Hz, 1 H), 7.43 (t, J=1.6 Hz, 1H), 7.29-7.39 (m, 7H), 6.28 (d, J=1.0 Hz, 1H), 4.39 (dd, J=10.6, 3.0 Hz, 1H), 4.25 (dd, J=10.6, 5.6 Hz, 1H), 3.95 (m, 1H), 3.15 (d, J=7.6 Hz, 1H), 2.21-2.28 (m, 1H), 0.97-1.07 (m, 4H); MS: 451.2.

### Example 35

### Preparation of [(1S)-2-{[5-(3-methyl-1H-indazol-5-yl)-6-(1-methyl-1H-pyrazol-4-yl)-3-pyridinyl]oxy}-1-(phenylmethyl)ethyl]amine

a) 1-methyl-4-(4,4,5,5-tetramethyl-1,3,2-dioxaborolan-2-yl)-1H-pyrazole

To a solution of 4-(4,4,5,5-tetramethyl-1,3,2-dioxaborolan-2-yl)-1H-pyrazole(0.19 g, 1.0 mmol) in 4 ml DMF was added Mel(0.067 ml, 1.1 eq) and CS₂CO₃(0.39 g, 1.2 eq). The reaction mixture was stirred at RT for 3 h. The solution was taken up into EtOAc, washed with water, brine, dried over Na₂SO₄ and concentrated. 150 mg crude product was obtained(yield 72%).

b) [(1S)-2-{[5-(3-methyl-1H-indazol-5-yl)-6-(1-methyl-1H-pyrazol-4-yl)-3-pyridinyl]oxy}-1-(phenylmethyl)ethyl]amine

Following the procedure of Example 1(a)-1(f), except substituting 1-methyl-4-(4,4,5,5-tetramethyl-1,3,2-dioxaborolan-2-yl)-1H-pyrazole for phenylboronic acid, the title compound was prepared. ¹H NMR (CD³OD, 400 MHz) δ 8.46 (d, 1H), 7.91 (d, 1H), 7.79 (d, 1H), 7.55-7.41 (m, 2H), 7.38-7.24 (m, 7H), 4.43 (dd, 1H), 4.28 (dd, 1H), 3.99 (m, 1H), 3.80 (s, 3H), 3.19 (d, 2H), 2.59 (s, 3H). MS (M+H): 439.2.

### Example 36

### Preparation of [(1S)-2-{[6-{1-[(3-fluorophenyl)methyl]-1H-pyrazol-4-yl}-5-(3-methyl-1H-indazol-5-yl)-3-pyridinyl]oxy}-1-(phenylmethyl)ethyl]amine

a) 1-[(3-fluorophenyl)methyl]-4-(4,4,5,5-tetramethyl-1,3,2-dioxaborolan-2-yl)-1H-pyrazole

Following the procedure of Example 35(a), except substituting 1-(bromomethyl)-3-fluorobenzene for methyl iodide, the title compound was prepared.

b)[(1S)-2-{[6-{1-[(3-fluorophenyl)methyl]-1H-pyrazol-4-yl}-5-(3-methyl-1H-indazol-5-yl)-3-pyridinyl]oxy}-1-(phenylmethyl)ethyl]amine

Following the procedure of Example 1(a)-1(f), except substituting 1-[(3-fluorophenyl)methyl]-4-(4,4,5,5-tetramethyl-1,3,2-dioxaborolan-2-yl)-1H-pyrazole for phenylboronic acid, the title compound was prepared. ¹H NMR (CD₃OD, 400 MHz) δ 8.47 (d, 1H), 7.92 (d, 1H), 7.73 (d, 1H), 7.54 (s, 1H), 7.46 (d, 1H), 7.38-7.20 (m, 8H), 7.02 (dd, 1H), 6.88 (d, 1H), 6.82 (d, 1H), 5.22 (s, 2H), 4.43 (dd, 1H), 4.28 (dd, 1H), 3.99 (m, 1H), 3.18 (d, 2H), 2.54 (s, 3H). MS (M+H): 533.4.

### Example 37

### Preparation of ((1S)-2-phenyl-1-{[(6-phenyl-5-{3-[5-(1-piperazinylmethyl)-2-furanyl]-1H-indazol-5-yl}-3-pyridinyl)oxy]methyl}ethyl)amine

a) {5-[(4-{[(1,1-dimethylethyl)oxy]carbonyl}-1-piperazinyl)methyl]-2-furanyl}boronic acid

To a solution of 5-formyl-2-furanyl)boronic acid(0.034 g, 0.24 mmol) and 1-Boc-piperazine (0.037 g, 0.20 mmol) in CH₂Cl₂ was added NaBH(OAc)₃(0.064 g, 0.30 mmol). The reaction mixture was stirred at rt for a hour. The solution was concentrated and water was then added. The solution was extracted by CH₂Cl₂, dried over Na₂SO₄ and concentrated to give 0.045 g product(72%).

b) ((1S)-2-phenyl-1-{[(6-phenyl-5-{3-[5-(1-piperazinylmethyl)-2-furanyl]-1H-indazol-5-yl}-3-pyridinyl)oxy]methyl}ethyl)amine

Following the procedure of Example 23(a)-23(c), except the substituting {5-[(4-{[(1,1-dimethylethyl)oxy]carbonyl}-1-piperazinyl)methyl]-2-furanyl}boronic acid for phenylboronic acid, the title compound was prepared. ¹H NMR (CD₃OD, 400 MHz) δ 8.60 (d, 1H), 8.17 (d, 1H), 8.01 (d, 1H), 7.52 (d, 1H), 7.43-7.20 (m, 11H), 6.91 (d, 1H), 6.84 (d, 1H), 4.55 (dd, 1H), 4.43 (s, 2H), 4.28 (dd, 1H), 4.02 (m, 1H), 3.53 (br, 4H), 3.42 (br, 4H), 3.20 (d, 2H), MS (M+H): 585.4.

### Example 38

### Preparation of [(1S)-2-({6-(3-furanyl)-5-[3-(2-furanyl)-1H-indazol-5-yl]-3-pyridinyl}oxy)-1-(phenylmethyl)ethyl]amine

a) 1,1-dimethylethyl [(1S)-2-{[6-chloro-5-(1H-indazol-5-yl)-3-pyridinyl]oxy}-1-(phenylmethyl)ethyl]carbamate

Following the procedure of Example 1(a)-1(d), except substituting 5-(4,4,5,5-tetramethyl-[1,3,2]dioxaborolan-2-yl)-indazole-1-carboxylic acid tert-butyl ester for the compound of Example 1(c), the title compound was prepared.

b) 1,1-dimethylethyl [(1S)-2-{[6-chloro-5-(3-iodo-1H-indazol-5-yl)-3-pyridinyl]oxy}-1-(phenylmethyl)ethyl]carbamate

Following the procedure of Example 23(a)-23(b), except the substituting compound of Example 38(a) for the compound of Example 23(a), the title compound was prepared.

c) 1,1-dimethylethyl [(1R)-2-({6-chloro-5-[3-(2-furanyl)-1H-indazol-5-yl]-3-pyridinyl}oxy)-1-(phenylmethyl)ethyl]carbamate

Following the procedure of Example 23(b)-23(c), except the substituting 2-furanylboronic acid for phenylboronic acid and substituting the compound in Example 38(b) for the compound in Example 23(b), the title compound was prepared.

d) 1,1-dimethylethyl acetate-[(1R)-2-({6-(3-furanyl)-5-[3-(2-furanyl)-1H-indazol-5-yl]-3-pyridinyl}oxy)-1-(phenylmethyl)ethyl]amine

Following the procedure of Example 1(d)-1(f), except the substituting the compound 38(c) for the compound 1(d) and substituting 3-furanylboronic acid for phenylboronic acid, the title compound was prepared.

e) [(1S)-2-({6-(3-furanyl)-5-[3-(2-furanyl)-1H-indazol-5-yl]-3-pyridinyl}oxy)-1-(phenylmethyl)ethyl]amine

The compound in Example 38(d) (0.100 g) was dissolved in 5 ml CH₂Cl₂, TFA(1 ml) was added. The mixture was stirred at room temperature for 2 h. Solvent was removed and the residue was purified by reverse HPLC to give 0.042 g product. ¹H NMR (CD₃OD, 400 MHz) δ8.42(d, 1H), 8.10 (d, 1H), 7.65 (d, 1H), 7.60 (d, 1H), 7.48 (d, 1H), 7.44-7.32 (m, 7H), 7.20 (d, 1H), 6.96 (d, 1H), 6.63 (d, 1H), 6.31 (d, 1H), 4.37 (dd, 1, H), 4.21 (dd, 1H), 3.96 (m, 1H), 3.16 (d, 2H). MS (M+H): 477.2.

### Example 39

### Preparation of [(1S)-2-({5-(3-methyl-1H-indazol-5-yl)-6-[3-(phenyloxy)phenyl]-3-pyridinyl}oxy)-1-(phenylmethyl)ethyl]amine

a) 3-(phenyloxy)phenyl trifluoroacetate

Et₃N (0.48 ml, 1.1 eq.) was added to a solution of m-phenoxyphenol (0.5 mL, 3.11 mmol) and PhNTf₂ (1.22 g, 1.1 eq.) in DCM (5 mL). The resulting mixture was stirred at rt for 3 hr, washed with water, brine, and dried (Na₂SO₄). Removal of the solvent followed by flash column chromatographic purification of the residue on silica gel (hexane/EtOAc 95:5) afforded the product as a light yellow clear oil (0.98 g, 99%).

b) 4,4,5,5-tetramethyl-2-[3-(phenyloxy)phenyl]-1,3,2-dioxaborolane

Following the procedure of Example 1(c), except the substituting substituting 3-(phenyloxy)phenyl trifluoroacetate for N-Boc-3-methyl-5-bromoindazole, the title compound was prepared.

c) [(1S)-2-({5-(3-methyl-1H-indazol-5-yl)-6-[3-(phenyloxy)phenyl]-3-pyridinyl}oxy)-1-(phenylmethyl)ethyl]amine

Following the procedure of Example 1(d)-1(f), except the substituting substituting 4,4,5,5-tetramethyl-2-[3-(phenyloxy)phenyl]-1,3,2-dioxaborolane for phenylboronic acid, the title compound was prepared. ¹H NMR (CD₃OD, 400 MHz) δ 8.47(d, 1H), 7.87(d, 1H), 7.60(d, 1H), 7.44-7.28 (m, 8H), 7.11-7.08 (m, 2H), 6.98-6.95 (m, 3H), 6.60 (d, 1H), 6.52-6.47 (m, 2H), 4.44 (dd, 1H), 4.25(dd, 1H), 3.96 (m, 1H), 3.17 (d, 2H), 2.51 (s, 3H). MS (M+H): 527.4.

### Example 40

### Preparation of 3-[({5-[5-(5-{[(2S)-2-amino-3-phenylpropy]oxy}-2-phenyl-3-pyridinyl)-1H-indazol-3-yl]-2-furanyl}methyl)amino]propanenitrile

a) (5-{[(2-cyanoethyl)amino]methyl}-2-furanyl)boronic acid

Following the procedure of Example 37(a), except the substituting 3-aminopropionitrile for 1-Boc-piperazine, the title compound was prepared.

b) 3-[({5-[5-(5-{[(2S)-2-amino-3-phenylpropyl]oxy}-2-phenyl-3-pyridinyl)-1H-indazol-3-yl]-2-furanyl}methyl)amino]propanenitrile

Following the procedure of Example 23(a)-23(c), except the substituting (5-{[(2-cyanoethyl)amino]methyl}-2-furanyl)boronic acid for phenylboronic acid, the title compound was prepared. ¹H NMR (CD₃OD, 400 MHz) δ 8.54 (d, 1H), 8.00 (dd, 2H), 7.52 (d, 1H), 7.40-7.35 (m, 10H), 7.24 (d, 1H), 6.89 (dd, 2H), 4.51-4.47 (m, 3H), 4.34 (dd, 1H), 4.02 (m, 1H), 3.47 (t, 2H), 3.35 (d, 2H), 3.00 (t, 2H). MS (M+H): 569.4.

### Example 41

### Preparation of [(1S)-2-({6-(2-furanyl)-5-[3-(2-furanyl)-1H-indazol-5-yl]-3-pyridinyl}oxy)-1-(phenylmethyl)ethyl]amine

Following the procedure of Example 38(c)-38(d) except substituting 2-furanylboronic acid for 3-furanylboronic acid. the title compound was prepared. ¹H NMR (CD₃OD, 400 MHz) δ 8.54 (d, 1H), 8.12 (d, 1H), 7.64 (d, 1H), 7.60-7.55 (m, 2 H), 7.41 (d, 1H), 7.40-7.30 (m, 6H), 6.97 (d, 1H), 6.63 (d, 1H), 6.37 (d, 1H), 5.99 (d, 1H), 4.40 (dd, 1H), 4.36 (dd, 1H), 3.99 (m, 1H), 3.16 (d, 2H). MS (M+H): 477.0.

### Example 42

### Preparation of {5-[5-{[(2S)-2-amino-3-phenylpropyl]oxy}-3-(3-methyl-1H-indazol-5-yl)-2-pyridinyl]-2-thienyl}methanol

a) [5-(hydroxymethyl)-2-thienyl]boronic acid

To a solution of (5-formyl-2-thienyl)boronic acid(31 mg, 0.20 mmol) in MeOH(1 ml) was added NaBH₄(7.8 mg, 0.20 mmol). ). The resulting mixture was stirred at rt for 1 hr and filtered through celite. The solution was concentrated and the residue was purified by FCC to give 10 mg product.

b) {5-[5-{[(2S)-2-amino-3-phenylpropyl]oxy}-3-(3-methyl-1H-indazol-5-yl)-2-pyridinyl]-2-thienyl}methanol

Following the procedure of Example 1(a)-1(f), except substituting [5-(hydroxymethyl)-2-thienyl]boronic acid for phenylboronic acid, the title compound was prepared. ¹H NMR (CD₃OD, 400 MHz) δ 8.37 (d, 1H), 7.73 (d, 1H), 7.49-7.25 (m, 8H), 6.70 (d, 1H), 6.50 (d, 1H), 4.64 (d, 2H), 4.34 (dd, 1H), 4.19 (dd, 1H), 3.95 (m, 1H), 3.19 (d, 2H), 2.57 (s, 3H). MS (M+H): 471.2.

### Example 43

### Preparation of {(1S)-2-phenyl-1-[({6-phenyl-5-[3-(phenylmethyl)-1H-indazol-5-yl]-3-pyridinyl}oxy)methyl]ethyl}amine

BnZnBr (0.6 mL, 3.0 eq., 0.5 M in THF) was added to a suspension of the compound in Example23(b) (75 mg, 0.10 mmol) and Pd(Ph₃P)₄ (11.6 mg, 10 mol %) at 0 C. The resulting mixture was heated at 50 C for 48 hr, cooled down to rt, and neutralized with saturated NH₄Cl aqueous solution, which was extracted with DCM. The combined organic layers were dried (Na₂SO₄), concentrated and the residue was purified by FCC to give the mono-boc prod as a white foamy solid (14 mg, 23%) and the amine (23 mg, 45%). ¹H NMR (CD₃OD, 400 MHz) 68.46 (d, 1H), 7.74 (d, 1H), 7.43-7.15 (m, 18H), 4.40 (dd, 1H), 4.27 (dd, 1H), 4.24 (s, 2H), 3.98 (m, 1H), 3.17 (d, 2H). MS (M+H): 511.4.

### Example 44

### Preparation of [(1S)-2-{[5-(3-methyl-1H-indazol-5-yl)-6-(1-methyl-1H-pyrrol-2-yl)-3-pyridinyl]oxy}-1-(phenylmethyl)ethyl]amine

A mixture of the compound in Example 1(d) (60 mg, 0.1 mmol), the stannane reagent (41 mg, 1.1 eq.), CsF (33 mg, 2.2 eq.), Pd(tBu₃P)₂ (2.6 mg, 5 mol %) and 1,4-dioxane was degassed, sealed and heated at 100 C overnight. The resulting mixture was filtered through celite, which was rinsed with EtOAc. The combined organic layers were dried (Na₂SO₄), concentrated and the residue was purified by FCC to give the product as a light brown oil (40 mg, 63%). ¹H NMR (CD₃OD, 400 MHz) δ 8.53 (d, 1H), 8.32 (d, 1H), 7.61 (d, 1H), 7.48 (d, 1H), 7.40-7.28 (m, 6H), 6.80 (dd, 1H), 6.55 (dd, 1H), 6.30 (dd, 1H), 4.56 (dd, 1H), 4.40 (dd, 1H), 4.06 (m, 1H), 3.20 (d, 2H), 2.95 (s, 3H), 2.50 (s, 3H). MS (M+H): 438.2.

### Example 45

### Preparation of 5-(5-{[(2S)-2-amino-3-phenylpropyl]oxy}-2-phenyl-3-pyridinyl)-1H -indazol-3-amine

a) 1,1-dimethylethyl 5-(5-{[(2S)-2-({[(1,1-dimethylethyl)oxy]carbonyl}amino)-3-phenylpropyl]oxy}-2-phenyl-3-pyridinyl)-3-[(diphenylmethylidene)amino]-1H -indazole-1-carboxylate

To a solution of 23(b)(76 mg, 0.1 mmol), Pd₂dba₃(2%, 1.8 mg), Xantphos(6%, 3.5 mg) and CS₂CO₃(45.6 mg, 1.4 eq) in 0.5 ml dioxane was added 1,1-diphenylmethanimine(0.024 ml, 1.4 eq). The reaction mixture was stirred at 100° C. for 20 min. The solution was concentrated and purified by FCC to give 24 mg product(30%).
   b) 1,1-dimethylethyl 3-amino-5-(5-{[(2S)-2-amino-3-phenylpropyl]oxy}-2-phenyl-3-pyridinyl)-1H-indazole-1-carboxylate
To a solution of Example 50(a)(24 mg, 0.030 mmol) in 0.3 ml MeOH was added NH₂OHHCl(2.3 mg, 1.1 eq). The resulting mixture was stirred at rt for overnight. Removed solvent and purified by FCC to give 16 mg product(84%).
   c) 5-(5-{[(2S)-2-amino-3-phenylpropyl]oxy}-2-phenyl-3-pyridinyl)-1H-indazol-3-amine
Following the procedure of Example 1(e)-1(f), except substituting the compound in Example 50(b) for the compound in Example 1(d), the title compound was prepared. ¹H NMR (CD₃OD, 400 MHz) δ 8.54 (d, 1H), 7.90 (dd, 2H), 7.41-7.30 (m, 12H), 4.44 (dd, 1H), 4.30 (dd, 1H), 4.00 (m, 1H), 3.32 (d, 2H). MS (M+H): 436.2.

### Example 46

### Preparation of [(1S)-2-({5-[3-(1-methylethenyl)-1H-indazol-5-yl]-6-phenyl-3-pyridinyl}oxy)-1-(phenylmethyl)ethyl]amine

0.6 Ml ZnCl2 solution(0.5 M in THF) was added to the 0.6 ml solution of bromo(1-methylethenyl)magnesium (0.5 M in THF) at 0° C. White precipitate formed in 5 min. The compound in Example 23(b)(0.075 mg, 0.1 mmol) and Pd(Ph₃P)₄ were added subsequently. The resulting mixture was heated up to 50° C. for 2.5 h.

The mixture was taken up in EtOAc, washed with water, brine and dried over Na2SO4. Removal of the solvent followed by flash column chromatographic purification of the residue on silica gel afforded the product as a light brown solid (0.044 g, 78%). ¹H NMR (CD₃OD, 400 MHz) δ 8.46 (d, 1H), 7.84 (d, 1H), 7.78 (d, 1H), 7.46-7.22 (m, 12H), 5.42 (d, 1H), 5.20 (d, 1H), 4.43 (dd, 1H), 4.29 (dd, 1H), 3.99 (m, 1H), 3.19 (d, 2H), 2.24 (s, 3H). MS (M+H): 461.2.

### Example 47

### Preparation of [(1S)-2-{[5-(3-methyl-1H-indazol-5-yl)-6-(1H-pyrazol-4-yl)-3-pyridinyl]oxy}-1-(phenylmethyl)ethyl]amine

Following the procedure of Example 1(a)-1(f), except substituting 4-(4,4,5,5-tetramethyl-1,3,2-dioxaborolan-2-yl)-1H-pyrazole for phenylboronic acid, the title compound was prepared. ¹H NMR (CD₃OD, 400 MHz) δ 8.44 (d, 1H), 7.81 (d, 1H), 7.77 (d, 1H), 7.53 (d, 1H), 7.41-7.24 (m, 8H), 4.40 (dd, 1H), 4.27 (dd, 1H), 3.96 (m, 1H), 3.16 (d, 2H), 2.62 (s, 3H). MS (M+H): 425.2.

### Example 48

### Preparation of (2S)-N,N-dimethyl-1-{[5-(3-methyl-1H-indazol-5-yl)-6-phenyl-3-pyridinyl]oxy}-3-phenyl-2-propanamine

To a solution of the compound in Example 1(f) (40 mg, 1.0 eq), in 2 ml MeOH was added formaldehyde(4.0 eq) and NaCNBH₃(4.0 eq). The reaction mixture was stirred at rt for 2 hours. The solvent was removed and EtOAc was added. The solution was washed with aq. NaHCO₃ and brine and dried over Na₂SO₄. Removal of the solvent followed by flash column chromatographic purification of the residue on silica gel afforded 31 mg product(70%).

1H NMR (CD₃OD, 400 MHz) δ 8.48 (d, 1H), 7.88 (d, 1H), 7.66 (d, 1H), 7.41-7.30 (m, 11H), 7.08 (d, 1H), 4.53 (dd, 1H), 4.41 (dd, 1H), 4.14 (m, 1H), 3.21 (d, 2H), 3.14 (s, 6H), 2.50 (s, 3H). MS (M+H): 463.0.

### Example 49

### Preparation of [(1S)-2-{[3-(3-methyl-1H-indazol-5-yl)-2,4′-bipyridin-5-yl]oxy}-1-(phenylmethyl)ethyl]amine

Following the procedure of Example 1(a)-1(f), except substituting 4-pyridinylboronic acid for phenylboronic acid, the title compound was prepared. ¹H NMR (CD₃OD, 400 MHz) δ 8.63-8.61 (m, 3H), 7.91 (d, 2H), 7.72 (d, 1H), 7.61 (d, 1H), 7.46 (d, 1H), 7.35-7.32 (m, 5H), 7.17 (d, 1H), 4.42 (dd, 1H), 4.28 (dd, 1H), 3.98 (m, 1H), 3.17 (d, 2H), 2.55 (s, 3H). MS (M+H): 436.2.

### Example 50

### Preparation of [(1S)-2-{[3-(3-methyl-1H-indazol-5-yl)-2,3′-bipyridin-5-yl]oxy}-1-(phenylmethyl)ethyl]amine

Following the procedure of Example 1(a)-1(f), except substituting 3-pyridinylboronic acid for phenylboronic acid, the title compound was prepared. ¹H NMR (CD₃OD, 400 MHz) δ 8.63-8.56 (m, 3H), 8.21 (d, 1H), 7.74-7.68 (m, 2H), 7.62 (d, 1H), 7.46-7.32 (m, 6H), 7.15 (d, 1H), 4.41 (dd, 1H), 4.25 (dd, 1H), 4.01 (m, 1H), 3.19 (d, 2H), 2.54 (s, 3H). MS (M+H): 436.2.

### Example 51

### Preparation of [(1S)-2-{[5-(3-iodo-1H-indazol-5-yl)-6-phenyl-3-pyridinyl]oxy}-1-(phenylmethyl)ethyl]amine

To a solution of 23(b) was added TFA in CH2Cl2 followed by reverse phase HPLC purification, the titled compound was prepared. ¹H NMR (CD₃OD, 400 MHz) δ 88.44 (d, 1H), 7.65 (d, 1H), 7.40-7.27 (m, 12H), 7.15 (d, 1H), 4.40 (dd, 1H), 4.25 (dd, 1H), 3.98 (m, 1H), 3.17 (d, 2H). MS (M+H): 547.2.

### Example 52

### Preparation of [(1S)-2-[(5-(3-methyl-1H-indazol-5-yl)-6-{3-[(trifluoromethyl)oxy]phenyl}-3-pyridinyl)oxy]-1-(phenylmethyl)ethyl]amine

Following the procedure of Example 1(a)-1(f), except substituting {3-[(trifluoromethyl)oxy]phenyl}boronic acid for phenylboronic acid, the title compound was prepared. ¹H NMR (CD₃OD, 400 MHz) δ 8.45 (d, 1H), 7.63-7.60 (m, 2H), 7.42-7.34 (m, 8H), 7.19-7.10 (m, 3H), 4.40 (dd, 1H), 4.24 (dd, 1H), 3.97 (m, 1H), 3.19 (d, 2H), 2.50 (s, 3H). MS (M+H): 519.2.

### Example 53

### Preparation of [(1S)-2-{[6-(3,5-dimethyl-4-isoxazolyl)-5-(3-methyl-1H-indazol-5-yl)-3-pyridinyl]oxy}-1-(phenylmethyl)ethyl]amine

Following the procedure of Example 1(a)-1(f), except substituting (3,5-dimethyl-4-isoxazolyl)boronic acid for phenylboronic acid, the title compound was prepared. ¹H NMR (CD₃OD, 400 MHz) δ 8.47 (d, 1H), 7.70 (dd, 2H), 7.44 (d, 1H), 7.39-7.32 (m, 5H), 7.17 (d, 1H), 4.43 (dd, 1H), 4.25 (dd, 1H), 3.98 (m, 1H), 3.20 (d, 2H), 2.55 (s, 3H), 2.00 (s, 3H), 1.92 (s, 3H). MS (M+H): 454.2.

### Example 54

### Preparation of 4-[5-{[(2S)-2-amino-3-phenylpropyl]oxy}-3-(3-methyl-1H-indazol-5-yl)-2-pyridinyl]phenol

Following the procedure of Example 1(a)-1(f), except substituting 4-(4,4,5,5-tetramethyl-1,3,2-dioxaborolan-2-yl)phenol for phenylboronic acid, the title compound was prepared. ¹H NMR (CD₃OD, 400 MHz) δ 8.48 (d, 1H), 8.09 (d, 1H), 7.73 (d, 1H), 7.42-7.32 (m, 6H), 7.18-7.11 (m, 3H), 6.75 (d, 2H), 4.48 (dd, 1H), 4.36 (dd, 1H), 4.02 (m, 1H), 3.19 (d, 2H), 2.54 (s, 3H). MS (M+H): 451.4.

### Example 55

### Preparation of 2-[5-{[(2S)-2-amino-3-phenylpropyl]oxy}-3-(3-methyl-1H-indazol-5-yl)-2-pyridinyl]phenol

Following the procedure of Example 1(a)-1(f), except substituting 2-(4,4,5,5-tetramethyl-1,3,2-dioxaborolan-2-yl)phenol for phenylboronic acid, the title compound was prepared. ¹H NMR (CD₃OD, 400 MHz) δ 8.56 (d, 1H), 8.24 (d, 1H), 7.68 (s, 1H), 7.43-7.29 (m, 7H), 7.24 (d, 1H), 7.08 (d, 1H), 6.90 (d, 1H), 6.79 (dd, 1H), 4.52 (dd, 1H), 4.50 (dd, 1H), 4.02 (m, 1H), 3.19 (d, 2H), 2.48 (s, 3H). MS (M+H): 451.2.

### Example 56

### Preparation of [(1S)-2-{[6-[3-(ethyloxy)phenyl]-5-(3-methyl-1H-indazol-5-yl)-3-pyridinyl]oxy}-1-(phenylmethyl)ethyl]amine

Following the procedure of Example 1(a)-1(f), except substituting [3-(ethyloxy)phenyl]boronic acid for phenylboronic acid, the title compound was prepared. ¹H NMR (CD₃OD, 400 MHz) δ 8.42 (d, 1H), 7.72 (d, 1H), 7.65 (d, 1H), 7.42-7.36 (m, 6H), 7.19 (dd, 1H), 7.17 (d, 1H), 6.87-6.82 (m, 3H), 4.41 (dd, 1H), 4.26 (dd, 1H), 4.00 (m, 1H), 3.83 (q, 2H), 3.16 (d, 2H), 2.52 (s, 3H), 1.22 (t, 3H). MS (M+H): 479.2.

### Example 57

### Preparation of [(1S)-2-({5-(3-methyl-1H-indazol-5-yl)-6-[3-(methyloxy)phenyl]-3-pyridinyl}oxy)-1-(phenylmethyl)ethyl]amine

Following the procedure of Example 1(a)-1(f), except substituting [3-(methoxy)phenyl]boronic acid for phenylboronic acid, the title compound was prepared. ¹H NMR (CD₃OD, 400 MHz) δ 8.46 (d, 1H), 7.82 (d, 1H), 7.68 (d, 1H), 7.41-7.30 (m, 6H), 7.19 (dd, 1H), 7.11 (d, 1H), 6.92-6.85 (m, 3H), 4.45 (dd, 1H), 4.30 (dd, 1H), 4.02 (m, 1H), 3.62 (s, 3H), 3.19 (d, 2H), 2.53 (s, 3H). MS (M+H): 465.4.

### Example 58

### Preparation of {3-[5-{[(2S)-2-amino-3-phenylpropyl]oxy}-3-(3-methyl-1H-indazol-5-yl)-2-pyridinyl]phenyl}(phenyl)methanone

a) Phenyl[3-(4,4,5,5-tetramethyl-1,3,2-dioxaborolan-2-yl)phenyl]methanone

Following the procedure of Example 44(a)-44(b), except the substituting substituting (3-hydroxyphenyl)(phenyl)methanone for m-phenoxyphenol, the title compound was prepared.
   b) {3-[5-{[(2S)-2-amino-3-phenylpropyl]oxy}-3-(3-methyl-1H-indazol-5-yl)-2-pyridinyl]phenyl}(phenyl)methanone
Following the procedure of Example 1(a)-1(f), except substituting phenyl[3-(4,4,5,5-tetramethyl-1,3,2-dioxaborolan-2-yl)phenyl]methanone for phenylboronic acid, the title compound was prepared. ¹H NMR (CD₃OD, 400 MHz) δ 8.44 (d, 1H), 7.80 (d, 2H), 7.58-7.40 (m, 3H), 7.46-7.24 (m, 10H), 7.12-7.04 (m, 3H), 4.40 (dd, 1H), 7.24 (dd, 1H), 3.92 (m, 1H), 3.18 (d, 2H), 2.54 (s, 3H). MS (M+H): 539.4.

### Example 59

### Preparation of [(1S)-2-{[6-{3-[(1-methylethyl)oxy]phenyl}-5-(3-methyl-1H-indazol-5-yl)-3-pyridinyl]oxy}1-(phenylmethyl)ethyl]amine

Following the procedure of Example 1(a)-1(f), except substituting [3-( methylethyl)oxy)phenyl]boronic acid for phenylboronic acid, the title compound was prepared. ¹H NMR (CD₃OD, 400 MHz) δ 8.44 (d, 1H), 7.76 (m, 1H), 7.65 (d, 1H), 7.40-7.33 (m, 6H), 7.22 (dd, 1H), 7.14 (dd, 1H), 6.94 (d, 1H), 6.83 (d, 1H), 6.74 (s, 1H), 4.41 (dd, 1H), 4.29-4.23 (m, 2H), 3.98 (m, 1H), 3.19 (d, 2H), 2.51 (s, 3H). 1.04 (d, 6H) MS (M+H): 493.2.

### Example 60

### Preparation of [(1S)-2-{[5-[3-(2-furanyl)-1H-indazol-5-yl]-6-(1H-pyrrol-2-yl)-3-pyridinyl]oxy}-1-(phenylmethyl)ethyl]amine

Following the procedure of Example 1(a)-1(f), except substituting (1-{[(1,1-dimethylethyl)oxy]carbonyl}-1H-pyrrol-2-yl)boronic acid for phenylboronic acid, the title compound was prepared. ¹H NMR (CD₃OD, 400 MHz) δ 8.45 (d, 1H), 8.12 (d, 1H), 7.60-7.52 (m, 3H), 7.40-7.28 (m, 6H), 6.96 (d, 1H), 6.81 (d, 1H), 6.62 (d, 1H), 5.97 (d, 1H), 5.61 (d, 1H), 4.37 (dd, 1H), 4.18 (dd, 1H), 4.00 (m, 1H), 3-19 (d, 2H). MS (M+H): 476.2.

### Example 61

### Preparation of [(1S)-2-{[6-(2-{[(3-fluorophenyl)methyl]oxy}phenyl)-5-(3-methyl-1H-indazol-5-yl)-3-pyridinyl]oxy}-1-(phenylmethyl)ethyl]amine

a) 2-(2-{[(3-fluorophenyl)methyl]oxy}phenyl)-4,4,5,5-tetramethyl-1,3,2-dioxaborolane

2-(4,4,5,5-tetramethyl-1,3,2-dioxaborolan-2-yl)phenol(0.14 g, 0.64 mmol) and Cs₂CO₃(0.26 g, 0.80 mmol) were added to a solution of 1-(bromomethyl)-3-fluorobenzene(0.10 g, 0.53 mmol) in DMF (5 ml). The reaction mixture was stirred at rt for 1 h. Removed DMF. The residue was diluted with EtOAc, washed with aq NaHCO₃ and brine. Purification by flash column chromatography gave 0.12 g product(yield 71%).

b) [(1S)-2-{[6-(2-{[(3-fluorophenyl)methyl]oxy}phenyl)-5-(3-methyl-1H -indazol-5-yl)-3-pyridinyl]oxy}-1-(phenylmethyl)ethyl]amine

Following the procedure of Example 1(a)-1(f), except substituting 2-(2-{[(3-fluorophenyl)methyl]oxy}phenyl)-4,4,5,5-tetramethyl-1,3,2-dioxaborolane for phenylboronic acid, the title compound was prepared. ¹H NMR (CD₃OD, 400 MHz) δ 8.52 (d, 1H), 7.99 (d, 1H), 7.45-7.15 (m, 10H), 7.07-6.95 (m, 4H), 6.77-6.70 (m, 2H), 4.76 (s, 2H), 4.45 (dd, 1H), 4.28 (dd, 1 h), 3.99 (m, 1H), 3.18 (d, 2), 2.37 (s, 3H). MS (M+H): 559.2.

### Example 62

### Preparation of [(1s)-2-{[6-(4-{[(3-fluorophenyl)methyl]oxy}phenyl)-5-(3-methyl-1H-indazol-5-yl)-3-pyridinyl]oxy}-1-(phenylmethyl)ethyl]amine

a) 2-(4-{[(3-fluorophenyl)methyl]oxy}phenyl)-4,4,5,5-tetramethyl-1,3,2-dioxaborolane

Following the procedure of Example 66(a), except substituting 4-(4,4,5,5-tetramethyl-1,3,2-dioxaborolan-2-yl)phenol for 2-(4,4,5,5-tetramethyl-1,3,2-dioxaborolan-2-yl)phenol, the title compound was prepared.

b) [(1S)-2-{[6-(4-{[(3-fluorophenyl)methyl]oxy}phenyl)-5-(3-methyl-1H-indazol-5-yl)-3-pyridinyl]oxy}-1-(phenylmethyl)ethyl]amine

Following the procedure of Example 1(a)-1(f), except substituting 2-(4-{[(3-fluorophenyl)methyl]oxy}phenyl)-4,4,5,5-tetramethyl-1,3,2-dioxaborolane for phenylboronic acid, the title compound was prepared.¹H NMR (CD₃OD, 400 MHz) δ 8.40 (d, 1H), 7.65 (dd, 2H), 7.44-7.05 (m, 13H), 6.90 (d, 2H), 5.09 (s, 2H), 4.38 (dd, 1H), 4.24 (dd, 1H), 3.96 (m, 1H), 3.18 (d, 2H), 2.52 (s, 3H). MS (M+H): 559.2.

### Example 63

### Preparation of [(1S)-2-({5-[3-(5-chloro-2-thienyl)-1H-indazol-5-yl]-6-phenyl-3-pyridinyl}oxy)-1-(phenylmethyl)ethyl]amine

Following the procedure of Example 23(a)-23(c), except substituting (5-chloro-2-thienyl)boronic acid for phenylboronic acid, the title compound was prepared. ¹H NMR (CD₃OD, 400 MHz) δ 8.45 (d, 1H), 7.83 (d, 1H), 7.75 (d, 1H), 7.49-7.23 (m, 13H), 7.03 (d, 1H), 4.43 (dd, 1H), 4.26 (dd, 1H), 4.00 (m, 1H), 3.23 (d, 2H). MS (M+H): 537.2.

### Example 64

### Preparation of [(1S)-2-({5-[3-(4-methyl-2-thienyl)-1H-indazol-5-yl]-6-phenyl-3-pyridinyl}oxy)-1-(phenylmethyl)ethyl]amine

Following the procedure of Example 23(a)-23(c), except substituting (4-methyl-2-thienyl)boronic acid for phenylboronic acid, the title compound was prepared. ¹H NMR (CD₃OD, 400 MHz) δ 8.43 (d, 1H), 7.81 (d, 1H), 7.69 (d, 1H), 7.47 (d, 1H), 7.44-7.26 (m, 11H), 7.14 (s, 1H), 7.02 (s, 1H), 4.40 (dd, 1H), 4.24 (dd, 1H), 3.97 (m, 1H), 3.20 (d, 2H), 2.33 (s, 3H). MS (M+H): 517.2.

### Example 65

### Preparation of [(1S)-2-({5-[3-(5-methyl-2-furanyl)-1H-indazol-5-yl]-6-phenyl-3-pyridinyl}oxy)-1-(phenylmethyl)ethyl]amine

Following the procedure of Example 23(a)-23(c), except substituting (5-methyl-2-furanyl)boronic acid for phenylboronic acid, the title compound was prepared. ¹H NMR (CD₃OD, 400 MHz) 68.45 (d, 1H), 7.93 (d, 1H), 7.73 (d, 1H), 7.45-7.29 (m, 12H), 6.71 (d, 1H), 6.19 (d, 1H), 4.40 (dd, 1H), 4.25 (dd, 1H), 3.98 (m, 1H), 3.23 (d, 2H), 2.40 (s, 3H). MS (M+H): 501.4.

### Example 66

### Preparation of [(1S)-2-({5-[3-(5-methyl-2-thienyl)-1H-indazol-5-yl]-6-phenyl-3-pyridinyl}oxy)-1-(phenylmethyl)ethyl]amine

Following the procedure of Example 23(a)-23(c), except substituting (5-methyl-2-thienyl)boronic acid for phenylboronic acid, the title compound was prepared. ¹H NMR (CD₃OD, 400 MHz) δ 8.43 (d, 1H), 7.84 (s, 1H), 7.69 (d, 1H), 7.40-7.18 (m, 13H), 6.83 (dd, 1H), 4.41 (dd, 1H), 4.25 (dd, 1H), 3.96 (m, 1H), 3.19 (d, 2H), 2.54 (s, 3H). MS (M+H): 517.2.

### Example 67

### Preparation of [(1S)-2-{[6-ethenyl-5-(3-methyl-1H-indazol-5-yl)-3-pyridinyl]oxy}-1-(phenylmethyl)ethyl]amine

Following the procedure of Example 1(a)-1(f), except substituting triethenylboroxin for phenylboronic acid, the title compound was prepared. ¹H NMR (CD₃OD, 400 MHz) δ 8.44 (d, 1H), 7.78 (dd, 2H), 7.63 (d, 1H), 7.42-7.37 (m, 6H), 6.78 (dd, 1H), 6.23 (dd, 1H), 5.61 (dd, 1H), 4.42 (dd, 1H), 4.27 (dd, 1H), 3.96 (m, 1H), 3.15 (d, 2H), 2.61 (s, 3H). MS (M+H): 385.2.

### Example 68

### Preparation of {(1S)-2-phenyl-1-[({6-phenyl-5-[3-(1H-pyrrol-2-yl)-1H-indazol-5-yl]-3-pyridinyl}oxy)methyl]ethyl}amine

Following the procedure of Example 23(a)-23(c), except substituting (1-{[(1,1-dimethylethyl)oxy]carbonyl}-1H-pyrrol-2-yl)boronic acid for phenylboronic acid, the title compound was prepared. ¹H NMR (CD₃OD, 400 MHz) δ 8.47 (d, 1H), 7.87 (dd, 2H), 7.41-7.29 (m, 11H), 7.15 (dd, 1H), 6.90 (d, 1H), 6.48 (d, 1H), 6.23 (d, 1H), 4.46 (dd, 1H), 4.31 (dd, 1H), 3.99 (m, 1 h), 3.19 (d, 2H). MS (M+H): 586.4.

### Example 69

### Preparation of [(1S)-2-(1H-indol-3-yl)-1-({[5-(3-methyl-1H-indazol-5-yl)-6-phenyl-3-pyridinyl]oxy}methyl)ethyl]amine

a) 1,1-dimethylethyl [(1S)-2-[(5-bromo-6-chloro-3-pyridinyl)oxy]-1-(1H-indol-3-ylmethyl)ethyl]carbamate

Following the procedure of Example 1(a)-1(b), except substituting 1,1-dimethylethyl [(1S)-2-hydroxy-1-(1H-indol-3-ylmethyl)ethyl]carbamate for ((S)-1-Hydroxymethyl-2-phenyl-ethyl)-carbamic acid tert-butyl ester, the title compound was prepared.

b) 1,1-dimethylethyl [(1S)-2-(1H-indol-3-yl)-1-({[5-(3-methyl-1H-indazol-5-yl)-6-phenyl-3-pyridinyl]oxy}methyl)ethyl]carbamate

A solution of the compound of Example 69(a)(100 mg, 1.0 eq), 3-Methyl-5-(4,4,5,5-tetramethyl-[1,3,2]dioxaborolan-2-yl)-indazole-1-carboxylic acid tert-butyl ester(1c)(105 mg, 1.1 eq), Pd(PPh₃)₄(0.05 eq) and 0.5 ml 5% aqueous NaHCO₃ in dioxane was heated at 150° C. for 10 min in microwave. To the reaction mixture was added another 0.2 ml 5% aqueous NaHCO₃, 0.05 eq of Pd(PPh₃)₄ and phenylboronic acid(135 mg, 1.2 eq). The reaction mixture was heated at 150° C. for 10 min in microwave. The reaction mixture was concentrated and purified by flash column chromatography (30%-50%-60% hexane/EtOAc) to give 86 mg product (yield 72%).

c) [(1S)-2-(1H-indol-3-yl)-1-({[5-(3-methyl-1H-indazol-5-yl)-6-phenyl-3-pyridinyl]oxy}methyl)ethyl]amine

The solution of 69(b) in 5 ml CH₂Cl₂ was added 1 ml TFA. The reaction mixture was stirred at room temperature for 1 h. The solution was concentrated and crude product was purified by reverse phase HPLC. ¹H NMR (CD₃OD, 400 MHz) δ 8.45 (d, 1H), 7.83 (d, 1H), 7.63-7.61 (m, 2H), 7.41-7.27 (m, 9H), 7.16 (dd, 1H), 7.13-7.03 (m, 2H), 4.50 (dd, 1H), 4.38 (dd, 1H), 4.04 (m, 1H), 3.36 (d, 2H), 2.50 (s, 3H). MS (M+H): 474.4.

### Example 70

### Preparation of 5-(3-methyl-1H-indazol-5-yl)-6-phenyl-N-(3-phenylpropyl)-3-pyridinamine

a) 5-bromo-6-chloro-N-(3-phenylpropyl)-3-pyridinamine

To the solution of 5-bromo-6-chloro-3-pyridinamine(0.200 g, 0.97 mmol) in 5 ml CH₂Cl₂ was added 3-phenylpropanal(0.195 g, 1.45 mmol) followed by Na(OAc)₃BH(0.411 g, 1.94 mmol). The reaction mixture was stirred at room temperature for 1 h. The solution was quenched with water (5 ml) and product was extracted with CH₂Cl₂ (5 ml×3). The organic layer was dried over Na₂SO₄, concentrated. The compound was purified by flash column chromatography to give 0.136 g product (yield 50%).

b) 5-(3-methyl-1H-indazol-5-yl)-6-phenyl-N-(3-phenylpropyl)-3-pyridinamine

Following the procedure of Example 69(a)-69(c), except substituting the compound in Example 70(a) for the compound in Example 69(a), the title compound was prepared. ¹H NMR (CD₃OD, 400 MHz) 68.54 (s, 1H), 7.56 (d, 1H), 7.38-7.06 (m, 12H), 7.04 (d, 1H), 3.29 (t, 2H), 2.80 (t, 2H), 2.58 (s, 3H), 2.07 (m, 2H). MS (M+H): 419.2.

### Example 71

### Preparation of 5-(3-methyl-1H-indazol-5-yl)-6-phenyl-N-(3-phenylbutyl)-3-pyridinamine

Following the procedure of Example 70 except substituting 3-phenylbutanal for 3-phenylpropanal, the title compound was prepared. ¹H NMR (CD₃OD, 400 MHz) δ 8.46 (s, 1H), 7.53 (s, 1H), 7.38-7.16 (m, 12H), 7.02 (d, 1H), 3.15 (dt, 2H), 2.89 (m, 1H), 2.58 (s, 3H), 2.02 (m, 2H), 1.33 (d, 3H). MS (M+H): 433.4.

### Example 72

### Preparation of [(2S)-2-amino-3-phenylpropyl][5-(3-methyl-1H-indazol-5-yl)-6-phenyl-3-pyridinyl]amine

a) 1,1-dimethylethyl [(1S)-2-[(5-bromo-6-chloro-3-pyridinyl)amino]-1-(phenylmethyl)ethyl]carbamate

Following the procedure of Example 70(a) except for substituting N-Boc -(2S)-2-amino-3-phenylpropanal for 3-phenylpropanal, the title compound was prepared.

b) 1,1-dimethylethyl [(1S)-2-{[5-(3-methyl-1H-indazol-5-yl)-6-phenyl-3-pyridinyl]amino}-1-(phenylmethyl)ethyl]carbamate

Following the procedure of Example 70(b) except for substituting the compound in Example 77(a) for the compound in Example 70(a), the title compound was prepared.

c) [(2S)-2-amino-3-phenylpropyl][5-(3-methyl-1H-indazol-5-yl)-6-phenyl-3-pyridinyl]amine

To a solution of compound in Example 72(b)(0.102 g) in 5 ml CH₂Cl₂ was added 1 ml TFA. The reaction mixture was stirred at room temperature for 1 h. The solution was concentrated under vacumm and crude product was purified by reverse phase HPLC. 0.080 g product was obtained (yield 46%, 2 steps). ¹H NMR (CD₃OD, 400 MHz) δ 8.04 (d, 1H), 7.64 (d, 2H), 7.45-7.08 (m, 11H), 7.06 (d, 1H), 3.06 (m, 1H), 3.60 (m, 2H), 3.11 (m, 2H), 2.51 (s, 3H). MS (M+H): 434.2.

### Example 73

### Preparation of [(2S)-2-amino-3-phenylpropyl][6-(3-furanyl)-5-(3-methyl-1H-indazol -5-yl)-3-pyridinyl]amine

Following the procedure of Example 72 except for substituting 3-furanboronic acid for phenylboronic acid, the title compound was prepared. ¹H NMR (CD₃OD, 400 MHz) δ 7.98 (d, 1H), 7.73 (s, 1H), 7.55-7.50 (m, 4H), 7.33-7.12 (6 h), 6.26 (d, 1H), 3.79 (m, 2H), 3.08 (m, 2H), 2.59 (s, 3H). MS (M+H): 424.2.

### Example 74

### Preparation of ((1S)-2-{[6-(3-furanyl)-5-(3-methyl-1H-indazol-5-yl)-3-pyridinyl]oxy}-1-{[(phenylmethyl)oxy]methyl}ethyl)amine

Following the procedure of Example 70(a)-70(b), except substituting phenoxy acetaldehyde for 3-phenylpropanal and substituting 3-furanboronic acid for phenylboronic acid, the title compound was prepared. ¹H NMR (CD₃OD, 400 MHz) δ 8.41 (d, 1H), 7.74 (d, 1H), 7.70 (d, 1H), 7.54 (d, 1H), 7.41-7.26 (8H), 6.31 (dd, 1H), 4.65 (s, 2H), 4.47 (m, 1H), 4.40 (m, 1H), 3.90-3.81 (m, 3H), 2.58 (s, 3H). MS (M+H): 455.0.

### Example 75

### Preparation of N-[(2S)-2-amino-3-phenylpropyl]-N-[5-(3-methyl-1H-indazol-5-yl)-6-phenyl-3-pyridinyl]methanesulfonamide

a) 1,1-dimethylethyl [(1S)-2-[(5-bromo-6-chloro-3-pyridinyl)(phenylsulfonyl)amino]-1-(phenylmethyl)ethyl]carbamate

To a solution of the compound in Example 72(a)(0.150 g, 0.34 mmol) in 3 ml CH₂Cl₂ was added 0.1 ml Et₃N(0.70 mmol) followed by 0.052 ml benzosulfonic acid(0.41 mmol). The reaction mixture was stirred at room temperature for 1 h, and taken up into CH₂Cl₂ and water. The organic layer was separated, washed with brine, dried over Na₂SO₄ and concentrated. The residue was purified by flash column chromatography on silica gel to give 0.160 g product(yield 81%).

b) N-[(2S)-2-amino-3-phenylpropyl]-N-[5-(3-methyl-1H-indazol-5-yl)-6-phenyl-3-pyridinyl]methanesulfonamide

Following the procedure of Example 72(b)-72(c) except for substituting the compound in Example 80(a) for the compound in Example 77(a), the title compound was prepared. ¹H NMR (CD₃OD, 400 MHz) δ 8.73 (d, 1H), 7.92 (d, 1H), 7.50 (s, 1H), 7.36-7.01 (m, 11H), 6.99 (d, 1H). 4.07 (d, 2H), 3.71 (m, 1H), 3.11-2.95 (m, 4H), 2.92 (m, 1H), 2.51 (s, 3H). MS (M+H): 512.4.

### Example 76

### Preparation of 5-(3-methyl-1H-indazol-5-yl)-N-[2-methyl-2-(phenylthio)propyl]-6-phenyl-3-pyridinamine

Following the procedure of Example 70(a)-70(b), except substituting 2-methyl-2-(phenylthio)propanal for 3-phenylpropanal, the title compound was prepared. ¹H NMR (CD₃OD, 400 MHz) δ 8.52 (d, 1H), 7.59-7.26 (m, 13H), 7.03 (dd, 1H), 3.10 (s, 2H), 2.55 (s, 3H), 1.38 (s, 6H). MS (M+H): 465.2.

### Example 77

### Preparation of [(1S)-2-{[6-(3-furanyl)-5-(3-methyl-1H-indazol-5-yl)-3-pyridinyl]oxy}-1-(1H-indol-3-ylmethyl)ethyl]amine

Following the procedure of Example 69(a)-69(c), except substituting 3-furanboronic acid for phenylboronic acid, the title compound was prepared. ¹H NMR (CD₃OD, 400 MHz) δ 8.38 (d, 1H), 7.68 (d, 1H), 7.59-7.57 (m, 2H), 7.52 (d, 1H), 7.40-7.35 (m, 2H), 7.25-7.22 (m, 3H), 7.14 (dd, 1H), 7.04 (dd, 1H), 6.30 (dd, 1H), 4.41 (dd, 1H), 4.28 (dd, 1H), 4.00 (m, 1H), 3.37 (d, 2H), 2.57 (s, 3H). MS (M+H): 464.4.

### Example 78

### Preparation of ((1S)-2-{[5-(3-methyl-1H-indazol-5-yl)-6-phenyl-3-pyridinyl]oxy}-1-{[(phenylmethyl)oxy]methyl}ethyl)amine

Following the procedure of Example 1(a)-1(f), except substituting 1,1-dimethylethyl ((1S)-2-hydroxy-1-{[(phenylmethyl)oxy]methyl}ethyl)carbamate for 1,1-dimethylethyl [(1R)-2-hydroxy-1-(phenylmethyl)ethyl]carbamate, the title compound was prepared. ¹H NMR (CD₃OD, 400 MHz) δ 8.45 (d, 1H), 7.80 (d, 1H), 7.66 (d, 1H), 7.43-7.29 (m, 11H), 7.12 (dd, 1H), 4.66 (s, 2H), 4.54-4.43 (m, 2H), 3.94-3.93 (m, 1H), 3.90-3.82 (m, 2H), 2.50 (s, 3H). MS (M+H): 465.4.

### Example 79

### Preparation of (2S)-2-amino-3-{[5-(3-methyl-1H-indazol-5-yl)-6-phenyl-3-pyridinyl}oxy-1-propanol

To a solution of the compound in Example 78 (250 mg) in 5 ml EtOH was added Pd/C(200 mg). The reaction mixture was charged with vac/H₂/vac/H₂/vac/H₂. The reaction mixture eas heated at 50° C. overnight. The mixture was then filtered. The resulted organic solution was concentrated in vacuo. Separation by flash column chromatography provided 188 mg product(yield 87%). ¹H NMR (CD₃OD, 400 MHz) δ 8.44 (d, 1H), 7.75 (d, 1H), 7.65 (s, 1H), 7.35-7.28 (m, 6H), 7.10 (dd, 1H), 4:52-4.40 (m, 2H), 3.95-3.85 (m, 2H), 3.83 (m, 1H), 2.51 (s, 3H). MS (M+H): 375.4.

### Example 80

### Preparation of 5-(3-methyl-1H-indazol-5-yl)-6-phenyl-N-[(2S)-2-pyrrolidinylmethyl]-3-pyridinamine

Following the procedure of Example 72 except for substituting N-Boc-(2S) -2-pyrrolidinylacetaldehyde for N-Boc-(2S)-2-amino-3-phenylpropanal, the title compound was prepared. ¹H NMR (CD₃OD, 400 MHz) δ 8.16 (d, 1H), 7.75 (d, 1H), 7.68 (d, 1H), 7.40-7.28 (m, 6H), 7.12 (d, 1H), 3.98 (m, 1H), 3.67 (m, 2H), 3.39 (m, 1H), 3.32 (s, 3H), 2.46 (m, 1H), 2.17 (m, 2H), 1.88 (m, 1H), 1.32 (m, 1H). MS (M+H): 384.2.

### Example 81

### Preparation of ((2S)-2-amino-3-{4-[(phenylmethyl)oxy]phenyl}propyl)[5-(3-methyl -1H-indazol-5-yl)-6-phenyl-3-pyridinyl]amine

Following the procedure of Example 70(a)-70(b), except substituting Boc -tyr(bzl)-aldehyde for 3-phenylpropanal, the title compound was prepared. ¹H NMR (CD₃OD, 400 MHz) δ 8.03 (d, 1H), 7.67 (d, 1H), 7.63 (d, 1H), 7.44-7.32 (m, 13H), 7.26 (dd, 1H), 6.93 (d, 2H), 4.89 (s, 2H), 4.00 (m, 1H), 3.60 (m, 2H), 3.02 (m, 2H), 2.51 (s, 3H). MS (M+H): 540.6.

### Example 82

### Preparation of [(2S)-2-amino-3-phenylpropyl][5-(1H-indazol-5-yl)-6-phenyl-3-pyridinyl]amine

a) 1,1-dimethylethyl [(1S)-2-[(5-bromo-6-chloro-3-pyridinyl)amino]-1-(phenylmethyl)ethyl]carbamate

Following the procedure of Example 70(a) except for substituting N-Boc -(2S)-2-amino-3-phenylpropanal for 3-phenylpropanal, the title compound was prepared.

b) [(2S)-2-amino-3-phenylpropyl][5-(1H-indazol-5-yl)-6-phenyl-3-pyridinyl]amine

A solution of the compound in Example 82(a)(116 mg, 1.0 eq), 5-(4,4,5,5-tetramethyl-[1,3,2]dioxaborolan-2-yl)-indazole-1-carboxylic acid tert-butyl ester(105 mg, 1.1 eq), Pd(PPh₃)₄ (0.05 eq) and 0.5 ml 5% aqueous Na₂CO₃ in dioxane was heated at 150° C. for 10 min in microwave. To the reaction mixture was added another 0.2 ml 5% aqueous NaHCO₃, 0.05 eq of Pd(PPh₃)₄ and phenylboronic acid(135 mg, 1.2 eq). The reaction mixture was heated at 150° C. for another 10 min in microwave. The solution was concentrated and purified by flash column chromatography to give 81 mg of the title compound(yield 72%).

c) [(2S)-2-amino-3-phenylpropyl][5-(1H-indazol-5-yl)-6-phenyl-3-pyridinyl]amine

The solution of 82(b)(81 mg) in 5 ml CH₂Cl₂ was added 1 ml TFA. The reaction mixture was stirred at room temperature for 1 h. The solution was concentrated and crude product was purified by reverse phase HPLC to give 30 mg of the title compound. ¹H NMR (CD₃OD, 400 MHz) δ 8.11 (d, 1H), 8.03 (d, 1H), 7.66 (d, 1H), 7.59 (d, 1H), 7.47-7.29 (m, 1OH), 7.11 (dd, 2H), 3.84 (m, 1H), 3.54 (m, 2H), 3.13 (m, 2H). MS (M+H): 420.2.

### Example 83

### Preparation of [(2S)-2-amino-3-phenylpropyl[f6-(3-furanyl)-5-(1H-indazol-5-yl)-3-pyridinyl]amine

Following the procedure of Example 82 except for substituting 3-furanylboronic acid for phenylboronic acid, the title compound was prepared. ¹H NMR (CD₃OD, 400 MHz) 68.16 (d, 1H), 8.00 (d, 1H), 7.79 (d, 1H), 7.63 (dd, 1H), 7.51 (m, 3H), 7.30-7.20 (m, 6H), 6.25 (d, 1H), 4.00 (m, 1H), 3.57-3.54 (m, 2 H), 3.13-3.01 (m, 2H). MS (M+H): 410.2.

### Example 84

### Preparation of [(2S)-2-amino-3-phenylpropyl][5-(1H-indazol-5-yl)-6-(3-thienyl)-3-pyridinyl]amine

Following the procedure of Example 82 except for substituting 3-thienylboronic acid for phenylboronic acid, the title compound was prepared. ¹H NMR (CD₃OD, 400 MHz) 68.12 (d, 1H), 7.99 (d, 1H), 7.75 (d, 1H), 7.61-7.51 (m, 3H), 7.40-7.15 (m, 6H), 6.79 (dd, 1H), 3.82 (m, 1H), 3.62-3.53 (m, 2H), 3.15-3.02 (m, 2H), MS (M+H): 426.2.

### Example 85

### Preparation of 2-[5-{[(2S)-2-amino-3-phenylpropyl]amino}-3-(1H-indazol-5-yl)-2-pyridinyl]phenol

Following the procedure of Example 82 except for substituting 2-(4,4,5,5-tetramethyl-1,3,2-dioxaborolan-2-yl)phenol for phenylboronic acid, the title compound was prepared. ¹H NMR (CD₃OD, 400 MHz) δ 8.06 (d, 1H), 7.99 (d, 1H), 7.69 (d, 1H), 7.63 (d, 1H), 7.43 (dd, 1H), 7.37-7.15 (m, 6H), 6.98 (dd, 1H), 6.88 (dd, 1H), 6.74 (t, 1H), 3.82 (m, 1H), 3.63-3.52 (m, 2H), 3.14-3.03 (m, 2H). MS (M+H): 436.2.

### Example 86

### Preparation of 2-[5-{[(2S)-2-amino-3-phenylpropyl]amino}-3-(3-methyl-1H-indazol -5-yl)-2-pyridinyl]phenol

Following the procedure of Example 82 except for substituting 3-methyl-5-(4,4,5,5-tetramethyl-[1,3,2]dioxaborolan-2-yl)-indazole-1-carboxylic acid tert-butyl ester(1c) for 5-(4,4,5,5-tetramethyl-[1,3,2]dioxaborolan-2-yl)-indazole-1-carboxylic acid tert -butyl ester and substituting 2-(4,4,5,5-tetramethyl-1,3,2-dioxaborolan-2-yl)phenol for phenylboronic acid, the title compound was prepared. ¹H NMR (CD₃OD, 400 MHz) δ 7.99 (d, 1H), 7.67 (d, 1H), 7.61 (d, 1H), 7.37-7.20 (8H), 6.99 (d, 1H), 6.89 (d, 1H), 3.85 (m, 1H), 3.60-3.57 (m, 2H), 3.11-3.07 (m, 2H), 2.49 (s, 3H). MS (M+H): 450.2.

### Example 87

### Preparation of [(2S)-2-amino-3-phenylpropyl][5-(3-methyl-1H-indazol-5-yl)-6-(1H -pyrrol-2-yl)-3-pyridinyl]amine

Following the procedure of Example 82 except for substituting 3-methyl-5-(4,4,5,5-tetramethyl-[1,3,2]dioxaborolan-2-yl)-indazole-1-carboxylic acid tert-butyl ester(1c) for 5-(4,4,5,5-tetramethyl-[1,3,2]dioxaborolan-2-yl)-indazole-1-carboxylic acid tert-butyl ester and substituting 1H-pyrrol-2-ylboronic acid for phenylboronic acid, the title compound was prepared. ¹H NMR (CD₃OD, 400 MHz) δ 7.88 (d, 1H), 7.69 (d, 1H), 7.55 (d, 1H), 7.47 (d, 1H), 7.30-7.15 (m, 6H), 6.86 (dd, 1H), 6.29 (dd, 1H), 6.20 (dd, 1H), 3.79 (m, 1H), 3.56-3.52 (m, 2H), 3.10-3.05 (m, 2H), 2.57 (s, 3H). MS (M+H): 423.0.

### Example 88

### Preparation of [(2S)-2-amino-3-phenylpropyl][5-(3-methyl-1H-indazol-5-yl)-6-(5-methyl-2-thienyl)-3-pyridinyl]amine

Following the procedure of Example 82 except for substituting 3-methyl-5-(4,4,5,5-tetramethyl-[1,3,2]dioxaborolan-2-yl)-indazole-1-carboxylic acid tert-butyl ester(1c) for 5-(4,4,5,5-tetramethyl-[1,3,2]dioxaborolan-2-yl)-indazole-1-carboxylic acid tert-butyl ester and substituting (5-methyl-2-thienyl)boronic acid for phenylboronic acid, the title compound was prepared. ¹H NMR (CD₃OD, 400 MHz) δ 7.97 (d, 1H), 7.71 (d, 1H), 7.49 (d, 1H), 7.40-7.18 (m, 8H), 7.03 (d, 1H), 6.73 (d, 1H), 3.78 (m, 1H), 3.55-3.37 (m, 2H), 3.09-3.02 (m, 2H), 2.58 (s, 3H), 2.38 (s, 3H). MS (M+H): 454.0.

### Example 89

### Preparation of [(2R)-2-amino-3-phenylpropyl][5-(1H-indazol-5-yl)-6-(3-thienyl)-3-pyridinyl]amine

Following the procedure of Example 82 except for substituting N-Boc-(2R) -2-amino-3-phenylpropanal for N-Boc-(2S)-2-amino-3-phenylpropanal and substituting 3-thienylboronic acid for phenylboronic acid, the title compound was prepared. ¹H NMR (CD₃OD, 400 MHz) δ 8.12 (d, 1H), 8.00 (d, 1H), 7.75 (d, 1H), 7.61-7.51 (m, 3H), 7.40-7.15 (m, 6H), 6.79 (dd, 1H), 3.80 (m, 1H), 3.62-3.53 (m, 2H), 3.15-3.02 (m, 2H), MS (M+H): 426.2.

### Example 90

### Preparation of 2-[5-{[(2S)-2-amino-3-(1H-indol-3-yl)propyl]oxy}-3-(3-methyl-1H-indazol-5-yl)-2-pyridinyl]phenol

Following the procedure of Example 69 except for substituting 2-(4,4,5,5-tetramethyl-1,3,2-dioxaborolan-2-yl)phenol for phenylboronic acid, the title compound was prepared. ¹H NMR (CD₃OD, 400 MHz) δ 8.54 (d, 1H), 8.24 (d, 1H), 7.63 (m, 2H), 7.48-7.05 (m, 7H), 6.90 (d, 1H), 6.78 (dd, 1H), 4.58 (dd, 1H), 4.48 (dd, 1H), 4.05 (m, 1H), 3.32 (d, 2H), 2.48 (s, 3H). MS (M+H): 490.2.

### Example 91

### Preparation of [(1S)-2-(1H-indol-3-yl)-1-({[5-(3-methyl-1H-indazol-5-yl)-6-(1-H-pyrrol-2-yl)-3-pyridinyl]oxy}methyl)ethyl]amine

Following the procedure of Example 69 except for substituting 1H-pyrrol-2-ylboronic acid for phenylboronic acid, the title compound was prepared. ¹H NMR (CD₃OD, 400 MHz) δ 8.34 (d, 1H), 7.70 (dd, 2H), 7.62 (d, 1H), 7.50 (d, 1H), 7.38 (d, 1H), 7.23 (m, 1H), 7.17 (dd, 1H), 7.04 (dd, 1H), 6.83 (dd, 1H), 6.08 (dd, 1H), 5.81 (dd, 1H), 4.44 (dd, 1H), 4.32 (dd, 1H), 4.02 (m, 1H), 3.30 (d, 2H), 2.57 (s, 3H). MS (M+H): 463.2.

### Example 92

### Preparation of [(1S)-2-(1H-indol-3-yl)-1-({[5-(3-methyl-1H-indazol-5-yl)-6-(5-methyl-2-thienyl)-3-pyridinyl]oxy}methyl)ethyl]amine

Following the procedure of Example 69 except for substituting (5-methyl-2-thienyl)boronic acid for phenylboronic acid, the title compound was prepared. ¹H NMR (CD₃OD, 400 MHz) δ 8.38 (d, 1H), 7.68 (d, 1H), 7.60 (dd, 2H), 7.45 (m, 2H), 7.32 (d, 1H), 7.25 (m, 2H), 7.12 (dd, 1H), 7.05 (dd, 1H), 6.53 (dd, 1H), 6.49 (dd, 1H), 4.38 (dd, 1H), 4.29 (dd, 1H), 4.00 (m, 1H), 3.28 (d, 2H), 2.60 (s, 3H), 2.39 (s, 3H). MS (M+H): 493.2.

### Example 93

### Preparation of [(1S)-2-{[6-ethyl-5-(3-methyl-1H-indazol-5-yl)-3-pyridinyl]oxy}-1-(phenylmethyl)ethyl]amine

In a solution of the compound in Example 67 (100 mg) in 10 ml EtOH was added 20 mg 10% Pd/C. The solution was then charged with H₂ under 1 atm(ballon) and stirred at room temperature for 5 h. The mixture was then filtered by celite. The resulted organic solution was concentrated in vacuo. Separation by flash column chromatography provided 88 mg product. ¹H NMR (CD₃OD, 400 MHz) δ 8.48 (d, 1H), 7.81 (m, 1H), 7.66 (s, 1H), 7.65 (d, 1H), 7.45-7.31 (m, 6H), 4.42 (dd, 1H), 4.28 (m, 1H), 3.97 (m, 1H), 3.15 (d, 2H), 2.97 (m, 2H), 2.61 (s, 3H), 1.20 (t, 3H). MS (M+H): 387.4.

### Example 94

### Preparation of [(1S)-2-{[6-(3-furanyl)-5-(1H-indazol-5-yl)-3-pyridinyl]oxy}-1-(phenylmethyl)ethyl]amine

Following the procedure of Example 1(a)-1(f) except for substituting 5-(4,4,5,5-tetramethyl-[1,3,2]dioxaborolan-2-yl)-indazole-1-carboxylic acid tert-butyl ester for the compound in Example 1(c) and substituting 3-furanylboronic acid for phenylboronic acid, the title compound was prepared. ¹H NMR (CD₃OD, 400 MHz) δ 8.38 (d, 1H), 8.12 (s, 1H), 7.76 (s, 1H), 7.60 (d, 1H), 7.45 (d, 1H), 7.34-7.27 (m, 7H), 7.15 (s, 1H), 6.29 (dd, 1H), 4.33 (dd, 1H), 4.18 (dd, 1H), 3.95 (m, 1H), 3.16 (dd, 2H). MS (M+H): 411.2.

### Example 95

### Preparation of [(1S)-2-{[5-(3-ethenyl-1H-indazol-5-yl)-6-(3-furanyl)-3-pyridinyl]oxy}-1-(phenylmethyl)ethyl]amine

Following the procedure of Example 23(a)-23(c) except for substituting 3-furanylboronic acid for phenylboronic acid in Example 23(a) and substituting triethenylboroxin for phenylboronic acid in Example 23(b)-23(c), the title compound was prepared. ¹H NMR (CD₃OD, 400 MHz) δ 8.41 (d, 1H), 7.95 (s, 1H), 7.62-7.57 (m, 2H), 7.40-7.26 (m, 8H), 7.06 (dd, 1H), 6.29 (d, 1H), 6.05 (dd, 1H), 5.53 (d, 1H), 4.39 (dd, 1), 4.22 (dd, 1H), 3.96 (m, 1H), 3.32 (d, 2H). MS (M+H): 437.4.

### Example 96

### Preparation of [(1S)-2-{[5-(3-ethyl-1H-indazol-5-yl)-6-(3-furanyl)-3-pyridinyl]oxy}-1-(phenylmethyl)ethyl]amine

Following the procedure of Example 93 except for substituting the compound in Example 95 for the compound in Example 67, the title compound was prepared. ¹H NMR (CD₃OD, 400 MHz) δ 8.41 (d, 1H), 7.74 (s, 1H), 7.69 (d, 1H), 7.54 (d, 1H), 7.42-7.28 (m, 8H), 6.29 (dd, 1H), 4.38 (dd, 1H), 4.23 (dd, 1H), 3.96 (m, 1H), 3.18 (d, 2H), 3.01 (q, 2H), 1.38 (t, 1H). MS (M+H): 439.4.

### Example 97

### Preparation of [(1S)-2-({6-(3-furanyl)-5-[3-(3-pyridinyl)-1H-indazol-5-yl]-3-pyridinyl}oxy)-1-(phenylmethyl)ethyl]amine

Following the procedure of Example 23(a)-23(c) except for substituting 3-furanylboronic acid for phenylboronic acid in Example 23(a) and substituting 3-pyridinylboronic acid for phenylboronic acid in Example 23(b)-23(c), the title compound was prepared. ¹H NMR (CD₃OD, 400 MHz) δ 9.29 (s, 1H), 8.84 (s, 1H), 8.73 (d, 1H), 8.40 (d, 1H), 8.09 (s, 1H), 7.93 (d, 1H), 7.72 (d, 1H), 7.53 (d, 1H), 7.42-7.28 (m, 7H), 7.20 (d, 1H), 6.33 (dd, 1H), 4.34 (dd, 1H), 4.19 (dd, 1H), 3.94 (m, 1H), 3.16 (d, 2H). MS (M+H): 488.2.

### Example 98

### Preparation of [(1S)-2-{[6-methyl-5-(3-methyl-1H-indazol-5-yl)-3-pyridinyl]oxy}-1-(phenylmethyl)ethyl]amine

Following the procedure of Example 1(a)-1(f) except for substituting Methylboronic acid for phenylboronic acid, the title compound was prepared. ¹H NMR (CD₃OD, 400 MHz) δ 8.51 (d, 1H), 8.12 (d, 1H), 7.86 (d, 1H), 7.67 (d, 1H), 7.48 (d, 1H), 7.40-7.31 (m, 5H), 4,45 (dd, 1H), 4,32 (dd, 1H), 4.00 (m, 1H), 3.16 (d, 2H), 2.67 (s, 3H), 2.62 (s, 3H). MS (M+H): 373.0.

### Example 99

### Preparation of [(1S)-2-({5-(3-methyl-1H-indazol-5-yl)-6-[2-(methyloxy)phenyl]-3-pyridinyl}oxy)-1-(phenylmethyl)ethyl]amine

Following the procedure of Example 1(a)-1(f) except for substituting 2-methoxyphenylboronic acid for phenylboronic acid, the title compound was prepared. ¹H NMR (CD₃OD, 400 MHz) δ 8.46 (d, 1H), 7.93 (d, 1H), 7.42 (d, 1H), 7.41-7.28 (m, 7H), 7.36 (d, 1H), 7.20 (d, 1H), 7.01 (dd, 1H), 6.92 (d, 1H), 4.45 (dd, 1H), 4.30 (dd, 1H), 4.00 (m, 1H), 3.50 (s, 3H), 3.20 (d, 2H), 2.45 (s, 3H). MS (M+H): 465.2.

### Example 100

### Preparation of [(1S)-2-{[6-[2-(ethyloxy)phenyl]-5-(3-methyl-1H-indazol-5-yl)-3-pyridinyl]oxy}-1-(phenylmethyl)ethyl]amine

Following the procedure of Example 1(a)-1(f) except for substituting 2-ethyloxyphenylboronic acid for phenylboronic acid, the title compound was prepared. ¹H NMR (CD₃OD, 400 MHz) δ 8.49 (d, 1H), 8.01 (d, 1H), 7.57 (s, 17.42-7.29 (m, 8H), 7.20 (d, 1H), 7.02 (dd, 1H), 6.90 (d, 1H), 4.47 (dd, 1H), 4.33 (dd, 1H), 4.01 (m, 1H), 3.69 (q, 2H), 3.32 (d, 2H), 2.45 (s, 3H), 1.10 (t, 3H). MS (M+H): 479.4.

### Example 101

### Preparation of [(1S)-2-{[6-[5-chloro-2-(methyloxy)phenyl]-5-(3-methyl-1H-indazol-5-yl)-3-pyridinyl]oxy}-1-(phenylmethyl)ethyl]amine

Following the procedure of Example 1(a)-1(f) except for substituting 5-chloro-2-(methyloxy)phenylboronic acid for phenylboronic acid, the title compound was prepared. ¹H NMR (CD₃OD, 400 MHz) δ 8.41 (d, 1H), 7.72 (d, 1H), 7.53 (d, 1H), 7.43-7.33 (m, 8H), 7.19 (d, 1H), 6.82 (d, 1H), 4.43 (dd, 1H), 4.25 (dd, 1H), 3.96 (m, 1H), 3.19 (d, 2H), 2.47 (s, 3H). MS (M+H): 499.4.

### Example 102

### Preparation of [(1S)-2-{[6-[5-fluoro-2-(propyloxy)phenyl]-5-(3-methyl-1H-indazol-5-yl)-3-pyridinyl]oxy}-1-(phenylmethyl)ethyl]amine

Following the procedure of Example 1(a)-1(f) except for substituting 5-fluoro-2-(propyloxy)phenylboronic acid for phenylboronic acid, the title compound was prepared. ¹H NMR (CD₃OD, 400 MHz) δ 8.46 (d, 1H), 7.84 (d, 1H), 7.59 (d, 1H), 7.42-7.33 (m, 6H), 7.19 (dd, 1H), 7.09 (m, 2H), 6.87 (dd, 1H), 4.43 (dd, 1H), 4.28 (dd, 1H), 4.00 (m, 1H), 3.53 (t, 2H), 3.18 (d, 2H), 2.48 (s, 3H), 1.51 (m, 2H), 0.78 (t, 3H). MS (M+H): 511.4.

### Example 103

### Preparation of [(1S)-2-({5-[3-(1-methylethyl)-1H-indazol-5-yl]-6-phenyl-3-pyridinyl}oxy)-1-(phenylmethyl)ethyl]amine

Following the procedure of Example 93 except for substituting the compound in Example 46 for the compound in Example 67, the title compound was prepared. ¹H NMR (CD₃OD, 400 MHz) δ 8.47 (d, 1H), 7.88 (d, 1H), 7.60 (s, 1H), 7.45-7.24 (m, 12H), 4.44 (dd, 1H), 4.28 (dd, 1H), 3.99 (m, 1H), 3.28 (m, 1 H), 3.18 (d, 2H), 1.31 (d, 6H). MS (M+H): 463.4.

### Example 104

### Preparation of [(1S)-2-{[5-(6-fluoro-3-methyl-1H-indazol-5-yl)-6-(3-furanyl)-3-pyridinyl]oxy}-1-(phenylmethyl)ethyl]amine

a) 1-(5-bromo-2,4-difluorophenyl)ethanone

To a solution of 1,5-dibromo-2,4-difluorobenzene(Nucleosides, Nucleotides & nucleic Acid, 201 (1&2), 11-40(2001)) (8.8 g, 32.4 mmol) in diethylether(60 ml), 1.6 M n-BuLi in hexane(24.3 ml, 1.2 eq) was added at −78° C. under N₂ atmosphere. After stirring the reaction mixture at −78° C. for 30 min, N-methyl-N -(methyloxy)acetamide (5.0 g, 1.5 eq) was dropped into to quench the reaction. The reaction mixture was stirred at the same temperature for further 30 min. After added acetic acid((5.2 ml), water (78 ml), the reaction mixture was extracted with diethylether. The obtained organic phase was washed by 0.2 N HCl aqueous, water, saturated NaHCO₃ aqueous and saturated NaCl aqueous, and dried over MgSO₄. After removing the solvent under reduced pressure, the residue was purified by Silica gel chromatography (n-Hexane/EtOAc=49/1). Desired compound was obtained as pale yellow oil (4.94 g, 65%).

b) 1-(5-bromo-2,4-difluorophenyl)ethanone hydrazone

H₂NNH₂ (0.80 ml, 25.5 mmol) was added to a solution of 1-(5-bromo-2,4-difluorophenyl)ethanone (4.72 g, 20.3 mmol) in EtOH (50 ml). The resulting reaction mixture was stirred at RT overnight and evaporated to give dried light yellow solid, which was recrystalized in MeOH to give 1.8 g white crystaline. Mother liquid was concentrated and purified by flash column chromatography to give a total of 3.85 g solid (76%)

c) 5-bromo-6-fluoro-3-methyl-1H-indazole

A solution of 1-(5-bromo-2,4-difluorophenyl)ethanone hydrazone (2.16 g, 8.7 mmol) in pyridine (87 ml) was heated up in a sealed flask at 120° C. overnight. The resulting mixture was taken up into ice-cold HCl (6 N), which was extracted with EtOAc. The solution was concentrated and purified by flash column chromatography to give 1.6 g light brown solid (80%).

d) 6-fluoro-3-methyl-5-(4,4,5,5-tetramethyl-1,3,2-dioxaborolan-2-yl)-1H-indazole

Following the procedure of Example 1(c) except for substituting 5-bromo-6-fluoro-3-methyl-1H-indazole for N-Boc-3-methyl-5-bromoindazole, the title compound was prepared.

e) [(1S)-2-{[5-(6-fluoro-3-methyl-1H-indazol-5-yl)-6-(3-furanyl)-3-pyridinyl]oxy}-1-(phenylmethyl)ethyl]amine

Following the procedure of Example 1(a)-1(f) except for substituting 6-fluoro-3-methyl-5-(4,4,5,5-tetramethyl-1,3,2-dioxaborolan-2-yl)-1H-indazole for 3-methyl-5-(4,4,5,5-tetramethyl-[1,3,2]dioxaborolan-2-yl)-indazole-1-carboxylic acid tert-butyl ester in Example 1(b)-1(c) and substituting 3-furanylboronic acid for phenylboronic acid in Example 1(d)-1(e), the title compound was prepared. ¹H NMR (CD₃OD, 400 MHz) δ 8.50 (d, 1H), 7.77 (d, 2H), 7.46-7.26 (m, 8H), 6.41 (dd, 1H), 4.42 (dd, 1H), 4.29 (m, 1H), 3.99 (m, 1H), 3.16 (d, 2H), 2.58 (s, 3H). MS (M+H): 443.2.

### Example 105

### Preparation of N-[6-(3-furanyl)-5-(3-methyl-1H-indazol-5-yl)-3-pyridinyl]-L -phenylalaninamide

a) N-(5-bromo-6-chloro-3-pyridinyl)-N-{[(1,1-dimethylethyl)oxy]carbonyl}-L -phenylalaninamide

A mixture of N-carboxy-L-phenylalanine (1.0 g, 3.77 mmol), EDC (0.94 g, 4.9 mmol), HOAT (0.7 g, 5.14 mmol) in THF solvent was heated at reflux for 2 hrs. Then added 5-bromo-6-chloro-3-pyridinamine (0.65 g, 3.13 mmol) to the above mixture and continued refluxing for another hour. The reaction mixture was then cooled down to RT, the solvent was removed and the mixture was diluted with dichloromethane, and washed with water. The combined organic layers were dried over MgSO₄, filtered, concentrated and purified by Biotage (5% to 25% ethyl acetate/hexane) to provide 0.85 g yellowish solid (59.4%).

b) N-[6-chloro-5-(3-methyl-1H-indazol-5-yl)-3-pyridinyl]-N-{[(1,1-dimethylethyl)oxy]carbonyl}-L-phenylalaninamide

A solution of compound Example 105(a) (0.81 g, 1.79 mmol), 3-methyl-5-(4,4,5,5-tetramethyl-1,3,2-dioxaborolan-2-yl)-1H-indazole (0.584 g, 1.63 mmol), and catalytic amount of Pd(PPh₃)₄ was irradiated with microwave at 150° C. for 10 min. The reaction mixture was purified by column chromatography (40%-60% EtOAc/Hexane) to get 614 mg of the titled compound. (74.6%)

c) N-{[(1,1-dimethylethyl)oxy]carbonyl}-N-[6-(3-furanyl)-5-(3-methyl-1H-indazol-5-yl)-3-pyridinyl]-L-phenylalaninamide

A solution of the compound of Example 105(b)(100 mg, 0.198 mmol), 3-furanylboronic acid (26.6 mg, 0.24 mmol), Pd(PPh₃)₄ catalytic amount and 0.5 ml aqueous Na₂CO₃ in dioxane was heated at 150° C. for 10 min in microwave. The reaction mixture was concentrated and purified by flash column chromatography (50%-60% EtOAc/Hexane) to give 80 mg product (yield 75.5%).

d) N-[6-(3-furanyl)-5-(3-methyl-1H-indazol-5-yl)-3-pyridinyl]-L-phenylalaninamide

The solution of 105(c) in 5 ml CH₂Cl₂ was added 1 ml TFA. The reaction mixture was stirred at room temperature for 1 h. The solution was concentrated and crude product was purified by reverse phase HPLC to provide 62 mg the titled compound (98%). ¹H NMR (CD₃OD, 400 MHz) δ 8.75 (s, 1H), 8.16 (s, 1H), 7.66 (s, 1H), 7.11-7.68 (m, 8H), 7.17 (d, 1H), 6.31 (d, 1H), 4.25 (dd, 1H), 3.35 (dd, 1H), 3.21 (dd, 1H), 2.52 (s, 3H). MS (M+H): 438.2.

### Example 106

### Preparation of N-[6-(2-hydroxyphenyl)-5-(3-methyl-1H-indazol-5-yl)-3-pyridinyl]-L -phenylalaninamide

Following the procedure of Example 105(a)-105(d), except substituting 2-(4,4,5-trimethyl-1,3,2-dioxaborolan-2-yl)phenol ethane for 3-furanylboronic acid, the title compound was prepared. ¹H NMR (CD₃OD, 400 MHz) δ 9.21 (s, 1H), 8.48 (s, 1H), 7.65 (s, 1H), 6.81-7.40 (m, 11H), 4.42 (dd, 1H), 3.45 (dd, 1H), 3.25 (dd, 1H), 2.48 (s, 3H). MS (M+H): 464.6.

### Example 107

### Preparation of 2-[5-{[(2S)-2-amino-3-(1-benzothien-3-yl)propyl]oxy}-3-(1H-indazol-5-yl)-2-pyridinyl]phenol

a) 3-(1-benzothien-3-yl)-N-{[(1,1-dimethylethyl)oxy]carbonyl}-L-alanine

To a solution of K₂CO₃ (0.75 g, 5.43 mmol), 3-(1-benzothien-3-yl)-L-alanine (1.0 g, 4.52 mmol) in THF:H₂O (1:1) was added (Boc)₂O (1.09 g, 5 mmol) at 0° C. The reaction mixture was then warmed up to RT and stirred overnight. Concentrated down the mixture and then dissolved into EtOAc and H₂O. The aqueous layer was adjusted to PH 1-2 by 6N HCl and the mixture was extracted with EtOAc three times. The organic layer was dried, filtered and concentrated to provide 1.22 g product as a white foam (84%).

b) 1,1-dimethylethyl [(1S)-2-(1-benzothien-3-yl)-1-(hydroxymethyl)ethyl]carbamate

To a solution of 107(a)(1.22 g, 3.8 mmol) in THF at −10° C. was added BH₃.THF (22.8 ml, 22.8 mmol) dropwise. The reaction mixture was stirred at −10° C. for 3 hrs. Then mixture was concentrated down to one-third of the original volumn. Quenched with 9 ml MeOH: acetic acid (9:1). Concentrated down and the resultant was dissolved in EtOAc, washed by 1N HCl, aqueous saturated NaHCO₃, brine, and dried over MgSO₄. Concentrated down to provide 1.04 g of the product as white solid.

c) 1,1-dimethylethyl ((1S)-2-(1-benzothien-3-yl)-1-{[(5-bromo-6-chloro-3-pyridinyl)oxy]methyl}ethyl)carbamate

To a solution of 107(c) (1.04 g, 3.38 mmol), 5-bromo-6-chloro-3-pyridinol (0.78 g, 3.74 mmol), PPh₃ (1.37 g, 5.07 mmol) in THF was added DEAD (0.85 g, 4.89 mmol) at 0° C. The reaction mixture was warmed up to RT and stirred overnight. The residue was purified by biotage chromatography (15%-20% EtOAc/Hexane) to provide 1.30 g of the product.

d) 1,1-dimethylethyl [(1S)-2-(1-benzothien-3-yl)-1-({[6-chloro-5-(1H-indazol-5-yl)-3-pyridinyl]oxy}methyl)ethyl]carbamate

A solution of the compound of Example 107(c)(1.30 g, 2.61 mmol), 5-(4,4,5,5-tetramethyl-1,3,2-dioxaborolan-2-yl)-1H-indazole (0.7 g, 2.87 mmol), Pd(PPh₃)₄ catalytic amount and 2M aqueous Na₂CO₃ (3.2 ml, 6.5 mmol) was heated at 150° C. for 30 min in microwave. The reaction mixture was filtered by celite, concentrated and purified by flash column chromatography (20%-60% EtOAc/Hexane) to give 1.09 g product (yield 96%).

e) 2-[5-{[(2S)-2-amino-3-(1-benzothien-3-yl)propyl]oxy}-3-(1H-indazol-5-yl)-2-pyridinyl]phenol

A solution of the compound of Example 107(d)(100 mg, 0.186 mmol), 2-(4,4,5,5-tetramethyl-1,3,2-dioxaborolan-2-yl)phenol (62 mg, 0.28 mmol), Pd(PPh₃)₄ catalytic amount and 2M aqueous Na₂CO₃ was heated at 150° C. for 30 min in microwave. The reaction mixture was concentrated and purified by flash column chromatography (50% EtOAc/Hexane) to give white solid. To the above product was added 1 ml TFA. The reaction mixture was stirred at room temperature for 1 h. The solution was concentrated and crude product was purified by reverse phase HPLC to provide 47.2 mg the titled compound (52%). ¹H NMR (CD₃OD, 400 MHz) δ 8.56 (s, 1H), 8.25 (s, 1H), 6.72-8.04 (m, 13H), 4.51 (dd, 2H), 4.12-4.21 (m, 1H), 3.52 (dd, 2H). MS (M+H): 493.4.

### Example 108

### Preparation of [(1S)-2-(1-benzothien-3-yl)-1-({[6-(2-furanyl)-5-(1H-indazol-5-yl)-3-pyridinyl]oxy}methyl)ethyl]amine

Following the procedure of Example 107(a)-107(e), except substituting 2-(2-furanyl)-4,4,5,5-tetramethyl-1,3,2-dioxaborolane for 2-(4,4,5,5-tetramethyl-1,3,2-dioxaborolan-2-yl)phenol, the title compound was prepared. ¹H NMR (CD₃OD, 400 MHz) δ 8.40 (s, 1H), 8.15 (s, 1H), 7.21-7.93 (m, 11H), 6.45 (d, 1H), 5.88 (d, 1H), 4.40-4.42 (m, 1H), 4.21-4.30 (m, 1H), 4.08-4.15 (m, 1H), 3.50 (dd, 1H), 3.42 (dd, 1H). MS (M+H): 467.4.

### Example 109

### Preparation of [(1S)-2-{[5-(3-methyl-1H-indazol-5-yl)-6-phenyl-3-pyridinyl]oxy}-1-(2-naphthalenylmethyl)ethyl]amine

a) 1,1-dimethylethyl [(1R)-2-[(5-bromo-6-chloro-3-pyridinyl)oxy]-1-({[(1,1-dimethylethyl)(dimethyl)silyl]oxy}methyl)ethyl]carbamate

To a 500 ml round botton flask was charged with 5-bromo-6-chloro-3-pyridinol (10.23 g, 49.2 mmol), 1,1-dimethylethyl [(1R)-2-{[(1,1-dimethylethyl)(dimethyl)silyl]oxy}-1-(hydroxymethyl)ethyl]carbamate (15 g, 49.2 mmol), PPh₃ (17.23 g, 63.8 mmol) in THF. The mixture was cooled to 0° C. and kept stirring for 10 min. Then DEAD (10.05 ml, 63.8 mmol) was added via syringe. The reaction mixture was stirred at 0° C. for 2 hrs and warmed up to RT and kept stirring overnight. Concentrated down and residue was seperated by Biotage (5%-20% EtOAc/Hexane) to provide 20 g of the title product as a colorless oil. (83%).

b) 1,1-dimethylethyl [(1R)-2-{[6-chloro-5-(3-methyl-1H-indazol-5-yl)-3-pyridinyl]oxy}-1-({[(1,1-dimethylethyl)(dimethyl)silyl]oxy}methyl)ethyl]carbamate

A solution of the compound of Example 109(a)(2.0 g, 4.03 mmol), 1,1-dimethylethyl 3-methyl-5-(4,4,5,5-tetramethyl-1,3,2-dioxaborolan-2-yl)-1H-indazole-1-carboxylate (1.58 g, 4.41 mmol), Pd(PPh₃)₄ catalytic amount and 2M aqueous Na₂CO₃ (5.0 ml, 10 mmol) was heated at 150° C. for 30 min in microwave. The reaction mixture was filtered by celite, concentrated and purified by flash column chromatography (20%-60% EtOAc/Hexane) to give 1.0 g product (yield 45.4%).

c) 1,1-dimethylethyl [(1R)-2-{[(1,1-dimethylethyl)(dimethyl)silyl]oxy}-1-({[5-(3-methyl-1H-indazol-5-yl)-6-phenyl-3-pyridinyl]oxy}methyl)ethyl]carbamate

A solution of the compound of Example 109(b)(0.5 g, 0.91 mmol), phenylboronic acid (0.167 g, 1.37 mmol), Pd(PPh₃)₄ catalytic amount and 2M aqueous Na₂CO₃ (1.1 ml, 2.27 mmol) was heated at 150° C. for 30 min in microwave. The reaction mixture was filtered by celite, concentrated and purified by flash column chromatography (20%-60% EtOAc/Hexane) to give 0.44 g product (yield 81.8%).

d) 1,1-dimethylethyl 5-(5-{[(2R)-3-{[(1,1-dimethylethyl)(dimethyl)silyl]oxy}-2-({[(1,1-dimethylethyl)oxy]carbonyl}amino)propyl]oxy}-2-phenyl-3-pyridinyl)-3-methyl-1H -indazole-1-carboxylate

To the solution of 109(c) (1.76 g, 3.0 mmol) in THF was added (Boc)₂0 (1.3 g, 6 mmol), DMAP (183 mg, 1.5 mmol). The reaction mixture was stirred at RT overnight. Concentrated down and residue was purified by Biotage (20%-30% EtOAc/Hexane) to provide 1.87 g product.(91%).

e) 1,1-dimethylethyl 5-(5-{[(2S)-2-({[(1,1-dimethylethyl)oxy]carbonyl}amino)-3-hydroxypropyl]oxy}-2-phenyl-3-pyridinyl)-3-methyl-1H-indazole-1-carboxylate

To a solution of 109(d)(1.07 g, 1.55 mmol) in THF was added TBAF (1.86 ml, 1.86 mmol). The reaction mixture was stirred at RT for 1 hr. Concentrated down and the residue was purified by Biotage (70% EtOAc/Hexane) to provide 0.75 g product.(83.5%).

f) 1,1-dimethylethyl 5-(5-{[((2S)-1-{[(1,1-dimethylethyl)oxy]carbonyl}-2-aziridinyl)methyl]oxy}-2-phenyl-3-pyridinyl)-3-methyl-1H-indazole-1-carboxylate

To the ice-colded solution of PPh₃ (0.141 g, 0.52 mmol) in THF/CH₃CN(9:1) was slowly added DIAD(0.161 g, 0.8 mmol). The reaction mixture was stirred at 0° C. for 20 min. Then followed by the addition of 109(e)(0.2 g, 0.34 mmol). The reaction mixture was allowed to warm up to RT and stirred overnight. Purified the crude material to provide 0.18 g of the titled compound.(93%)

g) [(1S)-2-{[5-(3-methyl-1H-indazol-5-yl)-6-phenyl-3-pyridinyl]oxy}-1-(2-naphthalenylmethyl)ethyl]amine

A suspension of bromo(2-naphthalenyl)magnesium (3.0 ml, 1.5 mmol) and CuBr. Me₂S (55.5 mg, 0.26 mmol) was stirred at −40° C. Then 109(f)(100 mg, 0.18 mmol) was added slowly into the above solution. The reaction mixture was warmed up to −20° C. in 30 min and then warmed up to RT and stirred for 1 hr. Concentrated down and used directly for the next step. The crude material was dissolved in dichloromethane, stirred at room temperature for I h. The solution was concentrated and crude product was purified by reverse phase HPLC to provide 43 mg titled final product. (51%). ¹H NMR (CD₃OD, 400 MHz) δ 8.51 (s, 1H), 8.10 (s, 1H), 7.11-7.68 (m, 15H), 4.65 (dd, 2H), 4.13 (dd, 1H), 3.71-3.94 (m, 2H), 2.50 (s, 3H). MS (M+H): 485.6.

### Example 110

### Preparation of N-[5-(3-methyl-1H-indazol-5-yl)-6-(1H-pyrrol-2-yl)-3-pyridinyl]-L -phenylalaninamide

Following the procedure of Example 105(a)-105(d), except substituting (1-{[(1,1-dimethylethyl)oxy]carbonyl}-1H-pyrrol-2-yl)boronic acid for 3-furanylboronic acid, the title compound was prepared. ¹H NMR (CD₃OD, 400 MHz) δ 8.85 (s, 1H), 8.05 (s, 1H), 7.72 (s, 1H), 7.22-7.44 (m, 7H), 6.06 (d, 1H), 5.76 (d, 1H), 4.30 (dd, 1H), 3.35 (dd, 1H), 3.21 (dd, 1H), 2.58 (s, 3H). MS (M+H): 437.4.

### Example 111

### Preparation of [(2S)-2-amino-3-(1H-indol-3-yl)propyl][6-(3-furanyl)-5-(3-methyl-1H-indazol-5-yl)-3-pyridinyl]amine

a) 1,1-dimethylethyl [(1S)-1-formyl-2-(1H-indol-3-yl)ethyl]carbamate

To a solution of N-{[(1,1-dimethylethyl)oxy]carbonyl}-N-methyl-N-(methyloxy) -L-tryptophanamide (3.0 g, 8.64 mmol) in 100 ml THF was added DIBAL (2.3 ml, 13 mmol) dropwise at −78° C. After 3 hrs, the reaction mixture was warmed up to RT and 50 ml of 1M Rochelles salt (Na/K tartrate) was added, stirred overnight. The mixture was extracted with Et₂O 3 times. The combined organic layer was washed by brine, dried over Na₂SO₄. Concentrated down and the residue was purified by Biotage (50%-60% EtoAc/Hexane) to provide 1.66 g of the titiled compound as white foam.(66%)

b) 1,1-dimethylethyl [(1S)-2-[(5-bromo-6-chloro-3-pyridinyl)amino]-1-(1H-indol-3-ylmethyl)ethyl]carbamate

To the solution of 111(a)(1.66 g, 5.76 mmol), 5-bromo-6-chloro-3-pyridinamine (1.31 g, 6.32 mmol) in dichloromethane was added NaBH(OAc)₃ (3.66 g, 17.3 mmol). The reaction mixture was stirred at RT overnight. Quenched the reaction with water, and then organic layer was washed with saturated NaHCO₃, brine, dried over Na₂SO₄. Concentrated down and the residue was purified by Biotage (40%-60% EtoAc/Hexane) to provide 2.07 g of the titiled compound.(75%)

c) 1,1-dimethylethyl [(1S)-2-{[6-chloro-5-(3-methyl-1H-indazol-5-yl)-3-pyridinyl]amino}-1-(1H-indol-3-ylmethyl)ethyl]carbamate

Follow the Suzuki coupling procedure of 109 (b) to provide 675 mg of the titled compound.(80.3%)

d) [(2S)-2-amino-3-(1H-indol-3-yl)propyl][6-(3-furanyl)-5-(3-methyl-1-H-indazol-5-yl)-3-pyridinyl]amine

Follow the similar procedure as 105(c), 105(d) to provide the titled final compound (65%). ¹H NMR (CD₃OD, 400 MHz) δ 7.91 (s, 1H), 7.62 (s, 1H), 7.58 (d, 1H), 7.49 (d, 2H), 6.91-7.48 (m, 7H), 6.20 (d, 1H), 3.85-3.96 (m, 1H), 3.52-3.68 (m, 2H), 3.20-3.28 (m, 2H), 2.54 (s, 3H). MS (M+H): 463.4.

### Example 112

### Preparation of (2S)-1-{[6-(3-furanyl)-5-(3-methyl-1H-indazol-5-yl)-3-pyridinyl]oxy}-3-phenyl-2-propanol

a) 3-bromo-2-chloro-5-{[(2S)-2-oxiranylmethyl]oxy}pyridine

A solution of 5-bromo-6-chloro-3-pyridinol (482 mg, 2.32 mmol), (2S)-2-oxiranylmethyl 2-nitrobenzenesulfonate (600 mg, 2.32 mmol), K₂CO₃ (600 mg) in acetone was heated at reflux overnight. Cooled down the reaction mixture, filtered and concentrated the mixture. The residue was purified by Biotage (20%-40% EtoAc/Hexane) to provide 460 mg of the titiled compound.(80%)

b) (2S)-1-[(5-bromo-6-chloro-3-pyridinyl)oxy]-3-phenyl-2-propanol

To the solution of 112(a)(511.7 mg, 1.93 mmol), catalytic amount of Cul in THF at −78° C. was added chloro(phenyl)magnesium (2.12 ml, 4.25 mmol). The mixture then was warmed up to 0° C. and stirred for 30 mins. Quenched the reaction mixture with saturated NaHCO₃ aqueous solution, extracted with dichloromethane, and dried over Na₂SO₄. The residue was purified by Biotage to provide 216 mg of the titiled compound.(32.6%)

c) (2S)-1-{[6-(3-furanyl)-5-(3-methyl-1H-indazol-5-yl)-3-pyridinyl]oxy}-3-phenyl-2-propanol

Follow the similar procedure as 105(c), 105(d) to provide the titled final compound (38%). ¹H NMR (CD₃OD, 400 MHz) δ 8.49 (s, 1H), 8.13 (s, 1H), 7.80 (s, 1H), 7.49-7.60 (m, 3H), 7.15-7.35 (m, 6H), 6.30 (d, 1H), 4.20-4.36 (m, 1H), 3.30 (dd, 2H), 2.95-3.12 (d, 2H), 2.54 (s, 3H). MS (M+H): 426.2.

### Example 113

### Preparation of 1-3-[5-{[(2S)-2-amino-3-(1H-indol-3-yl)propyl]oxy}-2-(3-furanyl)-3-pyridinyl[phenyl]ethanone

Following the procedure of Example 69, except substituting 3-(acetylphenyl)boronic acid for Example 1(c), the title compound was prepared. ¹H NMR (400 MHz, MeOD) δ ppm 8.38 (d, J=2.8 Hz, 1H), 8.07 (dt, J=7.8, 1.4 Hz, 1H), 7.87-7.92 (m, 1H), 7.50-7.60 (m, 3H), 7.46 (d, J=2.8 Hz, 1H), 7.37-7.41 (m, 2H), 7.23 (s, 2H), 7.09-7.16 (m, 1H), 6.99-7.07 (m, 1H), 6.27 (d, J=1.0 Hz, 1H), 4.38 (dd, J=10.5, 3.2 Hz, 1H), 4.24 (dd, J=10.4, 5.8 Hz, 1H), 3.93-4.01 (m, 1H), 3.31-3.33 (m, 2H), 2.59 (s, 3H); MS: 452.2.

### Example 114

### Preparation of [(1S)-2-{[6-cyclopentyl-5-(3-methyl-1H-indazol-5-yl)-3-pyridinyl]oxy}-1-(1H-indol-3-ylmethyl)ethyl]amine

Follow the similar procedure as 69(a), 69(b) except substituting 1-cyclopenten-1-ylboronic acid for phenylboronic acid, then followed by Pd catalyzed hydrogenation, de-Boc by TFA to provide the desired titled product. ¹H NMR (CD₃OD, 400 MHz) δ 8.45 (s, 1H), 8.01 (s, 1H), 7.01-7.72 (m, 8H), 4.40 (dd, 2H), 3.99-4.08 (m, 1H), 3.21-3.40 (m, 3H), 2.60 (s, 3H), 1.58-2.12 (m, 8H). MS (M+H): 466.2.

### Example 115

### Preparation of [(1S)-2-(1-benzothien-3-yl)-1-({[5-(1H-indazol-5-yl)-6-phenyl-3-pyridinyl]oxy}methyl)ethyl]amine

Following the procedure of Example 107(a)-107(e), except substituting phenylboronic acid for 2-(4,4,5,5-tetramethyl-1,3,2-dioxaborolan-2-yl)phenol, the title compound was prepared. ¹H NMR (CD₃OD, 400 MHz) δ 8.51 (s, 1H), 8.09 (s, 1H), 7.11-7.98 (m, 14H), 4.40 (dd, 2H), 4.11-4.18 (m, 1H), 3.50 (dd, 2H). MS (M+H): 477.2.

### Example 116

### Preparation of [(1S)-2-(1-benzothien-3-yl)-1-({[6-(3-furanyl)-5-(1H-indazol-5-yl)-3-pyridinyl]oxy}methyl)ethyl]amine

Following the procedure of Example 107(a)-107(e), except substituting 3-furanylboronic acid for 2-(4,4,5,5-tetramethyl-1,3,2-dioxaborolan-2-yl)phenol, the title compound was prepared. ¹H NMR (CD₃OD, 400 MHz) δ 8.40 (s, 1H), 8.18 (s, 1H), 7.11-7.95 (m, 11H), 6.30 (s, 1H), 4.40 (dd, 2H), 4.11-4.18 (m, 1H), 3.50 (dd, 2H). MS (M+H): 467.0.

### Example 117

### Preparation of [(1S)-2-(1-benzothien-3-yl)-1-({[5-(1H-indazol-5-yl)-6-(3-thienyl)-3-pyridinyl]oxy}methyl)ethyl]amine

Following the procedure of Example 107(a)-107(e), except substituting 3-thienylboronic acid for 2-(4,4,5,5-tetramethyl-1,3,2-dioxaborolan-2-yl)phenol, the title compound was prepared. ¹H NMR (CD₃OD, 400 MHz) δ 8.39 (s, 1H), 8.12 (s, 1H), 7.21-7.95 (m, 10H), 4.30 (dd, 2H), 4.07-4.16 (m, 1H), 3.48 (dd, 2H). MS (M+H): 482.8.

### Example 118

### Preparation of [(1S)-2-(1-benzothien-3-yl)-1-({[5-(1H-indazol-5-yl)-6-(1H-pyrrol-2-yl)-3-pyridinyl]oxy}methyl)ethyl]amine

Following the procedure of Example 107(a)-107(e), except substituting 1H -pyrrol-2-ylboronic acid for 2-(4,4,5,5-tetramethyl-1,3,2-dioxaborolan-2-yl)phenol, the title compound was prepared. ¹H NMR (CD₃OD, 400 MHz) δ 8.39 (s, 1H), 8.12 (s, 1H), 7.21-7.96 (m, 9H), 6.86 (d, 1H), 6.06 (d, 1H), 5.90 (s, 1H), 4.35 (dd, 2H), 4.07-4.15 (m, 1H), 3.48 (dd, 2H). MS (M+H): 466.0.

### Example 119

### Preparation of [(1S)-2-{[5-(1H-indazol-5-yl)-6-phenyl-3-pyridinyl]oxy}-1-(1-H-pyrazol-1-ylmethyl)ethyl]amine

Following the procedure of Example 109(a)-109(g), except substituting 5-(4,4,5,5-tetramethyl-1,3,2-dioxaborolan-2-yl)-1H-indazole for 1,1-dimethylethyl 3-methyl-5-(4,4,5,5-tetramethyl-1,3,2-dioxaborolan-2-yl)-1H-indazole-1-carboxylate, and substituting bromo(2-naphthalenyl)magnesium for 1H-pyrazole, and reflux the reaction mixture in toluene in sealed tube for 48 hrs. ¹H NMR (CD₃OD, 400 MHz) δ 8.56 (s, 1H), 8.10 (s, 1H), 7.12-8.00 (m, 11H), 6.40 (d, 1H), 4.68 (dd, 2H), 4.43 (dd, 2H), 4.20-4.28 (m, 1H). MS (M+H): 411.0.

### Example 120

### Preparation of [(1S)-2-(1-benzothien-3-yl)-1-({[5-(1H-indazol-5-yl)-6-(5-methyl-2-thienyl)-3-pyridinyl]oxy}methyl)ethyl]amine

Following the procedure of Example 107(a)-107(e), except (5-methyl-2-thienyl)boronic acid for 2-(4,4,5,5-tetramethyl-1,3,2-dioxaborolan-2-yl)phenol, the title compound was prepared. ¹H NMR (CD₃OD, 400 MHz) δ 8.31 (s, 1H), 8.06 (s, 1H), 7.00-7.92 (m, 9H), 6.50 (d, 1H), 6.43 (d, 1H), 4.45 (dd, 1H), 4.25 (dd, 1H), 4.00-4.18 (m, 1H), 3.32-3.51 (dd, 2H), 2.35 (s, 3H). MS (M+H): 497.2.

### Example 121

### Preparation of [(1S)-2-{[6-(3-furanyl)-5-(3-methyl-1H-thieno[3,2-c]pyrazol-5-yl)-3-pyridinyl]oxy}-1-(1H-indol-3-ylmethyl)ethyl]amine

a) 1-(3,5-dibromo-2-thienyl)ethanone

A solution of BuLi (7.0 mL, 1.6 M in hexane) was added to a solution of 2,3,5-tribromothiophene (3.2 g, 10 mmol) in ether (100 mL) at −78° C. The resulting mixture was stirred at −78° C. for 30 min. N-Methoxy,N-methylactamide (1.2 g, 1.2 eq.) was added dropwise. The resulting reaction mixture was stirred at −78° C. for 30 min, and warmed up to 25° C. Ice cold water and saturated ammonium chloride aqueous solution were added. The organic layer was separated, dried (Na₂SO₄), and concentrated. The residue was purified by flash column chromatography on silica gel (hexane/EtOAc 9:1 to 3:1) to give 1.84 g of titled compound as the light yellow solid (65%).

b) 1,1-dimethylethyl (2E)-2-[1-(3,5-dibromo-2-thienyl)ethylidene]hydrazinecarboxylate

A solution of 121(a) (1.84 g, 6.48 mmol), NH₂NHBoc (1.03 g, 1.2 eq.) and 3 drops of concentrated HCl in 50 mL of THF was stirred at 25° C. overnight and evaporated under vacuum to dryness. The crude product was purified by flash column chromatography on silica gel (hexane/EtOAc 9:1 to 3:1) to give 1.9 g of white solid 121(b) (74%).

c) 1,1-dimethylethyl 5-bromo-3-methyl-1H-thieno[3,2-c]pyrazole-1-carboxylate

A mixture of 121(b)(1.9 g, 4.77 mmol), Cul (45 mg, 5 mol %), 1,10-phenanthroline (86 mg, 10 mol %), Cs₂CO₃ (2.17 g, 1.4 eq.) and 100 mL of 1,4-dioxane was degassed and heated at 100° C. under N₂ for 60 h. The reaction mixture was filtered through celite, which was rinsed with EtOAc. The combined filtrates were concentrated and the residue was purified by flash column chromatography on silica gel (hexane/EtOAc 4:1 to 1:1) to give 590 mg of light brown solid 121(c) (39%) and 340 mg of dark brown solid 121(d) (33%).

e) 1,1-dimethylethyl 3-methyl-5-(trimethylstannanyl)-1H-thieno[3,2-c]pyrazole-1-carboxylate

A mixture of 121(c) (590 mg, 1.86 mmol), hexamethylditin (1 g, 1.64 eq.), Pd(Ph₃P)₄ (107 mg, 5 mol %) and 10 mL of toluene was degassed and heated at 110° C. under N₂ overnight. The reaction mixture was filtered through celite, which was rinsed with EtOAc. The combined filtrates were concentrated and the residue was purified by flash column chromatography on silica gel (hexane/EtOAc 7:1 to 1:1) to give 367 mg of light brown oil 121(e) (49%) and 50 mg of light brown oil 121(f) (9%).

g) 1,1-dimethylethyl 5-(2-chloro-5-{[(2S)-2-({[(1,1-dimethylethyl)oxy]carbonyl}amino)-3-(1H-indol-3-yl)propyl]oxy}-3-pyridinyl)-3-methyl-1H-thieno[3,2-c]pyrazole-1-carboxylate

A mixture of 121(e) (367 mg, 0.91 mmol), 69(a) (438 mg, 1.0 eq.), Pd(Ph₃P)₄ (105 mg, 10 mol %), Et₃N (0.38 mL, 3.0 eq.) and 5 mL of 1,4-dioxane was degassed and heated at 100° C. under N₂ overnight. The reaction mixture was filtered through celite, which was rinsed with EtOAc. The combined filtrates were concentrated and the residue was purified by flash column chromatography on silica gel (hexane/EtOAc 7:1 to 1:1) to give 330 mg of light yellow solid 121(g) (57%) and 105 mg of yellow solid 121(h) (20%).

i) [(1S)-2-{[6-(3-furanyl)-5-(3-methyl-1H-thieno]3,2-c[pyrazol-5-yl)-3-pyridinyl]oxy}-1-(1H-indol-3-ylmethyl)ethyl]amine

Applied the standard TFA de-boc procedure to 121(g), 121(h) to provide the final titled compound. ¹H NMR (CD₃OD, 400 MHz) δ 8.40 (s, 1H), 6.99-7.60 (m, 9H), 6.50 (d, 1H), 4.40 (dd, 1H), 4.20 (dd, 1H), 3.90-4.00 (m, 1H), 3.30 (dd, 2H), 2.50 (s, 3H). MS (M+H): 470.2.

### Example 122

### Preparation of 5-[5-{[(2S)-2-amino-3-phenylpropyl]oxy}-2-(3-furanyl)-3-pyridinyl]-N-4-pyridinyl-1H-indazol-3-amine

a) 1,1-dimethylethyl [(1S)-2-{[6-(3-furanyl)-5-(3-iodo-1H-indazol-5-yl)-3-pyridinyl]oxy}-1-(phenylmethyl)ethyl]carbamate

Following the procedure of making 23(a) except substituting 3-furanylboronic acid for phenylboronic acid, then carried the standard lodination reaction to provide the above titled compound.

b) 1,1-dimethylethyl [(1S)-2-{[6-(3-furanyl)-5-(3-iodo-1-{[4-(methyloxy)phenyl]methyl}-1H-indazol-5-yl)-3-pyridinyl]oxy}-1-(phenylmethyl)ethyl]carbamate

A mixture of 122(a) (127 mg, 0.2 mmol), para-methoxybenzyl chloride (32.6 μl, 1.2 eq.), Cs₂CO₃ (78 mg, 1.2 eq.), Nal (6 mg, 20 mol %) and 1 mL of DMF was stirred at 25° C. overnight. The reaction mixture was taken up into EtOAc, which was washed with water, brine, and dried (Na₂SO₄). Solvent was removed and the residue was purified by flash column chromatography on silica gel (hexane/EtOAc 2:1) to give 117 mg of off-white foamy solid 122(b) (77%).

c) 1,1-dimethylethyl [(1S)-2-({6-(3-furanyl)-5-[1-{[4-(methyloxy)phenyl]methyl}-3-(4-pyridinylamino)-1H-indazol-5-yl]-3-pyridinyl}oxy)-1-(phenylmethyl)ethyl]carbamate

A mixture of 122(b) (58.5 mg, 0.077 mmol), 4-aminopyridine (10 mg, 1.4 eq.), Pd₂ dba₃ (1.4 mg, 2 mol %), xantphos (2.7 mg, 6 mol %), Cs₂CO₃ (35 mg, 1.4 eq.) and 0.7 mL of 1,4-dioxane was charged with N₂, sealed and heated at 100° C. overnight. The reaction mixture was filtered through celite, which was rinsed with EtOAc. The combined filtrates were concentrated and the residue was purified by flash column chromatography on silica gel (hexane/EtOAc 3:1) to give 20 mg of light brown foamy solid titled compound(36%).

d) 5-[5-{[(2S)-2-amino-3-phenylpropyl]oxy}-2-(3-furanyl)-3-pyridinyl]-N-4-pyridinyl-1H-indazol-3-amine

A solution of 122(c) (20 mg, 0.028 mmol) in 0.5 mL of TFA was heated at 65° C. for 24 h and concentrated. The residue was purified with reversed phase HPLC to give 17.0 mg titled final compound as the light yellow solid (73%). ¹H NMR (CD₃OD, 400 MHz) δ 8.45 (s, 1H), 7.25-8.30 (m, 15H), 6.30 (d, 1H), 4.40 (dd, 1H), 4.26 (dd, 1H), 3.90-4.00 (m, 1H), 3.15 (dd, 2H). MS (M+H): 503.2.

### Example 123

### Preparation of N-{5-[5-{[(2S)-2-amino-3-phenylpropyl]oxy}-2-(3-furanyl)-3-pyridinyl]-1H-indazol-3-yl]benzamide

a) 1,1-dimethylethyl [(1S)-2-[(6-(3-furanyl)-5-{1-{[4-(methyloxy)phenyl]methyl}-3-[(phenylcarbonyl)amino]-1H-indazol-5-yl}-3-pyridinyl)oxy]-1-(phenylmethyl)ethyl]carbamate

A mixture of 122(b)(58.5 mg, 0.077 mmol), benzamide (11.2 mg, 1.2 eq.), Cul (1.5 mg, 10 mol %), 1,10-phenanthroline (2.8 mg, 20 mol %), K₂CO₃ (16.6 mg, 2.0 eq.) and 0.7 mL of 1,4-dioxane was charged with N₂, sealed and heated at 100° C. overnight. The reaction mixture was filtered through celite, which was rinsed with EtOAc. The combined filtrates were concentrated and the residue was purified by flash column chromatography on silica gel (hexane/EtOAc 2:1) to give 19.5 mg of light brown foamy solid titled compound (34%, 53% based on recovered starting material).

b) N-{5-[5-{[(2S)-2-amino-3-phenylpropyl]oxy}-2-(3-furanyl)-3-pyridinyl]-1H-indazol-3-yl}benzamide

Following the standard TFA De-boc procedure to provide the titled final product. ¹H NMR (CD₃OD, 400 MHz) δ 7.80-8.45 (m, 5H), 7.20-7.68 (m, 12H), 6.38 (d, 1H), 4.40 (dd, 1H), 4.26 (dd, 1H), 3.90-4.00 (m, 1H), 3.15 (dd, 2H). MS (M+H): 530.2.

### Example 124

### Preparation of (1E)-1-{3-[5-{[(2S)-2-amino-3-(1H-indol-3-yl)propyl]oxy}-2-(3-furanyl)-3-pyridinyl[phenyl]ethanone oxime

To a solution of Example 113 (before De-Boc) (100 mg, 0.18 mmol) and NaOAc (30 mg, 0.36 mmol) in EtOH (3 ml), H₂NOH HCl (25 mg, 0.36 mmol) was added. The reaction was stirred at room temperature overnight. Removed solvent, the reaction mixture was washed with NaCl and dried over MgSO4. Concentrated and purified by flash column chromatography (1:1 hexene/EtOAc) to give 96 mg (91%) solid, which was treated with TFA/CH2Cl2 and purified by reverse phase HPLC to give the title compound. ¹H NMR (400 MHz, MeOD) δ ppm 8.40 (t, J=3.0 Hz, 1H), 7.75 (d, J=7.8 Hz, 1H), 7.57-7.64 (m, 3H), 7.42-7.50 (m, 2H), 7.39 (d, J=8.1 Hz, 1H), 7.24-7.33 (m, 3H), 7.11-7.17 (m, 1H), 7.02-7.07 (m, 1H), 6.33 (d, J=1.0 Hz, 1H), 4.42 (dd, J=10.5, 2.9 Hz, 1H), 4.28 (dd, J=10.4, 5.8 Hz, 1H), 4.00 (m, 1H), 3.33-3.35 (m, 2H), 2.22 (s, 3H); MS: 467.2

### Example 125

### Preparation of [(1S)-2-{[6-(3-furanyl)-5-(3-methyl-1H-indazol-5-yl)-3-pyridinyl]oxy}-1-(phenylmethyl)propyl]amine

Following the procedure of Example 1(a)-1(f), except substituting 4,4,5,5-tetramethyl-2-phenyl-1,3,2-dioxaborolane for 2-(3-furanyl)-4,4,5,5-tetramethyl-1,3,2-dioxaborolane, and substituting 1(a) with 1,1-dimethylethyl [(1S)-2-hydroxy-1-(phenylmethyl)propyl]carbamate. The procedure to make 1,1-dimethylethyl [(1S)-2-hydroxy-1-(phenylmethyl)propyl]carbamate is as following: A solution of MeMgBr (0.97 ml, 3M in diethyl ether was added to a solution of (S)-(−)-2(tert-butoxycarbonylamino)-3-phenylpropanal (320 mg, 1.29 mmol) at −78° C. The resulting reaction mixture was warmed up to 0° C. and stirred at this temperature for 30 min. The reaction was quenched with saturated NH₄Cl aqueous solution and the organic layer was separated, dried (Na₂SO₄) and concentrated. The residue was purified by flash column chromatography on silica gel (2:1 hexane/EtOAc) to give the product as white solid (335 mg, 98%). ¹H NMR (CD₃OD, 400 MHz) δ 7.45 (s, 1H), 7.18-7.75 (m, 11H), 6.30 (d, 1H), 4.75-5.02 (m, 1H), 3.78-3.96 (m, 1H), 3.06-3.22 (m, 2H), 2.56 (s, 3H), 1.56 (d, 3H). MS (M+H): 439.4.

### Example 126

### Preparation of (2S)-N-methyl-1-{[5-(3-methyl-1H-indazol-5-yl)-6-phenyl-3-pyridinyl]oxy}-3-phenyl-2-propanamine

Following the procedure of Example 1(a)-1(f), except carrying the methylation reaction before the first Suzuki coupling reaction. The methylation step was carried as following: To the solution of 1(b) (200 mg, 0.46 mmol) in dry THF at 0° C. under N₂ was added NaH (35 mg, 1.4 mmol), and Mel (98 mg, 0.70 mmol). The reaction was stirred at 0° C. for an hour, then gradually warmed up to RT. Dissolve the mixture in EtOAc, then washed by NaHCO₃ and brine. After concentrated down, the residue was purified by Biotage to provide 131 mg of 1,1-dimethylethyl [(1S)-2-[(5-bromo-6-chloro-3-pyridinyl)oxy]-1-(phenylmethyl)ethyl]methylcarbamate (64%).

1H NMR (CD₃OD, 400 MHz) δ 8.48 (s, 1H), 7.83 (s, 1H), 7.65 (s, 1H), 7.10-7.41 (m, 12H), 4.50 (dd, 1H), 4.31 (dd, 1H), 3.90-3.99 (m, 1H), 3.25 (dd, 2H), 2.90 (s, 3H), 2.52 (s, 3H). MS (M+H): 449.2.

### Example 127

### Preparation of [(1S)-2-{[6-[5-fluoro-2-(methyloxy)phenyl]-5-(3-methyl-1H-indazol-5-yl)-3-pyridinyl]oxy}-1-(phenylmethyl)ethyl]amine

Following the procedure of Example 1(a)-1(f), except substituting [5-fluoro-2-(methyloxy)phenyl]boronic acid for phenylboronic acid, the titled compound was prepared. ¹H NMR (CD₃OD, 400 MHz) δ 8.52 (s, 1H), 8.00 (s, 1H), 7.61 (s, 1H), 6.85-7.43 (m, 10H), 4.50 (dd, 1H), 4.35 (dd, 1H), 3.95-4.05 (m, 1H), 3.45 (s, 3H), 3.19 (dd, 2H), 2.49 (s, 3H). MS (M+H): 483.2.

### Example 128

### Preparation of [(1S)-2-{[6-[3,5-difluoro-2-(methyloxy)phenyl]-5-(3-methyl-1H -indazol-5-yl)-3-pyridinyl]oxy}-1-(phenylmethyl)ethyl]amine

Following the procedure of Example 1(a)-1(f), except substituting 3-methyl -5-(4,4,5,5-tetramethyl-1,3,2-dioxaborolan-2-yl)-1H-indazole for 1,1-dimethylethyl 3-methyl-5-(4,4,5,5-tetramethyl-1,3,2-dioxaborolan-2-yl)-1H-indazole-1-carboxylate, and substituting [3,5-difluoro-2-(methyloxy)phenyl]boronic acid for phenylboronic acid, the title compound was prepared. ¹H NMR (CD₃OD, 400 MHz) δ 8.41 (s, 1H), 7.69 (s, 1H), 7.60 (s, 1H), 6.88-7.40 (m, 9H), 4.40 (dd, 1H), 4.25 (dd, 1H), 3.95-4.05 (m, 1H), 3.45 (s, 3H), 3.18 (dd, 2H), 2.50 (s, 3H). MS (M+H): 501.2.

### Example 129

### Preparation of [(1S)-2-(}6-(3-furanyl)-5-[3-(4-pyridinyl)-1H-indazol-5-yl]-3-pyridinyl}oxy)-1-(phenylmethyl)ethyl]amine

Following the procedure of Example 23(a)-23(c), except substituting 4-pyridinylboronic acid for phenylboronic acid, the title compound was prepared. ¹H NMR (CD₃OD, 400 MHz) δ 8.85 (d, 2H), 8.68 (d, 2H), 8.46 (s, 1H), 8.29 (s, 1H), 7.20-7.65 (m, 10H), 6.35 (s, 1H), 4.40 (dd, 1H), 4.25 (dd, 1H), 3.90-4.00 (m, 1H), 3.18 (dd, 2H). MS (M+H): 488.2.

### Example 130

### Preparation of 2-[5-{[(2S)-2-amino-3-phenylpropyl]oxy}-3-(3-methyl-1H-indazol-5-yl)-2-pyridinyl]-4-fluorophenol

Started from the final product of example 127, following the standard BBr₃ to remove the methyl group to provide the titled final product. ¹H NMR (CD₃OD, 400 MHz) δ 8.48 (s, 1H), 7.89 (s, 1H), 7.62 (s, 1H), 6.70-7.39 (m, 10H), 4.45 (dd, 1H), 4.30 (dd, 1H), 3.91-4.03 (m, 1H), 3.18 (dd, 2H), 2.50 (s, 3H). MS (M+H): 469.2.

### Example 131

### Preparation of 2-[5-{[(2S)-2-amino-3-phenylpropyl]oxy}-3-(3-methyl-1H-indazol-5-yl)-2-pyridinyl]-4,6-difluorophenol

Started from the final product of example 128, following the standard BBr₃ to remove the methyl group to provide the titled final product. ¹H NMR (CD₃OD, 400 MHz) δ 8.40 (s, 1H), 6.65-7.68 (m, 11H), 4.40 (dd, 1H), 4.25 (dd, 1H), 3.91-4.03 (m, 1H), 3.18 (dd, 2H), 2.50 (s, 3H). MS (M+H): 487.2.

### Example 132

### Preparation of 2-[5-{[(2S)-2-amino-3-(1H-indol-3-yl)propyl]oxy}-3-(6-fluoro-3-methyl-1H-indazol-5-yl)-2-pyridinyl]phenol

Following the procedure of 69(a)-69(c) except substituting 6-fluoro-3-methyl-5-(4,4,5,5-tetramethyl-1,3,2-dioxaborolan-2-yl)-1H-indazole (Example 104(d)) for 3-methyl-5-(4,4,5,5-tetramethyl-1,3,2-dioxaborolan-2-yl)-1H-indazole, and substituting 2-(4,4,5,5-tetramethyl-1,3,2-dioxaborolan-2-yl)phenol for phenylboronic acid. The titled compound was prepared. ¹H NMR (CD₃OD, 400 MHz) δ 8.50 (s, 1H), 7.95 (s, 1H), 6.69-7.62 (m, 11H), 4.50 (dd, 1H), 4.35 (dd, 1H), 3.98-4.08 (m, 1H), 3.31 (dd, 2H), 2.49 (s, 3H). MS (M+H): 508.2.

### Example 133

### Preparation of 2-[5-{[(2S)-2-amino-3-(1H-indol-3-yl)propyl]oxy}-3-(3-ethyl-1H-indazol-5-yl)-2-pyridinyl]phenol

a) 1-(5-bromo-2-fluorophenyl)-1-propanol

To the solution of 5-bromo-2-fluorobenzaldehyde (4.06 g, 20 mmol) in ether (100 ml) at 0° C. was slowly added EtMgBr. The reaction mixture was stirred at 0° C. overnight. Then added 1 N HCl slowly to the mixture, washed with NH₄Cl and brine solution, dried over MgSO₄. The yellow oilish crude material was used in the next step without further purification.

b) 1-(5-bromo-2-fluorophenyl)-1-propanone

The solution of 133(a)(1.8 g, 7.7 mmol) and Dess-Martin reagent (5.0 g, 11.8 mmol) in dichloromethane was stirred at RT for 5 hrs. Then ether was added, followed by the addition of Na₂S₂0₃. Removed the solvent, diluted the residue with EtOAc, then washed with NaHCO₃ and brine solution. The residue was purified by Biotage to give 1.67 g titled compound (93%).

c) 5-bromo-3-ethyl-1H-indazole

The solution of 133(b)(1.0 g, 4.32 mmol) in dry hydrazine (2.5 ml, 80 mmol) was heated to 115° C. overnight. Cooled down the mixture, and added water, then extracted with dichloromethane. Concentrated down and the residue was purified by Biotage to provide 0.93 g of the titled compound (95%).

d) 3-ethyl-5-(4,4,5,5-tetramethyl-1,3,2-dioxaborolan-2-yl)-1H-indazole

The mixture of PCy₃ (0.95 g, 3.38 mmol) and Pd2 dba3 (0.52 g, 0.564 mmol) in dry dioxane (40 ml) was stirred under N₂ at RT for 3 hrs. Compound 133(c)(4.2 g, 18.8 mmol), 4,4,4′,4′,5,5,5′,5′-octamethyl-2,2′-bi-1,3,2-dioxaborolane (5.72 g, 22.5 mmol), KOAc (2.8 g, 28.2 mmol) were dissolved in dioxane (40 ml), and added the pre-mixed catalyst. The reaction mixture was heated at 80° C. under N₂ for 24 h. Cooled down and the mixture was filtered through celite, which was rinsed with EtOAc. The combined filtrates were concentrated and the residue was purified by flash column chromatography (hexane/EtOAc 9:1) to give 4.58 g titled compound 133(d) (90%).

e) 2-[5-{[(2S)-2-amino-3-(1H-indol-3-yl)propyl]oxy}-3-(3-ethyl-1H-indazol-5-yl)-2-pyridinyl]phenol

Started from compound 69(a), following by first Suzuki coupling with 133(d), second Suzuki coupling with (2-hydroxyphenyl)boronic acid, then De-boc with TFA provided with the titled final compound. ¹H NMR (CD₃OD, 400 MHz) δ 8.50 (s, 1H), 8.16 (s, 1H), 6.72-7.61 (m, 12H), 4.54 (dd, 1H), 4.40 (dd, 1H), 4.00-4.11 (m, 1H), 3.33 (dd, 2H), 2.89 (q, 2H), 1.35 (t, 3H). MS (M+H): 504.4.

### Example 134

### Preparation of [(1S)-2-{[5-(3-ethyl-1H-indazol-5-yl)-6-(3-furanyl)-3-pyridinyl]oxy}-1-(1H-indol-3-ylmethyl)ethyl]amine

Following the procedure of example 133 except substituting 3-furanylboronic acid for (2-hydroxyphenyl)boronic acid, the titled final compound was prepared. ¹H NMR (CD₃OD, 400 MHz) δ 8.41 (s, 1H), 7.01-7.70 (m, 11H), 6.25 (d, 1H), 4.45 (dd, 1H), 4.25 (dd, 1H), 3.94-4.09 (m, 1H), 3.30 (dd, 2H), 3.00 (q, 2H), 1.45 (t, 3H). MS (M+H): 478.2.

### Example 135

### Preparation of [(1S)-2-{[5-(3-ethyl-1H-indazol-5-yl)-6-(2-furanyl)-3-pyridinyl]oxy}-1-(1H-indol-3-ylmethyl)ethyl]amine

Following the procedure of example 133 except substituting 2-furanylboronic acid for (2-hydroxyphenyl)boronic acid, the titled final compound was prepared. ¹H NMR (CD₃OD, 400 MHz) δ 8.38 (s, 1H), 7.68 (s, 1H), 6.98-7.60 (m, 9H), 6.35 (d, 1H), 5.90 (d, 1H), 4.40 (dd, 1H), 4.25 (dd, 1H), 3.94-4.01 (m, 1H), 3.30 (dd, 2 H), 3.00 (q, 2H), 1.45 (t, 3H). MS (M+H): 478.2.

### Example 136

### Preparation of [(1S)-2-{8 5-(3-ethyl-1H-indazol-5-yl)-6-(1H-pyrrol-2-yl)-3-pyridinyl]oxy}-1-(1H-indol-3-ylmethyl)ethyl]amine

Following the procedure of example 133 except substituting (1-{[(1,1-dimethylethyl)oxy]carbonyl}-1H-pyrrol-2-yl)boronic acid for (2-hydroxyphenyl)boronic acid, the titled final compound was prepared. ¹H NMR (CD₃OD, 400 MHz) δ 8.35 (s, 1H), 6.82-7.71 (m, 10H), 6.09 (d, 1H), 5.89 (d, 1H), 4.44 (dd, 1H), 4.30 (dd, 1H), 3.96-4.03 (m, 1H), 3.30 (dd, 2H), 3.00 (q, 2H), 1.40 (t, 3H). MS (M+H): 477.2.

### Example 137

### Preparation of [(1S)-2-({6-(3-furanyl)-5-[3-(1-methyl-1H-pyrazol-4-yl)-1H-indazol-5-yl]-3-pyridinyl}oxy)-1-(phenylmethyl)ethyl]amine

Started from 122(a), following by standard Suzuki coupling procedure with 1-methyl-4-(4,4,5,5-tetramethyl-1,3,2-dioxaborolan-2-yl)-1H-pyrazole, then continued with standard De-boc method to provide the titled compound. ¹H NMR (CD₃OD, 400 MHz) δ 8.45 (s, 1H), 8.12 (s, 1H), 7.20-8.00 (m, 12H), 6.29 (d, 1H), 4.36 (dd, 1H), 4.26 (dd, 1H), 3.90-4.00 (m, 4H), 3.30 (dd, 2H). MS (M+H): 491.0.

### Example 138

### Preparation of [(1S)-2-({6-(3-furanyl)-5-[3-(1H-pyrrol-2-yl)-1H-indazol-5-yl]-3-pyridinyl}oxy)-1-(phenylmethyl)ethyl]amine

Following the procedure for example 137 except substituting (1-{[(1,1-dimethylethyl)oxy]carbonyl}-1H-pyrrol-2-yl)boronic acid for 1-methyl-4-(4,4,5,5-tetramethyl-1,3,2-dioxaborolan-2-yl)-1H-pyrazole to provide the titled compound.

¹H NMR (CD₃OD, 400 MHz) δ 8.46 (s, 1H), 8.00 (s, 1H), 6.68-7.72 (m, 12H), 6.35 (d, 1H), 6.25 (d, 1H), 4.40 (dd, 1H), 4.22 (dd, 1H), 3.85-4.00 (m, 1H), 3.15 (d, 2H). MS (M+H): 476.2.

### Example 139

### Preparation of [(1S)-2-({6-(3-furanyl)-5-[3-(1H-pyrazol-4-yl)-1H-indazol-5-yl]-3-pyridinyl}oxy)-1-(phenylmethyl)ethyl]amine

Following the procedure for example 129 except substituting 4-(4,4,5,5-tetramethyl-1,3,2-dioxaborolan-2-yl)-1H-pyrazole for 4-pyridinylboronic acid to provide the titled compound. ¹H NMR (CD₃OD, 400 MHz) δ 8.40 (s, 1H), 7.20-8.16 (m, 13H), 6.35 (d, 1H), 4.40 (dd, 1H), 4.20 (dd, 1H), 3.90-4.00 (m, 1H), 3.15 (d, 2H). MS (M+H): 477.2.

### Example 140

### Preparation of [(1S)-2-{[5-(6-fluoro-3-methyl-1H-indazol-5-yl)-6-(2-furanyl)-3-pyridinyl]oxy}-1-(1H-indol-3-ylmethyl)ethyl]amine

Following the procedure of example 132 except substituting 2-furanylboronic acid for 2-(4,4,5,5-tetramethyl-1,3,2-dioxaborolan-2-yl)phenol to provide the title compound. ¹H NMR (CD₃OD, 400 MHz) δ 8.40 (s, 1H), 7.02-7.58 (m, 9H), 6.35 (d, 1H), 6.02 (d, 1H), 4.40 (dd, 1H), 4.25 (dd, 1H), 3.90-4.00 (m, 1H), 3.30 (d, 2H), 2.55 (s, 3H). MS (M+H): 482.2.

### Example 141

### Preparation of [(1S)-2-{[5-(6-fluoro-3-methyl-1H-indazol-5-yl)-6-(1H-pyrrol-2-yl)-3-pyridinyl]oxy}-1-(1H-indol-3-ylmethyl)ethyl]amine

Following the procedure of example 132 except substituting (1-{[(1,1-dimethylethyl)oxy]carbonyl}-1H-pyrrol-2-yl)boronic acid for 2-(4,4,5,5-tetramethyl -1,3,2-dioxaborolan-2-yl)phenol to provide the title compound. ¹H NMR (CD₃OD, 400 MHz) δ 8.36 (s, 1H), 6.80-7.68 (m, 9H), 5.90 (d, 1H), 5.38 (d, 1H), 4.40 (dd, 1H), 4.25 (dd, 1H), 3.90-4.00 (m, 1H), 3.30 (dd, 2H), 2.55 (s, 3H). MS (M+H): 481.2.

### Example 142

### Preparation of [(1s)-2-{[5-(6-fluoro-3-methyl-1H-indazol-5-yl)-6-(3-furanyl)-3-pyridinyl]oxy}-1-(1H-indol-3-ylmethyl)ethyl]amine

Following the procedure of example 132 except substituting 3-furanylboronic acid for 2-(4,4,5,5-tetramethyl-1,3,2-dioxaborolan-2-yl)phenol to provide the title compound. ¹H NMR (CD₃OD, 400 MHz) δ 8.45 (s, 1H), 6.98-7.70 (m, 10H), 6.38 (d, 1H), 4.40 (dd, 1H), 4.25 (dd, 1H), 3.95-4.05 (m, 1H), 3.30 (dd, 2H), 2.55 (s, 3H). MS (M+H): 482.0.

### Example 143

### Preparation of [(1S)-2-{[6-(1-benzothien-2-yl)-5-(3-methyl-1H-indazol-5-yl)-3-pyridinyl]oxy}-1-(phenylmethyl)ethyl]amine

Following the procedure of example 128, except substituting 1-benzothien-2-ylboronic acid for [3,5-difluoro-2-(methyloxy)phenyl]boronic acid, the title compound was prepared. ¹H NMR (CD₃OD, 400 MHz) δ 8.45 (s, 1H), 7.20-7.75 (m, 13H), 6.72 (s, 1H), 4.38 (dd, 1H), 4.20 (dd, 1H), 3.90-4.00 (m, 1H), 3.15 (dd, 2H), 2.55 (s, 3H). MS (M+H): 491.0.

### Example 144

### Preparation of [(1S)-2-{[6-(1-benzofuran-2-yl)-5-(3-methyl-1H-indazol-5-yl)-3-pyridinyl]oxy}-1-(phenylmethyl)ethyl]amine

Following the procedure of example 128, except substituting 1-benzofuran-2-ylboronic acid for [3,5-difluoro-2-(methyloxy)phenyl]boronic acid, the title compound was prepared. ¹H NMR (CD₃OD, 400 MHz) δ 8.48 (s, 1H), 7.10-7.75 (m, 13H), 6.30 (s, 1H), 4.38 (dd, 1H), 4.20 (dd, 1H), 3.90-4.00 (m, 1H), 3.18 (dd, 2H), 2.58 (s, 3H). MS (M+H): 475.2.

### Example 145

### Preparation of [(1S)-2-({6-(3-furanyl)-5-[3-(methylsulfonyl)phenyl]-3-pyridinyl}oxy)-1-(1H-indol-3-ylmethyl)ethyl]amine

Following the procedure of Example 69, except substituting [3-(methylsulfonyl)phenyl]boronic acid for Example 1(c), the title compound was prepared. ¹H NMR (400 MHz, MeOD) δ ppm 8.39-8.43 (m, 1H), 8.02 (d, J=7.8 Hz, 1H), 7.85 (s, 1H), 7.70 (t, J=7.7 Hz, 1H), 7.57-7.65 (m, 2H), 7.45-7.51 (m, 1H), 7.36-7.43 (m, 2H), 7.29 (d, J=7.3 Hz, 1H), 7.23 (s, 1H), 7.12 (t, J=7.6 Hz, 1H), 7.02 (t, J=7.5 Hz, 1H), 6.23 (s, 1H), 4.38 (d, J=10.4 Hz, 1H), 4.22-4.28 (m, 1H), 3.98 (m, 1H), 3.33-3.35 (m, 2H), 3.10 (s, 3H); MS: 488.2.

### Example 146

### Preparation of 5-[5-{[(2S)-2-(1-azetidinyl)-3-(1H-indol-3-yl)propyl]oxy}-2-(3-furanyl)-3-pyridinyl]-3-methyl-1H-indazole

Following the procedure of example 69(a)-69(c) except substituting 3-furanylboronic acid for phenylboronic acid to provide [(1S)-2-{[6-(3-furanyl)-5-(3-methyl-1H-indazol-5-yl)-3-pyridinyl]oxy}-1-(1H-indol-3-ylmethyl)ethyl]amine intermediate. Then mixed this intermediate (70 mg, 0.1 mmol), 1,3-dibromopropane (22 mg, 0.11 mmol), Na₂CO₃ (106 mg, 1.0 mmol) in EtOH (5 ml), and refluxed overnight, cooled down and crude mixture was purified by HPLC to provide 7.2 mg the titled final compound (10%). ¹H NMR (CD₃OD, 400 MHz) δ 8.39 (s, 1H), 7.20-7.68 (m, 9H), 7.10-7.16 (m, 1H), 6.95-7.04 (m, 1H), 6.30 (d, 1H), 4.36-4.55 (m, 2H), 4.20-4.28 (m, 2H), 4.06-4.14 (m, 1H), 3.15-3.25 (m, 4H), 2.62-2.79 (m, 1H), 2.60 (s, 3H), 2.32-2.43 (m, 1H). MS (M+H): 504.2

### Example 147

### Preparation of [(1S)-2-({6-(3-furanyl)-5-[3-(1H-pyrazol-4-yl)-1H-indazol-5-yl]-3-pyridinyl}oxy)-1-(1H-indol-3-ylmethyl)ethyl]amine

Following the procedure of Example 139, except substituting N-(tert-butoxycarbonyl)-L-tryptophanol for (2S)-N-(tert-butoxycarbonyl)-2-amino-3-phenyl -1-propanol, the title compound was prepared. ¹H NMR (CD₃OD, 400 MHz) 68.49 (d, 1H), 8.35 (br, 1H), 7.99 (s, 1H), 7.86 (s, 1H), 7.62-26 (m, 8H), 7.20-7.10 (m, 2H), 6.30 (s, 1H), 4.48 (dd, 1H), 4.33 (dd, 1H), 4.05-3.99 (m, 1H), 3.32 (m, 2H); MS (M+H): 516.2

### Example 148

### Preparation of 3-[5-{[(2S)-2-amino-3-(1H-indol-3-yl)propyl]oxy}-2-(3-furyl)pyridin-3-yl]benzamide

Following the procedure of Example 73 except for substituting [3-(aminocarbonyl)phenyl]boronic acid for 3-methyl-5-(4,4,5,5-tetramethyl-1,3,2-dioxaborolan-2-yl)-1H-indazole, the title compound was prepared. ¹H NMR (CD₃OD, 400 MHz) δ 8.41 (d, 1H), 7.98 (d, 1H), 7.85 (d, 1H), 7.62-7.52 (m, 3H), 7.46-7.38 (m, 3H), 7.26 (s, 1H), 7.22 (s, 1H), 7.12 (t, 1H), 7.04 (t, 1H), 6.30 (s, 1H), 4.43 (dd, 1H), 4.28 (dd, 1H), 4.00 (m, 1H), 3.36 (m, 2H) MS (M+H): 453.2.

### Example 149

### Preparation of 4-[5-{[(2S)-2-amino-3-(1H-indol-3-yl)propyl]oxy}-2-(3-furyl)pyridin-3-yl]benzamide

Following the procedure of Example 73 except for substituting [4-(aminocarbonyl)phenyl]boronic acid for 3-methyl-5-(4,4,5,5-tetramethyl-1,3,2-dioxaborolan-2-yl)-1H-indazole, the title compound was prepared. ¹H NMR (CD₃OD, 400 MHz) δ 8.40 (d, 1H), 7.96 (d, 2H), 7.60 (d, 1H), 7.49 (d, 1H), 7.48-7.38 (m, 4H), 7.28 (s, 1H), 7.24 (s, 1H), 7.15 (t, 1H), 7.06 (t, 1H), 6.30 (s, 1H), 4.40 (dd, 1H), 4.26 (dd, 1H), 4.00 (m, 1H), 3.36 (m, 2H) MS (M+H): 453.2.

### Example 150

### Preparation of 5-(5-{[(2S)-3-(1H-indol-3-yl)-2-(1-piperidinyl)propyl]oxy}-2-phenyl-3-pyridinyl)-3-methyl-1H-indazole

A solution of the compound of Example 69 (30 mg, 0.037 mmol), 1,5-dibromopentane (8.5 mg, 0.037 mmol) and Na₂CO₃ (39 mg, 0.37 mmol) were mixed in the mixture of 1 ml DMF and 6 ml CH₃CN. The solution was heated at 100° C. overnight. After cooled to room temperature, 50 ml EtOAc was added to the mixture and washed with brine. The organic layer was concentrated and purified by reverse phase HPLC. Got Example 150 7.3 mg as solid in 36% yield. 1H NMR (CD₃OD, 400 MHz) δ 8.43 (d, 1H), 7.70 (d, 1H), 7.65-7.60 (m, 2H), 7.49-7.25 (m, 8H), 7.20-7.00 (m, 3H), 4.65 (dd, 1H), 4.50 (dd, 1H), 4.10-4.05 (m, 1H), 3.95-3.85 (m, 2H), 3.60-3.45 (m, 4H), 2.50 (s, 3H), 2.20-1.60 (m, 6H); MS (M+H): 542.4.

### Example 151

### Preparation of 5-(2-(3-furanyl)-5-{[(2S)-3-(1H-indol-3-yl)-2-(4-morpholinyl)propyl]oxy}-3-pyridinyl)-3-methyl-1H-indazole

Following the procedure of Example 150 except substituting Example 77 for Example 69 and substituting bis(2-bromoethyl) ether for 1,5-dibromopentane, the title compound was prepared. 1H NMR (CD₃OD, 400 MHz) δ 8.38 (d, 1H), 7.65 (d, 1H), 7.60-7.49 (m, 3H), 7.42 (s, 1H), 7.45 (d, 1H), 7.35-6.96 (m, 5H), 6.28 (dd, 1H), 4.60 (dd, 1H), 4.38 (dd, 1H), 4.30-3.50 (m, 11H), 2.57 (s, 3H). MS (M+H): 534.4.

### Example 152

### Preparation of [(1S)-2-({6-(3-furanyl)-5-[3-(1H-pyrazol-4-yl)-1H-indazol-5-yl]-3-pyridinyl}oxy)-1-(1H-indol-3-ylmethyl)ethyl]amine

Following the procedure of Example 139 except substituting the compound of Example 70(a) for Example 1(b), the title compound was prepared. 1H NMR (CD₃OD, 400 MHz) δ 8.48 (d, 1H), 8.25 (br, 1H), 7.98 (d, 1H), 7.85 (d, 1H), 7.65-7.34 (m, 8H), 7.20-6.95 (m, 2H), 6.32 (s, 1H), 4.53-4.30 (m, 2H), 4.03-4.00 (m, 1H), 3.33 (m, 2H); MS (M+H): 516.2.

### Example 153

### Preparation of [(1S)-2-{[6-(3-furanyl)-5-(3-methyl-1H-indazol-5-yl)-3-pyridinyl]oxy}-1-(1H-indol-3-ylmethyl)ethyl]dimethylamine

Following the procedure of Example 48 except substituting the compound of Example 70(a) for Example 1(a) and substituting 3-furoboronic acid for phenylboronic acid, the title compound was prepared. 1H NMR (CD₃OD, 400 MHz) δ 8.41 (d, 1H), 7.76 (d, 1H), 7.69 (s, 1H), 7.62 (d, 1H), 7.51 (d, 1H), 7.46 (s, 1H), 7.30-7.40 (m, 2H), 7.25 (s, 1H), 7.23 (dd, 1H), 7.13 (dd, 1H), 7.00 (dd, 1H), 6.28 (dd, 1H), 4.54-4.50 (m, 2H), 4.23-4.10 (m, 1H), 3.60-3.35 (m, 2H), 3.16 (s, 6H), 2.57 (s, 3H); MS (M+H): 492.2.

### Example 154

### Preparation of (3S)-3-({[6-(3-furanyl)-5-(3-methyl-1H-indazol-5-yl)-3-pyridinyl]oxy}methyl)-2-methyl-2,3,4,9-tetrahydro-1H-carboline

Following the procedure of Example 153, the title compound was separated as a by-product from reverse phase HPLC. 1H NMR (CD₃OD, 400 MHz) δ 8.54 (d, 1H), 7.97 (d, 1H), 7.80 (s, 1H), 7.54-7.10 (m, 8H), 6.31 (dd, 1H), 4.82-4.60 (m, 4H), 4.40-4.30 (m, 1H), 3.25 (s, 3H), 3.25-3.10 (m, 2H), 3.16 (s, 6H), 2.57 (s, 3H); MS (M+H): 490.2.

### Example 155

### Preparation of 1-{5-[5-{[(2S)-2-amino-3-(1H-indol-3-yl)propyl]oxy}-2-(3-furanyl)-3-pyridinyl]-2-thienyl}ethanone

Following the procedure of Example 77, except substituting (5-acetyl-2-thienyl)boronic acid for 1,1-dimethylethyl 3-methyl-5-(4,4,5,5-tetramethyl-1,3,2-dioxaborolan-2-yl)-1H-indazole-1-carboxylate, the title compound was prepared. 1H NMR (CD₃OD, 400 MHz) 8 ppm8.40 (d, J=2.78 Hz, 1H), 7.83 (d, J=3.79 Hz, 1H), 7.61 (d, J=7.83 Hz, 1H), 7.51-7.44 (m, 3H), 7.40 (d, J=8.08 Hz, 1H), 7.25 (s, 1H), 7.11-7.09 (m, 2H), 7.05 (t, J=7.58 Hz, 1H), 6.41 (d, J=1.01 Hz, 1H), 4.39 (dd, J=10.48, 3.16Hz, 1H), 4.24 (dd, J=10.48, 5.94 Hz, 1H), 4.02-3.95 (m, 1H), 3.32-3.30 (m, 2H), 2.59 (s, 3H); MS (M+H): 458.2

### Example 156

### Preparation of (2S)-1-{[6-(3-furanyl)-5-(3-methyl-1H-indazol-5-yl)-3-pyridinyl]oxy}-3-(1H-indol-3-yl)-N-methyl-2-propanamine

a) N-[(1S)-2-[(5-bromo-6-chloro-3-pyridinyl)oxy]-1-(1H-indol-3-ylmethyl)ethyl]-2-nitrobenzenesulfonamide

To a solution of the compound of Example 70(a) (1.28 g) in 3 ml CH₂Cl₂ was added 0.5 ml TFA and then 2 ml MeOH. The reaction mixture was stirred at room temperature for 1 hr. Solvent was removed under vacuum. The residue (0.9 g, 1.48 mmol) was dissolved in 50 ml CH₂Cl₂ and cooled to 0° C. To this solution was added 2-nitrobenzenesulfonyl chloride (0.36 g, 1.63 mmol) and Et₃N (0.78 ml, 5.9 mmol). The mixture was stirred at 0° C. for 1 hr. 100 ml water was added and organic layer was separated and concentrated. The crude compound was purified by flash chromatography to give 400 mg the title compound as white solid. (Yield 46%). MH+ 565.2/567.2

b) N-[(1S)-2-[(5-bromo-6-chloro-3-pyridinyl)oxy]-1-(1H-indol-3-ylmethyl)ethyl]-N -methyl-2-nitrobenzenesulfonamide

To a solution of the compound of Example 156(a) (200 mg, 0.35 mmol) and potassium carbonate (97 mg, 0.7 mmol) in 2 ml DMF and 1 ml CH₃CN was added Mel (50 mg, 0.35 mmol). The mixture was stirred at room temperature for 3 hrs. Removed the solvents under vacuum. The residue was dissolved in EtOAc, washed with water and brine. Organic layer was concentrated to give the title compound. MH+ 579.2/581.2.

c) N-[(1S)-2-{[6-(3-furanyl)-5-(3-methyl-1H-indazol-5-yl)-3-pyridinyl]oxy}-1-(1H-indol-3-ylmethyl)ethyl]-N-methyl-2-nitrobenzenesulfonamide

Following the procedure of Example 105(b) and 105(c), except substituting the compound of Example 156(a) for Example 105(a), the title compound was prepared. MH+ 663.4

d) (2S)-1-{[6-(3-furanyl)-5-(3-methyl-1H-indazol-5-yl)-3-pyridinyl]oxy}-3-(1H-indol-3-yl)-N-methyl-2-propanamine

To a solution of the compound of Example 156(c) (130 mg, 0.196 mmol) in 5 ml DMF was added benzenethio (43 mg, 0.39 mmol) and potassium carbonate (83 mg, 0.6 mmol). The mixture was stirred at room temperature for 2 hrs. After removal the solvent, the residue was purified by reverse phase HPLC to give the title compound 90 mg (yield 97%) 1H NMR (CD₃OD, 400 MHz) δ ppm 8.40 (dd, 1H), 7.68 (d, 1H), 7.60-7.58 (d, 2H), 7.51 (d, 1H), 7.43-7.38 (m, 2H), 7.30-7.20 (m, 3H), 7.11 (t, 1H), 7.02 (t, 1H), 6.41 (d, 1H), 4.50-4.40 (m, 1H), 4.38-4.30 (m, 1H), 4.02-3.90 (m, 1H), 3.40-3.30 (m, 2H), 2.91 (s, 3H), 2.57 (s, 3H); MS (M+H): 478.2

### Example 157

### Preparation of 5-[5-{[(2S)-2-amino-3-(1H-indol-3-yl)propyl]oxy}-2-(3-furanyl)-3-pyridinyl]-N, N-dimethyl-2-furancarboxamide

a) [(1S)-2-{[6-chloro-5-(5,5-dimethyl-1,3,2-dioxaborinan-2-yl)-3-pyridinyl]oxy}-1-(1H-indol-3-ylmethyl)ethyl]amine

A mixture of the compound of Example 70(a) (960 mg, 2.00 mmol), 5,5,5′,5′-tetramethyl-2,2′-bi-1,3,2-dioxaborinane (542 mg, 2.4 mmol.), PddppfCl2.9CH2Cl2 (100 mg, 5 mol %), KOAc (294 mg, 3.00 mmol.) and dioxane (20 mL) was heated at 80° C. under N₂ protection for 3 hrs. Reaction mixture was concentrated and purified on Biotage column (20% to 50% EtOAc/CH₂Cl₂ with 1% HOAc) to give the title compound as yellow solid 800 mg. (yield 78%).

b) 5-[5-{[(2S)-2-amino-3-(1H-indol-3-yl)propyl]oxy}-2-(3-furanyl)-3-pyridinyl]-N,N-dimethyl-2-furancarboxamide

A solution of the compound of Example 157(a) (78 mg, 0.15 mmol), 5-bromo -N,N-dimethyl-2-furancarboxamide (50 mg, 0.23 mmol), Pd(Ph₃P)₄ (17 mg, 10 mol %) and Na₂CO₃ (0.15 mL, 2N) in 2 ml dioxane was sealed and subjected to microwave irradiation at 150° C. for 10 min. To this reaction mixture was then added 3-furanboronic acid (30 mg, 0.27 mmol), Pd(Ph₃P)₄ (17 mg, 10 mol %) and Na₂CO₃ (0.15 mL, 2N).The reaction mixture was sealed and subjected to microwave irradiation at 160° C. for 10 min and then filtered on celite, which was rinsed with EtOAc. The combined filtrates were concentrated and the residue was dissolved in the mixture of 2 ml CH2Cl2 and 1 ml TFA. After stirring at room temperature for 30 mins, the mixture was concentrated and purified by reverse phase HPLC to give the title compound. (37 mg, 35%) 1H NMR (400 MHz, MeOD) δ ppm 8.38 (d, J=3.0 Hz, 1H), 7.80 (d, J=2.8 Hz, 1H), 7.59-7.67 (m, 3H), 7.40 (d, J=8.1 Hz, 1H), 7.25 (s, 1H), 7.11-7.17 (m, 2H), 7.03-7.08 (m, 1H), 6.53 (d, J=3.5 Hz, 1H), 6.47 (d, J=1.0 Hz, 1H), 4.40 (dd, J=10.5, 3.2 Hz, 1H), 4.27 (dd, J=10.5, 5.7 Hz, 1H), 3.96-4.04 (m, 1H), 3.27-3.38 (m, 2H), 3.20 (s, 3H), 3.11 (s, 3H); MS (M+H): 471.2

### Example 158

### Preparation of 5-[5-{[(2S)-2-amino-3-(1H-indol-3-yl)propyl]oxy}-2-(3-furanyl)-3-pyridinyl]-N-methyl-2-furancarboxamide

a) 5-[5-{[(2S)-2-amino-3-(1H-indol-3-yl)propyl]oxy}-2-(3-furanyl)-3-pyridinyl]-2-furancarboxylic acid

Following the procedure of Example 157, except substituting (2Z, 4E)-5-bromo-2-(methyloxy)-2,4-pentadienoic acid for 5-bromo-N,N-dimethyl-2-furancarboxamide, the title compound was prepared.

b) 5-[5-{[(2S)-2-amino-3-(1H-indol-3-yl)propyl]oxy}-2-(3-furanyl)-3-pyridinyl]-N-methyl-2-furancarboxamide

A solution of the compound of Example 158(a) (20 mg, 0.037 mmol), 2.0 M methylamine in THF (0.06 ml, 0.11 mmol), EDC (8.5 mg, 0.044 mmol) and HOBT (6.0 mg, 0.044 mmol) in CH2Cl2 was stirred at room temperature over night. The mixture was diluted in 20 ml CH2Cl2 and washed with water and brine. The organic layer was concentrated and the residue was dissolved in 2 ml CH2Cl2 and 1 ml TFA. The resulted mixture was stirred at room temperature for 30 min and then concentrated and purified by reverse phase HPLC to give the title compound as yellow solid. (12 mg, 48%) 1H NMR (400 MHz, MeOD) δ ppm 8.39 (d, 1H), 7.90 (d, 1H), 7.65 (s, 1H), 7.63-7.60 (m, 2H), 7.40 (d, 1H), 7.25 (s, 1H), 7.18-7.10 (m, 2H), 7.05 (t, 1H), 6.48 (s, 1H), 6.44 (d, 1H), 4.43 (dd, 1H), 4.28 (dd, 1H), 4.08-4.00 (m, 1H), 3.36-3.20 (m, 2H) 2.91 (s, 3H); MS (M+H): 457.2

### Example 159

### Preparation of 5-[5-{[(2S)-2-amino-3-(1H-indol-3-yl)propyl]oxy}-2-(3-furanyl)-3-pyridinyl]-2-furancarboxamide

Following the procedure of Example 158, except substituting amine for methylamine, the title compound was prepared. 1H NMR (400 MHz, MeOD) δ ppm 8.39 (d, 1H), 7.93 (d, 1H), 7.70 (s, 1H), 7.65-7.60 (m, 2H), 7.40 (d, 1H), 7.25 (s, 1H), 7.20-7.10 (m, 2H), 7.05 (t, 1H), 6.50 (s, 1H), 6.45 (d, 1H), 4.43 (dd, 1H), 4.30 (dd, 1H), 4.04-4.00 (m, 1H), 3.39-3.27 (m, 2H); MS (M+H): 443.2

### Example 160

### Preparation of [(2S)-2-amino-3-phenylpropyl][6-(3-furanyl)-5-(3-methyl-1H-indazol -5-yl)-3-pyridinyl]methylamine

a) [(2S)-2-amino-3-phenylpropyl](5-bromo-6-chloro-3-pyridinyl)methylamine

To a solution of the compound of Example 82(a) (460 mg, 1.05 mmol) and 11 ml Formaldehyde in 10 ml acetonitrile was added NaCNBH3. After 10 min, 0.1 ml HOAc was added and stirred for 2 hr. The reaction was monitored by TLC. Repeated adding Formaldehyde, NaCNBH3 and HOAc for 2 times. Removed the solvent and extracted the residue with EtOAc. The organic layer was washed with water and brine and concentrated and purified by Biotage column. (200 mg, 42%)

b) [(2S)-2-amino-3-phenylpropyl][6-(3-furanyl)-5-(3-methyl-1H-indazol-5-yl)-3-pyridinyl]methylamine

Following the procedure of Example 82, except substituting Example 160(a) for Example 82(a) and substituting 1,1-dimethylethyl 3-methyl-5-(4,4,5,5-tetramethyl-1,3,2-dioxaborolan-2-yl)-1H-indazole-1-carboxylate the for 1,1-dimethylethyl 5-(4,4,5,5-tetramethyl-1,3,2-dioxaborolan-2-yl)-1H-indazole-1-carboxylate, the title compound was prepared. 1H NMR (400 MHz, MeOD) δ ppm 8.02 (d, J=3.0 Hz, 1H), 7.73 (s, 1H), 7.47-7.52 (m, 4H), 7.29 (d, J=7.3 Hz, 2H), 7.18-7.24 (m, 3H), 7.10-7.15 (m, 1H), 6.27 (d, J=2.8 Hz, 1H), 3.92-4.00 (m, 1H), 3.69-3.79 (m, 2H), 3.18 (s, 3H), 3.10-3.17 (m, 1H), 3.08-3.19 (m, 1H), 2.60 (s, 3H); MS (M+H): 438.2

### Example 161

### Preparation of [(1S)-2-(3,4-dichlorophenyl)-1-({[5-(3-methyl-1H-indazol-5-yl)-6-phenyl-3-pyridinyl]oxy}methyl)ethyl]amine

Following the procedure of Example 109(a)-109(g), except substituting bromo(3,4-dichlorophenyl)magnesium for bromo(2-naphthalenyl)magnesium, the title compound was prepared. ¹H NMR (CD₃OD, 400 MHz) δ 8.51 (s, 1H), 8.10 (s, 1H), 7.70 (s 1H), 7.30-7.40 (m, 8H), 7.20 (m, 1 H ), 7.00 (m, 1H), 4.65 (dd, 1H), 4.55 (dd, 1H), 4.13 (dd, 1H), 3.71-3.94 (m, 2H), 2.50 (s, 3H). MS (M+H): 504.4.

### Example 162

### Preparation of N-[5-(3-methyl-1H-indazol-5-yl)-6-phenyl-3-pyridinyl]-L -phenylalaninamide

Following the procedure of Example 105(a)-105(d), except substituting phenylboronic acid for 3-furanylboronic acid, the title compound was prepared. ¹H NMR (CD₃OD, 400 MHz) δ 8.75 (s, 1H), 8.12 (s, 1H), 7.60-7.70 (m, 3H), 7.48-7.52 (m, 3H), 7.30 (s 1H), 7.10-7.28 (m, 5H), 6.95 (m, 1H), 4.60 (dd, 1H), 3.18 (dd, 1H), 3.00 (dd, 1H), 2.48 (s, 3H). MS (M+H): 448.4

### Example 163

### Preparation of N-[5-(3-methyl-1H-indazol-5-yl)-6-phenyl-3-pyridinyl]-L -phenylalaninamide

Following the procedure of Example 105(a)-105(d), except substituting 2-furanylboronic acid for 3-furanylboronic acid, the title compound was prepared. ¹H NMR (CD₃OD, 400 MHz) δ 8.70 (s, 1H), 8.10 (s, 1H), 7.60-7.70 (m, 2H), 7.55 (m, 1H), 7.40-7.50 (m, 2H), 7.10-7.28 (m, 4H), 6.30 (s, 1H), 5.90 (s, 1H), 4.50 (dd, 1H), 3.15 (dd, 1H), 2.95 (dd, 1H), 2.48 (s, 3H). MS (M+H): 438.6

### Example 164

### Preparation of 2-[5-{[(2S)-2-amino-3-(1H-indol-3-yl)propyl]amino}-3-(3-methyl-1H-indazol-5-yl)-2-pyridinyl]-4-fluorophenol

Following the procedure of Example 111(d), except substituting (5-fluoro-2-hydroxyphenyl)boronic acid for 3-furanylboronic acid, the title compound was prepared. ¹H NMR (CD₃OD, 400 MHz) δ 8.00 (s, 1H), 7.62 (m, 2H), 7.58 (s, 1H), 7.40 (d, 1H), 7.34 (d, 1H), 7.30 (s, 1H), 6.90-7.10 (m, 4H), 6.85 (dd, 1H), 6.72 (dd, 1H), 4.92 (m, 1H), 3.70 (dd, 1H), 3.60 (dd, 1H), 3.25 (m, 2H), 2.50 (s, 3H). MS (M+H): 507.6.

### Example 165

### Preparation of ((1S)-3-{[6-(3-furanyl)-5-(3-methyl-1H-indazol-5-yl)-3-pyridinyl]oxy}-1-{[4-(trifluoromethyl)phenyl]methyl}propyl)amine

a) 1,1-dimethylethyl ((1S)-3-hydroxy-1-{[4-(trifluoromethyl)phenyl]methyl}propyl)carbamate

To a solution of (3S)-3-({[(1,1-dimethylethyl)oxy]carbonyl}amino)-4-[4-(trifluoromethyl)phenyl]butanoic acid (1.0 g, 2.9 mmol) in THF at −10° C. was added BH₃.THF (17.3 mL, 17.3 mmol) dropwise. The reaction mixture was stirred at −10° C. for 3 hrs at which time, LCMS showed the complete consumption of the starting material. The mixture was then concentrated down to one-third of the original volume and quenched with 8 mL MeOH: acetic acid (9:1). The reaction mixture was then concentrated and the resultant was dissolved in EtOAc (600 mL), washed with 1N HCl, aqueous saturated NaHCO₃, brine, and dried over MgSO₄. The organic was concentrated to provide 0.74 g of the product as white solid (76

b) 1,1-dimethylethyl ((1S)-3-[(5-bromo-6-chloro-3-pyridinyl)oxy]-1-{[4-(trifluoromethyl)phenyl]methyl}propyl)carbamate

To a solution of Example 165(a) (0.74 g, 2.1 mmol), 5-bromo-6-chloro-3-pyridinol (0.44 g, 2.1 mmol), PPh₃ (0.85 g, 3.15 mmol) in THF was added DEAD (0.55 g, 3.15 mmol) at 0° C. The reaction mixture was warmed up to r.t. and stirred overnight. The residue was purified by Biotage chromatography (20%-50% EtOAc/Hexane) to provide 0.9 g of the product (82%).

c) 1,1-dimethylethyl ((1S)-3-{[6-chloro-5-(3-methyl-1H-indazol-5-yl)-3-pyridinyl]oxy}-1-{[4-(trifluoromethyl)phenyl]methyl}butyl)carbamate

A solution of the compound Example 165(b) (0.8 g, 1.50 mmol), 1,1-dimethylethyl 3-methyl-5-(4,4,5,5-tetramethyl-1,3,2-dioxaborolan-2-yl)-1H-indazole-1-carboxylatee (0.43 g, 1.68 mmol), Pd(PPh₃)₄ catalytic amount and 2M aqueous Na₂CO₃ (2.1 ml, 4.21 mmol) in 3 mL of dioxane was heated at 160° C. for 30 min in microwave. The reaction mixture was filtered by celite, concentrated and purified by Biotage flash chromatography (20%-60% EtOAc/Hexane) to give 0.6 g of the titled product (70%).

d) ((1S)-3-{[6-(3-furanyl)-5-(3-methyl-1H-indazol-5-yl)-3-pyridinyl]oxy}-1-{[4-(trifluoromethyl)phenyl]methyl}propyl)amine

A solution of the compound Example 165(c) (602 mg, 1.04 mmol), 3-furanylboronic acid (234 mg, 2.08 mmol), Pd(PPh₃)₄ catalytic amount and 2M aqueous Na₂CO₃ (1.3 mL, 2.6 mmol) in 3 mL of dioxane was heated at 170° C. for 30 min in microwave. The reaction mixture was concentrated and purified on Biotage flash chromatography (50% EtOAc/Hexane) to give a off-white solid. To the above product was added I ml TFA in CH₂Cl₂. The reaction mixture was stirred at room temperature for 1 h. The solution was concentrated and crude product was dissolved in 1 mL of MeOH and purified by reverse phase HPLC to provide 255 mg the titled compound (48%). ¹H NMR (CD₃OD, 400 MHz) δ 8.45 (s, 1H), 8.02 (s, 1H), 7.75-7.85 (m, 2H), 7.70 (dd, 2H), 7.50-7.58 (m, 4H), 7.30 (dd, 1H), 6.30 (s, 1H), 4.45 (m, 1H), 4.35 (m, 1H), 3.92 (dd, 1H), 3.20 (m, 2H), 2.51(s, 3H), 2.25 (m 2H). MS (M+H): 507.0.

### Example 166

### Preparation of [(1S)-3-{[6-(3-furanyl)-5-(3-methyl-1H-indazol-5-yl)-3-pyridinyl]oxy}-1-(1H-indol-3-ylmethyl)propyl]amine

Following the similar procedure of Example 165(a)-(d) substituting (3S)-3-({[(1,1-dimethylethyl)oxy]carbonyl}amino)-4-(1H-indol-3-yl)butanoic acid for (3S)-3-({[(1,1-dimethylethyl)oxy]carbonyl}amino)-4-[4-(trifluoromethyl)phenyl]butanoic acid, the title compound was prepared. ¹H NMR (CD₃OD, 400 MHz) δ 8.25 (s, 1H), 7.74 (m, 2H), 7.62 (dd, 1H), 7.48-7.54 (m, 3H), 7.32 (dd, 1H), 7.22 (m, 2H), 7.06 (m, 2H), 6.30 (s, 1H), 4.40 (m, 1H), 4.30 (m, 1H), 3.96 (dd, 1H), 3.20 (m, 2H), 2.51 (s, 3H), 2.20-2.28 (m 2H). MS (M+H): 478.0.

### Example 167

### Preparation of {(1S)-2-{[6-(3-furanyl)-5-(3-methyl-1H-indazol-5-yl)-3-pyridinyl]oxy}-1-[(5-methyl-1H-indol-3-yl)methyl]ethyl}amine

a) 1,1-dimethylethyl [(1S)-1-(hydroxymethyl)-3-butyn-1-yl]carbamate

To a solution of LiAlH₄ in THF (56.3 mL, 56.3 mmol) was added (2S)-2-({[(1,1-dimethylethyl)oxy]carbonyl}amino)-4-pentynoic acid (3.0 g, 14.1 mmol) in portions at r.t. The reaction mixture was stirred overnight. The mixture was then quenched carefully by dropwise addition of 1N HCl at 0° C. until the bubble eruption stopped. The resultant was extracted by ethyl ether 3 times and the organic layers were washed with sat. aqueous NaHCO₃, brine and dried over Na₂SO₄. The organic was concentrated and provided a light yellow oil and used for the next step without further purification.

b) 1,1-dimethylethyl ((1S)-1-{[(5-bromo-6-chloro-3-pyridinyl)oxy]methyl}-3-butyn-1-yl)carbamate

To a solution of Example 167(a) (2.8 g, 14.08 mmol), 5-bromo-6-chloro-3-pyridinol (3.48 g, 14.08 mmol), PPh₃ (5.70 g, 21.12 mmol) in THF was added DEAD (3.32 mL g, 21.12 mmol) at 0° C. The reaction mixture was warmed up to r.t. and stirred overnight. The residue was purified by Biotage chromatography (10%-15% EtOAc/Hexane) to provide 4.0 g of the product (82% for two steps).

c) 1,1-dimethylethyl [(1 S)-1-{[(5-bromo-6-chloro-3-pyridinyl)oxy]methyl}-4-(trimethylsilyl)-3-butyn-1-yl]carbamate

To a solution of compound Example 167(b) (1.06 g, 2.72 mmol) in THF was added EtMgBr (3.0 M in Et₂O, 2 mL, 6.0 mmol) at −36° C., followed by the addition of TMSCl (1.0 M in THF, 6 mL, 6.0 mmol). The reaction was warmed up to r.t. and stirred overnight. The reaction was then concentrated and diluted with CH₂Cl₂. The organic layer was washed with aqueous sat. NH₄Cl solution, brine and dried over Na₂SO₄. The organic was concentrated and purified by Biotage flash chromatography (10-20% EtOAc/Hexanes). 0.9 g (72%) titled product was obtained as a colorless oil.

d) 1,1-dimethylethyl [(1 S)-1-({[6-chloro-5-(3-methyl-1H-indazol-5-yl)-3-pyridinyl]oxy}methyl)-4-(trimethylsilyl)-3-butyn-1-yl]carbamate

A solution of the compound Example 167(c) (0.783 g, 1.90 mmol), 1,1-dimethylethyl 3-methyl-5-(4,4,5,5-tetramethyl-1,3,2-dioxaborolan-2-yl)-1H-indazole-1-carboxylate (0.75 g, 2.08 mmol), Pd(PPh₃)₄ catalytic amount and 2M aqueous Na₂CO₃ (2.4 ml, 4.8 mmol) in 3 mL of dioxane was heated at 160° C. for 30 min in microwave. The reaction mixture was filtered by celite, concentrated and purified by Biotage flash chromatography (20%-60% EtOAc/Hexane) to give 0.8 g of the titled product (83%) as white foam.

e) {(1S)-2-{[6-(3-furanyl)-5-(3-methyl-1H-indazol-5-yl)-3-pyridinyl]oxy}-1-[(5-methyl-1H-indol-3-yl)methyl]ethyl}amine

A solution of the compound Example 167(d) (100 mg, 0.2 mmol), 2-bromo-4-methylaniline (40 mg, 0.21 mmol), Pd(PPh₃)₄ catalytic amount and 2M aqueous Na₂CO₃ (0.25 mL, 0.5 mmol) in 3 mL of dioxane was heated at 170° C. for 30 min in microwave. The eaction was cooled down and 3-furanylboronic acid (71 mg, 0.59 mmol) was added to the reaction mixture, followed by the tion of catalytic Pd(PPh₃)₄ and 2M aqueous Na₂CO₃ (0.25 mL). The reaction mixture was then heated at 170° C. for 30 min in microwave. The reaction mixture was concentrated and purified by reverse phase HPLC to give a white solid as TFA salt 35 mg (38%). ¹H NMR (CD₃OD, 400 MHz) δ 8.42 (s, 1H), 7.70 (s, 1H), 7.52 (m, 2H), 7.48 (s, 1H), 7.34 (m, 2H), 7.18-7.28 (m, 3H), 6.90 (d, 1H), 6.30 (s, 1H), 4.42 (dd, 1H), 4.30 (dd, 1H), 3.96 (m, 1H), 3.28 (m, 2H), 2.51 (s, 3H), 2.30 (s, 3H). MS (M+H): 478.2.

### Example 168

### Preparation of [(1S)-2-(1H-indol-3-yl)-1-({[5-(3-methyl-1H-indazol-5-yl)-6]-(1H-pyrrol-3-yl)pyridin-3-yl]oxy}methyl)ethyl]amine

a) 1,1-dimethylethyl [(1S)-2-(1H-indol-3-yl)-1-({[5-(3-methyl-1H-indazol-5-yl)-6-(1H-pyrrol-3-yl)-3-pyridinyl]oxy}methyl)ethyl]carbamate

A mixture of Example 69(d)(100 mg, 0.2 mmol), 1-(triisopropylsilyl)pyrrole-3-boronic acid (80 mg, 1.5 eq.), Pd(Ph3P)4 (11.6 mg, 5 mol%), 2N Na2CO3 (0.3 mL) and dioxane (1 mL) was purged with N2, sealed and subjected to microwave irradiation at 160° C. for 10 min. TBAF (0.3 mL, 1N in THF) was added and the resulting mixture was stirred at room temperature for 1 h. The reaction mixture was filtered on celite, which was rinsed with EtOAc. The combined filtrates were concentrated and the residue was purified by flash column chromatography on silica gel (EtOAc) to afford light yellow solid (100 mg, 94%).

b) [(1S)-2-(1H-indol-3-yl)-1-({[5-(3-methyl-1H-indazol-5-yl)-6-(1H-pyrrol-3-yl)pyridin-3-yl]oxy}methyl)ethyl]amine

The title compound was prepared following Example 69(c), except substituting 168 (a) for example 69(b). H NMR 11.10 (br s, 1H), 8.27 (d, J=2.8 Hz, 1H), 8.07 (d, J=2.8 Hz, 1H), 7.79 (s, 1H), 7.61 (d, J=7.9 Hz, 1H), 7.53-7.55 (m, 1H), 7.39 (d, J=8.1 Hz, 1H), 7.29-7.26 (m, 2H), 7.16-7.12 (m, 2H), 7.06-7.02 (m, 2H), 6.80-6.78 (m, 1H), 6.64-6.63 (m, 1), 6.21-6.20 (m, 1H), 4.52-4.48 (m, 1H), 4.40-4.35 (m, 1H), 4.06-4.04 (m, 1H), 3.4-3.3 (m, 2H), 2.59 (s, 3H); MS: 463.2

### Example 169

### Preparation of [(1S)-2-{[6-(3-furanyl)-5-(3-methyl-1H-pyrazolor4,3-bipyridin-5-yl)-3-pyridinyl]oxy}-1-(1H-indol-3-ylmethyl)ethyl]amine

a) 1-(3-fluoro-2-pyridinyl)ethanone 169 (a) and 1-(3-fluoro-4-pyridinyl)ethanone 169(a)′

A solution of 3-fluoropyridine (0.86 mL, 10 mmol) in THF (5 mL) was added to a solution of BuLi (4.8 mL, 2.5 M in hexane, 1.2 eq.) in THF (25 mL) at −78° C. The resulting mixture was stirred at −78° C. for 30 min. N-Methyl-N-methoxyacetamide (1.5 g, 1.5 eq.) was added at −78° C. The resulting mixture was slowly warmed up to room temperature and stirred for 1 h. The reaction was quenched with ice-cold saturated aqueous ammonium chloride solution. The organic layer was separated and the aqueous layer was extracted with ether. The combined organic layers were washed with NaHCO3, brine, and dried (Na2CO3). Removal of the solvent followed by the purification of the residue by flash column chromatography on silica gel (hexane/EtOAc 3:1) afforded a 1:1 mixture of 169(a) and 169(a)′ as yellow liquid (1.18 g, 84%).

b) 3-methyl-1H-pyrazolo[4,3-b]pyridine

A mixture of 169(a) and (a)′ (1.18 g) and hydrazine (2 mL, anhydrous) was heated at 120° C. overnight, cooled down to room temperature and taken up into H2O, which was extracted with EtOAc. Removal of the solvent followed by the purification of the residue by flash column chromatography on silica gel (EtOAc) afforded 169(b) as light yellow solid (486 mg, 43%).

c) 3-methyl-1-(triphenylmethyl)-1H-pyrazolo[4,3-b]pyridine (169(c)) and 3-methyl-2-(triphenylmethyl)-2H-pyrazolo[4,3-b]pyridine 169(c)′

NaH (219 mg, 60%, 1.5 eq.) was added to a solution of 169(b) (486 mg, 3.65 mmol) in DMF (10 mL) at 0° C. The resulting reaction mixture was stirred at 0° C. for 30 min. Triphenylmethyl chloride (1.12 g, 1.1 eq) was added in one portion. The resulting reaction mixture was stirred at room temperature for 2 h and taken up into EtOAc, which was washed with H20 (3×), brine, and dried (Na2SO4). Removal of the solvent followed by the purification of the residue by flash column chromatography on silica gel (hexane/EtOAc 3:1) afforded light yellow solid 169(c) (611 mg, 44.6%) and light yellow oil 169(c)′ (205 mg, 15%).

d) 3-methyl-1-(triphenylmethyl)-1H-pyrazolo[4,3-b]pyridine N-oxide

mCPBA (308 mg, 77%, 1.1 eq.) was added to a solution of 169(c) (470 mg, 1.25 mmol) in CH₂Cl₂ (10 mL) at 0° C. The resulting reaction mixture was stirred at room temperature for 12 h, washed with NaHCO3, brine, and dried (Na₂SO₄). Removal of the solvent followed by the purification of the residue by flash column chromatography on silica gel (CH2Cl2/EtOAc 1:1) afforded white solid 169(d) (480 mg, 98%).

e) 5-chloro-3-methyl-1H-pyrazolo[4,3-b]pyridine

A mixture of 169(d) (277 mg, 0.70 mmol) and POCl3 (1 mL) was heated in a sealed tube at 120° C. for 1 h, cooled down, and poured onto a mixture of ice and CH₂Cl₂. The resulting mixture was neutralized with 6N NaOH aqueous solution. The organic layer was dried (Na2SO4) and concentrated. The residue was purified by flash column chromatography on silica gel (CH2Cl2/MeOH 95:5) to give light brown solid 169(e) (100.5 mg, 86%).

f) 1,1-dimethylethyl [(1S)-2-{[6-chloro-5-(3-methyl-1H-pyrazolo[4,3-b]pyridin-5-yl)-3-pyridinyl]oxy}-1-(1H-indol-3-ylmethyl)ethyl]carbamate (169(f)) and 1,1-dimethylethyl [(1S)-2-(1H-indol-3-yl)-1-({[5-(3-methyl-1H-pyrazolo[4,3-b]pyridin-5-yl)-3-pyridinyl]oxy}methyl)ethyl]carbamate (169(f)′)

A mixture of 69(a) (480 mg, 1.00 mmol), 5,5,5′,5′-tetramethyl-2,2′-bi-1,3,2-dioxaborinane (271 mg, 1.2 eq.), PddppfCl2.CH₂Cl₂ (82 mg, 10 mol%), KOAc (147 mg, 1.5 eq.) and dioxane (4 mL) was purged with N2, sealed and heated at 80° C. overnight. To this reaction mixture were added 169(e) (60.5 mg, 0.36 mmol), Pd(Ph3P)4 (21 mg, 5 mol%) and Na2CO3 (1 mL, 2N). The resulting mixture was purged with N2, sealed and subjected to microwave irradiation at 150° C. for 10 min. The reaction mixture was filtered on celite, which was rinsed with EtOAc. The combined filtrates were concentrated and the residue was purified by flash column chromatography on silica gel (CH2Cl2/MeOH 95:5) to afford brown foamy solid 169(f) (78 mg, 40%) and 169(f)′ (20 mg, 11%).

g) [(1S)-2-{[6-(3-furanyl)-5-(3-methyl-1H-pyrazolo[4,3-b]pyridin-5-yl)-3-pyridinyl]oxy}-1-(1H-indol-3-ylmethyl)ethyl]amine

The title compound was prepared as described in 38(e) except substituting 38(c) with 169(f) and converting the TFA salt to the HCl salt with 4N HCl in dioxane.

H NMR 8.65 (d, J=2.8 Hz, 1H), 8.34 (d, J=2.8 Hz, 1H), 8.12 (d, J=8.7 Hz, 1H), 7.73 (dd, J=1.4, 1.0 Hz, 1H), 7.64-7.59 (m, 2H), 7.49 (d, J=8.7 Hz, 1H), 7.38 (d, J=8.1 Hz, 1H), 7.30 (s, 1H), 7.13 (dd, J=8.1, 7.0 Hz, 1H), 7.04 (dd, J=8.0, 7.0 Hz, 1H), 6.32 (dd, J=1.9, 0.8 Hz, 1H), 4.61-4.57 (m, 1H), 4.47-4.43 (m, 1H), 4.13-4.08 (m, 1H), 3.4-3.3 (m, 2H), 2.67 (s, 3H); MS: 365.2

### Example 170

### Preparation of [(1S)-2-(1H-indol-3-yl)-1-({[5-(3-methyl-1H-pyrazolo[4,3-b]pyridin-5-yl)-3-pyridinyl]oxy}methyl)ethyl]amine

The title compound was prepared as described in 38(e) except substituting 38(c) with 169(f)′ and converting the TFA salt to the HCl salt with 4N HCl in dioxane. H NMR 9.30 (s, 1H), 8.92 (s, 1H), 8.68 (d, J=1.6 Hz), 8.16 (s, 2H), 7.66 (d, J=7.9 Hz, 1H), 7.39 (d, J=8.1, Hz, 1H), 7.33 (s, 1H), 7.13 (dd, J=7.4, 7.2 Hz, 1H), 7.05 (dd, J=7.7, 7.2 Hz, 1H), 4.65-4.61 (m, 1H), 4.54-4.49 (m, 1H), 4.15-4.10 (m, 1H), 3.4-3.3 (m, 2H), 2.72 (s, 3H); MS: 399.2

### Example 171 and 172

### Preparation of 5-[5-{[(2S)-2-amino-3-phenylpropyl]oxy}-2-(3-furanyl)-3-pyridinyl]-1H-indazole-3-carbonitrile and 5-[5-{[(2S)-2-amino-3-phenylpropyl]oxy}-2-(3-furanyl)-3-pyridinyl]-1H-indazole-3-carboxamide

a) 1,1-dimethylethyl [(1S)-2-{[5-(3-cyano-1-{[4-(methyloxy)phenyl]methyl}-1H-indazol-5-yl)-6-(3-furanyl)-3-pyridinyl]oxy}-1-(phenylmethyl)ethyl]carbamate

A mixture of Example 122(b) (151 mg, 0.2 mmol), Zn(CN)2 (26 mg, 1.1 eq.), Pd(Ph3P)4 (12 mg, 5 mmol%) and DMF was purged with N2, sealed and subjected to microwave irradiation at 120° C. for 20 min. The reaction mixture was taken up into EtOAc, which was washed with H2O (3×), brine, and dried (Na2SO4). Removal of the solvent followed by the purification of the residue by flash column chromatography on silica gel (hexane/EtOAc 3:1) afforded light yellow solid Example 171(a) (128 mg, 98%).

b) 5-[5-{[(2S)-2-amino-3-phenylpropyl]oxy}-2-(3-furanyl)-3-pyridinyl]-1H-indazole-3-carbonitrile (171(b)) and 5-[5-{[(2S)-2-amino-3-phenylpropyl]oxy}-2-(3-furanyl)-3-pyridinyl]-1H-indazole-3-carboxamide (172)

A solution of 171(a)(57 mg, 0.087 mmol) in TFA (1 mL) was subjected to microwave irradiation at 140° C. for 10 min. The reaction mixture was concentrated and the residue was purified by reversed phase HPLC (0.1% TFA in CH3CN and 0.1% TFA in H20) to give 171(b) as a off-white solid (5.8 mg, 12%) and 172 as a off-white solid (18.1 mg, 36%). H NMR (171(b)) 8.40 (d, J=2.9 Hz, 1H), 7.84 (dd, J=1.5, 0.8 Hz, 1H), 7.73 (dd, J=8.8, 0.8 Hz, 1H), 7.48 (d, J=2.9 Hz, 1H), 7.42-7.30 (m, 7), 7.18 (dd, J=1.5, 0.8 Hz, 1H), 6.28 (dd, J=1.8, 0.8 Hz, 1H), 4.34 (dd, J=10.6, 3.0 Hz, 1H), 4.19 (dd, J=10.6, 5.6 Hz, 1H), 3.95 (m, 1H), 3.14-3.16 (m, 2H); MS: 436.0; H NMR (172) 8.41 (d, J=2.8 Hz), 8.26 (dd, J=1.5, 0.8 Hz, 1H), 7.64-7.61 (m, 2H), 7.41-7.28 (m, 7H), 7.22 (d, J=1.2 Hz, 1H), 6.31 (dd, J=1.9, 0.8 Hz, 1H)), 4.37 (dd, J=10.6, 3.7 Hz, 1H), 4.21 (dd, 1, J=10.6, 5.6 Hz, 1H), 3.95 (m, 1H), 3.17-3.13 (m, 2H); MS: 454.2.

### Example 173

### Preparation of (2S)-1-{[6-(2-furanyl)-5-(3-methyl-1H-indazol-5-yl)-3-pyridinyl]oxy}-3-(1H-indol-3-yl)-2-propanamine

Following the example 69(d) by substituting 2-furanyl boronic acid for phenyl boronic acid, the titled compound was obtained. 1H-NMR (MeOD): δ 8.30 (1s, 1H), 7.67-7.53 (m, 5H), 7.37 (d, 1H), 7.25-7.23 (m, 2H), 7.14-7.10 (t, 1H), 7.05-7.01 (t, 1H), 6.36-6.35 (d, 1H), 5.90-5.89 (d, 1H), 4.43-4.26 (dt, 2H), 4.007-4.000(m, 1H), 3.31-3.30 (m, 2H), 2.57 (s, 3H). MS (M+H): 464.2

### Example 174

### Preparation of 2-[5-{[(2S)-2-amino-3-(1-benzothien-3-yl)-3-propyl]oxy}-3-(1H-indazol-5-yl)-2-pyridinyl]-4-flurophenol

Following example 107(e) by substituting 2-fluro-2-methoxyphenylboronic acid for (2-hydroxyphenyl)boronic acid, the titled compound was prepared as a yellow solid (60%).

1H-NMR (MeOD): δ 7.90 (1s, 1H), 7.924 (d, 2H), 7.923-7.915 (m, 2H), 7.74 (s, 1H), 7.73 (s, 1H), 7.49-7.37 (m, 3H), 7.23-7.20 (d, 1H), 7.05-6.86 (m, 1H), 6.85-6.80 (m, 2H), 4.56-4.40(m, 2H), 4.17-4.16 (m, 1H), 3.35-3.49 (m, 2H). MS (M+H): 511.5

### Example 175

### Preparation of 2-[5-{[(2S)-2-amino-3-(1-benzothien-3-yl)-3-propyl]oxy}-3-(1H-indazol-5-yl)-2-pyridinvyl-4,6-diflurophenol

Following example 107(e) by substituting 3,5-difluro-2-methoxyphenylboronic acid for (2-hydroxyphenyl)boronic acid, the titled compound was prepared as a white solid (37%)

1H-NMR (MeOD): δ 8.60 (1s, 1H), 8.09-8.08 (d, 2H), 7.94-7.90 (m, 2H), 7.59 (s, 1H), 7.59 (s, 1H), 7.51-7.49 (d, 1H), 7.43-7.38 (m, 2H), 7.234-7.230 (d, 1H), 7.21 (m, 1H), 7.20 (m, 1H), 4.6-4.7 (m, 2H), 4.2 (m, 1H), 3.5 (m, 2H). MS (M+H): 529.4

### Example 176

### Preparation of [(1S)-2-(1-benzothien-3-yl)-1-({[5,6-bis(3-methyl-1H-indazol-5-yl)-3-pyridinyl]oxy}methyl)ethyl]amine

To example 107(c) (100 mg, 0.201 mmol) dissolved in dioxane was added methylindazole boronic ester (86 mg, 0.241 mmol), followed by the catalyst, aq Na₂CO₃ (250 μL). The reaction mixture was then heated for 20 min at 160° C. in a microwave reactor. The crude mixture was purified on a silica gel coloumn (50% EtOAc/Hex) to obtain the product which was then treated with TFA and further purified on a reverse phase HPLC (MeCN, H₂O, 0.1% TFA) to give the title compound as a yellow solid (65. Omg, 60%)

1H-NMR (MeOD): δ 8.57 (1s, 1H), 8.18 (S, 1H), 7.96-7.89 (m, 3H), 7.76 (s, 1H), 7.60 (s, 1H), 7.43-7.31 (M, 4H), 7.14-7.12 (d, 1H), 7.07-7.05 (d, 1H), 4.61-4.59 (m, 1H), 4.50-4.49 (m, 1H), 4.205-4.200 (m, 1H), 3.5 (m, 2H), 2.5 (s, 6H). MS (M+H): 545.0

### Example 177

### Preparation of [(1S)-2-(1-benzothien-3-yl)-1-({[4-(3-furanyl)-3-(3-methyl-1H-indazol-5-yl)phenyl]oxy}methyl)ethyl]amine

Following example 107 (e) by substituting 3-methyl-5-(4,4,5,5-tetramethyl-1,3,2-dioxaborolan-2-yl)-1H-indazole for 5-(4,4,5,5-tetramethyl-1,3,2-dioxaborolan-2-yl)-1H-indazole and 3-furanyl boronic acid for (2-hydroxyphenyl)boronic acid, the title compound was prepared as a white solid. 1H-NMR (MeOD): δ 8.44 (s, 1H), 8.40-7.9 (t, 2H), 7.90 (s, 1H), 7.70 (s, 1H), 7.69-7.62 (m, 2H), 7.43-7.39 (m, 3H), 7.25-7.23 (t, 2H), 6.30-6.29 (d, 1H), 4.44-4.42 (m, 1H), 4.31-4.27 (m, 1H), 3.49 (m, 2H), 2.5 (s, 1H). MS (M+H): 481.2

### Example 178

### Preparation of 4′-{[(2S)-2-amino-3-(1-benzothien-3-yl)propyl]oxy}-3,5-difluoro-2′-(3-methyl-1H-indazol-5-yl)-2-biphenylol

Following Example 177 by substituting [3,5-difluoro-2-(methyloxy)phenyl]boronic acid for 3-furanylboronic acid, followed by BBr₃ demethylation, TFA de-Boc and further purification on a reverse phase HPLC (MeCN, H₂O, 0.1% TFA), the title compound was prepared as a white solid (14%).

1H-NMR (MeOD): δ 8.44 (d, 1H), 7.93-7.90 (d, 2H), 7.72 (s, 1H), 7.91 (s, 1H), 7.57 (s, 1H), 7.45-7.36 (m, 3H), 7.19-7.16 (d, 1H), 6.95-6.94 (m, 1H), 6.69-6.66 (m, 1H), 4.49-4.45 (m, 1H), 4.35-4.32 (m, 1H), 4.13 (m, 1H), 3.5 (m, 2H), 2.5 (s, 3H).). MS (M+H): 543.4

### Example 179

### Preparation of 4′-{[(2S)-2-amino-3-(1-benzothien-3-yl)propyl]oxy}-5-fluoro-2′-(3-methyl-1H-indazol-5-yl)-2-biphenylol

Following Example 178 by substituting [5-fluoro-2-(methyloxy)phenyl]boronic acid for [3,5-difluoro-2-(methyloxy)phenyl]boronic acid, the title compound was prepared as a white solid (32%). 1H-NMR (MeOD): δ 8.10 (s, 1H), 7.94 (s, 1H), 7.91 (m, 2H), 7.67 (s, 1H), 7.44 (s, 1H), 7.39 (m, 3H), 7.21-7.18 (d, 1H), 7.06-7.01 (m, 1H), 6.86-6.82 (m, 2H), 4.57 (m, 1H), 4.42(m, 1H), 4.18 (m, 1H), 3.34 (m, 2H), 2.51 (s, 3H). MS (M+H): 524.6

### Example 180

### Preparation of 2-[5-{[(2S)-2-amino-3-(1H-indol-3-yl)propyl]oxy}-3-(3-methyl-1H-indazol-5-yl)-2-pyridinyl]-4,6-difluorophenol

Following the Example 177 by substituting [3,5-difluoro-2-(methyloxy)phenyl]boronic acid for 3-furanyl boronic acid, followed by BBr3 demethylation and purification, the titled compound was obtained. 1H-NMR (MeOD): δ 8.45 (s, 1H), 7.76 (m, 1H), 7.62 (m, 2H), 7.39 (m, 2H), 7.37 (s, 1H), 7.01-6.68 (m, 5H), 4.50-4.46 (m, 1H), 4.36-4.33 (1H), 4.03-4.02 (m, 1H), 3.36 (m, 2H), 2.50 (s, 3H). MS (M+H): 526.4

### Example 181

### Preparation of [(2S)-2-amino-3-(1H-indol-3-yl)propyl][5-(3-methyl-1H-indazol-5-yl)-6-(1H-pyrrol-2-yl)-3-pyridinyl]amine

Following Example 111(d) by substituting 1H-pyrrol-2-ylboronic acid for 3-furanylboronic acid, the above titled compound was obtained (32%).

1H-NMR (MeOD): δ 7.82 (s, 1H), 7.821 (s, 1H), 7.58 (m, 1H), 7.520 (m, 1H), 7.33 (d, 1H), 7.11 (d, 1H), 7.03 (s, 1H), 6.98-6.83 (m, 4H), 6.27 (d, 1H), 6.18 (d, 1H), 3.9 (m, 1H), 3.68 (m, 1H), 3.59 (m 1H), 3.32 (m, 2H), 2.54 (s, 3H), MS (M+H): 462.4

### Example 182

### Preparation of [(2S)-2-amino-3-(1H-indol-3-yl)propyl][6-[5-fluoro-2-(methyloxy)phenyl]-5-(3-methyl-1H-indazol-5-yl)-3-pyridinyl]amine

Following Example 111(d) by substituting [5-fluoro-2-(methyloxy)phenyl]boronic acid for 3-furanylboronic acid, the above titled compound was obtained (10%)

1H-NMR (MeOD): δ 8.00 (s, 1H), 7.99 (m, 2H), 7.48 (s, 1H), 7.33-7.17 (m, 4H), 7.06-6.95 (m, 5H), 3.99 (m, 1H), 3.73-3.69 (m, 1H), 3.63-3.54 (m, 1H), 3.58 (s, 3H), 3.25(m, 2H), 2.48 (s, 3H). MS (M+H): 507.6

### Example 183

### Preparation of 2-[5-{[(2S)-2-amino-3-(1H-indol-3-yl)propyl]amino}-3-(3-methyl-1H-indazol-5-yl)-2-pyridinyl]phenol

Following example 111(d) by substituting (2-hydroxyphenyl)boronic acid for 3-furanylboronic acid, the above titled compound was obtained (32%)

1H-NMR (MeOD): δ 7.95 (s, 1H), 7.62 (m, 2H), 7.52)s, 1H), 7.36 (m, 2H), 7.28 (s, 1H), 7.00-7.01 (m, 2H), 6.94 (m, 2H), 6.86 (d, 1H), 6.76 (m, 1H), 3.90 (m, 1H), 3.68 (dd, 1H), 3.60 (dd, 1H), 3.26 (m, 2H), 2.47 (s, 3H). MS (M+H): 489.4

### Example 184

### Preparation of 2-[5-{[(2S)-2-amino-3-(1H-indol-3-yl)propyl]amino}-3-(3-methyl-1H-indazol-5-yl)-2-pyridinyl]phenol

To 111 (c) (81 mg, 0.152 mmol) dissolved in dioxane was added 2-methyolxyphenol boronic acid (31 mg, 0.227 mmol), followed by the catalyst, aq Na₂CO₃ (250 pL). The reaction mixture was then heated for 20 min at 160° C. in a microwave reactor. The crude mixture was purified on a silica gel coloumn (50% EtOAc/Hex) to obtain the product, which was then treated with TFA and further purified on a reverse phase HPLC (MeCN, H₂O, 0.1% TFA) to give the title compound as a yellow solid (16.0 mg, 32%)

1H-NMR (MeOD): δ 7.98 (s, 1H), 7.60-7.62 (m, 2H), 7.40 (s, 1H), 7.28-7.44 (m, 6H), 7.20 (m, 1H), 6.90-7.01 (m, 3H), 3.99-3.95 (m, 1H), 3.75-3.59 (m, 2H), 3.27-3.26(m, 2H), 2.43 (s, 3H), 1.92-1.89 (s, 3H). MS (M+H): 487.4

### Example 185

### Preparation of [(2S)-2-amino-3-(5-fluoro-1H-indol-3-yl)propyl][6-(3-furanyl)-5-(3-methyl-1H-indazol-5-yl)-3-pyridinyl]amine

Following the procedure as that of Example 167 by substituting (2-bromo-4-fluorophenyl)amine for (2-bromo-4-methylphenyl)amine, the titled compound was made.

1H-NMR (MeOD): δ 8.39 (s, 1H), 7.69 (s, 1H), 7.49-7.38 (m, 2H), 7.26 (s, 1H), 7.23-712 (m, 4H), 6.87-6.81 (m, 1H), 6.39 (s, 1H), 6.29 (d, 1H), 4.45-4.42 (m, 1H), 4.32-4.27 (m, 1H), 4.1 (m, 1H), 3.32 (m, 2H), 2.58 (s, 3H). MS (M+H): 482.2

### Example 186

### Preparation of [(2S)-2-amino-4-pentyn-1-yl][6-(3-furanyl)-5-(3-methyl-1H-indazol-5-yl)-3-pyridinyl]amine

Following the procedure in Example 167, with two Suzuki couplings, followed by TFA treatment and reverse phase HPLC, the above title compound was prepared. 1H-NMR (MeOD): δ 8.47 (s, 1H), 7.79-7.76 (d, 2H), 7.54-7.52 (d, 1H), 7.42 (s, 1H), 7.30-7.28 (m, 2H), 6.31 (s, 1H), 4.56-4.52 (m, 1H), 4.48-4.44 (m, 1H), 3.90-3.88 (m, 1H), 2.86-2.82 (m, 2H), 2.70 (m, 1H), 2.57 (s, 3H). MS (M+H): 373.2

### Example 187

### Preparation of [(2S)-2-amino-3-(5,6,7-trifluoro-1H-indol-3-yl)propyl][6-(3-furanyl)-5-(3-methyl-1H-indazol-5-yl)-3-pyridinyl]amine

To compound Example 167(d) (a) (100 mg, 0.195 mmol) in dioxane was added 4,5,6-triflurobromoaniline (50 mg, 0.22 mmol), followed by the catalyst and aq Na₂CO₃ (250 μL). The reaction mixture was then heated for 30 min at 170° C. in a microwave reactor. To this reaction mixture was then added 3-furaneboronic acid (50 mg, 0.446 mmol) and subjected to the above mentioned microwave conditions. The product was then purified on a silica gel coloumn and treated with TFA which was further purified on a reverse phase HPLC (MeCN, H₂O, 0.1% TFA) to the title compound as a yellow solid (15.0 mg, 15.0%)

1H-NMR (MeOD): δ 8.44 (s, 1H), 7.77-7.72 (m, 2H), 7.53-7.71 (m, 1H), 7.34-7.24 (m, 5H), 6.30 (s, 1H), 4.47-4.45 (m, 1H), 4.31 (m, 1H), 3.99 (s, 1H), 3.26 (m, 2H), 2.57 (s, 3H). MS (M+H): 518.4

### Example 188

### Preparation of [(2S)-2-amino-3-(5,7-difluoro-1H-indol-3-yl)propyl][6-(3-furanyl)-5-(3-methyl-1H-indazol-5-yl)-3-pyridinyl]amine

To compound 167(d) (200 mg, 0.390 mmol) in dioxane was added 4,6-diflurobromoaniline (90 mg, 0.434 mmol), followed by the catalyst and aq Na₂CO₃ (250 μL). The reaction mixture was then heated for 30 min at 170° C. in a microwave reactor. To this reaction mixture was then added 3-furaneboronic acid (17 mg, 0.151 mmol) and subjected to the above mentioned microwave conditions. The product was then purified on a silica gel coloumn and treated with TFA which was further purified on a reverse phase HPLC (MeCN, H₂O, 0.1% TFA) to give the title compound as a yellow solid (10.0 mg, 6.0%)

1H-NMR (MeOD): δ 7.70 (s, 1H), 7.63-7.16 (m, 9H), 6.30 (s, 1H), 4.58-4.48 (m, 2H), 3.99-3.98 (m, 1H), 3.32 (m, 2H), 2.55 (s, 3H). MS (M+H): 500.2

### Example 189

### Preparation of [(1S)-2-{[6-(3-furanyl)-5-(3-methyl-1H-indazol-5-yl)-3-pyridinyl]oxy}-1-(1H-pyrrolo[2,3-b]pyridin-2-ylmethyl)ethyl]amine

To compound 186(b) (100 mg, 0.211 mmol) in dioxane was added 3-iodo-2-pyridinamine (116 mg, 0.527 mmol), followed by the catalyst and aq Na₂CO₃ (250 μL). The reaction mixture was then heated for 30 min at 170° C. in a microwave reactor. The product was then purified on a silica gel coloumn and treated with TFA which was further purified on a reverse phase HPLC (MeCN, H₂O, 0.1% TFA) to give the title compound as a yellow solid (10.0 mg, 10.2%)

1H-NMR (MeOD): δ 8.42-8.36 (m 3H), 7.71 (s, 1H), 7.62 (s, 1H), 7.52-7.50 (d, 1H), 7.41-7.38 (m, 2H), 7.27-7.20 (m, 2H), 7.72 (s, 1H), 6.30 (s, 1H), 4.51-4.71 (m, 1H), 4.37-4.34 (m, 1H), 4.20-4.18 (m, 1H), 3.49 (m, 2H), 2.56 (s, 3H). MS (M+H): 465.4

### Example 190

### Preparation of [(2R)-2-amino-3-phenylpropyl][3-fluoro-4-(3-furanyl)-5-(3-methyl-1H-indazol-5-yl)phenyl]amine

a) 2-bromo-6-fluoro-4-nitrophenol

To a solution of Floronitrophenol (1.6 g, 10 mmol) in 5 ml AcOH, was added Br2 (1.8 g, 11 mmol). The reaction mixture was stirred at room temperature for 1 hr and then diluted in 30 ml water and extracted with CH2Cl2. Organic layer concentrated and the solid was washed with hexane to give off-white solid (1.87 g, 79%).

b) 2-bromo-6-fluoro-4-nitrophenyl phenylmethyl ether

A mixture of 190(a) (236 mg, 1.0 mmol), BnBr (0.13 mL, 1.1 eq.), Cs2CO3 (489 mg, 1.5 eq.) and DMF (10 mL) was stirred at room temperature overnight, concentrated under vacuum and taken up into CH2Cl2, which was washed with 1N NaOH, brine, and dried (Na2SO4). The solvent was removed to afford product 190(b) as a yellow solid (260 mg, 80%).

c) 2-fluoro-6-(3-methyl-1H-indazol-5-yl)-4-nitrophenol

A mixture of 190 (b) (65 mg, 0.2 mmol), 3-methyl-5-(4,4,5,5-tetramethyl-1,3,2-dioxaborolan-2-yl)-1H-indazole (57 mg, 0.22 mmol), Pd(Ph3P)4 (23 mg, 10 mol%), 2N Na2CO3 (0.2 mL) and dioxane (1 mL) was purged with N2, sealed and subjected to microwave irradiation at 150° C. for 20 min. The reaction mixture was filtered on celite and the filtrate was concentrated. The residue was dissolved in a mixed solvent (10 mL of CH2Cl2/1 mL of MeOH). A yellow precipitate was formed upon standing and was collected by filtration to give product 190(c)(30 mg, 52%).

d) 2-fluoro-6-(3-methyl-1H-indazol-5-yl)-4-nitrophenyl trifluoromethanesulfonate (A

A suspension of 190(c) (100 mg, 0.35 mmol), Et3N (0.14 mL, 3.0 eq.) and PhTf2 (186 mg, 1.5 eq.) in CH2Cl2 (3.5 mL) was stirred at room temperature for 48 h. Another 1.5 eq of PhNTf2 was added and the resulting mixture was stirred at room temperature overnight. The solvent was removed and the residue was taken up into EtOAc, which was washed with water, brine, and dried (Na2SO4). Removal of the solvent followed by the purification of the residue by flash column chromatography on silica gel (hexane/EtOAc 1:1) afforded product 190(d) (70 mg, 48%).

e) [3-fluoro-4-(3-furanyl)-5-(3-methyl-1H-indazol-5-yl)phenyl]amine

A mixture of 190(d) (70 mg, 0.17 mmol), 3-furanylboronic acid (22.5 mg, 0.2 mmol), Pd(Ph3P)4 (20 mg, 10 mol%), Et3N (0.047 mL, 0.34 mmol) and DMF (1.7 mL) was purged with N2, sealed and subjected to microwave irradiation at 150° C. for 20 min. The reaction mixture was concentrated under vacuum and the residue was taken up into EtOAc, which was washed with water, brine, and dried (Na2SO4). Removal of the solvent followed by the purification of the residue by flash column chromatography on silica gel gave product 190(e) (40 mg, 78%).

f) 1,1-dimethylethyl [(1 R)-2-{[3-fluoro-4-(3-furanyl)-5-(3-methyl-1H-indazol-5-yl)phenyl]amino}-1-(phenylmethyl)ethyl]carbamate

A mixture of 190(e) (40 mg, 0.12 mmol), 1,1-dimethylethyl [(1R)-1-formyl-2-phenylethyl]carbamate (48 mg, 0.16 mmol), 4A MS and CH2Cl2 (1.2 mL) was stirred at room temperature overnight. NaCNBH3 (24 mg, 0.4 mmol) and HOAc (0,1 mL) were added and the resulting mixture was stirred at room temperature overnight, washed with water, and dried (Na2SO4). Removal of the solvent afforded the crude product 190(f).

g) [(2R)-2-amino-3-phenylpropyl][3-fluoro-4-(3-furanyl)-5-(3-methyl-1H-indazol-5-yl)phenyl]amine

The title compound was prepared following Example 1 (f), except substituting 190(f) for 1(e). 1H NMR (400 MHz, MeOD) δ ppm 7.54 (m, 1H), 7.20-7.37 (m, 7H), 7.13-7.17 (m, 2H), 6.38-6.44 (m, 2H), 5.90 (s, 1H), 3.68 (m, 1H), 3.37-3.45(m, 1H), 3.29-3.37, 2.98-3.09 (m, 2H), 2.55 (s, 3H); MS: 441.2.

### Example 191

### Preparation of [(2R)-2-amino-3-(1H-indol-3-yl)propyl][3-fluoro-4-(3-furanyl)-5-(3-methyl-1H-indazol-5-yl)phenyl]amine

Following procedure in Example 190(a)-(g), except substituting Example 111(a) for 1,1-dimethylethyl [(1R)-1-formyl-2-phenylethyl]carbamate in Example 190(f), the title compound was prepared. 1H NMR (400 MHz, MeOD) δ ppm 7.53 (d, J=7.8 Hz, 1H), 7.49 (s, 1H), 7.32-7.34 (m, 2H), 7.21-7.26 (m, 2H), 7.04-7.14 (m, 3H), 6.96 (t, J=7.5 Hz, 1H), 6.39-6.45 (m, 2H), 5.88 (s, 1H), 3.77 (m, 1H), 3.46-3.55 (m, 1H), 3.35-3.40 (m, 1H), 3.12-3.24 (m, 2H), 2.51 (s, 3H); MS: 480.2.

### Example 192

### Preparation of [(1S)-2-{[6-(3-furanyl)-5-(3-methyl-1H-pyrazolo[3,4-b]pyridin-5-yl)-3-pyridinyl]oxy}-1-(1H-indol-3-ylmethyl)ethyl]amine

a) 3-bromo-2-chloro-5-[(phenylmethyl)oxy]pyridine

A mixture 4-bromo-5-chloro-3-hydroxypyridine (Koch, V. Schnatterer, S. Synthesis, 1990, 499-501) (2.08 g, 10 mmol), BnBr (1.31 mL, 11 mmol), K2CO3 (1.66 g, 12 mmol) and 30 mL of acetone was stirred at reflux for 2 h, cooled down and filtered on celite, which was rinsed with EtOAc. The combined filtrates were concentrated and the residue was purified by flash column chromatography on silica gel (95:5 hexane/EtOAc) to give 3.0 g of light grey solid (100%).

b) 3-methyl-5-[(phenylmethyl)oxy]-1H-pyrazolo[3,4-b]pyridine

A mixture of 1 (3.0 g, 10 mmol), Pd₂dba₃(190 mg, 2%), Ph₃P (210 mg, 8%) and 50 mL of toluene was stirred under N₂ for 20 min. Vinyltributyltin (3.4 mL, 10 mmol) was added and the resulting mixture was heated at 110° C. for 2 h, cooled down, and 50 mL of 3N HCl was added. The resulting mixture was stirred at room temperature overnight and neutralized with ice-cold 6N NaOH (25 mL). The aqueous layer was extracted with EtOAc and the combined filtrates were dried (Na₂SO₄), concentrated and the residue was treated with 10 mL of anhydrous hydrazine at 120° C. overnight. The reaction mixture was cooled down, taken up into EtOAc and water. The organic layer was dried (Na₂SO₄), concentrated and the residue was purified by flash column chromatography on silica gel (3:1 hexane/EtOAc) to give 1.59 g of white solid (66.5%).

c) 1,1-dimethylethyl 3-methyl-5-[(phenylmethyl)oxy]-1H-pyrazolo[3,4-b]pyridine-1-carboxylate and regioisomers

A mixture of 192(b) (1.59 g, 6.65 mmol), Et₃N (1.39 mL, 1.5 eq.), DMAP (70 mg, 0.625 mmol), Boc₂O (1.74 g, 1.2 eq.) and 50 mL of CH₂Cl₂ was stirred at room temperature 60 h, concentrated and the residue was purified by flash column chromatography on silica gel (3:1 hexane/EtOAc) to give 1.81 g of white solid (80%) as a mixture of isomers. H NMR (CDCl3, 400 MHz, one regioisomer) δ 8.57 (d, J=2.7 Hz, 1H), 7.47-7.43 (m, 6H), 5.18 (s, 2H), 2.58 (s, 3H), 1.74 (s, 9H).

d) 1,1-dimethylethyl 5-hydroxy-3-methyl-1H-pyrazolo[3,4-b]pyridine-1-carboxylate and regioisomers

A mixture of 192 (c) (407.5 mg, 1.20 mmol), Pd/C (10%, 40 mg) and 10 mL of EtOH was stirred under a balloon pressure of H2 for 2 hr and filtered through celite, which was rinsed with EtOAc. The combined filtrates were concentrated and the residue was purified by flash column chromatography on silica gel (EtOAc) to give 298 mg of white solid (99%) as a mixture of isomers. H NMR (CDCl3, 400 MHz, one regioisomer) δ 8.44 (d, J=2.7 Hz, 1H), 7.48 (d, J=2.7 Hz), 2.47 (s, 3H), 1.65 (s, 9H).

e) 1,1-dimethylethyl 3-methyl-5-{[(trifluoromethyl)sulfonyl]oxy}-1H-pyrazolo[3,4-b]pyridine-1-carboxylate and regioisomers

A mixture of 192 (d) (250 mg, 1.0 mrnol), Tf₂NPh (540 mg, 1.5 mmol), TEA (0.42 ml, 3.0 mmol) and 5 ml dry CH₂Cl₂ was stirred at room temperature for 2 hrs. The reaction mixture was washed with water and brine. The organic layer was dried over MgSO₄, concentrated and the residue was purified by flash column chromatography on silica gel (1:1 EtOAc/Hexane) to give 260 mg of white solid (260 mg, 68%).

f) 1,1-dimethylethyl [(1S)-2-{[4-chloro-3-(3-methyl-1H-pyrazolo[3,4-b]pyridin-5-yl)phenyl]oxy}-1-(1H-indol-3-ylmethyl)ethyl]carbamate

A mixture of solid 192(e) (1.5 g, 3.94 mmol),

[1,1′bis(diphenylphosphino)ferrocene]

Dichlorophalladium (II) (173 mg, 0.236 mmol), 5,5,5′,5′-tetramethyl-2,2′-bi-1,2,3-triborinane (1.06 g, 4.72 mmol), potassium acetate (580 mg, 5.91 mmol) and 20 ml dry dioxane was heated up to 80 C under nitrogen for overnight. To this reaction mixture was added compound 69(a) (1.90 g, 3.97 mmol), Pd(PPh₃)₄ (220 mg, 0.19 mmol) and Na2CO3 (2M, 4.4 ml). The reaction was heated at 150 C for 15 min in microwave. The reaction mixture was washed with EtOAc and was concentrated. The residue was purified by flash column chromatography to give 1.5 g (75%) compound 192(f)

g) [(1S)-2-{[6-(3-furanyl)-5-(3-methyl-1H-pyrazolo[3,4-b]pyridin-5-yl)-3-pyridinyl]oxy}-1-(1H-indol-3-ylmethyl)ethyl]amine

Following the procedure in Example 38 (c) to (e), except substituting 192(f) for 38 (c), the title compound was prepared. 1H NMR (400 MHz, MeOD) δ ppm 8.41 (d, J=2.8 Hz, 1H), 8.31 (d, J=2.0 Hz, 1H), 8.16 (d, J=2.0 Hz, 1H), 7.57 (d, J=7.8 Hz, 1H), 7.53 (d, J=2.8 Hz, 1H), 7.41 (t, J=1.6 Hz, 1H), 7.35 (d, J=8.1 Hz, 1H), 7.28 (s, 1H), 7.23 (s, 1H), 7.09 (t, J=7.6 Hz, 1H), 6.98-7.03 (m, 1H), 6.25 (d, J=1.8 Hz, 1H), 4.40 (dd, J=10.6, 3.3 Hz, 1H), 4.26 (dd, J=10.7, 5.7 Hz, 1H), 3.96-4.02 (m, 1H), 3.32-3.34 (m, 2H), 2.57 (s, 3H); MS: 465.2.

### Example 193

### Preparation of [(1S)-2-(1H-indol-3-yl)-1-({[6-(2-methyl-3-furanyl)-5-(3-methyl-1H-pyrazolo[3,4-b]pyridin-5-yl)-3-pyridinyl]oxy}methyl)ethyl]amine

Following the procedure in Example 192, except substituting 4,4,5,5-tetramethyl-2-(2-methyl-3-furanyl)-1,3,2-dioxaborolane for 3-furanylboronic acid, the title compound was prepared. 1H NMR (400 MHz, MeOD) 6 ppm 8.43 (d, J=2.8 Hz, 1H), 8.26 (d, J=2.0 Hz, 1H), 8.11 (d, J=2.0 Hz, 1H), 7.63 (d, J=3.0 Hz, 1H), 7.59 (d, J=8.1 Hz, 1H), 7.36 (d, J=8.1 Hz, 1H), 7.31 (d, J=1.8 Hz, 1H), 7.24 (s, 1H), 7.08-7.12 (m, 1H), 7.01 (t, J=7.5 Hz, 1H), 6.09 (d, J=1.8 Hz, 1H), 4.43 (dd, J=10.6, 3.0 Hz, 1H), 4.29 (dd, J=10.6, 5.8 Hz, 1H), 4.00 (m, 1H), 3.31-3.33 (m, 2H), 2.57 (s, 3H), 2.00 (s, 3H); MS:479.2.

### Example 194

### Preparation of [(1S)-2-(1H-indol-3-yl)-1-({[5-(3-methyl-1H-pyrazolo[3,4-b]pyridin-5-yl)-6-phenyl-3-pyridinyl]oxy}methyl)ethyl]amine

The title compound was isolated as a by-product from the synthesis of Example 193.1H NMR (400 MHz, MeOD) 6 ppm 8.49 (d, J=2.8 Hz, 1H), 8.08-8.17 (m, 2H), 7.80 (d, J=2.5 Hz, 1H), 7.59 (d, J=7.8 Hz, 1H), 7.26-7.37 (m, 7H), 7.10 (t, J=7.6 Hz, 1H), 7.01 (t, J=7.5 Hz, 1H), 4.48 (dd, J=10.6, 3.0 Hz, 1H), 4.35 (dd, J=10.5, 5.7 Hz, 1H), 4.03 (m, 1H), 3.31-3.34 (m, 2H), 2.53 (s, 3H); MS: 475.2

### Example 195

### Preparation of [(1S)-2-{[6-(3-furanyl)-5-(3-methyl-1H-pyrazolo[3,4-b]pyridin-5-yl)-3-pyridinyl]oxy}-1-(1H-indol-3-ylmethyl)ethyl]methylamine

Following the procedure in Example 156, except substituting 192(e) for 1(c), the title compound was prepared. 1H NMR (400 MHz, MeOD) δ ppm 8.41 (d, J=2.5 Hz, 1H), 8.30 (s, 1H), 8.14 (d, J=1.8 Hz, 1H), 7.56 (d, J=8.1 Hz, 1H), 7.45 (s, 1H), 7.32-7.43 (m, 2H), 7.22-7.29 (m, 2H), 7.09 (t, J=7.2 Hz, 1H), 6.98 (t, J=7.5 Hz, 1H), 6.25 (d, J=1.3 Hz, 1H), 4.43 (dd, J=11.0, 2.7 Hz, 1H), 4.29 (dd, J=11.0, 4.2 Hz, 1H), 3.88-3.95 (m, 1H), 3.30-3.32 (m, 2H), 2.90 (s, 3H), 2.57 (s, 3H); MS: 479.2

### Example 196

### Preparation of 2-[5-{[(2S)-2-amino-3-(1H-indol-3-yl)propyl]oxy}-3-(3-methyl-1H-pyrazolo[3,4-b]pyridin-5-yl)-2-pyridinyl]phenol

Following the procedure in Example 192, except substituting 2-(4,4,5,5-tetramethyl-1,3,2-dioxaborolan-2-yl)phenol for 3-furanylboronic acid, the title compound was prepared. 1H NMR (400 MHz, MeOD) δ ppm 8.49 (d, J=2.5 Hz, 1H), 8.26 (d, J=2.0 Hz, 1H), 8.04 (d, J=2.0 Hz, 1H), 7.92 (d, J=2.8 Hz, 1H), 7.60 (d, J=7.8 Hz, 1H), 7.37 (d, J=8.1 Hz, 1H), 7.22-7.27 (m, 2H), 7.17 (dd, J=7.6, 1.5 Hz, 1H), 7.09-7.13 (m, 1H), 7.01-7.05 (m, 1H), 6.83-6.88 (m, 1H), 6.75 (d, J=7.6 Hz, 1H), 4.50 (dd, J=10.6, 3.3 Hz, 1H), 4.37 (dd, J=10.6, 5.8 Hz, 1H), 4.04 (m, 1H), 3.31-3.34 (m, 2H), 2.48 (s, 3H); MS: 491.2

### Example 197

### Preparation of 2-[5-{[(2S)-2-amino-3-(1H-indol-3-yl)propyl]oxy}-3-(3-methyl-1H-pyrazolo[3,4-b]pyridin-5-yl)-2-pyridinyl]-6-fluorophenol

Following the procedure in Example 192, except substituting [3-fluoro-2-(methyloxy)phenyl]boronic acid for 3-furanylboronic acid, the title compound was prepared. 1H NMR (400 MHz, MeOD) δ ppm 8.48 (s, 1H), 8.25 (s, 1H), 8.04 (d, J=2.0 Hz, 1H), 7.79 (s, 1H), 7.60 (d, J=7.8 Hz, 1H), 7.36 (d, J=8.3 Hz, 1H), 7.26 (s, 1H), 7.06-7.12 (m, 1H), 7.01-7.06 (m, 1H), 6.81 (m, 1H), 4.48 (dd, J=10.5, 2.7 Hz, 1H), 4.34 (dd, J=10.4, 5.8 Hz, 1H), 4.03 (m, 1H), 3.32-3.34 (m, 2H), 2.49 (s, 3H). MS: 509.2

### Example 198

### Preparation of [(1S)-2-{[5-[3-(3,5-dimethyl-4-isoxazolyl)-1H-indazol-5-yl]-6-(3-furanyl)-3-pyridinyl]oxy}-1-(phenylmethyl)ethyl]amine

Following the procedure of Example 38, except substituting (3,5-dimethyl-4-isoxazolyl)boronic acid for 2-furanylboronic acid, the title compound was prepared.

1H NMR (400 MHz, MeOD) δ ppm 8.36-8.41 (m, 1H), 7.69 (d, J=8.6 Hz, 1H), 7.50-7.58 (m, 2H), 7.42 (s, 1H), 7.27-7.40 (m, 7H), 6.24 (d, J=1.3 Hz, 1H), 4.30-4.36 (m, 1H), 4.18 (dd, J=10.6, 5.6 Hz, 1H), 3.86-3.96 (m, 1H), 3.13 (d, J=7.6 Hz, 2H), 2.34 (s, 3H), 2.22 (s, 3H); 506.2.

### Example 199

### Preparation of [(1S)-2-({6-(3-furanyl)-5-[3-(2-pyridinyl)-1H-indazol-5-yl]-3-pyridinyloxy)-1-(phenylmethyl)ethyl]amine

To a solution of compound 122(b) (80 mg, 0.106 mmol) in DMF (1 ml), 2-(tributylstannanyl)pyridine (78 mg, 0.212 mmol), TEA (0.06 ml, 0.424 mmol) and Pd(Ph3)4 (13 mg, 00010 mmol) were added. The reaction was heated at 100 C overnight. The reaction mixture was washed with EtOAc and concentrated. The residue was purified by flash column chromatography (1:1 hexene/EtoAc) to give 56 mg (76%) product, which was treated with TFA/CH2Cl2 to give the title compound.

1H NMR (400 MHz, MeOD) δ ppm 8.75 (m, 1H), 8.30-8.50 (m, 4H), 7.68-7.79 (m, 3H), 7.26-7.43 (m, 8H), 6.34 (d, J=1.3 Hz, 1H), 4.38 (dd, J=10.6, 2.5 Hz, 1H), 4.23 (dd, J=10.4, 5.6 Hz, 1H), 3.91-3.99 (m, 1H), 3.16 (d, J=7.6 Hz, 2H); MS: 488.2.

### Example 200

### Preparation of [(1S)-2-{[6-(2-chlorophenyl)-5-(3-methyl-1H-indazol-5-yl)-3-pyridinyl]oxy}-1-(phenylmethyl)ethyl]amine

Following the procedure of Example 1 (f), except substituting (2-chlorophenyl)boronic acid for phenyl boronic acid, the title compound was prepare.

1H NMR (400 MHz, MeOD) δ ppm 8.43 (d, J=2.5 Hz, 1H), 7.73 (d, J=2.8 Hz, 1H), 7.52 (s, 1H), 7.27-7.39 (m, 10H), 7.18 (d, J=8.8 Hz, 1H), 4.37-4.45 (m, 1H), 4.26 (dd, J=10.6, 5.6 Hz, 1H), 3.98 (m, 1H), 3.13-3.22 (m, 2H), 2.43 (s, 3H); MS: 469.2.

### Example 201

### Preparation of [(1S)-2-{[5-(3-methyl-1H-indazol-5-yl)-6-(2-methylphenyl)-3-pyridinyl]oxy-1-(phenylmethyl)ethyl]amine

Following the procedure of Example 1(f), except substituting (2-methylphenyl)boronic acid for phenylboronic acid, the title compound was prepare.

1H NMR (400 MHz, MeOD) δ ppm 8.49-8.56 (m, 1H), 8.11-8.03 (m, 1H), 7.55 (d, J=4.0 Hz, 1H), 7.28-7.39 (m, 9H), 7.15-7.20 (m, 2H), 4.45-4.53 (m, 1H), 4.30-4.39 (m, 1H), 3.97-4.04 (m, 1H), 3.18 (d, J=7.8 Hz, 2H), 2.43 (s, 3H), 1.93 (s, 3H); MS: 449.2.

### Example 202

### Preparation of [(1S)-2-{[6-(2-fluorophenyl)-5-(3-methyl-1H-indazol-5-yl)-3-pyridinyl]oxy}-1-(phenylmethyl)ethyl]amine

Following the procedure of Example 1(f), except substituting (2-fluorophenyl)boronic acid for phenylboronic acid, the title compound was prepare.

1H NMR (400 MHz, MeOD) δ ppm 8.45 (d, J=2.8 Hz, 1H), 7.73 (d, J=3.0 Hz, 1H), 7.54 (s, 1H), 7.29-7.40 (m, 8H), 7.13-7.21 (m, 2H), 6.93-7.00 (m, 1H), 4.41 (dd, J=10.6, 3.0 Hz, 1H), 4.26 (dd, J=10.6, 5.6 Hz, 1H), 3.97 (m, 1H), 3.12-3.22 (m, 2H), 2.44 (s, 3H); MS: 453.2.

### Example 203

### Preparation of 2-[5-{[(2S)-2-amino-3-phenylpropyl]oxy}-3-(3-methyl-1H-indazol-5-yl)-2-pyridinyl]-4-chlorophenol

Following the procedure of Example 1(e), except substituting [5-chloro-2-(methyloxy)phenyl]boronic acid for phenylboronic acid, and the resulted product was dissolved in CH2Cl2. After cooling to OC, 2.5 eq of BBr3 was added to the reaction mixture. Stirred at this temperature for 3 hrs, the mixture was filtered through celite. The crude product was purified on reverse phase HPLC to give the title compound. 1H NMR (400 MHz, MeOD) δ ppm 8.46 (d, J=2.8 Hz, 1H), 7.88 (d, J=2.8 Hz, 1H), 7.65 (s, 1H), 7.29-7.41 (m, 6H), 7.17-7.22 (m, 2H), 7.10 (d, J=2.8 Hz, 1H), 6.74 (d, J=8.6 Hz, 1H), 4.44 (dd, J=10.6, 3.0 Hz, 1H), 4.29 (dd, J=10.6, 5.6 Hz, 1H), 3.98 (m, 1H), 3.17 (d, J=7.1 Hz, 2H), 2.49 (s, 3H); MS: 485.2/487.2.

### Example 204

### Preparation of [(1S)-2-{[6-(1-benzothien-3-yl)-5-(3-methyl-1H-indazol-5-yl)-3-pyridinyl]oxy}-1-(phenylmethyl)ethyl]amine

Following the procedure of Example 1(f), except substituting 1-benzothien-3-ylboronic acid for phenylboronic acid, the title compound was prepare. 1H NMR (400 MHz, MeOD) δ ppm 8.52 (d, J=2.8 Hz, 1H), 7.93 (d, J=2.8 Hz, 1H), 7.87 (d, J=8.1 Hz, 1H), 7.65 (s, 1H), 7.53 (s, 1H), 7.47 (d, J=7.8 Hz, 1H), 7.36-7.42 (m, 4H), 7.28-7.34 (m, 2H), 7.20-7.26 (m, 2H), 7.14 (dd, J=8.6, 1.5 Hz, 1H), 4.47 (dd, J=10.7, 2.9 Hz, 1H), 4.32 (dd, J=10.6, 5.6 Hz, 1H), 4.00 (m, 1H), 3.15-3.24 (m, 2H), 2.42 (s, 3H); MS: 491.2.

### Example 205

### Preparation of 3-[5-{[(2S)-2-amino-3-phenylpropyl]oxy}-3-(3-methyl-1H-indazol-5-yl)-2-pyridinyl]benzamide

Following the procedure of Example 1(f), except substituting [3-(aminocarbonyl)phenyl]boronic acid for phenylboronic acid, the title compound was prepare. 1H NMR (400 MHz, MeOD) 6 ppm 8.47 (d, J=2.8 Hz, 1H), 7.94-7.97 (m, 1H), 7.80 (dt, J=7.4, 1.6 Hz, 1H), 7.74 (d, J=2.8 Hz, 1H), 7.66 (s, 1H), 7.30-7.39 (m, 8H), 7.08 (dd, J=8.6, 1.5 Hz, 1H), 4.41 (dd, J=10.6, 3.0 Hz, 1H), 4.26 (dd, J=10.6, 5.6 Hz, 1H), 3.97 (m, 1H), 3.13-3.22 (m, 1H), 2.49 (s, 3H); MS: 478.2.

### Example 206

### Preparation of 3-[5-{[(2S)-2-amino-3-phenylpropyl]oxy}-3-(3-methyl-1H-indazol-5-yl)-2-pyridinyl]benzonitrile

Following the procedure of Example 1(f), except (3-cyanophenyl)boronic acid for phenylboronic acid, the title compound was prepare. 1H NMR (400 MHz, MeOD) δ ppm 8.45 (d, J=2.8 Hz, 1H), 7.70 (s, 1H), 7.66 (s, 1H), 7.58-7.63 (m, 2H), 7.47-7.52 (m, 1H), 7.29-7.41 (m, 7H), 7.07 (dd, J=8.7, 1.6 Hz, 1H), 4.38 (dd, J=10.6, 3.0 Hz, 1H), 4.23 (dd, J=10.6, 5.6 Hz, 1H), 3.96 (m, 1H), 3.13-3.22 (m, 2H), 2.52 (s, 3H); MS: 460.4.

### Example 207

### Preparation of [(1S)-2-{[5-(3-methyl-1H-indazol-5-yl)-6-(3-nitrophenyl)-3-pyridinyl]oxy}-1-(phenylmethyl)ethyl]amine

Following the procedure of Example 1(f), except substituting (3-nitrophenyl)boronic acid for phenylboronic acid, the title compound was prepare.

1H NMR (400 MHz, MeOD) δ ppm 8.47 (d, J=2.8 Hz, 1H), 8.23-8.26 (m, 1H), 8.10-8.14 (m, 1H), 7.68 (s, 1H), 7.56-7.61 (m, 2H), 7.31-7.41 (m, 7H), 7.08 (dd, J=8.7, 1.6 Hz, 1H), 4.39 (dd, J=10.6, 3.0 Hz, 1H), 4.23 (dd, J=10.6, 5.3 Hz, 1H), 3.96 (m, 1H), 3.13-3.22 (m, 2H), 2.51 (s, 3H); MS: 480.4.

### Example 208

### Preparation of [(1S)-2-{[5-(3-methyl-1H-indazol-5-yl)-6-(4-methyl-2-thienyl)-3-pyridinyl]oxy}-1-(phenylmethyl)ethyl]amine

Following the procedure of Example 1(f), except substituting (4-methyl-2-thienyl)boronic acid for phenylboronic acid, the title compound was prepare. 1H NMR (400 MHz, MeOD) 6 ppm 8.33 (d, J=2.9 Hz, 1H), 7.71 (dd, J=1.4, 0.8 Hz, 1H), 7.50 (dd, J=8.6, 0.7 Hz, 1H), 7.43 (s, 1H), 7.43-7.31 (m, 5H), 7.25 (dd, 1, J=8.6, 1.6 Hz, 1H), 6.92 (m, 1H), 6.45 (d, J=1.3 Hz, 1H), 4.33 (dd, J=10.6, 3.0 Hz, 1H), 4.18 (dd, J=10.6, 5.5 Hz, 1H), 3.9-4.0 (m, 1H), 3.1-3.2 (m, 2H), 2.58 (s, 3 h), 2.01 (d, J=0.8 Hz, 3H); MS: 455.2.

### Example 209

### Preparation of N-{3-[5-{[(2S)-2-amino-3-phenylpropyl]oxy}-3-(3-methyl-1H-indazol-5-yl)-2-pyridinyl]phenyl}-N′-phenylurea

Following the procedure of Example 1(f), except substituting (3-{[(phenylamino)carbonyl]amino}phenyl)boronic acid for phenylboronic acid, the title compound was prepare. 1H NMR (400 MHz, MeOD) δ ppm 8.44 (s, 1H), 7.81 (s, 1H), 7.70 (s, 1H), 7.56 (d, J=1.5 Hz, 1H), 7.24-7.39 (m, 11H), 7.18 (t, J=7.8 Hz, 1H), 7.12 (dd, J=8.6, 1.5 Hz, 1H), 7.01 (t, J=7.3 Hz, 1H), 6.87 (d, J=7.8 Hz, 1H), 4.42 (dd, J=10.5, 2.1 Hz, 1H), 4.27 (dd, J=10.6, 5.6 Hz, 1H), 3.97 (m, 1H), 3.17 (d, J=7.3 Hz, 2H), 2.51 (s, 3H); MS: 569.4.

### Example 210

### Preparation of [(1S)-2-{[5-(3-methyl-1H-indazol-5-yl)-6-(2-thienyl)-3-pyridinyl]oxy-1-(phenylmethyl)ethyl]amine

Following the procedure of Example 1(f), except substituting 2-thienylboronic acid for phenylboronic acid, the title compound was prepare. 1H NMR (400 MHz, MeOD) 6 ppm 8.35 (d, J=2.8 Hz, 1H), 7.70 (s, 1H), 7.48 (d, J=8.6 Hz, 1H), 7.43-7.45 (m, 1H), 7.27-7.39 (m, 6H), 7.23 (dd, J=8.6, 1.5 Hz, 1H), 6.79 (dd, J=5.1, 3.8 Hz, 1H), 6.58 (d, J=3.5 Hz, 1H), 4.33 (dd, J=10.6, 3.0 Hz, 1H), 4.18 (dd, J=10.6, 5.6 Hz, 1H), 3.93 (m, 1H), 3.13-3.22 (m, 2H), 2.55 (s, 3H); MS: 441.2.

### Example 211

### Preparation of [(1S)-2-(1H-indol-3-yl)-1-({[6-(2-methyl-3-furanyl)-5-(3-methyl-1H-indazol-5-yl)-3-pyridinyl]oxy}methyl)ethyl]amine

a) 4,4,5,5-tetramethyl-2-(2-methyl-3-furanyl)-1,3,2-dioxaborolane

The title compound was prepared following the procedure 1(c)) except substituting N-Boc-3-methyl-5-bromoindazole with 3-bromo-2-methyl furan (Tett 52, (1996), 4065-4078)

b) [(1S)-2-(1H-indol-3-yl)-1-({[6-(2-methyl-3-furanyl)-5-(3-methyl-1H-indazol-5-yl)-3-pyridinyl]oxy}methyl)ethyl]amine

Following the procedure of Example 1(f), except substituting Example 211(a) for phenylboronic acid, the title compound was prepare. 1H NMR (400 MHz, MeOD) δ ppm 8.41 (d, J=2.8 Hz, 1H), 7.79 (d, J=2.8 Hz, 1H), 7.67 (s, 1H), 7.60 (d, J=8.1 Hz, 1H), 7.38-7.43 (m, 2H), 7.34 (d, J=2.0 Hz, 1H), 7.26 (s, 1H), 7.18 (dd, J=8.7, 1.6 Hz, 1H), 7.10-7.16 (m, 1H), 7.00-7.06 (m, 1H), 6.19 (d, J=1.8 Hz, 1H), 4.46 (dd, J=10.5, 3.2 Hz, 1H), 4.33 (dd, J=10.6, 5.8 Hz, 1H), 3.97-4.06 (m, 1H), 3.32-3.34 (m, 2H), 2.54 (s, 3H), 1.93 (3, 4H); MS: 478.4

### Example 212

### Preparation of {2-[5-{[(2S)-2-amino-3-(1H-indol-3-yl)propyl]oxy}-3-(3-methyl-1H-indazol-5-yl)-2-pyridinyl]phenyl}amine

Following the procedure of Example 1(f), except substituting 2-aminophenyl)boronic acid for phenylboronic acid, the title compound was prepare.

1H NMR (400 MHz, MeOD) δ ppm 8.49 (d, J=2.8 Hz, 1H), 7.79 (d, J=2.8 Hz, 1H), 7.66 (s, 1H), 7.62 (d, J=7.8 Hz, 1H), 7.40 (d, J=8.1 Hz, 1H), 7.35 (d, J=8.6 Hz, 1H), 7.26-7.32 (m, 2H), 7.12-7.23 (m, 3H), 7.04 (t, J=7.1 Hz, 1H), 6.88-6.93 (m, 2H), 4.48 (dd, J=10.5, 3.2 Hz, 1H), 4.35 (dd, J=10.6, 5.8 Hz, 1H), 4.04 (m, 1 H), 3.32-3.34 (m, 2H), 2.49 (s, 3H); MS: 489.2.

### Example 213

### Preparation of 2-[5-{[(2S)-2-amino-3-(1H-indol-3-yl)propyl]oxy}-3-(3-methyl-1H-indazol-5-yl)-2-pyridinyl]-6-fluorophenol

Following the procedure of Example 203, except substituting 3-fluoro-2-(methyloxy)phenyl]boronic acid for [5-chloro-2-(methyloxy)phenyl]boronic acid, the title compound was prepare. 1H NMR (400 MHz, MeOD) δ ppm 8.43 (d, J=2.8 Hz, 1H), 7.79 (d, J=2.8 Hz, 1H), 7.60-7.63 (m, 1H), 7.60 (s, 1H), 7.39 (d, J=8.1 Hz, 1H), 7.34 (d, J=8.8 Hz, 1H), 7.27 (s, 1H), 7.12-7.18 (m, 2H), 7.03-7.10 (m, 2H), 6.85 (d, J=7.8 Hz, 1H), 6.71 (td, J=8.0, 4.8 Hz, 1H), 4.48 (dd, J=10.5, 3.2 Hz, 1H), 4.34 (dd, J=10.6, 5.8 Hz, 1H), 4.03 (m, 1H), 3.32-3.34 (m, 2H), 2.49 (s, 3H); MS: 508.2.

### Example 214

### Preparation of 2-[5-{[(2S)-2-amino-3-(1H-indol-3-yl)propyl]oxy}-3-(3-methyl-1H-indazol-5-yl)-2-pyridinyl]-4-chlorophenol

Following the procedure of Example 203, except substituting 5-chloro-2-(methyloxy)phenyl]boronic acid for [5-chloro-2-(methyloxy)phenyl]boronic acid, the title compound was prepare. 1H NMR (400 MHz, MeOD) δ ppm 8.46 (d, J=2.5 Hz, 1H), 7.91 (d, J=2.8 Hz, 1H), 7.58-7.65 (m, 2H), 7.36-7.40 (m, 2H), 7.27 (s, 1H), 7.18-7.22 (m, 2H), 7.09-7.16 (m, 2H), 7.02-7.07 (m, 1H), 6.77 (d, J=8.8 Hz, 1H), 4.50 (dd, J=10.5, 3.2 Hz, 1H), 4.37 (dd, J=10.4, 5.8 Hz, 1H), 4.04 (m, 1H), 3.32-3.34 (m, 2H), 2.49 (s, 3H); MS: 524.2.

### Example 215

### Preparation of 2-[5-{[(2S)-2-amino-3-(1H-indol-3-yl)propyl]oxy}-3-(3-methyl-1H-indazol-5-yl)-2-pyridinyl]-4-fluorophenol

Following the procedure of Example 203, except substituting 5-fluoro-2-(methyloxy)phenyl]boronic acid for [5-chloro-2-(methyloxy)phenyl]boronic acid, the title compound was prepare. 1H NMR (400 MHz, MeOD) δ ppm 8.47 (d, J=2.8 Hz, 1H), 7.96 (d, J=2.8 Hz, 1H), 7.63 (s, 1H), 7.61 (d, J=7.8 Hz, 1H), 7.36-7.40 (m, 2H), 7.26 (s, 1H), 7.18 (dd, J=8.6, 1.5 Hz, 1H), 7.10-7.16 (m, 1H), 6.96-7.06 (m, 2H), 6.82 (dd, J=8.7, 3.2 Hz, 1H), 6.79 (dd, J=9.1, 4.5 Hz, 1H), 4.51 (dd, J=10.6, 3.0 Hz, 1H), 4.37 (dd, J=10.6, 5.8 Hz, 1H), 4.01-4.07 (m, 1H), 3.32-3.34 (m, 2H), 2.49 (s, 3H); MS: 508.2.

### Example 216

### Preparation of [(1S)-2-{[6-[3,5-difluoro-2-(methyloxy)phenyl]-5-(3-methyl-1H-thieno[3,2-c]pyrazol-5-yl)-3-pyridinyl]oxy}-1-(1H-indol-3-ylmethyl)ethyl]amine

Following the procedure of Example 121, except substituting 3,5-difluoro-2-(methyloxy)phenyl]boronic acid for 3-furanylboronic acid, the title compound was prepared. 1H NMR (400 MHz, MeOD) δ ppm 8.37 (d, J=2.8 Hz, 1H), 7.60-7.66 (m, 2H), 7.39 (d, J=8.1 Hz, 1H), 7.25 (s, 1H), 7.05-7.16 (m, 3H), 6.92 (ddd, J=8.3, 2.9, 1.6 Hz, 1H), 6.78 (s, 1H), 4.43 (dd, J=10.6, 3.0 Hz, 1H), 4.28 (dd, J=10.6, 5.8 Hz, 1H), 3.99 (m, 1H), 3.51 (d, J=1.8 Hz, 3H), 3.32-3.34 (m, 2H), 2.40 (s, 3H); MS: 546.2

### Example 217

### Preparation of 2-[5-{[(2S)-2-amino-3-(1H-indol-3-yl)propyl]oxy}-3-(3-methyl-1H-thieno[3,2-c]pyrazol-5-yl)-2-pyridinyl-4,6-difluorophenol

Using BBr3 to remove Methyl protecting group of Example 216 as described in Example 203, the title compound was prepared. 1H NMR (400 MHz, MeOD) δ ppm 8.37 (d, J=2.5 Hz, 1H), 7.59-7.67 (m, 2H), 7.39 (d, J=8.1 Hz, 1H), 7.25 (s, 1H), 7.13 (t, J=7.7 Hz, 1H), 6.98-7.08 (m, 2H), 6.80-6.85 (m, 2H), 4.43 (dd, J=10.4, 3.0 Hz, 1H), 4.28 (dd, J=10.5, 5.9 Hz, 1H), 3.96-4.03 (m, 1H), 3.31-3.33 (m, 2H), 2.40 (s, 3H); MS:532.2.

### Example 218

### Preparation of 2-[5-{[(2S)-2-amino-3-(1H-indol-3-yl)propyl]oxy}-3-(3-methyl-1H-thieno[3,2-c]pyrazol-5-yl)-2-pyridinyl]phenol

Following the procedure of Example 121, except substituting 2-(4,4,5,5-tetramethyl-1,3,2-dioxaborolan-2-yl)phenol for 3-furanylboronic acid, the title compound was prepared. 1H NMR (400 MHz, MeOD) δ ppm 8.42 (d, J=2.5 Hz, 1H), 7.94 (d, J=2.8 Hz, 1H), 7.61 (d, J=8.1 Hz, 1H), 7.38 (d, J=8.1 Hz, 1H), 7.29-7.35 (m, 1H), 7.26 (s, 1H), 7.22 (dd, J=7.6, 1.5 Hz, 1H), 7.10-7.18 (m, 1H), 7.03-7.08 (m, 1H), 6.84-6.94 (m, 3H), 4.49 (dd, J=10.5, 3.2 Hz, 1H), 4.34 (dd, J=10.6, 5.8 Hz, 1H), 4.03 (m, 1H), 3.31-3.34 (m, 2H), 2.38 (s, 3H); MS: 496.2.

### Example 219

### Preparation of 2-[5-{[(2S)-2-amino-3-(1H-indol-3-yl)propyl]oxy}-3-(3-methyl-1H-thieno[3,2-clpyrazol-5-yl)-2-pyridinyl]-4-chlorophenol

Following the procedure of Example 217, except substituting 5-chloro-2-(methyloxy)phenyl]boronic acid for 3,5-difluoro-2-(methyloxy)phenyl]boronic acid, the title compound was prepared. 1H NMR (400 MHz, MeOD) δ ppm 8.35 (d, J=2.8 Hz, 1H), 7.67 (d, J=2.8 Hz, 1H), 7.61 (d, J=8.1 Hz, 1H), 7.38 (d, J=8.1 Hz, 1H), 7.19-7.26 (m, 3H), 7.10-7.16 (m, 1H), 7.03-7.08 (m, 1H), 6.74-6.81 (m, 2H), 4.43 (dd, J=10.5, 3.2 Hz, 1H), 4.28 (dd, J=10.5, 5.9 Hz, 1H), 3.99 (m, 1H), 3.30-3.33 (m, 2H), 2.39 (s, 3H); MS: 530.0

### Example 220

### Preparation of 3-(5-{[(2S)-2-amino-3-(1H-indol-3-yl)propyl]oxy}-3-pyridinyl)benzamide

Following the procedure of Example 69, except substituting 5-chloro-3-pyridinol for 5-bromo-6-chloro-3-pyrid inol and 3-(aminocarbonyl)phenyl]boronic acid for Example 1(c), the title compound was prepared. 1H NMR (400 MHz, MeOD) δ ppm 8.62 (s, 1H), 8.40 (d, J=2.0 Hz, 1H), 8.18-8.21 (m, 1H), 7.98 (d, J=7.6 Hz, 1H), 7.82-7.88 (m, 2H), 7.60-7.67 (m, 2H), 7.40 (d, J=8.1 Hz, 1H), 7.26 (s, 1H), 7.15 (t, J=7.6 Hz, 1H), 7.05 (t, J=7.5 Hz, 1H), 4.44 (dd, J=10.5, 3.2 Hz, 1H), 4.30 (dd, J=10.5, 5.7 Hz, 1H), 4.01 (m, 1H), 3.32-3.34 (m, 2H); MS: 387.2.

### Example 221

### Preparation of 1-[3-(5-{[(2S)-2-amino-3-(1H-indol-3-yl)propyloxy}-3-pyridinyl)phenyl]ethanone

Following the procedure of Example 220, except substituting 3-acetylphenyl)boronic acid for 3-(aminocarbonyl)phenyl]boronic acid, the title compound was prepared. 1H NMR (400 MHz, MeOD) δ ppm 8.66 (m, 1H), 8.44 (m, 1H), 8.26 (s, 1H), 8.12 (d, J=7.8 Hz, 1H), 7.91-8.05 (m, 2H), 7.68 (t, J=7.7 Hz, 1H), 7.60 (d, J=7.8 Hz, 1H), 7.38 (d, J=8.1 Hz, 1H), 7.25 (s, 1H), 7.13 (t, J=7.6 Hz, 1H), 7.03 (t, J=7.5 Hz, 1H), 4.45 (m, 1H), 4.33 (rn, 1H), 4.02 (m, 1H), 3.31-3.34 (m, 2H), 2.69 (s, 3H); MS: 386.2.

### Example 222

### Preparation of 5-{[(2S)-2-amino-3-(1H-indol-3-yl)propyl]oxy}-2-(3-furanyl)-3,4′-bipyridine-2′-carboxamide

The title compound was prepared following (final steps in 7-azaindazole synthesis described in Example 170) except substituting 1,1-dimethylethyl 3-methyl-5-{[(trifluoromethyl)sulfonyl]oxy}-1H-pyrazolo[3,4-b]pyridine-1-carboxylate with 4-chloro-2-pyridinecarboxamide. H NMR 8.68 (d, J=4.6 Hz, 1H), 8.46 (d, J=2.8 Hz, 1H), 8.06 (s, 1H), 7.59 (d, J=7.9 Hz, 1H), 7.51-7.37 (m, 5H), 7.25 (s, 1H), 7.15-7.11 (m, 1H), 7.05-7.01 (m, 1H), 6.28 (d, J=1.2 Hz, 1H), 4.41 (dd, J=10.6, 3.2 Hz), 4.27 (dd, J=10.6, 5.8 Hz, 1H), 4.03-3.97 (m, 1H), 3.38-3.25 (m, 2H); MS: 454.2.

Example 223

### Capsule Composition

An oral dosage form for administering the present invention is produced by filing a standard two piece hard gelatin capsule with the ingredients in the proportions shown in Table I, below.

TABLE I

| INGREDIENTS | AMOUNTS |
|---|---|
| (S)-1-Benzyl-2-[5-(3-methyl-1H-indazol-5-yl)-6-phenyl- | 25 mg |
| pyridin-3-yloxy]-ethylamine | |
| Lactose | 55 mg |
| Talc | 16 mg |
| Magnesium Stearate | 4 mg |

### Example 224

### Injectable Parenteral Composition

An injectable form for administering the present invention is produced by stirring 1.5% by weight of (S)-1-Benzyl-2-[5-(3-methyl-1H-indazol-5-yl)-6-phenyl-pyridin-3-yloxy]-ethylamine in 10% by volume propylene glycol in water.

### Example 225

### Tablet Composition

The sucrose, calcium sulfate dihydrate and an Akt inhibitor as shown in Table II below, are mixed and granulated in the proportions shown with a 10% gelatin solution. The wet granules are screened, dried, mixed with the starch, talc and stearic acid;, screened and compressed into a tablet.

TABLE II

| INGREDIENTS | AMOUNTS | |
|---|---|---|
| (S)-1-Benzyl-2-[5-(3-methyl-1H-indazol-5-yl)-6-phenyl- | 20 | mg |
| pyridin-3-yloxy]-ethylamine | | |
| calcium sulfate dihydrate | 30 | mg |
| sucrose | 4 | mg |
| starch | 2 | mg |
| talc | 1 | mg |
| stearic acid | 0.5 | mg |

While the preferred embodiments of the invention are illustrated by the above, it is to be understood that the invention is not limited to the precise instructions herein disclosed and that the right to all modifications coming within the scope of the following claims is reserved.

1. A compound of Formula (I): 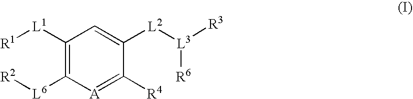
wherein:
A is selected from: nitrogen, —C-halogen and —CH;
L¹ is selected from the group consisting of a bond, —O—, —N(R⁵)—, —S—, —S(O)—, —S(O₂)—, alkyl, and —N(R⁵)C(O)—;
L² is selected from the group consisting of a bond, —O—, heterocycle, —N(R⁵)—, —N(R⁵)C(O)—, —S—, —S(O)—, —S(O₂)—, and —C(O)N(R⁵)—;
L³ is alkyl, wherein the alkyl is optionally substituted with one or two substituents independently selected from the group consisting of amino, methylamino, dimethylamino, oxo, and hydroxy;
L⁶ is selected from the group consisting of a bond, —O—, —N(R⁵)—, —S—, —S(O)—, —S(O₂)—, alkyl, and —N(R⁵)C(O)—;
R¹ is selected from the group consisting of aryl, substituted aryl, cycloalkyl, substituted cycloalkyl, heterocycle and substituted heterocycle;
R² is selected from alkyl, substituted alkyl, cycloalkyl, substituted cycloalkyl, heterocycle, substituted heterocycle, and a cyclic or polycyclic aromatic ring containing from 3 to 16 carbon atoms and optionally containing one or more heteroatoms, provided that when the number of carbon atoms is 3 the aromatic ring contains at least two heteroatoms and when the number of carbon atoms is 4 the aromatic ring contains at least one heteroatom, and optionally substituted with one or more substituents selected from the group consisting of: alkyl, substituted alkyl, trifluoroalkoxy, C₁-C₁₂aryl, aryloxy, —O(CH₂)_{q}R³¹, —NHC(O)—NHR⁴¹, —C(O)R⁴³, substituted cycloalkyl, substituted C₁-C₁₂aryl, heterocycle, substituted heterocycle, oxo, hydroxy, alkoxy, cycloalkyl, acyloxy, amino, N-acylamino, nitro, cyano, halogen, —C(O)OR⁷, —C(O)NR⁸R⁹, —S(O)₂NR⁸R⁹, and —S(O)ₙR⁷,
where n is 0-2, q is 1-6,
R⁷ is hydrogen, alkyl, cycloalkyl, C₁-C₁₂aryl, substituted alkyl, substituted cycloalkyl and substituted C₁-C₁₂aryl,
R³¹ is C₁-C₁₂aryl, cycloalkyl and heterocycle, each of which is optionally substituted with from 1 to 4 substituents selected from: halogen, alkyl, hydroxyalkyl, alkoxy, acyloxy, amino, methylamino, dimethylamino, N-acylamino, hydroxy, nitro, tetrazole, cyano, oxo and trifluoromethyl,
R⁴¹ is selected from hydrogen, C₁-C₁₂aryl, cycloalkyl and heterocycle, wherein C₁-C₁₂aryl, cycloalkyl and heterocycle are optionally substituted with from 1 to 4 substituents selected from: halogen, alkyl, hydroxyalkyl, alkoxy, amino, methylamino, dimethylamino, hydroxy, nitro, tetrazole, cyano, oxo and trifluoromethyl,
R⁴³ is selected from C₁-C₁₂aryl, cycloalkyl and heterocycle, each of which is optionally substituted with from 1 to 4 substituents selected from: halogen, hydroxyalkyl, alkoxy, amino, methylamino, dimethylamino, hydroxy, nitro, tetrazole, cyano, oxo and trifluoromethyl, and
R⁸ and R⁹ are independently hydrogen, cycloalkyl, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, —C(O)OR¹⁰, —S(O)ₙR¹⁰, —C(O)NR¹⁰R¹¹, —S(O)₂NR¹⁰R¹¹, nitro, cyano, cycloalkyl, substituted cycloalkyl, halogen, aryl, and substituted aryl,
or R⁸ and R⁹ taken together with the nitrogen to which they are attached represent a 5 to 6 member saturated ring containing up to one other heteroatom selected from oxygen and nitrogen, where the ring is optionally subtituted with one or more substituents selected from amino, methylamino and dimethylamino,
where R¹⁰ and R¹¹ are independently hydrogen, alkyl, cycloalkyl, C₁-C₁₂aryl, substituted alkyl, substituted cycloalkyl and substituted C₁-C₁₂aryl, and n is 0-2,
and when L⁶ is a bond, R² can additionally be halogen;
R³ and R⁶ are independently selected from the group consisting of hydrogen, amino, methylamino, dimethylamino, aryl, substituted aryl, heterocycle, substituted heterocycle, cycloalkyl, substituted cycloalkyl, —S—C₁-C₁₂aryl, —O—C₁-C₁₂aryl, —OalkylC₁-C₁₂aryl, aryloxy, substituted aryloxy and arylalkoxy; and
R⁴ is selected from the group consisting of hydrogen and halogen;
where R⁵ is selected from the group consisting of hydrogen, —S(O)₂CH₃, —S(O)₂H and alkyl;
provided that when,
R¹ is azaindazole, substituted azaindazole, 1H-thienopyrazole, substituted 1H-thienopyrazole, benzamide, substituted benzamide, phenylethanone, substituted phenylethanone, thiophene, substituted thiophene, furan or substituted furan,
R² may additionally be hydrogen;
further provided that when
R¹ is isoquinoline,
R² is not furyl or alkyl.

2. A pharmaceutically acceptable salt, hydrate, solvate or pro-drug of a compound of Formula (I), as described in claim 1.

3. The compound of Formula (I), as claimed in claim 1, wherein
A is selected from: nitrogen, —C-halogen and —CH;
L¹ is selected from the group consisting of a bond, —O—, —N(R⁵)—, —S—, —S(O)—, —S(O₂)—, alkyl, and —N(R⁵)C(O)—;
L² is selected from the group consisting of a bond, —O—, heterocycle, —N(R⁵)—, —N(R⁵)C(O)—, —S—, —S(O)—, —S(O₂)—, and —C(O)N(R⁵)—;
L³ is alkyl, wherein the alkyl is optionally substituted with one or two substituents independently selected from the group consisting of amino, methylamino, dimethylamino, oxo, and hydroxy;
L⁶ is a bond;
R¹ is selected from the group consisting of C₁-C₁₂aryl and substituted C₁-C₁₂aryl;
R² is selected from alkyl, substituted alkyl, halogen, cycloalkyl, substituted cycloalkyl, heterocycle, substituted heterocycle, and C₁-C₁₂aryl optionally substituted with one or more substituents selected from the group consisting of: alkyl, substituted alkyl, trifluoroalkoxy, C₁-C₁₂aryl, aryloxy, —O(CH₂)_{q}R³¹, —NHC(O)—NHR⁴¹, —C(O)R⁴³, hydroxy, alkoxy, cycloalkyl, N-acylamino, nitro and halogen,
where q is 1-6,
R³¹ is C₁-C₁₂aryl, cycloalkyl and heterocycle, each of which is optionally substituted with from 1 to 4 substituents selected from: halogen, alkyl, hydroxyalkyl, alkoxy, acyloxy, amino, methylamino, dimethylamino, N-acylamino, hydroxy, nitro, tetrazole, cyano, oxo and trifluoromethyl,
R⁴¹ is selected from hydrogen, C₁-C₁₂aryl, cycloalkyl and heterocycle,
wherein C₁-C₁₂aryl, cycloalkyl and heterocycle are optionally substituted with from 1 to 4 substituents selected from: halogen, alkyl, hydroxyalkyl, alkoxy, amino, methylamino, dimethylamino, hydroxy, nitro, tetrazole, cyano, oxo and trifluoromethyl,
R⁴³ is selected from C₁-C₁₂aryl, cycloalkyl and heterocycle, each of which is optionally substituted with from 1 to 4 substituents selected from: halogen, hydroxyalkyl, alkoxy, amino, methylamino, dimethylamino, hydroxyl, nitro, tetrazole, cyano, oxo and trifluoromethyl,
R³ and R⁶ are independently selected from the group consisting of hydrogen, amino, methylamino, dimethylamino, aryl, substituted aryl, heterocycle, substituted heterocycle, cycloalkyl, substituted cycloalkyl, —S—C₁-C₁₂aryl, aryloxy and arylalkoxy; and
R⁴ is selected from the group consisting of hydrogen and halogen;
where R⁵ is selected from the group consisting of hydrogen, —S(O)₂CH₃, —S(O)₂H and alkyl;
provided that when,
R¹ is azaindazole, substituted azaindazole, 1H-thienopyrazole, substituted 1H-thienopyrazole, benzamide, substituted benzamide, phenylethanone, substituted phenylethanone, thiophene, substituted thiophene, furan or substituted furan,
R² may additionally be hydrogen;
further provided that when
R¹ is isoquinoline,
R² is not furyl or alkyl.

4. A pharmaceutically acceptable salt, hydrate, solvate or pro-drug of a compound of Formula (I), as described in claim 3.

5. A compound of claim 1 represented by the following Formula (II): 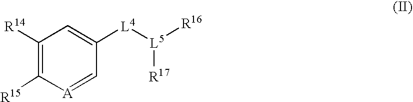
wherein:
A is selected from nitrogen, —CF and —CH;
L⁴ is selected from the group consisting of a bond, heterocycle, —O—, and —NH—;
L⁵ is alkyl, wherein the alkyl is optionally substituted with one or two substituents independently selected from the group consisting of amino, oxo, and hydroxy;
R¹⁴ is selected from the group consisting of C₁-C₁₂aryl, and substituted C₁-C₁₂aryl;
R¹⁵ is selected from alkyl, substituted alkyl, halogen, cycloalkyl, substituted cycloalkyl, heterocycle, substituted heterocycle, C₁-C₁₂aryl and C₁-C₁₂aryl optionally substituted with one or more substituents selected from the group consisting of: alkyl, substituted alkyl, trifluoroalkoxy, aryloxy, —O(CH₂)_{q}R³¹, —NHC(O)—NHR⁴¹, —C(O)R⁴³, hydroxy, alkoxy, acyloxy, amino, cycloalkyl, N-acylamino, nitro, cyano and halogen,
where q is 1-6,
R³¹ is C₁-C₁₂aryl optionally substituted with from 1 to 4 substituents selected from: halogen, alkyl, hydroxyalkyl, alkoxy, and hydroxy,
R⁴¹ is selected from hydrogen and C₁-C₁₂aryl optionally substituted with from 1 to 4 substituents selected from: halogen, alkyl, hydroxyalkyl, alkoxy, and hydroxy,
R⁴³ is C₁-C₁₂aryl substituted with from 1 to 4 substituents selected from: halogen, hydroxyalkyl, alkoxy, and hydroxy, and
R¹⁶ and R¹⁷ are independently selected from the group consisting of hydrogen, C₁-C₁₂aryl, substituted C₁-C₁₂aryl, heterocycle, cycloalkyl, —S—C₁-C₁₂aryl, and C₁-C₁₂arylalkoxy;
provided that when,
R¹⁴ is azaindazole, substituted azaindazole, 1H-thienopyrazole, substituted 1H-thienopyrazole, benzamide, substituted benzamide, phenylethanone, substituted phenylethanone, 2-pyridinecarboxamide, substituted 2-pyridinecarboxamide, (methylsulfonyl)benzene, substituted (methylsulfonyl)benzene, thiophene, substituted thiophene, furan or substituted furan,
R¹⁵ may additionally be hydrogen;
further provided that when
R¹⁴ is isoquinoline,
R¹⁵ is not furyl or alkyl.

6. A pharmaceutically acceptable salt, hydrate, solvate or pro-drug of a compound of Formula (II), as described in claim 5.

7. A compound of Formula (II), as described in claim 5:
wherein
A is selected from nitrogen, —CF and —CH;
L⁴ is selected from the group consisting of a bond, —O—, and —NH—;
L⁵ is alkyl, wherein the alkyl is substituted with one or two substituents independently selected from the group consisting of amino, oxo, and hydroxy;
R¹⁴ is selected from phenyl, pyridine, indazole, 7-azaindole, quinoline, isoquinoline, substituted phenyl, substituted pyridine, substituted indazole, substituted 7-azaindole, substituted quinoline and substituted isoquinoline;
R¹⁵ is selected from cycloalkyl, substituted cycloalkyl, phenyl, pyridine, thiophene, furan, pyrrole, indazole, quinoline, isoquinoline, 7-azaindole, substituted phenyl, substituted pyridine, substituted thiophene, substituted furan, substituted indazole, substituted quinoline, substituted 7-azaindole and substituted isoquinoline; and
R¹⁶ and R¹⁷ are independently selected from the group consisting of hydrogen, indole, substituted indole, azaindole, substituted azaindole, naphthalene, substituted naphthalene, benzofuran, substituted benzofuran, phenyl, pyridine, thiophene, furan, pyrrole, substituted phenyl, substituted pyridine, substituted thiophene, substituted furan, and substituted pyrrole;
provided that when,
R¹⁴ is 7-azaindazole, 4-azaindazole, 1H-thieno[3,2-c]pyrazole, benzamide, 1-phenylethanone, 2-furancarboxamide, 1-(2-furanyl)ethanone, 2-thienylcarboxamide, 1-(2-thienyl)ethanone, substituted 7-azaindazole, substituted 4-azaindazole, substituted 1H-thieno[3,2-c]pyrazole, substituted benzamide, substituted 1-phenylethanone, substituted 2-furancarboxamide, substituted 1-(2-furanyl)ethanone, substituted 2-thienylcarboxamide or substituted 1-(2-thienyl)ethanone, 2-pyridinecarboxamide, substituted 2-pyridinecarboxamide, (methylsulfonyl)benzene, substituted (methylsulfonyl)benzene,
R¹⁵ may additionally be hydrogen;
further provided that when
R¹⁴ is isoquinoline,
R¹⁵ is not furyl or alkyl.

8. A pharmaceutically acceptable salt, hydrate, solvate or pro-drug of a compound of Formula (II), as described in claim 7.

9. A compound of claim 1 selected from:
(S)-1-Benzyl-2-[5-(3-methyl-1H-indazol-5-yl)-6-phenyl-pyridin-3-yloxy]-ethylamine;
(S)-1-Benzyl-2-[6-furan-2-yl-5-(3-methyl-1H-indazol-5-yl)-pyridin-3-yloxy]-ethylamine;
(S)-1-Benzyl-2-[5,6-bis-(3-methyl-1H-indazol-5-yl)-pyridin-3-yloxy]-ethylamine;
(S)-1-Benzyl-2-[6-thiophen-2-yl-5-(3-methyl-1H-indazol-5-yl)-pyridin-3-yloxy]-ethylamine;
(S)-1-Benzyl-2-[6-(4-chlorophenyl)-5-(3-methyl-1H-indazol-5-yl)-pyridin-3-yloxy]-ethylamine;
(S)-1-Benzyl-2-[6-(3-chlorophenyl)-5-(3-methyl-1H-indazol-5-yl)-pyridin-3-yloxy]ethylamine;
(S)-1-Benzyl-2-[6-benzyl-5-(3-methyl-1H-indazol-5-yl)-pyridin-3-yloxy]-ethylamine;
(S)-1-Benzyl-2-[6-cyclopent-1-enyl-5-(3-methyl-1H-indazol-5-yl)-pyridin-3-yloxy]-ethylamine;
(S)-1-Benzyl-2-[6-cyclopentyl-5-(3-methyl-1H-indazol-5-yl)-pyridin-3-yloxy]-ethylamine;
(S)-1-Benzyl-2-[6-cyclohex-1-enyl-5-(3-methyl-1H-indazol-5-yl)-pyridin-3-yloxy]-ethylamine;
(S)-1-Benzyl-2-[6-cyclohexyl-5-(3-methyl-1H-indazol-5-yl)-pyridin-3-yloxy]-ethylamine;
3-Methyl-5-[2-phenyl-5-(piperidin-4-ylmethoxy)-pyridin-3-yl]-1H-indazole;
3-[5-(3-Methyl-1H-indazol-5-yl)-6-phenyl-pyridin-3-yloxy}-propylamine;
(S)-1-Benzyl-2-[5-(3-methyl-1H-indazol-5-yl)-6-(5-methyl-thiophen-2-yl)-pyridin-3-yloxy]-ethylamine;
(S)-1-Benzyl-2-[5-(3-methyl-1H-indazol-5-yl)-6-(5-methyl-furan-2-yl)-pyridin-3-yloxy]-ethylamine;
3-Methyl-5-[2-phenyl-5-(4-pyridin-3-yl-methyl-piperazin-1-yl)-pyridin-3-yl]-1H-indazole;
3-Methyl-5-[2-phenyl-5-(4-pyridin-4-ylmethyl-piperazin-1-yl)-pyridin-3-yl]-1H-indazole;
[(1S)-2-{[6-(3-furanyl)-5-(3-methyl-1H-indazol-5-yl)-3-pyridinyl]oxy}-1-(phenylmethyl)ethyl]amine;
[(1S)-2-{[5-(3-methyl-1H-indazol-5-yl)-6-(5-chloro-2-thienyl)-3-pyridinyl]oxy}-1-(phenylmethyl)ethyl]amine;
[(1S)-2-{[6-(3-aminophenyl)-5-(3-methyl-1H-indazol-5-yl)-3-pyridinyl]oxy}-1-(phenylmethyl)ethyl]amine;
(S)-1-Benzyl-2-[5-(1H-indazol-5-yl)-6-phenyl-pyridin-3-yloxy]-ethylamine;
(S)-1-Benzyl-2-{6-[3-(3-fluoro-benzyloxy)phenyl]-5-(3-methyl-1H-indazol-5-yl)-pyridin-3-yloxy}-ethylamine;
(S)-1-Benzyl-2-[5-(3-phenyl-1H-indazol-5-yl)-6-phenyl-pyridin-3-yloxy]-ethylamine;
[(1S)-2-{[5-(3-methyl-1H-indazol-5-yl)-6-(1H-pyrrol-2-yl)-3-pyridinyl]oxy}-1-(phenylmethyl)ethyl]amine;
N-{3-[5-{[(2S)-2-amino-3-phenylpropyl]oxy}-3-(3-methyl-1H-indazol-5-yl)-2-pyridinyl]phenyl}benzamide;
N-{3-[5-{[(2S)-2-amino-3-phenylpropyl]oxy}-3-(3-methyl-1H-indazol-5-yl)-2-pyridinyl]phenyl}-2,6-difluorobenzamide;
N-{3-[5-{[(2S)-2-amino-3-phenylpropyl]oxy}-3-(3-methyl-1H-indazol-5-yl)-2-pyridinyl]phenyl}cyclohexanecarboxamide;
[(1S)-2-({5-[3-(2-furanyl)-1H-indazol-5-yl]-6-phenyl-3-pyridinyl}oxy)-1-(phenylmethyl)ethyl]amine;
{(1S)-2-phenyl-1-[({6-phenyl-5-[3-(2-thienyl)-1H-indazol-5-yl]-3-pyridinyl}oxy)methyl]ethyl}amine;
[(1S)-2-({5-[3-(3-furanyl)-1H-indazol-5-yl]-6-phenyl-3-pyridinyl}oxy)-1-(phenylmethyl)ethyl]amine;
[(1S)-2-({5-[3-(3-thienyl)-1H-indazol-5-yl]-6-phenyl-3-pyridinyl}oxy)-1-(phenylmethyl)ethyl]amine;
3-[5-{[(2S)-2-amino-3-phenylpropyl]oxy}-3-(3-methyl-1H-indazol-5-yl)-2-pyridinyl]phenol;
[(1S)-2-{[5-(2,3-dimethyl-2H-indazol-5-yl)-6-phenyl-3-pyridinyl]oxy}-1-(phenylmethyl)ethyl]amine;
[(1S)-2-{[5-(3-cyclopropyl-1H-indazol-5-yl)-6-(3-furanyl)-3-pyridinyl]oxy}-1-(phenylmethyl)ethyl]amine;
[(1S)-2-{[5-(3-methyl-1H-indazol-5-yl)-6-(1-methyl-1H-pyrazol-4-yl)-3-pyridinyl]oxy}-1-(phenylmethyl)ethyl]amine;
[(1S)-2-{[6-{1-[(3-fluorophenyl)methyl]-1H-pyrazol-4-yl}-5-(3-methyl-1H-indazol-5-yl)-3-pyridinyl]oxy}-1-(phenylmethyl)ethyl]amine;
((1S)-2-phenyl-1-{[(6-phenyl-5-{3-[5-(1-piperazinylmethyl)-2-furanyl]-1H-indazol-5-yl}-3-pyridinyl)oxy]methyl}ethyl)amine;
[(1S)-2-({6-(3-furanyl)-5-[3-(2-furanyl)-1H-indazol-5-yl]-3-pyridinyl}oxy)-1-(phenylmethyl)ethyl]amine;
[(1S)-2-({5-(3-methyl-1H-indazol-5-yl)-6-[3-(phenyloxy)phenyl]-3-pyridinyl}oxy)-1-(phenylmethyl)ethyl]amine;
3-[({5-[5-(5-{[(2S)-2-amino-3-phenylpropyl]oxy}-2-phenyl-3-pyridinyl)-1H-indazol-3-yl]-2-furanyl}methyl)amino]propanenitrile;
[(1S)-2-({6-(2-furanyl)-5-[3-(2-furanyl)-1H-indazol-5-yl]-3-pyridinyl}oxy)-1-(phenylmethyl)ethyl]amine;
{5-[5-{[(2S)-2-amino-3-phenylpropyl]oxy}-3-(3-methyl-1H-indazol-5-yl)-2-pyridinyl]-2-thienyl}methanol;
{(1S)-2-phenyl-1-[({6-phenyl-5-[3-(phenylmethyl)-1H-indazol-5-yl]-3-pyridinyl}oxy)methyl]ethyl}amine;
[(1S)-2-{[5-(3-methyl-1H-indazol-5-yl)-6-(1-methyl-1H-pyrrol-2-yl)-3-pyridinyl]oxy}-1-(phenylmethyl)ethyl]amine;
5-(5-{[(2S)-2-amino-3-phenylpropyl]oxy}-2-phenyl-3-pyridinyl)-1H-indazol-3-amine;
[(1S)-2-({5-[3-(1-methylethenyl)-1H-indazol-5-yl]-6-phenyl-3-pyridinyl}oxy)-1-(phenylmethyl)ethyl]amine;
[(1S)-2-{[5-(3-methyl-1H-indazol-5-yl)-6-(1H-pyrazol-4-yl)-3-pyridinyl]oxy}-1-(phenylmethyl)ethyl]amine;
(2S)-N,N-dimethyl-1-{[5-(3-methyl-1H-indazol-5-yl)-6-phenyl-3-pyridinyl]oxy}-3-phenyl-2-propanamine;
[(1S)-2-{[3-(3-methyl-1H-indazol-5-yl)-2,4′-bipyridin-5-yl]oxy}-1-(phenylmethyl)ethyl]amine;
[(1S)-2-{[3-(3-methyl-1H-indazol-5-yl)-2,3′-bipyridin-5-yl]oxy}-1-(phenylmethyl)ethyl]amine;
[(1S)-2-{[5-(3-iodo-1H-indazol-5-yl)-6-phenyl-3-pyridinyl]oxy}-1-(phenylmethyl)ethyl]amine;
[(1S)-2-[(5-(3-methyl-1H-indazol-5-yl)-6-{3-[(trifluoromethyl)oxy]phenyl}-3-pyridinyl)oxy]-1-(phenylmethyl)ethyl]amine;
[(1S)-2-{[6-(3,5-dimethyl-4-isoxazolyl)-5-(3-methyl-1H-indazol-5-yl)-3-pyridinyl]oxy}-1-(phenylmethyl)ethyl]amine;
4-[5-{[(2S)-2-amino-3-phenylpropyl]oxy}-3-(3-methyl-1H-indazol-5-yl)-2-pyridinyl]phenol;
2-[5-{[(2S)-2-amino-3-phenylpropyl]oxy}-3-(3-methyl-1H-indazol-5-yl)-2-pyridinyl]phenol;
[(1S)-2-{[6-[3-(ethyloxy)phenyl]-5-(3-methyl-1H-indazol-5-yl)-3-pyridinyl]oxy}-1-(phenylmethyl)ethyl]amine;
[(1S)-2-({5-(3-methyl-1H-indazol-5-yl)-6-[3-(methyloxy)phenyl]-3-pyridinyl}oxy)-1-(phenylmethyl)ethyl]amine;
{3-[5-{[(2S)-2-amino-3-phenylpropyl]oxy}-3-(3-methyl-1H-indazol-5-yl)-2-pyridinyl]phenyl}(phenyl)methanone;
[(1S)-2-{[6-{3-[(1-methylethyl)oxy]phenyl}-5-(3-methyl-1H-indazol-5-yl)-3-pyridinyl]oxy}-1-(phenylmethyl)ethyl]amine;
[(1S)-2-{[5-[3-(2-furanyl)-1H-indazol-5-yl]-6-(1H-pyrrol-2-yl)-3-pyridinyl]oxy}-1-(phenylmethyl)ethyl]amine;
[(1S)-2-{[6-(2-{[(3-fluorophenyl)methyl]oxy}phenyl)-5-(3-methyl-1H-indazol-5-yl)-3-pyridinyl]oxy}-1-(phenylmethyl)ethyl]amine;
[(1S)-2-{(6-(4-{[(3-fluorophenyl)methyl]oxy}phenyl)-5-(3-methyl-1H-indazol-5-yl)-3-pyridinyl]oxy}-1-(phenylmethyl)ethyl]amine;
[(1S)-2-({5-[3-(5-chloro-2-thienyl)-1H-indazol-5-yl]-6-phenyl-3-pyridinyl}oxy)-1-(phenylmethyl)ethyl]amine;
[(1S)-2-({5-[3-(4-methyl-2-thienyl)-1H-indazol-5-yl]-6-phenyl-3-pyridinyl}oxy)-1-(phenylmethyl)ethyl]amine;
[(1S)-2-({5-[3-(5-methyl-2-furanyl)-1H-indazol-5-yl]-6-phenyl-3-pyridinyl}oxy)-1-(phenylmethyl)ethyl]amine;
[(1S)-2-({5-[3-(5-methyl-2-thienyl)-1H-indazol-5-yl]-6-phenyl-3-pyridinyl}oxy)-1-(phenylmethyl)ethyl]amine;
[(1S)-2-{[6-ethenyl-5-(3-methyl-1H-indazol-5-yl)-3-pyridinyl]oxy}-1-(phenylmethyl)ethyl]amine;
{(1S)-2-phenyl-1-[({6-phenyl-5-[3-(1H-pyrrol-2-yl)-1H-indazol-5-yl]-3-pyridinyl}oxy)methyl]ethyl}amine;
[(1S)-2-(1H-indol-3-yl)-1-({[5-(3-methyl-1H-indazol-5-yl)-6-phenyl-3-pyridinyl]oxy}methyl)ethyl]amine;
5-(3-methyl-1H-indazol-5-yl)-6-phenyl-N-(3-phenylpropyl)-3-pyridinamine;
5-(3-methyl-1H-indazol-5-yl)-6-phenyl-N-(3-phenylbutyl)-3-pyridinamine;
[(2S)-2-amino-3-phenylpropyl][5-(3-methyl-1H-indazol-5-yl)-6-phenyl-3-pyridinyl]amine;
[(2S)-2-amino-3-phenylpropyl][6-(3-furanyl)-5-(3-methyl-1H-indazol-5-yl)-3-pyridinyl]amine;
((1S)-2-{[6-(3-furanyl)-5-(3-methyl-1H-indazol-5-yl)-3-pyridinyl]oxy}-1-{[(phenylmethyl)oxy]methyl}ethyl)amine;
N-[(2S)-2-amino-3-phenylpropyl]-N-[5-(3-methyl-1H-indazol-5-yl)-6-phenyl-3-pyridinyl]methanesulfonamide;
5-(3-methyl-1H-indazol-5-yl)-N-[2-methyl-2-(phenylthio)propyl]-6-phenyl-3-pyridinamine;
[(1S)-2-{[6-(3-furanyl)-5-(3-methyl-1H-indazol-5-yl)-3-pyridinyl]oxy}-1-(1H-indol-3-ylmethyl)ethyl]amine;
((1S)-2-{[5-(3-methyl-1H-indazol-5-yl)-6-phenyl-3-pyridinyl]oxy}-1-{[(phenylmethyl)oxy]methyl}ethyl)amine;
(2S)-2-amino-3-{[5-(3-methyl-1H-indazol-5-yl)-6-phenyl-3-pyridinyl]oxy}-1-propanol;
5-(3-methyl-1H-indazol-5-yl)-6-phenyl-N-[(2S)-2-pyrrolidinylmethyl]-3-pyridinamine;
((2S)-2-amino-3-{4-[(phenylmethyl)oxy]phenyl}propyl)[5-(3-methyl-1H-indazol-5-yl)-6-phenyl-3-pyridinyl]amine;
[(2S)-2-amino-3-phenylpropyl][5-(1H-indazol-5-yl)-6-phenyl-3-pyridinyl]amine;
[(2S)-2-amino-3-phenylpropyl][6-(3-furanyl)-5-(1H-indazol-5-yl)-3-pyridinyl]amine;
[(2S)-2-amino-3-phenylpropyl][5-(1H-indazol-5-yl)-6-(3-thienyl)-3-pyridinyl]amine;
2-[5-{[(2S)-2-amino-3-phenylpropyl]amino}-3-(1H-indazol-5-yl)-2-pyridinyl]phenol;
2-[5-{[(2S)-2-amino-3-phenylpropyl]amino}-3-(3-methyl-1H-indazol-5-yl)-2-pyridinyl]phenol;
[(2S)-2-amino-3-phenylpropyl][5-(3-methyl-1H-indazol-5-yl)-6-(1H-pyrrol-2-yl)-3-pyridinyl]amine;
[(2S)-2-amino-3-phenylpropyl][5-(3-methyl-1H-indazol-5-yl)-6-(5-methyl-2-thienyl)-3-pyridinyl]amine;
[(2R)-2-amino-3-phenylpropyl][5-(1H-indazol-5-yl)-6-(3-thienyl)-3-pyridinyl]amine;
2-[5-{[(2S)-2-amino-3-(1H-indol-3-yl)propyl]oxy}-3-(3-methyl-1H-indazol-5-yl)-2-pyridinyl]phenol;
[(1S)-2-(1H-indol-3-yl)-1-({[5-(3-methyl-1H-indazol-5-yl)-6-(1H-pyrrol-2-yl)-3-pyridinyl]oxy}methyl)ethyl]amine;
[(1S)-2-(1H-indol-3-yl)-1-({[5-(3-methyl-1H-indazol-5-yl)-6-(5-methyl-2-thienyl)-3-pyridinyl]oxy}methyl)ethyl]amine;
[(1S)-2-{[6-ethyl-5-(3-methyl-1H-indazol-5-yl)-3-pyridinyl]oxy}-1-(phenylmethyl)ethyl]amine;
[(1S)-2-{[6-(3-furanyl)-5-(1H-indazol-5-yl)-3-pyridinyl]oxy}-1-(phenylmethyl)ethyl]amine;
[(1S)-2-{[5-(3-ethenyl-1H-indazol-5-yl)-6-(3-furanyl)-3-pyridinyl]oxy}-1-(phenylmethyl)ethyl]amine;
[(1S)-2-{[5-(3-ethyl-1H-indazol-5-yl)-6-(3-furanyl)-3-pyridinyl]oxy}-1-(phenylmethyl)ethyl]amine;
[(1S)-2-({6-(3-furanyl)-5-[3-(3-pyridinyl)-1H-indazol-5-yl]-3-pyridinyl]oxy)-1-(phenylmethyl)ethyl]amine;
[(1S)-2-{[6-methyl-5-(3-methyl-1H-indazol-5-yl)-3-pyridinyl]oxy}-1-(phenylmethyl)ethyl]amine;
[(1S)-2-({5-(3-methyl-1H-indazol-5-yl)-6-[2-(methyloxy)phenyl]-3-pyridinyl}oxy)-1-(phenylmethyl)ethyl]amine;
[(1S)-2-{[6-[2-(ethyloxy)phenyl]-5-(3-methyl-1H-indazol-5-yl)-3-pyridinyl]oxy}-1-(phenylmethyl)ethyl]amine;
[(1S)-2-{[6-[5-chloro-2-(methyloxy)phenyl]-5-(3-methyl-1H-indazol-5-yl)-3-pyridinyl]oxy}-1-(phenylmethyl)ethyl]amine;
[(1S)-2-{[6-[5-fluoro-2-(propyloxy)phenyl]-5-(3-methyl-1H-indazol-5-yl)-3-pyridinyl]oxy}-1-(phenylmethyl)ethyl]amine;
[(1S)-2-({5-[3-(1-methylethyl)-1H-indazol-5-yl]-6-phenyl-3-pyridinyl}oxy)-1-(phenylmethyl)ethyl]amine;
[(1S)-2-{[5-(6-fluoro-3-methyl-1H-indazol-5-yl)-6-(3-furanyl)-3-pyridinyl]oxy}-1-(phenylmethyl)ethyl]amine;
N-[6-(3-furanyl)-5-(3-methyl-1H-indazol-5-yl)-3-pyridinyl]-L-phenylalaninamide;
N-[6-(2-hydroxyphenyl)-5-(3-methyl-1H-indazol-5-yl)-3-pyridinyl]-L-phenylalaninamide;
2-[5-{([(2S)-2-amino-3-(1-benzothien-3-yl)propyl]oxy}-3-(1H-indazol-5-yl)-2-pyridinyl]phenol;
[(1S)-2-(1-benzothien-3-yl)-1-({[6-(2-furanyl)-5-(1H-indazol-5-yl)-3-pyridinyl]oxy}methyl)ethyl]amine;
[(1S)-2-{[5-(3-methyl-1H-indazol-5-yl)-6-phenyl-3-pyridinyl]oxy}-1-(2-naphthalenylmethyl)ethyl]amine;
N-[5-(3-methyl-1H-indazol-5-yl)-6-(1H-pyrrol-2-yl)-3-pyridinyl]-L-phenylalaninamide;
[(2S)-2-amino-3-(1H-indol-3-yl)propyl][6-(3-furanyl)-5-(3-methyl-1H-indazol-5-yl)-3-pyridinyl]amine;
(2S)-1-{[6-(3-furanyl)-5-(3-methyl-1H-indazol-5-yl)-3-pyridinyl]oxy}-3-phenyl-2-propanol;
1-{3-[5-{[(2S)-2-amino-3-(1H-indol-3-yl)propyl]oxy}-2-(3-furanyl)-3-pyridinyl]phenyl}ethanone;
[(1S)-2-{[6-cyclopentyl-5-(3-methyl-1H-indazol-5-yl)-3-pyridinyl]oxy}-1-(1H-indol-3-ylmethyl)ethyl]amine;
[(1S)-2-(1-benzothien-3-yl)-1-({[5-(1H-indazol-5-yl)-6-phenyl-3-pyridinyl]oxy}methyl)ethyl]amine;
[(1S)-2-(1-benzothien-3-yl)-1-({[6-(3-furanyl)-5-(1H-indazol-5-yl)-3-pyridinyl]oxy}methyl)ethyl]amine;
[(1S)-2-(1-benzothien-3-yl)-1-({[5-(1H-indazol-5-yl)-6-(3-thienyl)-3-pyridinyl]oxy}methyl)ethyl]amine;
[(1S)-2-(1-benzothien-3-yl)-1-({[5-(1H-indazol-5-yl)-6-(1H-pyrrol-2-yl)-3-pyridinyl]oxy}methyl)ethyl]amine;
[(1S)-2-{[5-(1H-indazol-5-yl)-6-phenyl-3-pyridinyl]oxy}-1-(1H-pyrazol-1-ylmethyl)ethyl]amine;
[(1S)-2-(1-benzothien-3-yl)-1-({[5-(1H-indazol-5-yl)-6-(5-methyl-2-thienyl)-3-pyridinyl]oxy}methyl)ethyl]amine;
[(1S)-2-{[6-(3-furanyl)-5-(3-methyl-1H-thieno[3,2-c]pyrazol-5-yl)-3-pyridinyl]oxy}-1-(1H-indol-3-ylmethyl)ethyl]amine;
5-[5-{[(2S)-2-amino-3-phenylpropyl]oxy}-2-(3-furanyl)-3-pyridinyl]-N-4-pyridinyl-1H-indazol-3-amine;
N-{5-[5-{[(2S)-2-amino-3-phenylpropyl]oxy}-2-(3-furanyl)-3-pyridinyl]-1H-indazol-3-yl}benzamide;
(1E)-1-{3-[5-{[(2S)-2-amino-3-(1H-indol-3-yl)propyl]oxy}-2-(3-furanyl)-3-pyridinyl]phenyl}ethanone oxime;
[(1S)-2-{[6-(3-furanyl)-5-(3-methyl-1H-indazol-5-yl)-3-pyridinyl]oxy}-1-(phenylmethyl)propyl]amine;
(2S)-N-methyl-1-{[5-(3-methyl-1H-indazol-5-yl)-6-phenyl-3-pyridinyl]oxy}-3-phenyl-2-propanamine;
[(1S)-2-{[6-[5-fluoro-2-(methyloxy)phenyl]-5-(3-methyl-1H-indazol-5-yl)-3-pyridinyl]oxy}-1-(phenylmethyl)ethyl]amine;
[(1S)-2-{[6-[3,5-difluoro-2-(methyloxy)phenyl]-5-(3-methyl-1H-indazol-5-yl)-3-pyridinyl]oxy}-1-(phenylmethyl)ethyl]amine;
[(1S)-2-({6-(3-furanyl)-5-[3-(4-pyridinyl)-1H-indazol-5-yl]-3-pyridinyl}oxy)-1-(phenylmethyl)ethyl]amine;
2-[5-{[(2S)-2-amino-3-phenylpropyl]oxy}-3-(3-methyl-1H-indazol-5-yl)-2-pyridinyl]-4-fluorophenol;
2-[5-{[(2S)-2-amino-3-phenylpropyl]oxy}-3-(3-methyl-1H-indazol-5-yl)-2-pyridinyl]-4,6-difluorophenol;
2-[5-{[(2S)-2-amino-3-(1H-indol-3-yl)propyl]oxy}-3-(6-fluoro-3-methyl-1H-indazol-5-yl)-2-pyridinyl]phenol;
2-[5-{[(2S)-2-amino-3-(1H-indol-3-yl)propyl]oxy}-3-(3-ethyl-1H-indazol-5-yl)-2-pyridinyl]phenol;
[(1S)-2-{[5-(3-ethyl-1H-indazol-5-yl)-6-(3-furanyl)-3-pyridinyl]oxy}-1-(1H-indol-3-ylmethyl)ethyl]amine;
[(1S)-2-{[5-(3-ethyl-1H-indazol-5-yl)-6-(2-furanyl)-3-pyridinyl]oxy}-1-(1H-indol-3-ylmethyl)ethyl]amine;
[(1S)-2-{[5-(3-ethyl-1H-indazol-5-yl)-6-(1H-pyrrol-2-yl)-3-pyridinyl]oxy}-1-(1H-indol-3-ylmethyl)ethyl]amine;
[(1S)-2-({6-(3-furanyl)-5-[3-(1-methyl-1H-pyrazol-4-yl)-1H-indazol-5-yl]-3-pyridinyl}oxy)-1-(phenylmethyl)ethyl]amine;
[(1S)-2-({6-(3-furanyl)-5-[3-(1H-pyrrol-2-yl)-1H-indazol-5-yl]-3-pyridinyl}oxy)-1-(phenylmethyl)ethyl]amine;
[(1S)-2-((6-(3-furanyl)-5-[3-(1H-pyrazol-4-yl)-1H-indazol-5-yl]-3-pyridinyl}oxy)-1-(phenylmethyl)ethyl]amine;
[(1S)-2-([5-(6-fluoro-3-methyl-1H-indazol-5-yl)-6-(2-furanyl)-3-pyridinyl]oxy}-1-(1H-indol-3-ylmethyl)ethyl]amine;
[(1S)-2-{[5-(6-fluoro-3-methyl-1H-indazol-5-yl)-6-(1H-pyrrol-2-yl)-3-pyridinyl]oxy}-1-(1H-indol-3-ylmethyl)ethyl]amine;
[(1S)-2-{[5-(6-fluoro-3-methyl-1H-indazol-5-yl)-6-(3-furanyl)-3-pyridinyl]oxy}-1-(1H-indol-3-ylmethyl)ethyl]amine;
[(1S)-2-{[6-(1-benzothien-2-yl)-5-(3-methyl-1H-indazol-5-yl)-3-pyridinyl]oxy}-1-(phenylmethyl)ethyl]amine;
[(1S)-2-{[6-(1-benzofuran-2-yl)-5-(3-methyl-1H-indazol-5-yl)-3-pyridinyl]oxy}-1-(phenylmethyl)ethyl]amine;
[(1S)-2-({6-(3-furanyl)-5-[3-(methylsulfonyl)phenyl]-3-pyridinyl}oxy)-1-(1H-indol-3-ylmethyl)ethyl]amine;
5-[5-{[(2S)-2-(1-azetidinyl)-3-(1H-indol-3-yl)propyl]oxy}-2-(3-furanyl)-3-pyridinyl]-3-methyl-1H-indazole;
[(1S)-2-({6-(3-furanyl)-5-[3-(1H-pyrazol-4-yl)-1H-indazol-5-yl]-3-pyridinyl}oxy)-1-(1H-indol-3-ylmethyl)ethyl]amine;
3-[5-{[(2S)-2-amino-3-(1H-indol-3-yl)propyl]oxy}-2-(3-furyl)pyridin-3-yl]benzamide;
4-[5-{[(2S)-2-amino-3-(1H-indol-3-yl)propyl]oxy}-2-(3-furyl)pyridin-3-yl]benzamide;
5-(5-{[(2S)-3-(1H-indol-3-yl)-2-(1-piperidinyl)propyl]oxy}-2-phenyl-3-pyridinyl)-3-methyl-1H-indazole;
5-(2-(3-furanyl)-5-{[(2S)-3-(1H-indol-3-yl)-2-(4-morpholinyl)propyl]oxy}-3-pyridinyl)-3-methyl-1H-indazole;
[(1S)-2-({6-(3-furanyl)-5-[3-(1H-pyrazol-4-yl)-1H-indazol-5-yl]-3-pyridinyl}oxy)-1-(1H-indol-3-ylmethyl)ethyl]amine;
[(1S)-2-{[6-(3-furanyl)-5-(3-methyl-1H-indazol-5-yl)-3-pyridinyl]oxy}-1-(1H-indol-3-ylmethyl)ethyl]dimethylamine;
(3S)-3-({[6-(3-furanyl)-5-(3-methyl-1H-indazol-5-yl)-3-pyridinyl]oxy}methyl)-2-methyl-2,3,4,9-tetrahydro-1H-carboline;
1-{5-[5-{[(2S)-2-amino-3-(1H-indol-3-yl)propyl]oxy}-2-(3-furanyl)-3-pyridinyl]-2-thienyl}ethanone;
(2S)-1-{[6-(3-furanyl)-5-(3-methyl-1H-indazol-5-yl)-3-pyridinyl]oxy}-3-(1H-indol-3-yl)-N-methyl-2-propanamine;
5-[5-{[(2S)-2-amino-3-(1H-indol-3-yl)propyl]oxy}-2-(3-furanyl)-3-pyridinyl]-N,N-dimethyl-2-furancarboxamide;
5-[5-{[(2S)-2-amino-3-(1H-indol-3-yl)propyl]oxy}-2-(3-furanyl)-3-pyridinyl]-N-methyl-2-furancarboxamide;
5-[5-{[(2S)-2-amino-3-(1H-indol-3-yl)propyl]oxy}-2-(3-furanyl)-3-pyridinyl]-2-furancarboxamide;
[(2S)-2-amino-3-phenylpropyl][6-(3-furanyl)-5-(3-methyl-1H-indazol-5-yl)-3-pyridinyl]methylamine;
[(1S)-2-(3,4-dichlorophenyl)-1-({[5-(3-methyl-1H-indazol-5-yl)-6-phenyl-3-pyridinyl]oxy}methyl)ethyl]amine;
N-[5-(3-methyl-1H-indazol-5-yl)-6-phenyl-3-pyridinyl]-L-phenylalaninamide;
N-[5-(3-methyl-1H-indazol-5-yl)-6-phenyl-3-pyridinyl]-L-phenylalaninamide;
2-[5-{[(2S)-2-amino-3-(1H-indol-3-yl)propyl]amino}-3-(3-methyl-1H-indazol-5-yl)-2-pyridinyl]-4-fluorophenol;
((1S)-3-{[6-(3-furanyl)-5-(3-methyl-1H-indazol-5-yl)-3-pyridinyl]oxy}-1-({[4-(trifluoromethyl)phenyl]methyl}propyl)amine;
[(1S)-3-{[6-(3-furanyl)-5-(3-methyl-1H-indazol-5-yl)-3-pyridinyl]oxy}-1-(1H-indol-3-ylmethyl)propyl]amine;
{(1S)-2-{[6-(3-furanyl)-5-(3-methyl-1H-indazol-5-yl)-3-pyridinyl]oxy}-1-[(5-methyl-1H-indol-3-yl)methyl]ethyl}amine;
[(1S)-2-(1H-indol-3-yl)-1-({[5-(3-methyl-1H-indazol-5-yl)-6-(1H-pyrrol-3-yl)pyridin-3-yl]oxy}methyl)ethyl]amine;
[(1S)-2-{[6-(3-furanyl)-5-(3-methyl-1H-pyrazolo[4,3-b]pyridin-5-yl)-3-pyridinyl]oxy}-1-(1H-indol-3-ylmethyl)ethyl]amine;
[(1S)-2-(1H-indol-3-yl)-1-({[5-(3-methyl-1H-pyrazolo[4,3-b]pyridin-5-yl)-3-pyridinyl]oxy}methyl)ethyl]amine;
5-[5-{[(2S)-2-amino-3-phenylpropyl]oxy}-2-(3-furanyl)-3-pyridinyl]-1H-indazole-3-carboxamide;
5-[5-{[(2S)-2-amino-3-phenylpropyl]oxy}-2-(3-furanyl)-3-pyridinyl]-1H-indazole-3-carbonitrile;
(2S)-1-{[6-(2-furanyl)-5-(3-methyl-1H-indazol-5-yl)-3-pyridinyl]oxy}-3-(1H-indol-3-yl)-2-propanamine;
2-[5-{[(2S)-2-amino-3-(1-benzothien-3-yl)-3-propyl]oxy}-3-(1H-indazol-5-yl)-2-pyridinyl]-4-flurophenol;
2-[5-{[(2S)-2-amino-3-(1-benzothien-3-yl)-3-propyl]oxy}-3-(1H-indazol-5-yl)-2-pyridinyl]-4,6-diflurophenol;
[(1S)-2-(1-benzothien-3-yl)-1-({[5,6-bis(3-methyl-1H-indazol-5-yl)-3-pyridinyl]oxy}methyl)ethyl]amine;
[(1S)-2-(1-benzothien-3-yl)-1-({[4-(3-furanyl)-3-(3-methyl-1H-indazol-5-yl)phenyl]oxy}methyl)ethyl]amine;
4′-{[(2S)-2-amino-3-(1-benzothien-3-yl)propyl]oxy}-3,5-difluoro-2′-(3-methyl-1H-indazol-5-yl)-2-biphenylol;
4′-{[(2S)-2-amino-3-(1-benzothien-3-yl)propyl]oxy}-5-fluoro-2′-(3-methyl-1H-indazol-5-yl)-2-biphenylol;
2-[5-{[(2S)-2-amino-3-(1H-indol-3-yl)propyl]oxy}-3-(3-methyl-1H-indazol-5-yl)-2-pyridinyl]-4,6-difluorophenol;
[(2S)-2-amino-3-(1H-indol-3-yl)propyl][5-(3-methyl-1H-indazol-5-yl)-6-(1H-pyrrol-2-yl)-3-pyridinyl]amine;
[(2S)-2-amino-3-(1H-indol-3-yl)propyl][6-[5-fluoro-2-(methyloxy)phenyl]-5-(3-methyl-1H-indazol-5-yl)-3-pyridinyl]amine;
2-[5-{[(2S)-2-amino-3-(1H-indol-3-yl)propyl]amino}-3-(3-methyl-1H-indazol-5-yl)-2-pyridinyl]phenol;
2-[5-([(2S)-2-amino-3-(1H-indol-3-yl)propyl]amino}-3-(3-methyl-1H-indazol-5-yl)-2-pyridinyl]phenol;
[(2S)-2-amino-3-(5-fluoro-1H-indol-3-yl)propyl][6-(3-furanyl)-5-(3-methyl-1H-indazol-5-yl)-3-pyridinyl]amine;
[(2S)-2-amino-4-pentyn-1-yl][6-(3-furanyl)-5-(3-methyl-1H-indazol-5-yl)-3-pyridinyl]amine;
[(2S)-2-amino-3-(5,6,7-trifluoro-1H-indol-3-yl)propyl][6-(3-furanyl)-5-(3-methyl-1H-indazol-5-yl)-3-pyridinyl]amine;
[(2S)-2-amino-3-(5,7-difluoro-1H-indol-3-yl)propyl][6-(3-furanyl)-5-(3-methyl-1H-indazol-5-yl)-3-pyridinyl]amine;
[(1S)-2-{[6-(3-furanyl)-5-(3-methyl-1H-indazol-5-yl)-3-pyridinyl]oxy}-1-(1H-pyrrolo[2,3-b]pyridin-2-ylmethyl)ethyl]amine;
[(2R)-2-amino-3-phenylpropyl][3-fluoro-4-(3-furanyl)-5-(3-methyl-1H-indazol-5-yl)phenyl]amine;
[(2R)-2-amino-3-(1H-indol-3-yl)propyl][3-fluoro-4-(3-furanyl)-5-(3-methyl-1H-indazol-5-yl)phenyl]amine;
[(1S)-2-{[6-(3-furanyl)-5-(3-methyl-1H-pyrazolo[3,4-b]pyridin-5-yl)-3-pyridinyl]oxy}-1-(1H-indol-3-ylmethyl)ethyl]amine;
[(1S)-2-(1H-indol-3-yl)-1-({[6-(2-methyl-3-furanyl)-5-(3-methyl-1H-pyrazolo[3,4-b]pyridin-5-yl)-3-pyridinyl]oxy}methyl)ethyl]amine;
[(1S)-2-(1H-indol-3-yl)-1-({[5-(3-methyl-1H-pyrazolo[3,4-b]pyridin-5-yl)-6-phenyl-3-pyridinyl]oxy}methyl)ethyl]amine;
[(1S)-2-{[6-(3-furanyl)-5-(3-methyl-1H-pyrazolo[3,4-b]pyridin-5-yl)-3-pyridinyl]oxy}-1-(1H-indol-3-ylmethyl)ethyl]methylamine;
2-[5-{[(2S)-2-amino-3-(1H-indol-3-yl)propyl]oxy}-3-(3-methyl-1H-pyrazolo[3,4-b]pyridin-5-yl)-2-pyridinyl]phenol;
2-[5-{[(2S)-2-amino-3-(1H-indol-3-yl)propyl]oxy}-3-(3-methyl-1H-pyrazolo[3,4-b]pyridin-5-yl)-2-pyridinyl]-6-fluorophenol;
[(1S)-2-{[5-[3-(3,5-dimethyl-4-isoxazolyl)-1H-indazol-5-yl]-6-(3-furanyl)-3-pyridinyl]oxy}-1-(phenylmethyl)ethyl]amine;
[(1S)-2-({6-(3-furanyl)-5-[3-(2-pyridinyl)-1H-indazol-5-yl]-3-pyridinyl}oxy)-1-(phenylmethyl)ethyl]amine;
[(1S)-2-{[6-(2-chlorophenyl)-5-(3-methyl-1H-indazol-5-yl)-3-pyridinyl]oxy}-1-(phenylmethyl)ethyl]amine;
[(1S)-2-{[5-(3-methyl-1H-indazol-5-yl)-6-(2-methylphenyl)-3-pyridinyl]oxy}-1-(phenylmethyl)ethyl]amine;
[(1S)-2-{[6-(2-fluorophenyl)-5-(3-methyl-1H-indazol-5-yl)-3-pyridinyl]oxy}-1-(phenylmethyl)ethyl]amine;
2-[5-{[(2S)-2-amino-3-phenylpropyl]oxy}-3-(3-methyl-1H-indazol-5-yl)-2-pyridinyl]-4-chlorophenol;
[(1S)-2-{[6-(1-benzothien-3-yl)-5-(3-methyl-1H-indazol-5-yl)-3-pyridinyl]oxy}-1-(phenylmethyl)ethyl]amine;
3-[5-{[(2S)-2-amino-3-phenylpropyl]oxy}-3-(3-methyl-1H-indazol-5-yl)-2-pyridinyl]benzamide;
3-[5-{[(2S)-2-amino-3-phenylpropyl]oxy}-3-(3-methyl-1H-indazol-5-yl)-2-pyridinyl]benzonitrile;
[(1S)-2-{[5-(3-methyl-1H-indazol-5-yl)-6-(3-nitrophenyl)-3-pyridinyl]oxy}-1-(phenylmethyl)ethyl]amine;
[(1S)-2-{[5-(3-methyl-1H-indazol-5-yl)-6-(4-methyl-2-thienyl)-3-pyridinyl]oxy}-1-(phenylmethyl)ethyl]amine;
N-{3-[5-{[(2S)-2-amino-3-phenylpropyl]oxy}-3-(3-methyl-1H-indazol-5-yl)-2-pyridinyl]phenyl}-N′-phenylurea;
[(1S)-2-{[5-(3-methyl-1H-indazol-5-yl)-6-(2-thienyl)-3-pyridinyl]oxy}-1-(phenylmethyl)ethyl]amine;
[(1S)-2-(1H-indol-3-yl)-1-({[6-(2-methyl-3-furanyl)-5-(3-methyl-1H-indazol-5-yl)-3-pyridinyl]oxy}methyl)ethyl]amine;
{2-[5-{[(2S)-2-amino-3-(1H-indol-3-yl)propyl]oxy}-3-(3-methyl-1H-indazol-5-yl)-2-pyridinyl]phenyl]amine;
2-[5-{[(2S)-2-amino-3-(1H-indol-3-yl)propyl]oxy}-3-(3-methyl-1H-indazol-5-yl)-2-pyridinyl]-6-fluorophenol;
2-[5-{[(2S)-2-amino-3-(1H-indol-3-yl)propyl]oxy}-3-(3-methyl-1H-indazol-5-yl)-2-pyridinyl]-4-chlorophenol;
2-[5-{[(2S)-2-amino-3-(1H-indol-3-yl)propyl]oxy}-3-(3-methyl-1H-indazol-5-yl)-2-pyridinyl]-4-fluorophenol;
[(1S)-2-{[6-[3,5-difluoro-2-(methyloxy)phenyl]-5-(3-methyl-1H-thieno[3,2-c]pyrazol-5-yl)-3-pyridinyl]oxy}-1-(1H-indol-3-ylmethyl)ethyl]amine;
2-[5-([(2S)-2-amino-3-(1H-indol-3-yl)propyl]oxy}-3-(3-methyl-1H-thieno[3,2-c]pyrazol-5-yl)-2-pyridinyl]-4,6-difluorophenol;
2-[5-{[(2S)-2-amino-3-(1H-indol-3-yl)propyl]oxy}-3-(3-methyl-1H-thieno[3,2-c]pyrazol-5-yl)-2-pyridinyl]phenol;
2-[5-{[(2S)-2-amino-3-(1H-indol-3-yl)propyl]oxy}-3-(3-methyl-1H-thieno[3,2-c]pyrazol-5-yl)-2-pyridinyl]-4-chlorophenol;
3-(5-{[(2S)-2-amino-3-(1H-indol-3-yl)propyl]oxy}-3-pyridinyl)benzamide;
1-[3-(5-{[(2S)-2-amino-3-(1H-indol-3-yl)propyl]oxy}-3-pyridinyl)phenyl]ethanone; and
5-{[(2S)-2-amino-3-(1H-indol-3-yl)propyl]oxy}-2-(3-furanyl)-3,4′-bipyridine-2′-carboxamide.

10. A pharmaceutically acceptable salt, hydrate, solvate or pro-drug of a compound of Formula (II), as described in claim 9.

11. A pharmaceutical composition comprising a compound according to claim 1, and/or a pharmaceutically acceptable salt, hydrate, solvate or pro-drug thereof and a pharmaceutically acceptable carrier.

12. A process for preparing a pharmaceutical composition containing a pharmaceutically acceptable carrier or diluent and an effective amount of a compound of Formula (I) as described in claim 1 and/or a pharmaceutically acceptable salt, hydrate, solvate or pro-drug thereof, which process comprises bringing the compound of Formula (I) and/or a pharmaceutically acceptable salt, hydrate, solvate or pro-drug thereof into association with a pharmaceutically acceptable carrier or diluent.

13. A method of treating or lessening the severity of cancer in a mammal in need thereof, which comprises administering to such mammal a therapeutically effective amount of a compound of Formula I, as described in claim 1 and/or a pharmaceutically acceptable salt, hydrate, solvate or pro-drug thereof.

14. The method of claim 13 wherein the mammal is a human.

15. A method of treating or lessening the severity of cancer in a mammal in need thereof, which comprises administering to such mammal a therapeutically effective amount of a compound of Formula II, as described in claim 5 and/or a pharmaceutically acceptable salt, hydrate, solvate or pro-drug thereof.

16. The method of claim 15 wherein the mammal is a human.

17. The method according to claim 13 wherein said cancer is selected from brain (gliomas), glioblastomas, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

18. The method according to claim 15 wherein said cancer is selected from brain (gliomas), glioblastomas, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

19. (canceled)

20. The method of inhibiting Akt activity in a mammal in need thereof, which comprises administering to such mammal a therapeutically effective amount of a compound of Formula I, as described in claim 1 and/or a pharmaceutically acceptable salt, hydrate, solvate or pro-drug thereof.

21. The method of claim 20 wherein the mammal is a human.

22. A method of treating cancer in a mammal in need thereof, which comprises: administering to such mammal a therapeutically effective amount of
a) a compound of Formula (I), as described in claim 1 and/or a pharmaceutically acceptable salt, hydrate, solvate or pro-drug thereof; and
b) at least one anti-neoplastic agent.

23. The method claim 22, wherein the at least one anti-neoplastic agent is selected from the group consisting essentially of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors; non-receptor tyrosine kinase angiogenesis inhibitors; immunotherapeutic agents; proapoptotic agents; and cell cycle signaling inhibitors.

24. The method of claim 22, wherein the at least one anti-neoplastic agent is an anti-microtubule agent selected from diterpenoids and vinca alkaloids.

25. The method of claim 22, wherein the at least one anti-neoplastic agent is a diterpenoid.

26. The method of claim 22, wherein the at least one anti-neoplastic agent is a vinca alkaloid.

27. The method of claim 22, wherein the at least one anti-neoplastic agent is a platinum coordination complex.

28. The method of claim 22, wherein the at least one anti-neoplastic agent is paclitaxel, carboplatin, or vinorelbine.

29. The method of claim 22, wherein the at least one anti-neoplastic agent is paclitaxel.

30. The method of claim 22, wherein the at least one anti-neoplastic agent is carboplatin.

31. The method of claim 22, wherein the at least one anti-neoplastic agent is vinorelbine.

32. The method of claim 22, wherein the at least one anti-neoplatic agent is a signal transduction pathway inhibitor.

33. The method of claim 32, wherein the signal transduction pathway inhibitor is an inhibitor of a growth factor receptor kinase selected from the group consisting of VEGFR2, TIE2, PDGFR, BTK, IGFR-1, TrkA, TrkB, TrkC, and c-fms.

34. The method of claim 32, wherein the signal transduction pathway inhibitor is an inhibitor of a serine/threonine kinase selected from the group consisting of rafk, akt, and PKC-zeta.

35. The method of claim 32, wherein the signal transduction pathway inhibitor is an inhibitor of a serine/threonine kinase selected from the src family of kinases.

36. The method of claim 35, wherein the signal transduction pathway inhibitor is an inhibitor of c-src.

37. The method of claim 32, wherein the signal transduction pathway inhibitor is an inhibitor of Ras oncogene selected from inhibitors of farnesyl transferase and geranylgeranyl transferase.

38. The method of claim 32, wherein the signal transduction pathway inhibitor is an inhibitor of a serine/threonine kinase selected from the group consisting of PI3K.

39. The method of claim 22, wherein the at least one anti-neoplastic agent is a cell cycle signaling inhibitor.

40. The method of claim 39, wherein the cell cycle signaling inhibitor is selected from inhibitors of the group CDK2, CDK4, and CDK6.

41-42. (canceled)

43. A method of treating or lessening the severity of arthritis in a mammal in need thereof, which comprises administering to such mammal a therapeutically effective amount of a compound of Formula I, as described in claim 1 and/or a pharmaceutically acceptable salt, hydrate, solvate or pro-drug thereof.

44. The method of claim 43 wherein the mammal is a human.

45. A method of treating or lessening the severity of arthritis in a mammal in need thereof, which comprises administering to such mammal a therapeutically effective amount of a compound of Formula II, as described in claim 5 and/or a pharmaceutically acceptable salt, hydrate, solvate or pro-drug thereof.

46. The method of claim 45 wherein the mammal is a human.

47. (canceled)
